SEQUENCE LISTING (17 sequences)

SEQ ID NO: 1 - protein, 30 aa, Artificial Sequence
  amphipathic peptide sequence (Table 2)
WEARLARALARALARHLARALARALRACEA

SEQ ID NO: 2 - protein, 29 aa, Artificial Sequence
  amphipathic peptide sequence (Table 2)
WEARLARALARALARLARALARALRACEA

SEQ ID NO: 3 - protein, 29 aa, Artificial Sequence
  amphipathic peptide sequence (Table 2)
WEARLARALARALARLARALARALRACEA

SEQ ID NO: 4 - protein, 30 aa, Artificial Sequence
  amphipathic peptide sequence (Table 2)
WEARLARALARALARELARALARALRACEA

SEQ ID NO: 5 - protein, 29 aa, Artificial Sequence
  amphipathic peptide sequence (Table 2)
REARLARALARALARLARALARALRACEA

SEQ ID NO: 6 - protein, 29 aa, Artificial Sequence
  amphipathic peptide sequence (Table 2)
REARLARALARALARLARALARALRAREA

SEQ ID NO: 7 - protein, 30 aa, Artificial Sequence
  amphipathic peptide sequence (Table 2)
REARLARALARALARELARALARALRAREA

SEQ ID NO: 8 - protein, 22 aa, Artificial Sequence
  22mer protein sequence (Table 2)
WEARLARALARALARHLRACEA

SEQ ID NO: 9 - protein, 30 aa, Artificial Sequence
  prior art GALA sequence (anionic)
WEAALAEALAEALAEHLAEALAEALEALAA

SEQ ID NO: 10 - protein, 30 aa, Artificial Sequence
  prior art KALA sequence (cationic)
WEAKLAKALAKALAKHLAKALAKALKACEA

SEQ ID NO: 11 - protein, 16 aa, Artificial Sequence
  prior art RALA 16mer sequence
RALARALARALRALAR

SEQ ID NO: 12 - protein, 5 aa, Artificial Sequence
  targeting peptide TMTP-1
NVVRQ

SEQ ID NO: 13 - protein, 5 aa, Artificial Sequence
  spacer
EAAAK

SEQ ID NO: 14 - protein, 60 aa, Artificial Sequence
  Targeting Peptide-alpha helical spacer (four repeats)-RALA
NVVRQLAEAAAKEAAAKEAAAKEAAAKAAAWEARLARALARALARHLARALARALRACEA

SEQ ID NO: 15 - protein, 14 aa, Artificial Sequence
  claim 6 consensus sequence
EARLARALARALAR

SEQ ID NO: 16 - protein, 11 aa, Artificial Sequence
  claim 9 consensus sequence
LARALARALRA

SEQ ID NO: 17 - protein, 30 aa, Artificial Sequence
  claim 10 consensus sequence
XEARLARALARALARXLARALARALRAXEA

(19) United States
(12) Patent Application Publication
(10) Pub. No.: US 2017/0112760 A1
(43) Pub. Date: Apr. 27, 2017

(54) CELL DELIVERY SYSTEM AND METHOD

(71) Applicant: The Queen's University of Belfast, Belfast Antrim (GB)

(72) Inventors: Helen McCarthy, Belfast Antrim (GB); Ryan Donnelly, Belfast Antrim (GB); Joanne McCaffrey, Belfast Antrim (GB)

(21) Appl. No.: 15/317,786

(22) PCT Filed: Jun. 9, 2015
(86) PCT No.: PCT/EP2015/062834

§ 371 Date: Dec. 9, 2016

(30) Foreign Application Priority Data
Jun. 10, 2014 (GB) 1410270.1

(51) Int. Cl. A61K 9/00; A61K 48/00; A61K 47/10; A61K 31/713; A61K 39/00; A61K 31/663; A61K 47/32; A61K 47/34; A61K 39/12; C12N 7/00; A61K 39/39; C12N 15/113

(52) U.S. Cl. CPC A61K 9/0021; A61K 39/39; A61K 48/0075; A61K 47/48246; A61K 47/10; A61K 48/0058; A61K 31/713; C12N 15/113; C12N 15/1136; A61K 31/663; A61K 47/32; A61K 47/34; A61K 39/12; C12N 7/00; A61K 39/0011; A61K 2039/53; A61K 2039/6031; C12N 2310/14; C12N 2320/32; A61K 2039/54; C12N 2710/20034; C12N 2710/20071; A61K 2039/575; A61K 2039/572

(57) ABSTRACT

The present invention is directed to a cell delivery system for the delivery of materials including nucleic acids across a biological barrier and methods of use thereof. The cell delivery system of the invention comprises a microprotrusion array composed of a swellable and/or dissolvable polymer composition for use in the transport of a material across a biological barrier, wherein the material comprises nanoparticles formed from a nucleic acid complexed with an amphipathic cell penetrating peptide and wherein the microprotrusion array is loaded with the nanoparticles.

### FIELD OF THE INVENTION

The present invention is directed to a cell delivery system for the delivery of materials including nucleic acids across a biological barrier and methods of use thereof.

### BACKGROUND TO THE INVENTION

Transdermal delivery offers advantages over conventional oral and parenteral administration, including prevention of drug degradation in the stomach, avoidance of first pass liver metabolism, possibility of improved bioavailability etc and for parenteral administration poor compliance with injections. However, at present there are only a handful of drugs offered for transdermal patch delivery. This is due to the excellent barrier function of the skin, which is accomplished almost entirely by the outermost 10-15 microns of tissue, the stratum corneum. Thus, to date the transdermal delivery and subsequent intracellular delivery of materials, such as nucleic acids, across a biological barrier such as the stratum corneum (SC) is problematic achieving only moderate success and the many different approaches encountering significant problems.

For example, the delivery of a vaccine across a biological barrier is problematic. Vaccination consists of stimulating the immune system with an infectious agent, or components of an infectious agent, modified in such a manner that no harm or disease is caused, but ensuring that when the host is confronted with that infectious agent, the immune system can adequately neutralize it before it causes any ill effect. Conventionally, vaccination has been effected by one of two approaches: either introducing specific antigens against which the immune system reacts directly; or introducing live attenuated infectious agents that replicate within the host without causing disease, synthesize the antigens that subsequently prime the immune system.

Due to safety issues and deleterious side effects, traditional pathogenic vaccines are almost obsolete. Vaccine development now focuses on vaccines developed from purified subunits, recombinant proteins or synthetic peptides. However, although these vaccines have an excellent safety profile, their immunogenic potency is significantly compromised with the induction of only an antibody response. To overcome this and to activate the cell-mediated immune response primarily through cytotoxic T lymphocytes (CTLs), DNA vaccination is now utilised in which plasmid DNA encodes for a particular antigen for a disease.

DNA vaccination is a radical new approach and involves the direct introduction into appropriate tissues of a plasmid containing the DNA sequence encoding the antigen(s) against which an immune response is sought, and relies on the in situ production of the target antigen. This approach offers a number of potential advantages over traditional approaches, including the stimulation of both B- and T-cell responses, improved vaccine stability, the absence of any infectious agent and the relative ease of large-scale manufacture.

However, in order for the desired antigen to be expressed the plasmid DNA must be delivered to the nucleus of cells to enable the production of the protein antigen in the cytoplasm of the host cell. In addition the DNA must then be delivered to the antigen presenting cell (APC's) via the MHC I pathway to result in an effective cell-mediated immune response. One of the advantages of using a DNA vaccine is that they can evoke a long term immune response without the need for adjuvants or repeated injections. Furthermore, in addition to a prophylactic response, DNA vaccines can also be administered to treat a pre-existing infection. Additionally, DNA vaccines are inexpensive to manufacture, heat stable and easy to store.

Despite these advantages, at present there is no highly effective delivery system to transport the DNA to the APC's to evoke the desired immune response. Current developments often employ physical methods such as intramuscular injection of ‘naked DNA’, coating of gold nanoparticles with DNA and using a gene gun or jet injectors. However, the use of these physical methods require large quantities of DNA and can damage the DNA being delivered and do not overcome the intracellular barriers for delivery of the DNA to the nucleus.

At present there are 3 licenced DNA vaccines (West Nile virus in Horses, DNA vaccine Apex®-IHN and ONCEPT™) commercially available and these utilise physical methods for delivery. For example, ONCEPT™ was developed with licensed delivery technology from Vical involving a physical Needle Free Injection Therapy System (NFITS) that forces macromolecules through the skin. All of these vaccines use physical forces to deliver the DNA and do not take account of the critical intercellular barriers in their systems.

Alternative delivery means, including microneedle technology has been used to deliver DNA either by coating metal microneedles or inserting DNA into hollow microneedles. However, these delivery methods have been found to be unsuccessful due to resultant low transfection rates. Although, DNA delivery to the skin is generally effective, this microneedle technology does not provide an effective delivery system to transport the DNA intracellularly to the nucleus.

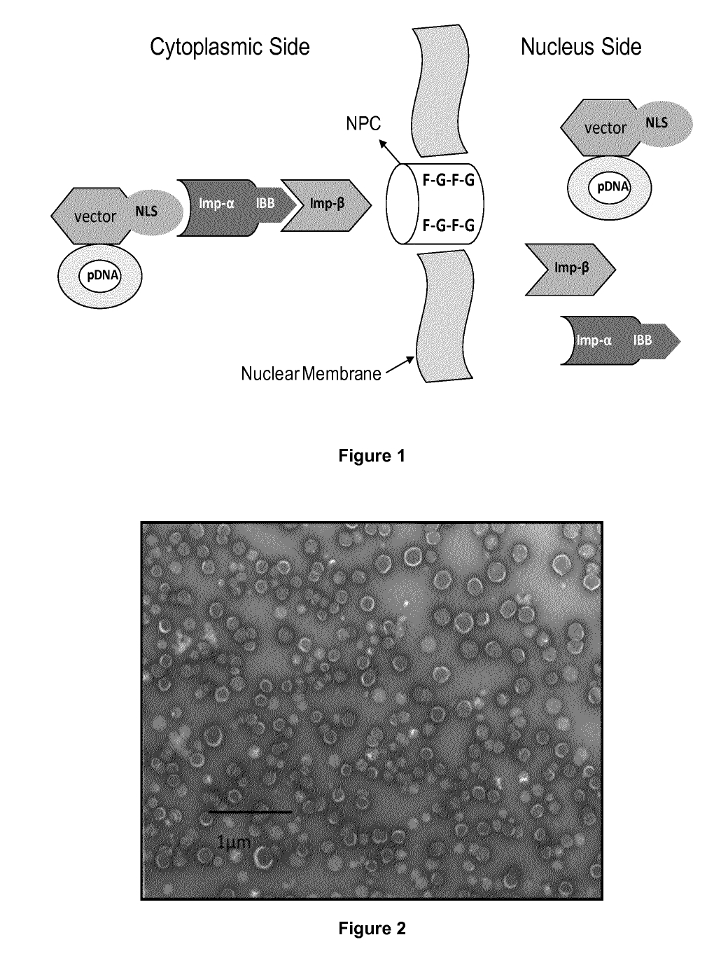

In the extracellular barrier described above, several biological barriers exist intracellularly. Upon systemic administration the delivery vector must not be degraded in the circulation and must be able to extravasate to surround tissues. Again stability is necessary in the extracellular matrix and the fibrous network of proteins must be navigated. Even when reaching the target tissue cellular entry must be achieved and this is dependent upon charge and size of the particle to be delivered. When foreign particles are endocytosed they become trapped in the endosome which is degraded into a lysozyme. Therefore endosomal escape is critical for successful delivery to the cytoplasm. However several studies have shown that the uptake of DNA into the cytoplasm does not correlate with efficient gene delivery and this is perhaps because the most important barrier is the one to the nucleus. If the final destination site is the nucleus then an active transport system is required otherwise entry into the nucleus is a chance effect during cellular division when the nuclear membrane dissolves. Translocation to the nucleus is dependent on the presence of basic amino acids known as a nuclear localisation signal. The nuclear localisation signal binds to the importin alpha protein which has an importin beta binding domain. The importin beta binding domain then recruits and binds importin beta which will transport the whole complex through the nuclear pore channel through the transient association and disassociation of the phenyalanine-glycine repeats (FIG. 1).

To overcome some of these problems, as disclosed in WO 2009/040548, a transdermal delivery means was developed comprising a microprotrusion-based device for the delivery of beneficial substances across or into the skin. WO 2009/040548 discloses the use of a swellable polymer composition in the microprotrusion array. The microprotrusion array is used in the delivery of an active agent transdermally, that is through the stratum corneum (SC). WO 2009/040548 is a general teaching outlining many potential active agents including beneficial substances such as a drug, a nutrient or a cosmetic agent. WO 2009/040548 defines the term drug to include ‘beneficial substances’ for the treatment or prophylaxis of disease, for example, drug substances, substances that may improve the general health of the skin, for example, vitamins and minerals, and substances that may improve the aesthetic appearance of the skin, for example, by reducing the appearance of wrinkles or improving the degree of hydration of the skin. However, the examples only disclose active agents such as fluorescent photosensitiser drug meso-Tetra (N-methyl-4-pyridyl) porphine tetra tosylate (MW 1363.6 Da) (TMP), 5-aminolevulinc acid (ALA, MW 167 Da), bovine serum albumin (BSA). Furthermore, WO 2009/040548 only enables the transport of these beneficial substances across the SC.

The present invention aims to overcome at least some of these problems to provide a more effective delivery technology for nucleic acids and other agents both transdermally and subsequently intracellularly.

### SUMMARY OF THE INVENTION

According to a first aspect of the invention, there is provided a cell delivery system comprising
a microprotrusion array for use in the transport of a material across a biological barrier in which the array comprises a plurality of microprotrusions composed of a swellable and/or dissolvable polymer composition;
the material comprises or consists of nanoparticles formed from a nucleic acid or other agent such as a negatively charged or hydrophilic compound, preferably a protein or drug, complexed with an amphipathic cell penetrating peptide;
wherein the microprotrusion array is loaded with the nanoparticles.

According to a second aspect of the invention, there is provided the cell delivery system of the invention for use in inducing an immune response against an antigen in a subject in need thereof. In this manner, the cell delivery system ideally comprises nucleic acid including plasmid DNA which encodes an antigen and the cell delivery system induces an immune response in a host against the antigen. Alternatively, the cell delivery system comprises an other agent such as a negatively charged or hydrophilic compound.

According to a third aspect of the invention, there is provided the cell delivery of the invention for use in gene therapy in a subject in need thereof. In this manner, ideally the cell delivery system is designed to deliver a nucleic acid encoding a functional gene or protein which is deficient or mutated in the subject. Alternatively, the cell delivery system is designed to deliver an inhibitory nucleic acid such as RNA including an siRNA/shRNA/miRNA molecule. Alternatively, the cell delivery system is designed to deliver an other agent such as a negatively charged or hydrophilic compound.

According to a fourth aspect of the invention, there is provided the cell delivery system of the invention for use in the treatment and/or prophylaxis of cancer in a subject in need thereof. In this manner, ideally the cell delivery system is designed to deliver a nucleic acid encoding a functional gene or protein which is deficient or mutated in the subject. Alternatively, the cell delivery system is designed to deliver an inhibitory nucleic acid such as RNA including an siRNA/shRNA/miRNA molecule. Still alternatively, the cell delivery system is designed to deliver an other agent such as a negatively charged or hydrophilic compound.

According to a fifth aspect of the invention, there is provided a method of inducing an immune response against an antigen in a subject comprising the administration of the cell delivery system of the invention to a subject in need thereof comprising the steps of
applying the microprotrusion array to the skin such that the microprotrusions protrude through or into the stratum corneum;
allowing the microprotrusions to swell; and
allowing the microprotrusions to dissolve and release the material into the skin.

According to a sixth aspect of the invention, there is provided a method for the treatment or prophylaxis of an infection or cancer comprising the administration of the cell delivery system of the invention to a subject in need thereof comprising the steps of
applying the microprotrusion array to the skin such that the microprotrusions protrude through or into the stratum corneum,
allowing the microprotrusions to swell,
allowing the microprotrusions to dissolve and release the material into the skin.

According to a seventh aspect of the invention, there is provided the use of the cell delivery system of the invention for the administration of the material to a cell or subject in need thereof.

According to an eighth aspect of the invention, there is provided a composition or nanoparticle comprising the amphipathic cell penetrating peptide of the invention and a DNA vaccine. Essentially, the nanoparticles are formed from the DNA vaccine complexed with an amphipathic cell penetrating peptide. Preferably, the DNA vaccine comprises plasmid DNA encoding an antigen for a disease. In this manner, the composition or nanoparticle acts as antigen and elicits an immune response against the antigen in a patient in need thereof. Accordingly. the cell delivery system of the invention comprises a material which itself comprises the composition or nanoparticle as defined herein.

### DETAILED DESCRIPTION

In this specification, the term “dissolvable” covers agents which are dissolvable in liquid, such as water and interstitial fluid. The microprotrusion array dissolves at a rate determined by the polymer used. In this manner on application to the skin, the microprotrusion array can initially increase in volume (swell) and then dissolve.

In this specification, the term “swellable” covers agents which swell or imbibe liquid, such as biological fluid when in contact with interstitial fluid for example. The microprotrusion array of the invention can initially increase in volume (swell) and then dissolve.

It will be understood that the polymer of the invention is a polymer that swells and/or dissolves in the presence of water and have sufficient mechanical strength to function as microprotrusions that can puncture the stratum corneum barrier. Ideally, the polymer should be non-toxic when used in vitro or in vivo.

In this specification, it will be understood the terms “complexed” and “condensing” are interchangeable. As such, the nanoparticle is formed when the nucleic acid or other agent is complexed with or condensed with the amphipathic cell penetrating peptide of the invention.

In this specification, the term “loaded with” relates to a microprotrusion array which encapsulates or incorporates the material comprising the amphipathic cell penetrating peptide of the invention, such as nanoparticles. In this manner, the microprotrusion array contains in its polymer matrix the material/nanoparticles of the invention. For example, the microprotrusion array is ideally prepared from a solution of the polymer which is mixed with or loaded with the material/nanoparticles of the invention, placed in a mould and dried to result in a microprotrusion array supported by a polymer substrate or base element. It will be understood that the nanoparticles of the invention may be freeze-dried or spray-dried before they are mixed the polymer solution to form the microneedle array. In this manner, the microprotrusion array comprises a base element and a plurality of microprotrusions which project from the base element. The microprotrusions and the base element may comprise the same or different polymer. For example, both the base element polymer and the microprotrusion polymer may be dissolvable. Alternatively, only the microprotrusion polymer may be dissolvable. Additionally, one or both of the plurality of microprotrusions and the base element may comprise the material/nanoparticles.

The present invention is directed to a cell delivery system comprising
a microprotrusion array for use in the transport of a material across a biological barrier in which the array comprises a plurality of microprotrusions composed of a swellable and/or dissolvable polymer composition;
the material comprises nanoparticles formed from a nucleic acid or other agent, including a protein or drug, complexed with an amphipathic cell penetrating peptide;
wherein the microprotrusion array is loaded with the nanoparticles to enable transport across a biological barrier.

Advantageously, the present invention overcomes the physical and biological barriers encountered in conventional nucleic acid delivery methods to facilitate both transdermal, intradermal and intracellular delivery of the material. This is enabled by the unique combination of a microprotrusion array loaded with material comprising or consisting of nanoparticles comprising an amphipathic cell penetrating peptide encapsulated nucleic acid, or other agent, including a protein or drug.

Advantageously, the microprotrusion array facilitates transport of the nanoparticles across the first biological barrier, for example the skin e.g. the subcutaneous layer (SC). Then, as the microprotrusion array is composed of a swellable and/or dissolvable polymer composition it dissolves upon contact with interstitial fluid enabling release of the material comprising or consisting of nanoparticles into the extracellular space. The nanoparticles then enables the transport of the nucleic acid, or other agent such as a negatively charged or hydrophilic compound, including a protein or drug, across cell membranes, out of the endosomes and to the nucleus ideally for presentation to the antigen presenting cells. Thus, the use of swellable and/or dissolvable polymer microprotrusion array combined with the nanoparticles ensures that the nanoparticles and its contents reach the high APC population in the skin cells to evoke maximum effect. We have shown that this specific combination of microprotrusion array/microneedle with nanoparticle provides unexpectedly superior results compared to microneedle delivery alone or nanoparticle delivery alone.

In the present invention, ideally the nanoparticles are formed from a nucleic acid or other agent complexed with an amphipathic cell penetrating peptide. The amphipathic cell penetrating peptide has been designed to condense the nucleic acid or other agent into nanoparticles, protect the nucleic acid or other agent from degradation and facilitate cellular entry through natural endocytosis because they are <100 nm and have a positive charge. The cell penetrating peptide of the invention had improved or equivalent cell penetration activity compared to conventional transfection reagents.

Ideally, nanoparticles have a N:P ratio from for example 0.5 to 12, preferably from 0.5 to 6. N:P ratio indicates the ratio of peptide (N) to the nucleic acid (P) or other agent. We have found that these N:P ratios maximise transfection efficiency in dendritic cells in-vivo. Other N:P ratios may also be contemplated.

After the microprotrusion array has facilitated transport of the nanoparticles across the biological barrier (SC) to the extracellular space, the drop in pH changes the conformation of the amphipathic cell penetrating peptide so that it disrupts the endosome and gets into the cytoplasm. The presence of arginine in particular in the peptide also facilitates active nuclear transport. This means that there is a greater chance of the nucleic acid reaching the nucleus, and when the nucleic acid is a DNA vaccine more antigen will be produced to give a more potent immune response.

Advantageously, the present inventors have found that nanoparticles can be loaded into the microprotrusion arrays without compromising the microprotrusion structure which is required for cell penetration for example.

Furthermore, the inventors have shown that nucleic acid (e.g. DNA) from the nanoparticles was released from the microprotrusion arrays across a biological barrier, namely skin. The integrity of the nucleic acid (e.g. DNA) was not compromised following an extended incubation period.

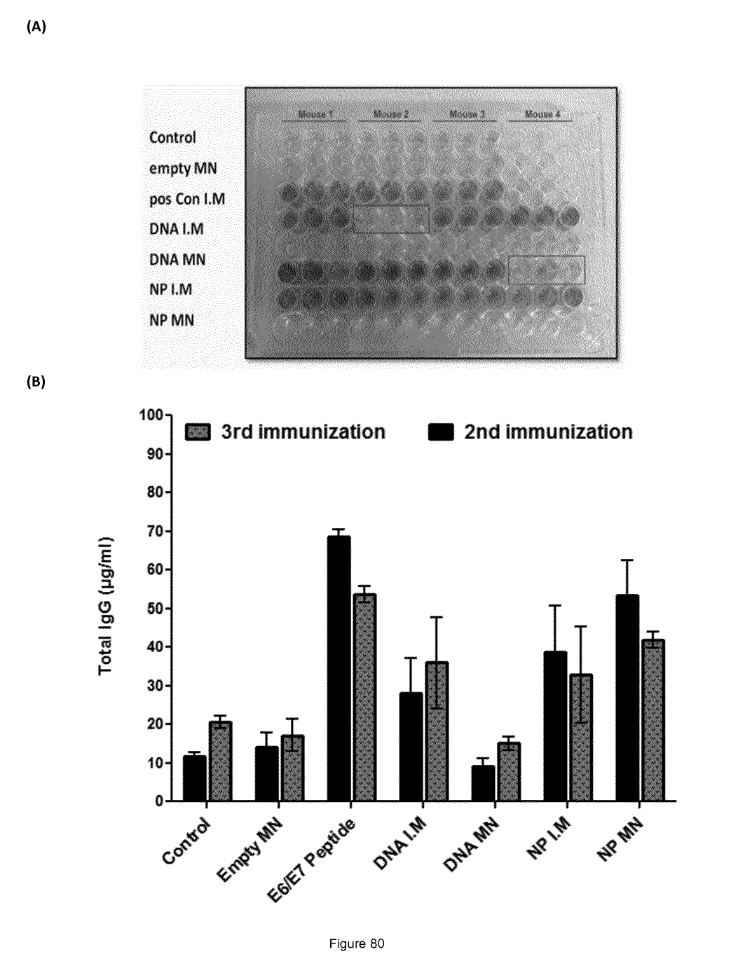

Importantly, in-vivo studies have shown expression of nanoparticle nucleic acid (e.g. DNA) illustrating the functionality of this technology. These advantages are clearly illustrated in FIGS. 80 to 84 in which the use of the cell delivery system of the invention (NP MN) provides greatly improved results compared to DNA adminstered intramuscularly (DNA I.M), DNA administered using microneedles (DNA MN), and nanoparticles delivered intramuscularly (NP I.M). In particular, the inventors have shown that the amphipathic cell penetrating peptide of the invention (the “RALA” peptide) condenses and delivers nucleic acid or other agents intracellularly in-vivo. When DNA vaccines are delivered in accordance with the invention, the inventors have observed the generation of antigen expression and resultant humoral immune responses.

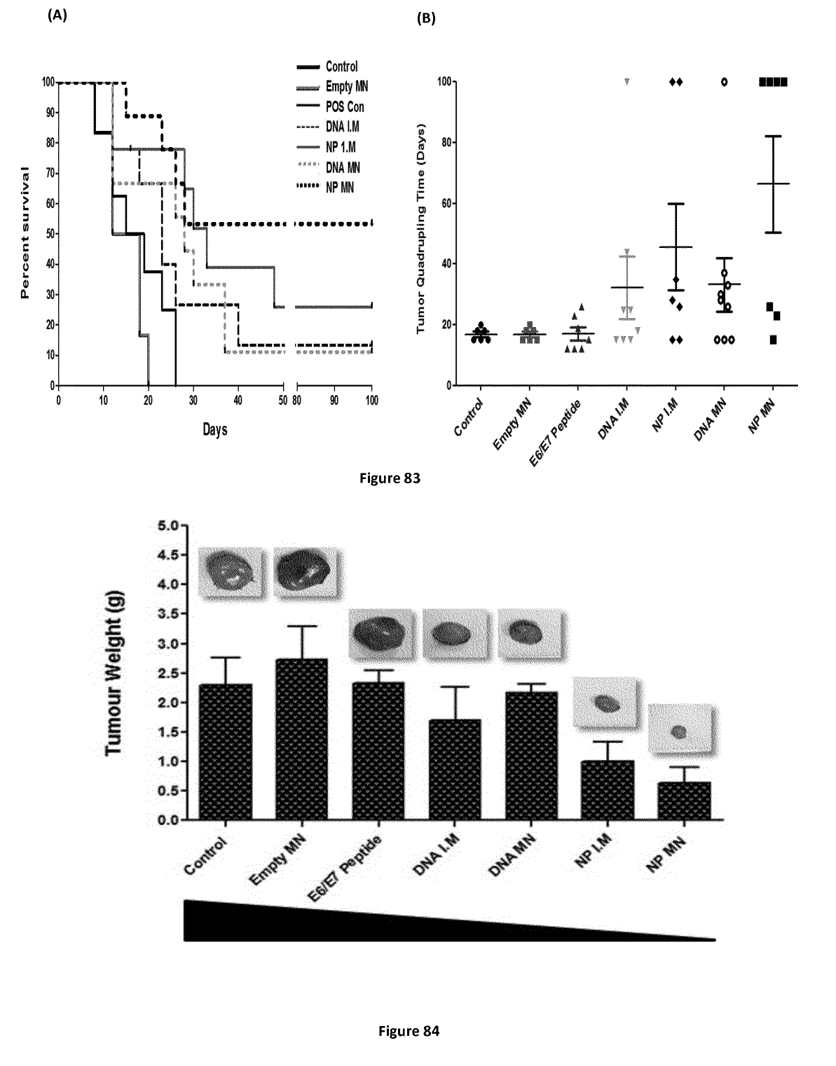

According to a preferred embodiment, the nucleic acid is a DNA vaccine, preferably plasmid DNA encoding an antigen for a disease. In this manner, the cell delivery system induces an immune response in a host against the antigen (see for example FIG. 83). At present, most techniques for DNA vaccine delivery to a cell involve physical techniques such as a gene gun or needle-free injection. These techniques are quite crude, and unlike the present invention which allows targeted delivery of a DNA vaccine or other agent to a cell or subject. This is a major advantage of the present invention.

According to an alternative embodiment, the nucleic acid is suitable for gene therapy, preferably in the form of DNA or RNA including mRNA, miRNA or siRNA. For example, the cell delivery system of the invention delivers a nucleic acid encoding a functional gene or protein which is deficient or mutated in the subject. Alternatively, the cell delivery system may deliver an inhibitory nucleic acid such as RNA including an siRNA/shRNA/miRNA molecule.

According to yet another embodiment, the other agent is a negatively charged or hydrophilic compound, such as a protein, drug or active agent. preferably a phosphate or lipophilic based drug, more preferably a bisphosphonate drug or gold.

According to one aspect of the invention, there is provided the cell delivery system for use in inducing an immune response (i.e. inducing a prophylactic effect) against an antigen in a subject in need thereof. In this embodiment, the material/nanoparticle comprises a DNA vaccine, preferably plasmid DNA encoding an antigen for a disease which when expressed induces an immune response against the antigen. The inventors have shown that the cell delivery system of the invention can be used to deliver a DNA vaccine to have both a prophylactic/immunization (e.g. FIG. 83) and/or a therapeutic effect (e.g. FIG. 84), such as anti-tumor activity. This is a significant advantage of the invention.

According to another aspect of the invention, there is provided the cell delivery system of the invention for use in gene therapy in a subject in need thereof. In this embodiment the cell delivery system is designed to deliver a nucleic acid encoding a gene which is deficient or mutated in the subject. Alternatively, the cell delivery system is designed to deliver an inhibitory nucleic acid such as RNA including an siRNA/shRNA/miRNA molecule.

According to another aspect of the invention, there is provided a cell delivery system of the invention for use in the treatment and/or prophylaxis of cancer in a subject in need thereof. In this manner, the cell delivery system is designed to deliver a nucleic acid encoding a gene which is deficient or mutated in the subject (therapeutic effect). Alternatively, the cell delivery system is designed to deliver an inhibitory nucleic acid such as RNA including an siRNA/shRNA/miRNA molecule.

According to yet another aspect of the invention, there is provided method of inducing an immune response against an antigen in a subject comprising the administration of the cell delivery system of the invention to a subject in need thereof comprising the steps of
applying the microprotrusion array to the skin such that the microprotrusions protrude through or into the stratum corneum;
allowing the microprotrusions to swell; and
allowing the microprotrusions to dissolve and release the material into the skin.

In a preferred embodiment, the material/nanoparticle comprises a DNA vaccine, preferably plasmid DNA encoding an antigen for a disease which when expressed induces an immune response against the antigen. It will be understood that the nanoparticle may comprise any nucleic acid or other agent as defined previously.

According to yet another aspect of the invention, there is provided a method for the treatment and/or prophylaxis of an infection or cancer comprising the administration of the cell delivery system of the invention to a subject in need thereof comprising the steps of
applying the microprotrusion array to the skin such that the microprotrusions protrude through or into the stratum corneum,
allowing the microprotrusions to swell,
allowing the microprotrusions to dissolve and release the material into the skin.

In a preferred embodiment, the cell delivery system is designed to deliver a nucleic acid encoding a gene which is deficient or mutated in the subject. Alternatively, the cell delivery system is designed to deliver an inhibitory nucleic acid such as RNA including an siRNA/shRNA/miRNA molecule. It will be understood that the cell delivery system is designed to deliver any nucleic acid or other agent as defined previously.

According to another aspect of the invention, there is provided the use of the cell delivery system according to any of the preceding claims for the transdermal, intradermal and/or intracellular administration of the material to a subject in need thereof. The inventors have shown that transdermal/intradermal administration of the cell delivery system of the invention is superior than intramuscular administration of the same nanoparticles (see e.g. FIG. 84). This is another advantage of the invention.

Further details on each component of the cell delivery system of the invention are expanded on below.

### Microprotrusion Array

Ideally, the microprotrusion array is prepared from a solution of a polymer which is mixed with or loaded with the nanoparticles of the invention, placed in a mould and dried to result in a microprotrusion array supported by a polymer substrate or base element. The nanoparticles may be freeze-dried or spray-dried before being loaded into the polymer solution.

Ideally, the microprotrusion array comprises a base element and a plurality of microprotrusions which project from the base element. The base element and a plurality of microprotrusions may be made from the same or different polymer materials. base element and a plurality of microprotrusions may both comprise nanoparticles of the invention or only one of these elements may comprise nanoparticles.

As disclosed in WO 2009/040548, the microprotrusions are composed of swellable and/or dissolvable polymers which can puncture the stratum corneum of mammalian skin without breaking upon insertion into the skin and can be used for efficient delivery of active substances through the stratum corneum without many of the problems associated with the use of conventional solid microneedles.

Any polymer which can penetrate the stratum corneum of the skin and which swells in the presence of liquid may be used.

Ideally, the microprotrusion array polymer is dissolvable in the natural cellular, interstitial environment. For example, FDA-approved hydrogel materials can be utilised to form the microprotrusion and such hydrogel materials can be inexpensive and biocompatible.

The material dose which can be provided by a microprotrusion may not necessarily limited by how much can be loaded into a microprotrusion, as the material could be contained in an attached material reservoir attached to the upper surface of the microprotrusion array.

It will be understood that the swelling of the microprotrusions on entry to the skin has a number of advantages over conventional microneedle arrays or indeed sugar microneedles. For example, where the microprotrusion array is used, the increased surface area of microprotrusion in contact with the epidermal layer underneath the stratum corneum resulting from the swelling of the microprotrusions enables enhanced delivery of a material to the epidermal layer underneath the stratum corneum. In particular embodiments arrays of swellable polymeric microprotrusions can absorb moisture upon insertion into the skin and swell to form continuous aqueous channels between the external environment and the dermal microcirculation, thus forming an ‘aqueous bridge’ across the lipophilic stratum corneum barrier. Such channels do not have the tendency to block on positioning of the array, in contrast to conventional silicon based microneedle devices having channels therein. Optionally, the microprotrusions can release the material from every point on the surface of the microprotrusion further minimising blockage of the microprotrusion by tissue. Optionally a hydrogel microprotrusion array could be integrated with a material reservoir to give a rapid bolus dose, achieving a therapeutic plasma level, followed by controlled, prolonged delivery to maintain this level. Optionally, swollen hydrogel materials can contain >70%, for example >80%, such as >90% water. By having a high water content, material diffusion is facilitated, as there will be less chance of impedance of material movement due to collision with polymer chains. In addition, water allows passage of ions and polar substances and facilitates electroosmotic flow under a potential gradient. Thus, conduction of charged and/or polar substances and fluid moving by electro-osmotic flow is possible.

### Polymers

Microprotrusions can be fabricated from any suitable swellable and/or dissolvable polymer, which in its dry state is hard and brittle to allow penetration of the stratum corneum, but then which, upon taking up moisture swells to allow diffusion of therapeutic active agents. The polymers of the invention swell and/or dissolve in the viable skin layers, where interstitial fluid is present.

The polymers that may be used in the present invention include, but are not limited to the following poly(vinylalcohol), poly(vinylpyrrolidone), poly(hydroxyethylmethacrylate) and derivatives thereof, poly(methylvinylether/maleic acid) and derivatives thereof, poly(methylvinylether/maleic anhydride) and derivatives thereof, poly(acrylic acid), poly(caprolactone, hydroxyethylcellulose and derivatives thereof, poly(ethyleneglycol) and derivatives thereof, hyaluronic acid, chitosan and carbohydrate materials (eg galactose, fructose etc.) and derivatives thereof. For in-vivo or in-vitro use the polymer chosen should be non-cytotoxic.

According to a preferred embodiment of the invention, the swellable polymer composition used in the microprotrusion array is polyvinylpyrrolidone (PVP). PVP is fully licensed and FDA-approved PVP is a water-soluble and biodegradable polymer which eliminates the risk of leaving biohazardous sharp waste in the skin. PVP is also a low cost material which provides for ease of fabrication in the micro-moulding process and enable the mass production of the arrays. We have found that when PVP is used the presence of the amphipathic cell penetrating peptide is essential for the protection of the nucleic acid or other agent within the nanoparticles to prevent the PVP interacting with the nucleic acid and having a detrimental effect.

According to another preferred embodiment of the invention, the polymer composition used in the microprotrusion array is poly(vinylalcohol) (PVA). This is a dissolvable polymer.

Whilst both PVP and PVA are suitable for in-vivo usage we have found that PVA is less toxic to cells and in some applications may be preferable.

Polymers, such as PVP and PVA, may be used with a molecular weight of less than approximately 400 KDa. Preferably, the polymers, such as PVP and PVA, with a molecular weight less than 60 KDa may be used. More preferably, the polymers, such as PVP and PVA, with a molecular weight less than 15 KDa may be used. Even more preferably, the polymers, such as PVP and PVA, with a molecular weight less than 10 KDa, typically from 8-10 KDa, may be used. Ideally, PVA with a molecular weight ranging from 9-23 KDa may be used, preferably 9-10 KDa PVA and 13-23 KDa PVA.

Optionally, from 15 to 40% w/w, preferably from 20 to 30% w/w, polymer may be used at varying molecular weights.

We have found that both PVP and PVA are preferred polymers which retain height on introduction of the nanoparticles and are strong enough to penetrate the skin and withstand forces of approximately 15 Newtons cm⁻². Furthermore, the nucleic acid/DNA or other agent within the nanoparticles retain their integrity and functionality within the microneedles.

In particular embodiments, the polymers of the microprotrusions are crosslinked, either physically, chemically or both. The microprotrusion array can comprise groups of microprotrusions wherein a first group comprises at least one different cross-linker to at least a second group.

In particular embodiments the microprotrusions may not be crosslinked and will dissolve following an initial swelling phase upon puncturing the stratum corneum and coming into contact with skin moisture. In this case, the material can be released into the skin at a rate determined by the rate of dissolution of the microprotrusions. The rate of dissolution of particular microprotrusions is dependent on their physicochemical properties which can be tailored to suit a given application or desired rate of material release.

Combinations of non-crosslinked, lightly crosslinked and extensively crosslinked microprotrusions can be combined in a single device so as to deliver a bolus dose of the material achieving a therapeutic plasma level, followed by controlled delivery to maintain this level. This strategy can be successfully employed whether the material is contained in the microprotrusions and base element or in an attached reservoir.

In further embodiments, the base element and microprotrusions may contain in their matrix, defined quantities of one or more water soluble excipients. Upon insertion into skin these excipients will dissolve leaving pores behind in the matrix of the base element and microprotrusions. This can enhance the rate of release, which can be further controlled by changing the excipient, its concentration and/or its particle size. Suitable excipients include, but are not limited to glucose, dextrose, dextran sulfate, sodium chloride and potassium chloride, sodium carbonate, sodium hydroxide, sodium hydrogen carbonate or other water soluble excipients known in the art. Other excipients include conventional pharmaceutical disintegrants used in solid dosage forms, including for example cross-carmellose or crospovidone.

As noted above, in order to be of use in transdermal delivery arrays of microprotrusions must be capable of creating openings in the stratum corneum barrier through which beneficial substances can move. Thus, the force of insertion is less than the force required to fracture the microprotrusions.

Suitably, the microprotrusions do not fracture when a pressure of insertion of less than 5.0 N cm⁻², for example less than 3.0 N cm⁻², such as less than 0.5 N cm⁻² is exerted on the microprotrusions along their length.

A microprotrusion can be any suitable size and shape for use in an array to puncture the stratum corneum. The microprotrusions are designed to pierce and optionally cross the stratum corneum. Suitably, the height of the microprotrusions can be altered so as to allow penetration into the upper epidermis, as far as the deep epidermis or even the upper dermis, but not allowing penetration deep enough into the skin to cause bleeding. In one embodiment, the microprotrusions are conical in shape with a circular base which tapers to a point at a height of the microprotrusion above the base.

In embodiments of the microprotrusion array the microprotrusions can be in the range of 1 μm to 3000 μm in height. For example, the microprotrusions can have heights in the range 50 μm to 400 μm, for example 50 to 100 μm. Suitably, in embodiments of the arrays of the invention, microprotrusions can have a width, e.g. diameter in the case of microprotrusions of circular cross-section diameter of 1-500 μm at their base. In one embodiment microprotrusions of and for use in the invention can have a diameter in the range 50-300 μm, for example 100-200 μm. In another embodiment, the microprotrusion of the invention may be of a diameter in the range of 1 μm to 50 μm, for example in the range 20-50 μm.

The apical separation distance between each of the individual microprotusions in an array can be modified to ensure penetration of the skin while having a sufficiently small separation distance to provide high transdermal transport rates. In embodiments of the device the range of apical separation distances between microprotrusions can be in the in the range 50-1000 μm, such as 100-300 μm, for example 100-200 μm. This allows a compromise to be achieved between efficient penetration of the stratum corneum and enhanced delivery of therapeutic active agents or passage of interstitial fluid or components thereof.

It will be apparent to those skilled in the art that the microprotrusions of the invention can take any reasonable shape, including, but not limited to, microneedles, cones, rods and/or pillars. As such, the microprotrusions may have the same diameter at the tip as at the base or may taper in diameter in the direction base to tip. The microprotrusions may have at least one sharp edge and may be sharp at the tips. The microprotrusions may be solid, have a hollow bore down at least one longitudinal axis at an angle to the base element and extending to the first side of the base element, they may be porous, or may have at least one channel running down at least one outer surface from tip to base element.

In use, the microprotrusions may be inserted into the skin by gentle applied pressure or by using a specially-designed mechanical applicator applying a pre-defined force. An additional device may be used to reduce the elasticity of skin by stretching, pinching or pulling the surface of the skin so as to facilitate insertion of the microprotrusions. This latter function could be usefully combined with the function of the applicator to produce a single integrated device for insertion of a microprotrusion array.

The material contained in the microprotrusions themselves will be rapidly released upon swelling, initially as a burst release due to material at the surface of the microprotrusions. The subsequent extent of release will be determined by crosslink density and the physicochemical properties of the material. Release of material from the drug reservoir will occur more slowly at first as a result of the time required to swell the microprotrusions up as far as the material reservoir, subsequent partitioning of the material into the swollen microprotrusions and diffusion of the material through the swollen matrix.

A number of applicators for microneedle based delivery are known in the art. For example, US20046743211 describes methods and devices for limiting the elasticity of skin by means of stretching, pulling or pinching the skin, so as to present a more rigid, less deformable surface in the area to which microneedle-array-based transdermal drug delivery systems are applied. US 20060200069 describes a spring-loaded impact applicator for the application of coated microprojection arrays to the skin. A further application known in the art is the Alza Macroflux® device which is applied to skin using a specially-designed spring-loaded applicator (Alza Corporation, 2007).

Manufacture of Microprotrusion Array Loaded with Material

Microprotrusions composed of polymers known to form hydrogels can be manufactured by any such methods known in the art. For example, they can be prepared by a micromoulding technique using a master template, such as a microprotrusion array made from one or more of a wide variety of materials, including for example, but not limited to; silicon, metal polymeric material. Master templates can be prepared by a number of methods, including, but not limited to, electrochemical etching, deep plasma etching of silicon, electroplating, wet etch processes, micromoulding, microembossing, “thread-forming” methods and by the use of repetitive sequential deposition and selective x-ray irradiation of radiosensitive polymers to yield solid microprotrusion arrays.

Micromoulds can be prepared by coating the master template with a liquid monomer or polymer which is then cured and the master template removed to leave a mould containing the detail of the master template. In the micromoulding technique, a liquid monomer, with or without initiator and/or crosslinking agent is placed in the mould, which is filled by means of gravitational flow, application of vacuum or centrifugal forces, by application of pressure or by injection moulding. The monomer may then be cured in the mould by means of heat or application of irradiation (for example, light, UV radiation, x-rays) and the formed microprotrusion array, which is an exact replicate of the master template is removed.

Alternatively, a solution of a polymer with or without crosslinking agent can be placed in the mould, which is filled by means of gravitational flow, application of vacuum or centrifugal forces, by application of pressure or by injection moulding. The solvent can then be evaporated to leave behind a dried microprotrusion array, which is an exact replicate of the master template, and can then be removed from the mould. The solvents that can be used include, but are not limited to, water, acetone, dichloromethane, ether, diethylether, ethyl acetate. Other suitable solvents will be obvious to one skilled in the art. Micromoulds can also be produced without the need for master templates by, for example, micromachining methods and also other methods that will be obvious to those skilled in the art.

For example, in one embodiment, the microprotrusion arrays may be prepared using micromoulds prepared using a method in which the shape of the desired microprotrusions are drilled into a suitable mould material, for example using a laser and the moulds are then filled using techniques known in the art or as described herein.

Microprotrusions composed of polymers known to form hydrogels can also be manufactured using a “self-moulding” method. In this method, the polymeric material is first made into a thin film using techniques well known in the art, including for example, but not limited to, casting, extrusion and moulding. The material may, or may not be crosslinked before the “self moulding” process. In this process, the thin film is placed on a previously-prepared microprotrusion array and heated. Plastic deformation due to gravity causes the polymeric film to deform and, upon hardening, create the desired microprojection structure.

Microprotrusions with a hollow bore can be manufactured by using moulds prepared from hollow master templates or suitably altering the micromachining methods or other methods used to prepare solid microprotrusions. Hollow bores can also be drilled mechanically or by laser into formed microprotrusions. Microprotrusions which have at least one channel running down at least one outer surface from tip to base element can also be produced by suitable modification of the method used to prepare solid microprotrusions. Such alterations will be obvious to those skilled in the art. Channels can also be drilled mechanically or by laser into formed microprotrusions.

Microprotrusions composed of polymers known to form hydrogels can also be manufactured using a “thread forming” method whereby a polymer solution spread on a flat surface has its surface contacted by a projection which is then moved upwards quickly forming a series of polymer “threads”, which then dry to form microprotrusions.

In all of the above methods, substances to be incorporated into the microprotrusions themselves (e.g., material/nanoparticles/freeze-dried or spray-dried nanoparticles/pore forming agents, enzymes etc.) can be added into the liquid monomer or polymer solution during the manufacturing process. Alternatively, such substances can be imbibed from their solution state in a solution used to swell the formed microprotrusion arrays and dried thereafter or the formed arrays can be dipped into a solution containing the agent of interest or sprayed with a solution containing the agent of interest. Solvents used to make these solutions include water, acetone, dichloromethane, ether, diethylether, ethyl acetate. Other suitable solvents will be obvious to those skilled in the art, as will the processes used to dry the microprotrusion arrays. If the microprotrusions and/or base elements are to be made adhesive, the formed arrays can be dipped into a solution containing an adhesive agent or sprayed with a solution containing an adhesive agent. The adhesive agents used can be a pressure sensitive adhesive or a bioadhesive. These substances are well known and will be obvious to those skilled in the art.

Alternatively, the substances to be incorporated into the microprotrusions themselves (e.g., material/nanoparticles/pore forming agents, enzymes etc.) are freeze-dried or spray-dried prior to incorporation within the swellable and/or dissolvable polymer composition. They are then reconstituted using one or more of water, trehalose and/or PVP.

The base element on which the microprotrusions are formed can be varied in thickness by suitable modification of the method of manufacture, including, for example, but not limited to increasing the quantity of liquid monomer or polymer solution used in the manufacturing process. In this way the barrier to diffusion/transport of therapeutic active agents and/or analytes of interest can be controlled so as to achieve, for example rapid delivery or sampling or sustained release. Where therapeutic active agent(s) is/are to be contained within the matrix of the microprotrusions and base element, the thickness of the base element can usefully be increased so as it functions as a fully integrated reservoir.

According to a preferred embodiment of the invention, the microprotrusion array is prepared from a solution of the polymer which is mixed with or loaded with the material of the invention, placed in a mould and dried to result in a microprotrusion array support by a polymer substrate or baseplate.

It will be understood that the material/nanoparticles may be loaded into the plurality of microprotrusions alone; or may be loaded into both the baseplate and the plurality of microprotusions microneedles; or may be loaded into the baseplate alone.

In an alternative embodiment, the microprotrusion array may be made from a swellable or dissolving polymer composition and the material/nanoparticles may be added separately as a reservoir in the form of an attached patch, semi-solid gel or liquid. In a preferred embodiment, the material is a nanoparticle comprising a nucleic acid complexed with a cell penetrating peptide.

Ideally the microprotrusion array of the invention is in the form of a patch. The patch is formed for transdermal delivery to facilitate transdermal and/or intradermal delivery of the material within the microprotrusion array to the cell and/or subject. Such a patch may adapted to adhere to the skin or cell surface and as explained below may comprise a backing layer with adhesive to adhere to the skin or cell surface or the microprotrusion array may itself adhere to the skin or cell surface.

The size of the patch will dictate the number of microprotrusions present in the array, which in turn dictates the amount of nucleic acid present in the nanoparticle comprising material that may be loaded into the microprotrusion array.

For example, we have found that a 1 cm² patch may be loaded with approximately 20 μg of DNA. Our initial experiments have found that a suitable average dosage may be approximately 10-50 μg DNA/cm², preferably 20 μg DNA/cm².

| | | | |
|---|---|---|---|
| Approx. Dose of | No. of | Size of | Size of |
| Nucleic Acid | Needles | Array | Patch |

| | | | | | | |
|---|---|---|---|---|---|---|
| 20 | μg | 722 | 1 | cm² | 1.5 | cm² |
| 50 | μg | 1,805 | 2.5 | cm² | 3.75 | cm² |
| 100 | μg | 3,610 | 5 | cm² | 7.5 | cm² |
| 200 | μg | 7,220 | 10 | cm² | 15 | cm² |
| 500 | μg | 18,050 | 25 | cm² | 37.5 | cm² |

In order to increase the amount of nucleic acid present in the nanoparticle comprising material that may be loaded into the microprotrusion array, the nanoparticles may be freeze-dried or spray-dried. We have found that up to 500 μg of freeze-dried nanoparticles per 1×1 cm patch can be loaded into the microneedles (i.e. 500 DNA/cm²). Ideally, 100 μg of freeze-dried nanoparticles per 1×1 cm patch can be loaded into the microneedles (ie. 100 DNA/cm²).

We have found that DNA delivery is approximately 60% of the total DNA loaded into the microneedles after 5 mins and this increases to approximately 90% of the total DNA loaded into the microneedles after 24 hours.

Thus, advantageously the freeze-drying of the nanoparticles increased the amount of nanoparticles in the microneedles and also increased the percentage DNA delivery after administration.

### Delivery Devices

One aspect of the present invention is directed to a transdermal drug delivery system for delivering a material) to a biological interface. As described above, such a system can comprise a base element and plurality of microprotrusions formed thereon. In particular embodiments said base element can have a first side and a second side; and said plurality of microprotrusions comprise a plurality of elements which project from the second side of said base element at an angle. In one embodiment with respect to said base element said angle is in the range 45° to 90°, for example in the range 70° to 90°. In a particular embodiment, said angle is about 90°. In particular embodiments of the device, said base element and plurality of microprotrusions can be formed of polymeric materials known to form hydrogels upon absorption of moisture. Suitably, in preferred embodiments, in use, upon insertion into skin the polymeric materials of said microprotrusions and base element can absorb moisture and increase in size to form swollen hydrogels; wherein the material can diffuse through said swollen base element and swollen hydrogel microprotrusions. In particular embodiments, the material can be provided from a reservoir; wherein said reservoir can be attached to the first side of the base element. In particular embodiments of the device, the reservoir can be a material dispersed in a suitable matrix material, for example a suitable adhesive or non-adhesive polymer matrix, or a material-containing reservoir.

In an alternative embodiment of a transdermal delivery device of the invention, an attached reservoir is not present. In such embodiments, the material may be contained within the swellable polymer composition of said base element and/or plurality of microprotrusions. Said substances can be either dissolved in the swellable polymer composition or suspended in particulate form. Upon insertion into skin and swelling of the microprotrusions, the material can be released into the skin at a rate determined by the degree of crosslinking of the microprotrusions and the material itself.

A backing layer with an adhesive border extending beyond the area of the base element of the microprotrusions may be used to keep microprotrusion-based devices in place on the skin surface for protracted periods of time, for example up to or greater than 72 hours. The surface of a base element of and, optionally, the microprotrusions themselves, may be coated with an adhesive material, so as to promote retention at the site of application.

In particular embodiments, the material can be chemically bonded to the polymer(s) making up the microprotrusions and base elements. In this case, the material can be released upon insertion into the skin by; dissolution of the microprotrusions, hydrolysis, enzymatic or spontaneous non-catalysed breakage of the bonds holding it to the polymer(s). The rate of material release can thus be determined by the rate of reaction/bond breakage.

In an alternative embodiment, the polymeric composition of the microprotrusions and/or base elements can be adjusted such that it can be stimulus-responsive. For example, local changes in pH or temperature can alter the properties (eg ability to swell upon imbibing moisture) of the microprotrusions and base elements, such that a change in the rate of delivery of material occurs. Alternatively, an external stimulus, such as light illumination, can be used to affect a change in the properties of the microprotrusions and base elements, such that a change in the rate of delivery of the material occurs.

In embodiments of arrays of the invention, the polymeric composition of the microprotrusions and base elements can be adjusted such that the surface properties of the device are altered, becoming more hydrophilic, lipophilic, anionic or cationic in character.

### Iontophoretic Devices

Another aspect of the present invention is directed to an iontophoretic transdermal drug delivery system for delivering a material to a biological interface. Such a system can comprise a cell delivery device of the invention. In specific embodiments the material can be provided from a reservoir. Said reservoir can be a matrix-type reservoir or a material-containing reservoir. In such embodiments the device may further comprise a first electrode and a second electrode at a location different to said first electrode, both electrodes being proximal to said reservoir, a power source, electronic controller and central processing circuit. Application of a potential difference between the electrodes facilitates delivery of the material from said reservoir into the skin by iontophoresis or electroosmotic flow through said swollen base element and microprotrusions.

### Material Comprising Nanoparticles

The present invention is directed to the transdermal, intradermal and intracellular transport of a material across a biological barrier.

According to the invention the material comprises or consists of a composition or nanoparticles formed from a nucleic acid or other agent complexed or condensed with the amphipathic cell penetrating peptide of the invention. In this specification, it will be understood the terms “complexed” and “condensing” are interchangeable.

Advantageously, the peptide of the invention condenses the nucleic acid or other agent, preferably a negatively charged or hydrophilic compound.

The other agent is preferably a negatively charged or hydrophilic compound, including a protein, drug or active agent. In this embodiment, nanoparticles may also be formed from a negatively charged or hydrophilic compound complexed with an amphipathic cell penetrating peptide. These negatively charged or hydrophilic compound include but are not limited to any phosphate or lipophilic based drug, preferably a bisphosphonate drug and gold for example. These are described in more detail below.

Advantageously, we have found that the nanoparticles of the invention, once administered transdermally through the microprotrusion array, facilitates intracellular transport and results in the nuclear localisation of the nucleic acids to cells, both in-vitro or in-vivo. This gives the amphipathic cell penetrating peptide of the invention a distinct advantage over conventional delivery systems which do not achieve nuclear localisation.

Advantageously, we have found that the claimed amphipathic cell penetrating peptide can create nanoparticles with a size less than 150 nm or even 100 nm with nucleic acids or other agents. This facilitates transport of these agents across cell membranes, out of the endosomes and to the nucleus. We have found that these nanoparticles are stable in serum and over a temperature range of 4 to 37° C.

According to one embodiment, the amphipathic cell penetrating peptide of the invention is complexed with a nucleic acid, preferably DNA, mRNA. miRNA or siRNA, to form discrete spherical nanoparticles, each nanoparticle with a diameter less than approximately 150 nm, preferably less than or equal to 100 nm.

This delivery system is applicable across a wide range of nucleic acids, including DNA, RNA, mRNA, miRNA, siRNA and/or shRNA, and other agents, preferably small molecule agents, such as proteins, drugs or other active agents.

For example, the nucleic acid may be a DNA vaccine in the form of plasmid DNA. Ideally, the DNA vaccine targets cancer, such as cervical, breast or prostate cancer. Other cancers may also be targeted.

For example, the plasmid DNA may provide protection against herpes simplex virus (HPV), namely, HPV-16 E6, HPV-16 E7 and HPV-16 E6.E7, which cause cervical cancer. For prostate cancer, DNA coding for the tumour associated antigens (TAAs) Prostatic Antigen Phosphatase (PAP), Prostate Specific Antigen (PSA) and Granulocyte Macrophage-Colony Stimulating Factor (GM-CSF) could be utilized in this system. While TAAs for breast cancer could use DNA coding for HER-2/neu or the membrane associated glycoprotein (MUC-1).

Alternatively, the nucleic acid may be miRNA, siRNA or shRNA and may inhibit the expression of a disease causing gene, including cancer causing genes. In this embodiment, the nanoparticles comprise the claimed amphipathic cell penetrating peptide and siRNA, and hence act as a siRNA transfection agent. The inventors have shown, with siRNA, there is a much higher level of cellular entry compared to commercially available transfection reagents e.g. Oligofectamine®. In-vivo tests have shown that successful gene delivery following systemic injection into the bloodstream. Importantly, repeated injection of the nanoparticles does not illicit a significant immune response, either adaptive (IgG or IgM) or inflammatory (IL-6, II-1b). Furthermore, the inventors have shown that the there is no neutralisation of the claimed amphipathic cell penetrating peptide following systemic delivery. This is another major advantage of the nanoparticles of the invention.

According to another embodiment, the amphipathic cell penetrating peptide of the invention is complexed with a nucleic used in gene therapy. The nucleic acid may encode a functional, therapeutic gene to replace a mutated gene. Alternatively, the nucleic acid may correct a mutation or encodes a therapeutic protein drug. In this manner, the nanoparticles of the invention may be used as adjuvant gene therapy treatment administered optionally prior to conventional treatments.

### Amphipathic Cell Penetrating Peptide

According to a general aspect of the invention, the cell penetrating peptide of the invention had improved or equivalent cell penetration activity compared to conventional transfection reagents. The cell penetrating peptide of the invention acts as a transfection reagent and enables intracellular delivery, ideally to the nucleus of the subject.

In this specification and particularly the examples, the amphipathic cell penetrating peptide of the invention may optionally be referred to as the “RALA peptide”.

According to a general aspect of the invention, the amphipathic cell penetrating peptide comprises or consists of an amphipathic cell penetrating peptide less than approximately 50 amino acid residues comprising at least 6 arginine residues (R), at least 12 Alanine Residues (A), at least 6 leucine residues (L), optionally at least one cysteine residue (C) and at least two but no more than three glutamic acids (E). Optionally,
a. The arginine (R) residues are evenly distributed along the length of the peptide;
b. the ratio of arginine (R) to negatively charged amino acid residues glutamic acid (E) is from at least 6:2 to 9:2 or 6:2 to 8:2; and/or
c. the ratio of hydrophilic amino acid residues to hydrophobic amino acid residues at pH 7 is at least 30:70 to 40:60 or 30:67 to 40:60.

According to a preferred embodiment, the amphipathic cell penetrating peptide comprises or consists of an amphipathic cell penetrating peptide less than approximately 50 amino acid residues comprising at least 6 arginine residues (R), at least 12 Alanine Residues (A), at least 6 leucine residues (L), optionally at least one cysteine residue (C) and at least two but no more than three glutamic acids (E) wherein
a. The arginine (R) residues are evenly distributed along the length of the peptide;
b. the ratio of arginine (R) to negatively charged amino acid residues glutamic acid (E) is from at least 6:2 to 9:2 or 6:2 to 8:2; and/or
c. the ratio of hydrophilic amino acid residues to hydrophobic amino acid residues at pH 7 is at least 30:70 to 40:60 or 30:67 to 40:60.

We have found that the presence of arginine (R) residues in the amphipathic cell penetrating peptide is essential. Ensuring an even distribution of arginine (R) residues along the length of the peptide facilitates delivery of the peptide across a cell membrane by condensing the negatively charged compound or nucleic acid through electrostatic interactions. The presence of arginine (R) enables nanoparticles less than 20 nm to form and ensures a positive zeta potential which enables internalisation into the cell. We have also found that the presence of arginine (R) residues also enhances nuclear localisation.

The ratio of the positively charged amino acid residues arginine (R) to negatively charged amino acid is also important because this is necessary to condense the payload into nanoparticles through electrostatic interactions. It is generally accepted that a nanoparticle less than <200 nm will be small enough to cross the cell membrane. In addition, the ratio of positively charged residues ensures an overall positively charged nanoparticle which has two main advantages. Firstly, that the particles will not aggregate and repel each other which aids in systemic delivery otherwise embolisms could occur. Secondly, as the cell membrane is negatively charged, nanoparticles that are either neutral or mildly positively charged will not enter the cell.

Finally, the peptide has a greater proportion of hydrophobic residues than hydrophilic residues (see table below) because this enables an amphipathic helical conformation and when the pH lowers in the endosome it is likely that RALA undergoes a conformational change to a mixture of alpha helix and random coil. This conformational change exposes the hydrophobic residues that can then fuse and destabilize the endosomal membrane enabling release to the cytosol. Having more hydrophobic residues increases the extent of membrane destabilisation.

| | | Polar/ | | |
|---|---|---|---|---|
| | ph7 | Non-Polar | Hydrophilic | Hydrophobic |
| R - Arg | Positive | Polar | Yes | |
| W - Trp | | Non-Polar | | Yes - very |
| E - Glu | Negative | Polar | Yes | |
| L- Leu | | Non-Polar | | Yes - very |
| A - Ala | | Non-Polar | | Yes - mildly |
| H - His | Positive | Polar | Yes | |
| C-Cys | Slightly Negative | Partially Polar | | Yes - mildly |

The peptide of the invention has improved cell penetration activity compared to, for example, KALA (see table below) for DNA delivery and conventional transfection reagents such as Oligofectamine® for siRNA delivery. Advantageously, the peptide of the invention is less toxic than another conventional transfection reagent such as, for example, Lipofectamine 2000®.

| NAME | AMINO ACID SEQUENCE |
|---|---|
| GALA (anionic) | WEAALAEALAEALAEHLAEALAEALEALAA |
| | (SEQ ID No. 9) |
| KALA (cationic) | WEAKLAKALAKALAKHLAKALAKALKACEA |
| | (SEQ ID No. 10) |
| RALA 16mer | RALARALARALRALAR |
| | (SEQ ID No. 11) |

According to a preferred embodiment of the invention, the arginine (R) residues are evenly distributed at every third and/or fourth amino acid position along the entire length of the peptide.

According to another preferred embodiment of the invention, the amount of hydrophilic amino acid residues in the peptide should not exceed approximately 40% or 37% and the ratio of hydrophilic amino acid residues to hydrophobic amino acid residues ratio at pH 7 is from 30:67 to 40:60, preferably 30:70 to 37:63.

According to another embodiment of the invention, the peptide comprises less than approximately 40 amino acid residues. Optionally, the peptide comprises 35, 34, 33, 32, 31, 30 amino acid residues, preferably 30, 29, 28, 27, 26, 25, 24 or 23 amino acid residues.

Ideally the amphipathic cell penetrating comprises at least 17, 18, 19, 20, 21, 22, 23, 24, 25, preferably at least 24 amino acids. According to a preferred embodiment, the peptide of the invention comprises at least 24 amino acids.

Ideally, the peptide comprises the consensus sequence EARLARALARALAR (SEQ ID No. 15).

Optionally, the peptide may comprise the consensus sequences EARLARALARALAR and/or LARALARALRA (SEQ ID No. 16) as highlighted in the preferred sequences according to the invention listed below:

(SEQ ID No. 1)
WEARLARALARALARHLARALARALRACEA
(SEQ ID No. 2)
WEARLARALARALARLARALARALRACEA
(SEQ ID No. 3)
WEARLARALARALARLARALARALRACEA
(SEQ ID No. 4)
WEARLARALARALARELARALARALRACEA
(SEQ ID No. 5)
REARLARALARALARLARALARALRACEA
(SEQ ID No. 6)
REARLARALARALARLARALARALRAREA
(SEQ ID No. 7)
REARLARALARALARELARALARALRAREA

Ideally, the present invention provides a peptide comprising the amino acid sequence

(SEQ ID No. 17)
X-EARLARALARALAR-Y-LARALARALRA-Z-EA

or a sequence at least 99%, 98%, 97%, 96%, 95%, 94%, 93%, 92%, 91%, 90%, 85%, 80% identical, wherein
X is W or R;
Y is optional and if present is selected from H or E; and
Z is C or R.

Preferably, the peptide comprises or consists of one of the following amino acid sequences:

(SEQ ID No. 1)
WEARLARALARALARHLARALARALRACEA
(SEQ ID No. 2)
WEARLARALARALARLARALARALRACEA
(SEQ ID No. 3)
WEARLARALARALARLARALARALRACEA
(SEQ ID No. 4)
WEARLARALARALARELARALARALRACEA
(SEQ ID No. 5)
REARLARALARALARLARALARALRACEA
(SEQ ID No. 6)
REARLARALARALARLARALARALRAREA
(SEQ ID No. 7)
REARLARALARALARELARALARALRAREA

or a fragment thereof. Ideally, the fragment comprises at least 23 amino acids from SEQ ID Nos. 1 to 7.

The table below provides further details of the several different examples amino acid sequences of preferred amphipathic cell penetrating peptides listed above.

| | | | % Ratio of | +/− |
|---|---|---|---|---|
| | | | Hydrophilic: | charged |
| | | | Hydrophobic: | amino |
| | SEQ | | Neutral amino | acid |
| Amino Acid Residue Sequence | ID No. | Length | acid residues | residues |
| “RALA” | 1 | 30mer | 30:67:1 | 8:2 |
| 1. WEARLARALARALARHLARALARALRACEA | | | | |
| H removed | 2 | 29mer | 31:70 | 7:2 |
| 2. WEARLARALARALARLARALARALRACEA | | | | |
| H replaced with glutamic acid (E) | 4 | 30mer | 33:67 | 7:3 |
| 3. WEARLARALARALARELARALARALRACEA | | | | |
| H Removed and W replaced with R | 5 | 29mer | 34:66 | 8:2 |
| 4. REARLARALARALARLARALARALRACEA | | | | |
| H Removed and W replaced with R and C replaced with R | 6 | 29mer | 37:63 | 9:2 |
| 5. REARLARALARALARLARALARALRAREA | | | | |
| H Replaced with E and W replaced with R and C replaced with R | 7 | 30mer | 40:60 | 9:3 |
| 6. REARLARALARALARELARALARALRAREA | | | | |
| 7. WEARLARALARALARHLRACEA (comparative peptide) | 8 | 22mer | | |

A most preferred sequence comprises/consists of the amino acid sequence WEARLARALARALARHLARALARALRACEA (herein referred to as “RALA”) (SEQ ID No. 1).

It will be understood, in this specification “RALA” is a generic term referring to the RALA sequence (SEQ ID No.1) or other similar sequences, including but not limited to SEQ ID Nos. 2 to 7, which also fall within the scope of the invention.

The invention also encompasses sequence with at least 90%, 95%, 96%, 97%, 98% or 99% sequence identity or sequence homology with SEQ ID Nos. 1 to 7.

Advantageously, the amphipathic cell penetrating peptides of the invention consists of arginine/alanine/leucine/alanine repeats that result in a specifically tailored hydrophobic and hydrophilic region facilitating interaction with the lipid bilayers enabling transport of the peptide across cellular membranes. As stated above, the presence of arginine (R) residues is an essential feature of the claimed peptide. There are two main advantages of using arginine. Firstly, arginine has consistently been shown to be the optimal amino acid for condensing DNA with arginine rich sequences binding in milliseconds. Secondly, arginine rich sequences based on the Rev sequence have the capacity to actively transport DNA into the nucleus of cells via the importin pathway.

In addition, there must be at least 2, but no more than 3, glutamate residues (E) to ensure pH-dependent solubility and protonation which facilitates endosomal disruption.

The present invention is also directed to modified peptides or peptide derivatives.

Optionally, the peptide according to any of the preceding claims is coupled or conjugated to a polyethylene glycol (PEG) molecule, such as RALA-PEG. Preferably, coupling takes place at the C-terminus of the peptide. The presence of the PEG molecule is advantageous because it increases circulation time of the peptide in vivo and provides for an enhanced permeation and retention effect of the peptide.

Alternatively, the peptide of the invention may comprise a cell targeting motif, preferably a motif which confers specificity to metastatic cell lines, conjugated to the N-terminus of the peptide through or via a spacer sequence. Ideally, the spacer sequence is an alpha helical spacer.

In this manner, the cell targeting motif may be the metastatic prostate cancer targeting peptide TMTP-1 (NVVRQ) and the spacer may be an alphahelical concatemeric spacer, preferably comprising 1 or more, preferably, 2, 3, or 4 repeats of the sequence EAAAK.

Advantageously, we have found that the claimed amphipathic cell penetrating peptide (RALA and similar sequences) or modified peptide/peptide derivative facilitates nuclear localisation. This gives the amphipathic cell penetrating peptide of the invention a distinct advantage over conventional non-viral and viral delivery systems. Surprisingly, the claimed amphipathic cell penetrating peptide has also been shown to form nanoparticles after 5 mins and be stable up to 48 hours at room temperature. The peptides of the present invention have been found to be stable as nanoparticles up to 5, 6 and 15 days after delivery.

Advantageously, we have found that the claimed amphipathic cell penetrating peptide can create nanoparticles with a size less than 150 nm or even 100 nm with nucleic acids or other agents. This facilitates transport of these agents across cell membranes, out of the endosomes and to the nucleus. We have found that these nanoparticles are stable in serum and over a temperature range of 4 to 37° C. These nanoparticles may be used as cell delivery systems themselves or together with a microprotrusion array of the invention for delivery of nucleic acids or other agents across cell membranes and/or nuclear localisation.

Accordingly, the peptide as defined above presents a viable alternative in the field of gene delivery and may be used as a transfection agent for siRNA.

According to this embodiment, the claimed amphipathic cell penetrating peptide may also be used in DNA gene therapy. Confocal imaging has clearly shown delivery of Cy3 labelled DNA to the nucleus of prostate cancer cells. This provides the opportunity for the delivery of any nucleic acid to a cell in vivo, in which the nucleic acid may be utilised for gene therapy.

The nucleic acid may encode a functional, therapeutic gene to replace a mutated gene. Alternatively, the nucleic acid may correct a mutation or encodes a therapeutic protein drug. In this manner, the nanoparticles of the invention may be used as adjuvant gene therapy treatment administered optionally prior to conventional treatments.

According to another embodiment of the invention, the nucleic acid may be DNA in the form of an iNOS (inducible nitric oxide synthase) plasmid DNA under control of a tumour specific promoter. The iNOS plasmid DNA may be condensed with or complexed with the peptide of the invention to form nanoparticles and delivered as nanoparticles in-vivo. This results in the inducible production of nitric oxide in-vivo which is detrimental to tumour metastasis. In this manner, the nanoparticles or cell delivery system of the invention comprise the claimed amphipathic cell penetrating peptide and iNOS plasmid DNA.

According to one embodiment the tumour specific promoter is the human osteocalcin (hOC) promoter. It will be understood that the hOC promoter is specific to ovarian, breast and prostate cancers and although the peptide of the invention will deliver to all tissues the use of this promoter will ensure transcriptional targeting and expression of the desired gene only in the tumours. However, other known promoters may be used which will be dependent on differential expression in tumour tissue. Examples include the osteopontin promoter known to be overexpressed in breast cancer, the prostate specific membrane antigen promoter for prostate cancer or radiation inducible promoters such as WAF1 or CARG. Both WAF1 and CARG have the added advantage of also being activated in hypoxic regions such as those found in the centre of tumours.

According to another embodiment of the invention, the tumour specific promoter may be a prostate specific promoter, such as the prostate membrane specific antigen promoter (PSMA).

The amphipathic cell penetrating peptide of the invention may also be used to deliver hOC-iNOS (inducible nitric oxide synthase) systemically in vivo to any tumour model that has been shown to metastasise to bone. In this manner iNOS plasmid DNA may be condensed with the peptide of the invention and delivered as nanoparticles in-vivo. Advantageously, the RALA/hOC-iNOS nanoparticles may be administered in tandem with the current recommended chemotherapy regimen of docetaxel. For those with bone metastases docetaxel remains the standard front-line treatment but increasingly many patients develop resistance to this drug. This new combination therapy provides an alternative strategy for treating bone metastases.

Alternatively, promoters specific for cardiovasculature may be used to increase the levels of iNOS to dilate blood vessels. One potential administration method includes the application of the nanoparticles as a coating for stents. A major unresolved issue following percutaneous transluminal coronary angioplasty (PTCA) is the physical injury to the blood vessel wall, which leads to vessel re-occlusion, i.e. restenosis. The endothelial denudation associated with this injury is accompanied by varying degrees of medial disruption and is followed by an inappropriate response-to-injury of vascular smooth muscle. Therefore using smooth muscle cell (SMC) (e.g. SM22 alpha promoters) promoters to drive expression of the iNOS transgene will confer tissue specific targeting at the site of injury either with or without stents.

The material of the invention, may comprise nanoparticles formed from other agents, preferably small molecule agents complexed with the amphipathic cell penetrating peptide. For example, the agents may comprise proteins or a therapeutic agent or drug.

These include but are not limited to any phosphate or lipophilic based drug, preferably a bisphosphonate drug and gold. Bisphosphonate drugs are characterised by a very low bioavailability, rapid excretion from the body, harsh side effects and poor patient compliance. Improving upon the delivery of this drug to where it is needed there provides a significant impact on patient health. As a lipophilic drug, bisphosphonates cannot cross the cell membrane to effect the therapy. Therefore there is a need for an effective delivery system to encapsulate the bisphosphonates and improve cellular entry and bioavailability in vivo.

The therapeutic agent may be a phosphate based drug, preferably a bisphosphonate drug including alendronate, etidronate, zolendrate or any other nitrogen or non-nitrogen based bisphosphonate drug. Bisphosphonate drugs have low bioavailability which can advantageously be enhanced when complexed with the peptide of the present invention. The cell delivery system of the invention will improve the bioavailability of a phosphate based drug, preferably a bisphosphonate drug. The cell penetrating peptide of the invention may be used for the condensation and delivery of the nitrogen bisphosphonate, Alendronate. N-BP nanoparticles were formed with sizes less than 100 nm and an overall positive charge facilitating cellular entry. The alendronate nanoparticles were spherical, uniform and did not aggregate as evidenced by TEM. More importantly, when the alendronate loaded nanoparticles were added to prostate cancer cells in vitro there was significantly greater cytotoxicity at lower concentrations compared to the alendronate only treated cells. Thus, the delivery system of the invention provides significant promise for improving the delivery and bioavailability of bisphosphonates patients with osteoporosis and cancer.

An alternative use involves the improvement of the delivery of gold particles. The effectiveness of many radiotherapy treatment plans are limited by normal tissue toxicity. Using gold nanoparticles (GNPs) can increase the therapeutic benefit by radiosensitising cancer cells. However, whenever these gold nanoparticles are delivered the majority remain trapped within the endosome creating an inhomogeneous distribution and limiting their full potential. We have found that when the GNPs are wrapped with the peptide of the invention there is a significant increase in endosomal escape which facilitates a marked increase in therapeutic efficacy.

According to another aspect of the invention, there is provided a transdermal patch comprising the cell delivery system according to the invention and a pharmaceutically acceptable excipient. In this manner the cell delivery system of the invention is adapted for transdermal delivery.

According to another important aspect of the invention there is provided freeze-dried or spray-dried nanoparticles. Standard/conventional freeze-drying and spray-drying techniques may be used. We have found that these nanoparticles are stable after lyophilisation with no reduction in transfection efficacy. They remain as discrete nanoparticles when reconstituted or rehydrated. Advantageously, one or more of water or trehalose may be used to reconstitute the freeze-dried or spray-dried nanoparticles. We have also found that PVP alone or in combination with trehalose may be used to reconstitute the freeze-dried or spray-dried nanoparticles. One the advantages of freeze-drying the nanoparticles is that significantly more nanoparticles can be loaded into the microprotrusion array/microneedles. We have found that up to 500 μg of nanoparticles per 1×1 cm patch can be loaded into the microneedles. Ideally, 100 μg of nanoparticles per 1×1 cm patch can be loaded into the microneedles.

The present invention will now be described with reference to the following non-limiting figures and examples.

FIG. 1: The nuclear localisation signal (NLS) dependent nuclear import of plasmid DNA is shown schematically. The arginine rich NLS recognises importin (IMP)-α protein. IMP-β binds the importin-β binding (IBB) domain of IMP-α to form the IMP-α/β heterodimer. Once docked to the nuclear pore complex through IMP-β, it binds to specific nucleoporins (Nups) on the cytoplasmic side of the NPC. The translocation of the importin/cargo complex through the NPC involves transient association/disassociation interactions of IMP-β with the phe-gly (F-G) repeats of Nups throughout the NPC central channel.

FIG. 2: Transmission electron microscope image highlighting that RALA can also condense siRNA to form spherical nanoparticles formed at N:P 12. Particles were accelerated at a voltage of 80 kV and viewed at a magnification of 40,000×.

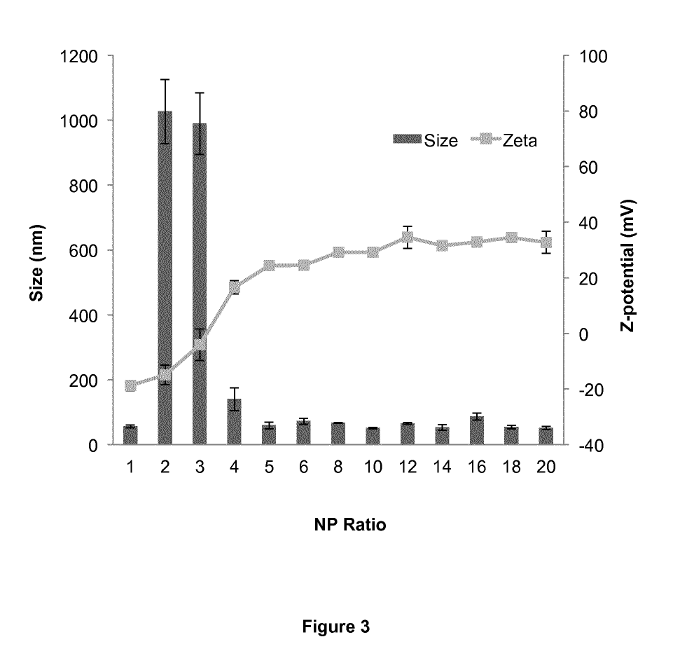

FIG. 3: Nanoparticle size and charge analysis of RALA/GFP with sizes less than 150 nm enabling transport across the cellular membrane. A positive charge of 20-30 mV indicates that the nanoparticles are stable. N:P ratio indicates the ratio of Peptide RALA (N) to pEGFP DNA (P). Data is the mean of three experiments+/−S.E.

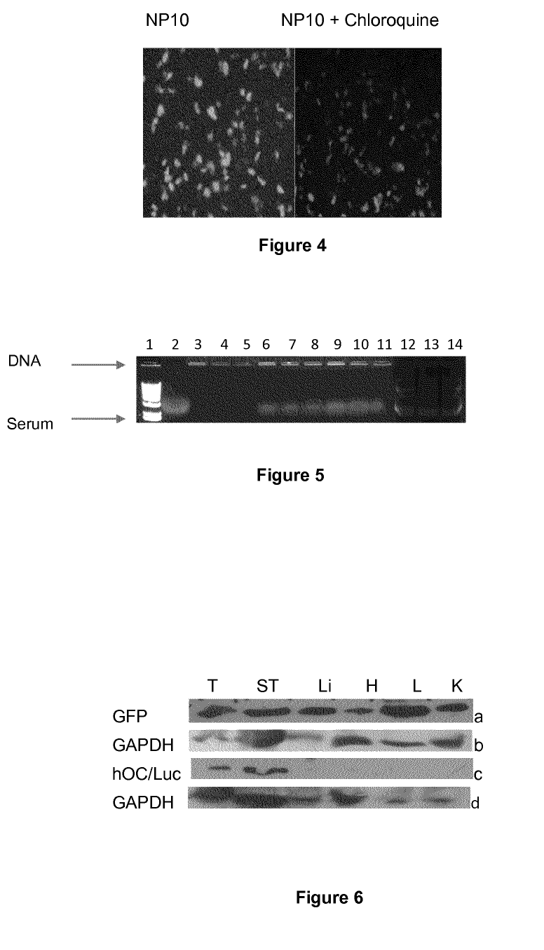

FIG. 4: Evaluation of the transfection efficiency of RALA/GFP nanoparticles in ZR-75-1 (breast cancer) cells transfected N:P ratio of 10. Chloroquine is a known endosomal disruption agent and transfection is not improved upon the addition of this agent indicating effective endosomal disruption with the RALA vector.

FIG. 5: Incubation of RALA/GFP nanoparticles in 10% serum for 30 mins. Particles remain complexed. 1—ladder, 2—serum only, 3-5 no serum, 6-8 5% serum, 9-11 10% serum, 12-14 Sodium Dodecyl Sulphate. SDS is used to de-complex the nanoparticles. Note that the nanoparticles remain condensed after incubation in 10% serum indicating stability.

FIG. 6: Western blots showing expression of GFP or luciferase in organs from a SCID mouse bearing a ZR-75 tumour. RALA/GFP or RALA/hOC-Luc nanoparticles N:P ratio of 10 were injected i.v. and 48 hours later the organs were excised and protein extracted. T-tumour, S.T.—surrounding tissue, Li—Liver, H—Heart, Lu—Lungs, K—Kidney. A total of 10 μg of DNA was delivered. Note this is 2.5 times less DNA than previous experiments using lipofectamine delivery injected i.t.

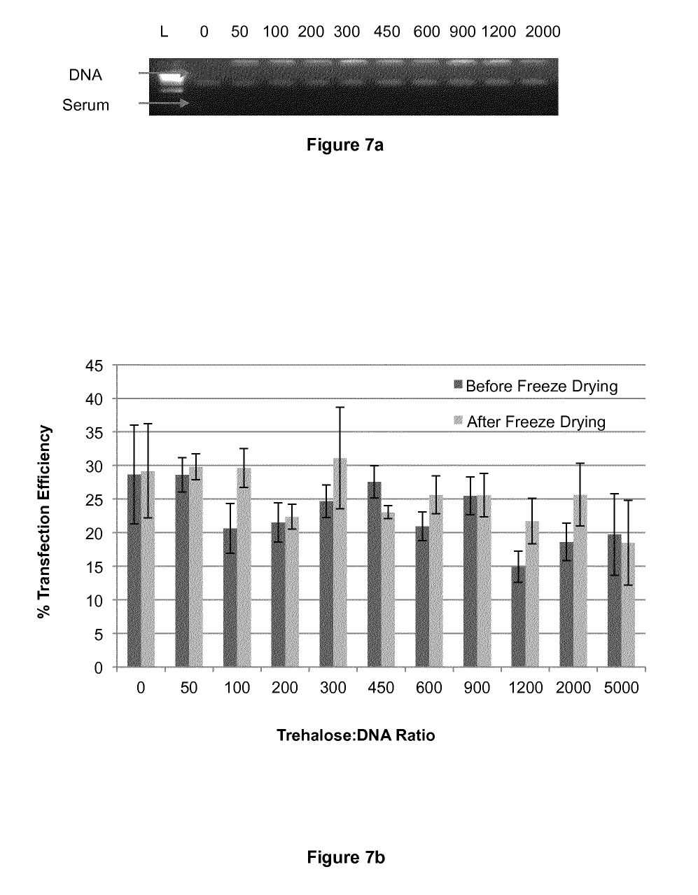

FIG. 7a: Incubation of RALA/GFP nanoparticles (N:P 10) in 10% serum and 1% SDS for one hour at 37° C. following freeze drying with the cryoprotectant trehalose. The numbers indicate the ratio of trehalose:DNA. 0 is serum only. The nanoparticles remain condensed after incubation in serum post freeze drying at all trehalose concentrations.

FIG. 7b: Transfection efficiencies of RALA/GFP nanoparticles (N:P 10) before freeze drying (fresh) and after (reconstituted) with trehalose. Transfection efficiency was measured via FACS analysis. Data is the mean of four experiments+/−S.E.

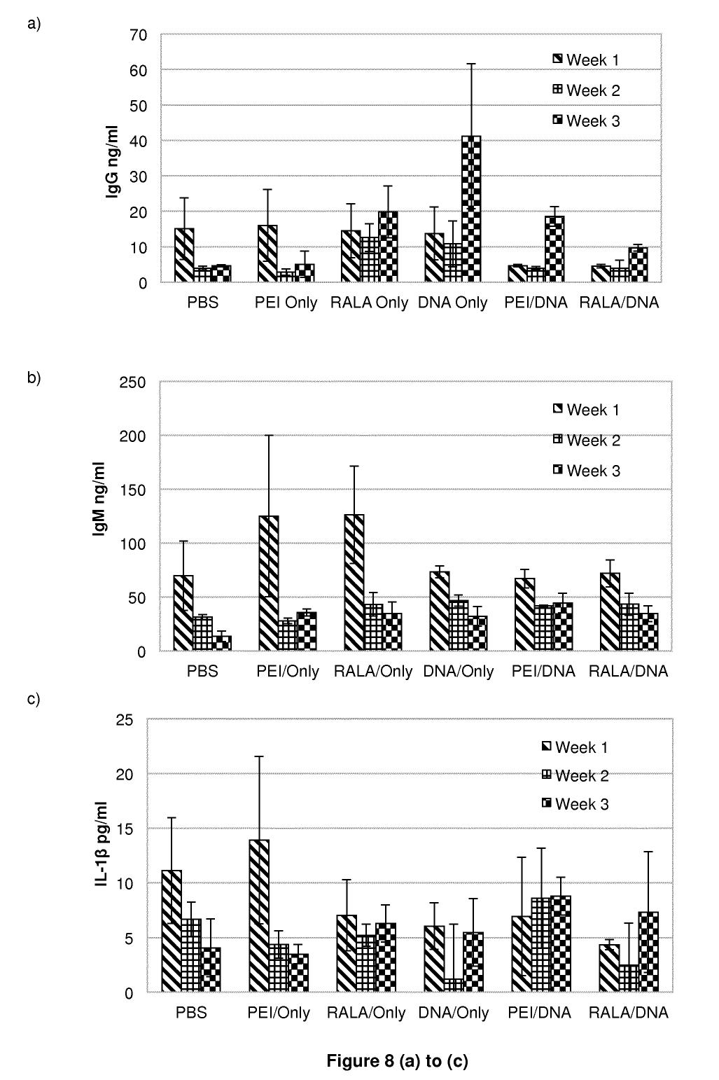

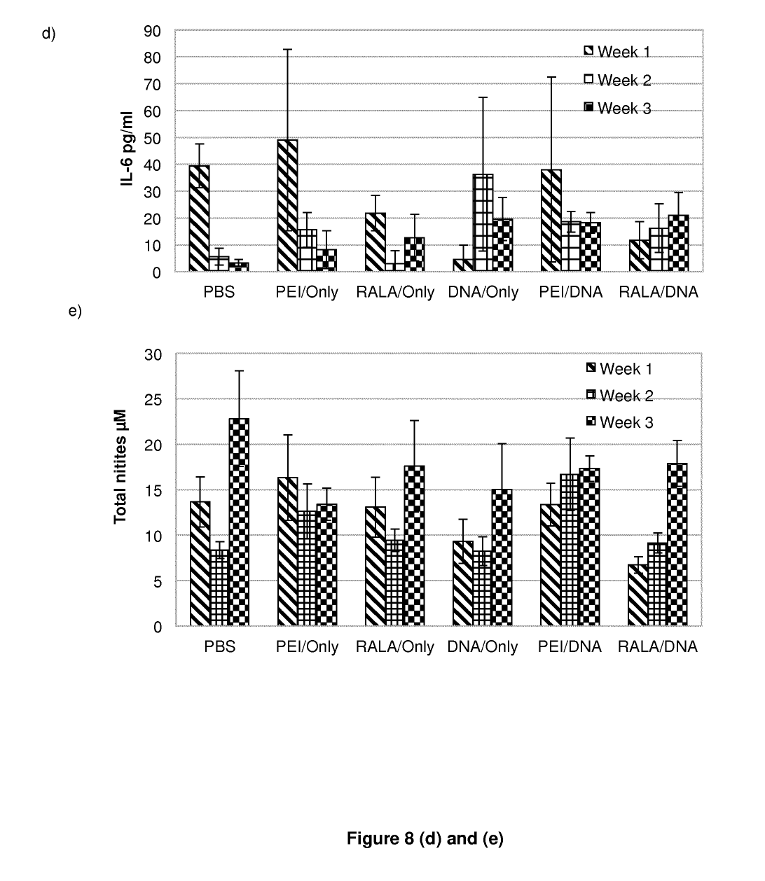

FIG. 8: Immune response of C57BL/6 mice injected with PBS only, PEI only, RALA only, DNA only, PEI/DNA or RALA/DNA nanoparticles. For each injection the equivalent to N:P 10 with 10 μg of DNA was injected. Mice received one injection per week for three weeks. 48 h after each injection three mice were sacrificed and the serum was extracted for analysis. a) IgG, b) IgM, c) IL-1β, d) IL-6 and e) Greiss test for total nitrites. Each data point is the mean of three independent mice sera+/−S.E. For each of the tests the RALA/DNA nanoparticles do not induce a significant immune response.

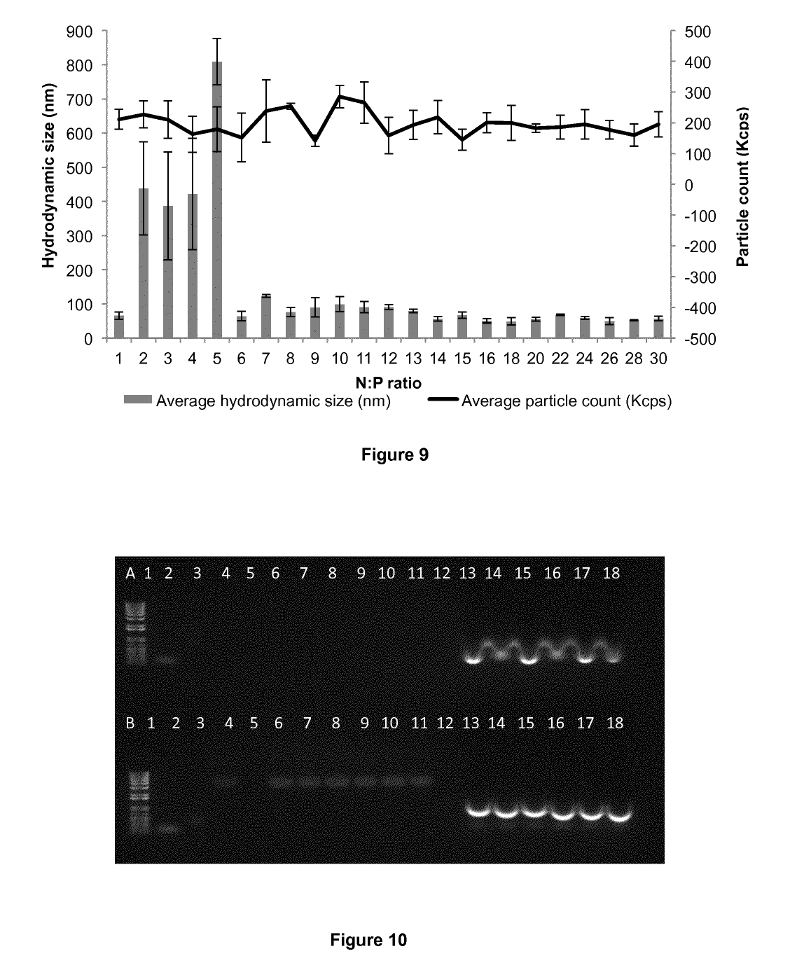

FIG. 9: Characterisation of particles using Malvern Zetasizer. Hydrodynamic size of the RALA/RUNX2 siRNA nanoparticles and their corresponding particle count over a range of N:P ratios. Particle count is fairly consistent and within the ideal range of 100-500 nm. From N:P 6 onwards sizes are consistently less than 150 nm which is within the desired boundary for successful delivery to cells.

FIG. 10: 1% agarose gel illustrating the stability of the RALA/RUNX2 siRNA nanoparticles at N:P ratio 12+/−serum. Nanoparticles were decomplexed using Sodium Dodecyl Sulphate to confirm integrity of siRNA. A: Lane 1: 1 Kb plus ladder Lane 2: RUNX2 siRNA only Lane 3: SDS only Lanes 6-11: RALA/RUNX2 siRNA nanoparticles incubated at 37° C. for 1-6 hours respectively Lanes 13-18: RALA/RUNX2 siRNA nanoparticles incubated at 37° C. for 1-6 hours respectively and decomplexed with SDS for 10 minutes B: Lane 1: 1 Kb plus ladder Lane 2: RUNX2 siRNA only Lane 3: SDS only Lane 4: Foetal calf serum only Lane 6-11: RALA/RUNX2 siRNA nanoparticles incubated in foetal calf serum at 37° C. for 1-6 hours respectively Lane 13-18: RALA/RUNX2 siRNA nanoparticles incubated in foetal calf serum at 37° C. for 1-6 hours respectively and decomplexed with SDS for 10 minutes.

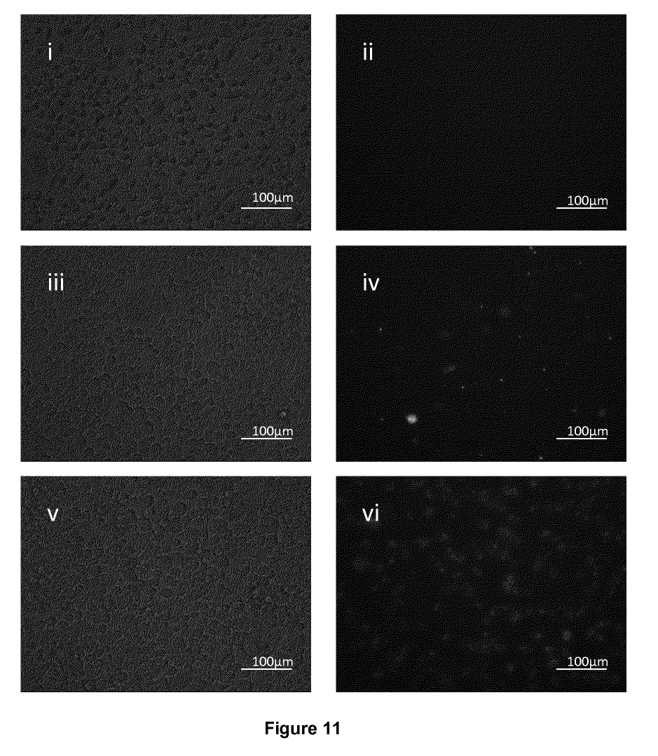

FIG. 11: PC3 cell line was transfected for 4 hours with RALA/control siRNA nanoparticles N:P 12 containing 0.125 ug siRNA, RALA only equivalent to N:P 12 and oligofectamine/control siRNA complexes and imaged immediately after transfection. (i): Light image of cells transfected with RALA only. (ii): Fluorescent image of cells transfected with RALA only. (iii): Light image of cells transfected with oligofectamine based complexes. (iv): Fluorescent image of cells transfected with oligofectamine based complexes. (v): Light image of cells transfected with RALA based nanoparticles. (vi): Fluorescent image of cells transfected with RALA based nanoparticles. Fluorescence was much greater following transfection with RALA/control siRNA compared to the fluorescence seen with oligofectamine/control siRNA under identical conditions.

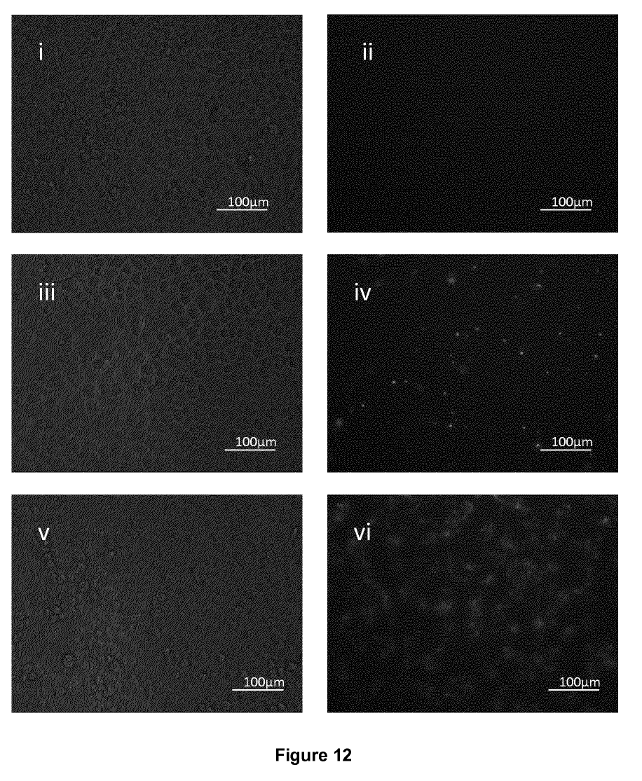

FIG. 12 MDA-MB-231 cell line was transfected for 4 hours with RALA/control siRNA nanoparticles at N:P 12 containing 0.125 μg siRNA, RALA only, equivalent to N:P 12, and oligofectamine/control siRNA complexes and imaged immediately after transfection. (i): Light image of cells transfected with RALA only. (ii): Fluorescent image of cells transfected with RALA only. (iii): Light image of cells transfected with oligofectamine based complexes. (iv): Fluorescent image of cells transfected with oligofectamine based complexes. (v): Light image of cells transfected with RALA based nanoparticles. (vi): Fluorescent image of cells transfected with RALA based nanoparticles. Fluorescence was much greater following transfection with RALA/control siRNA compared to the fluorescence seen with oligofectamine/control siRNA under identical conditions.

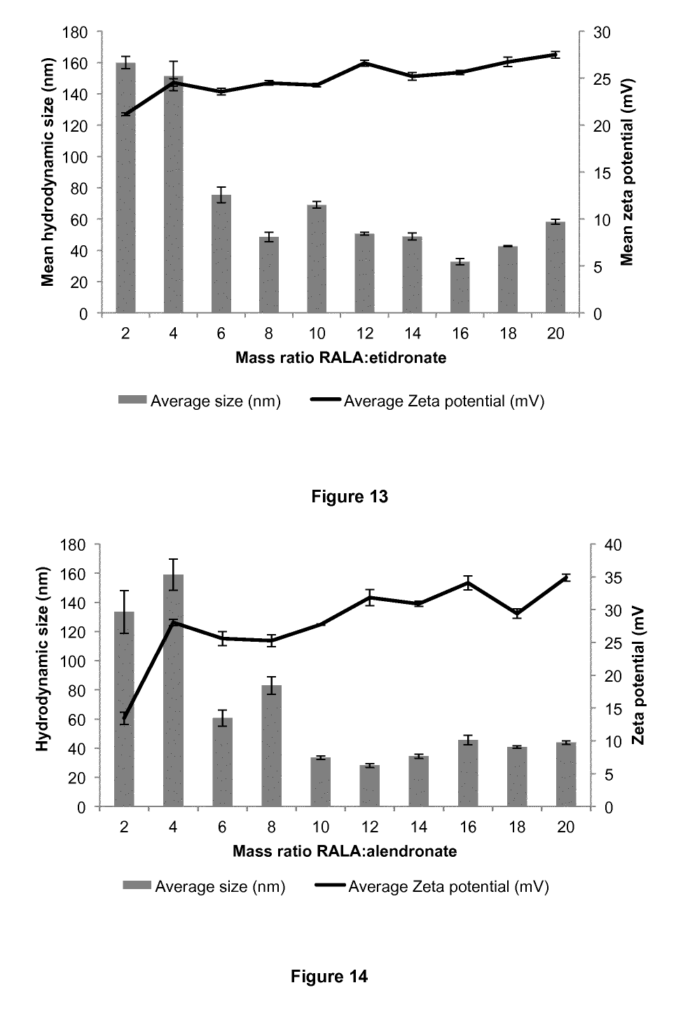

FIG. 13: Characterisation of RALA/etidronate nanoparticles using Malvern Zetasizer Hydrodynamic size of the RALA/etidronate nanoparticles and their corresponding zeta potential over a range of mass ratios. Particles are consistently less than the 150 nm boundary preferred to maximise transfection efficiency with a ratio of 10 producing the optimal hydrodynamic size.

FIG. 14 Nanoparticle size and charge analysis of RALA/Alendronate with sizes less than 150 nm enabling transport across the cellular membrane. A positive charge of 20-30 mV indicates that the nanoparticles are positively charged and will enter cells. Data is the mean of three experiments+/−S.E.

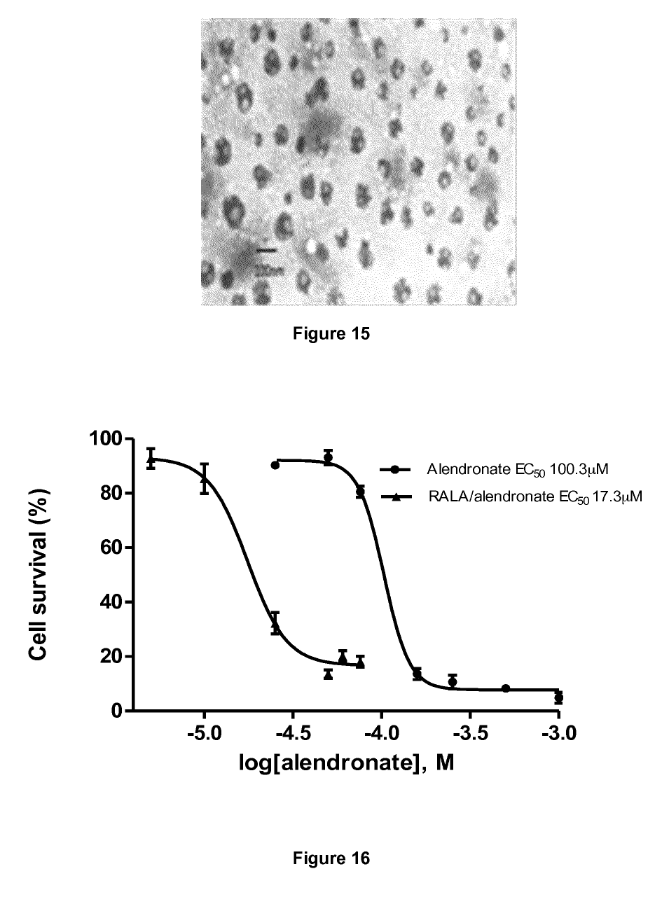

FIG. 15: Transmission electron microscope image highlighting that RALA can condense Alendronate to form spherical nanoparticles at N:P 32. Particles were stained with uranyl acetate at room temperature for 10 mins and accelerated at a voltage of 80 kV. 40,000×.

FIG. 16: Dose response curve based on manual cell counts using a haemocytometer. Cells were transfected with RALA/alendronate nanoparticles or treated with alendronate only at a range of concentrations between 10 μM and 250 μM for six hours and allowed to recover for 72 hours before analysis. The untreated control was taken as being 100% cell viability and percentage growth inhibition was determined based on this. The EC50 for alendronate only is 97.9 μM and for RALA/alendronate nanoparticles it is 14.3 μM. Data is the mean of three independent experiments+/−S.E.

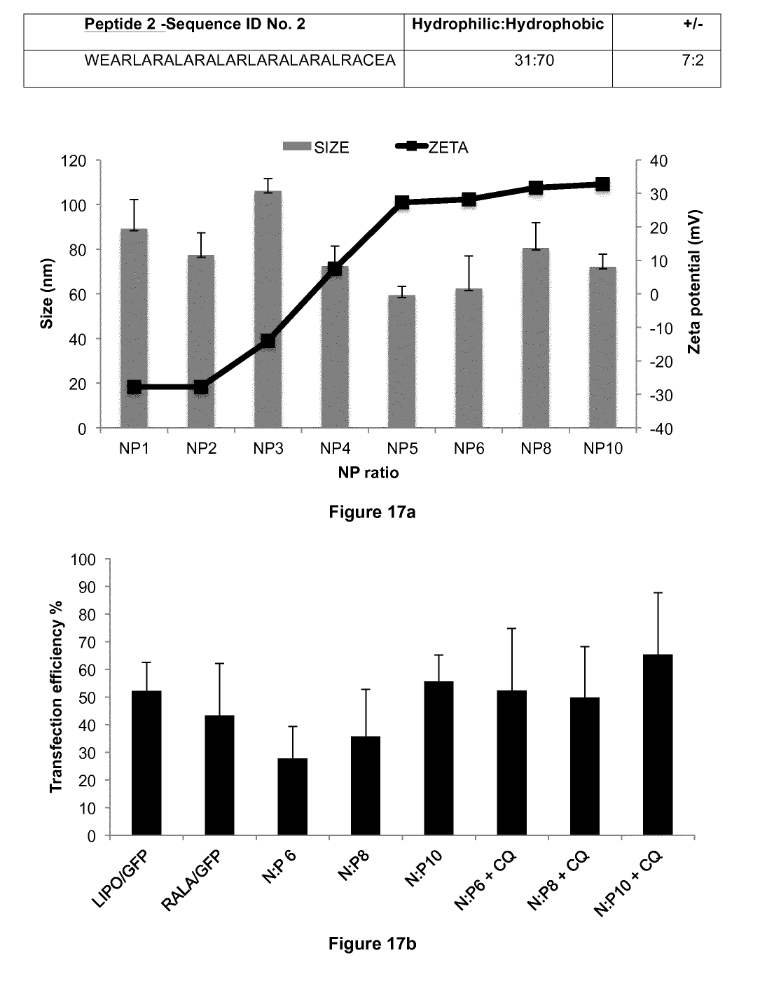

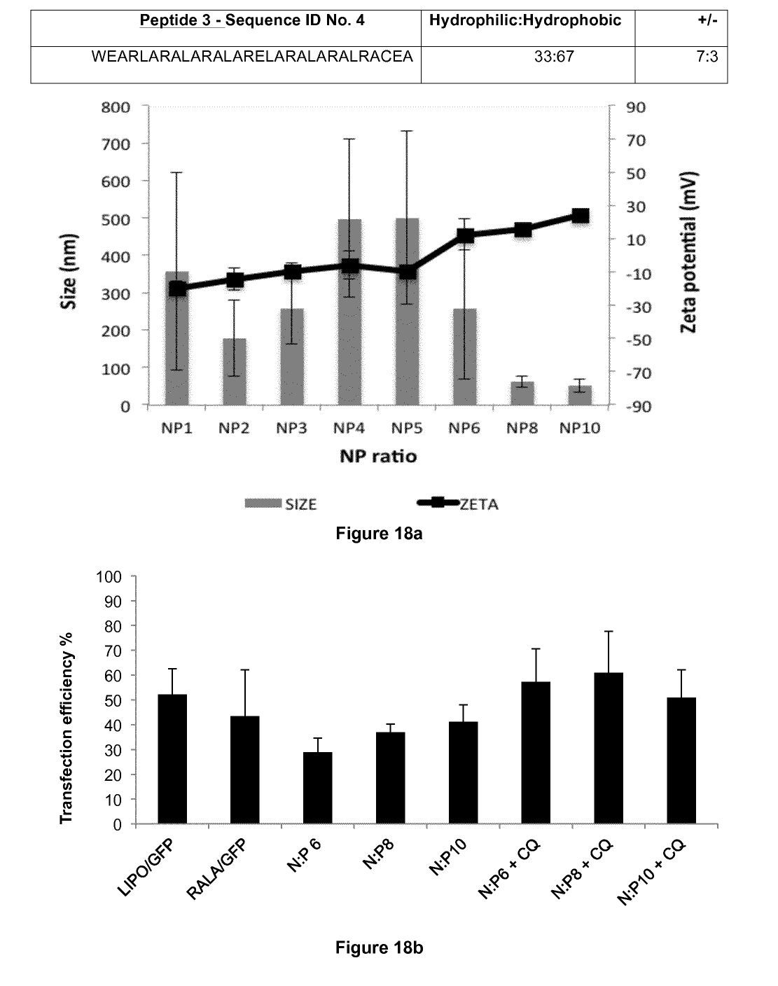

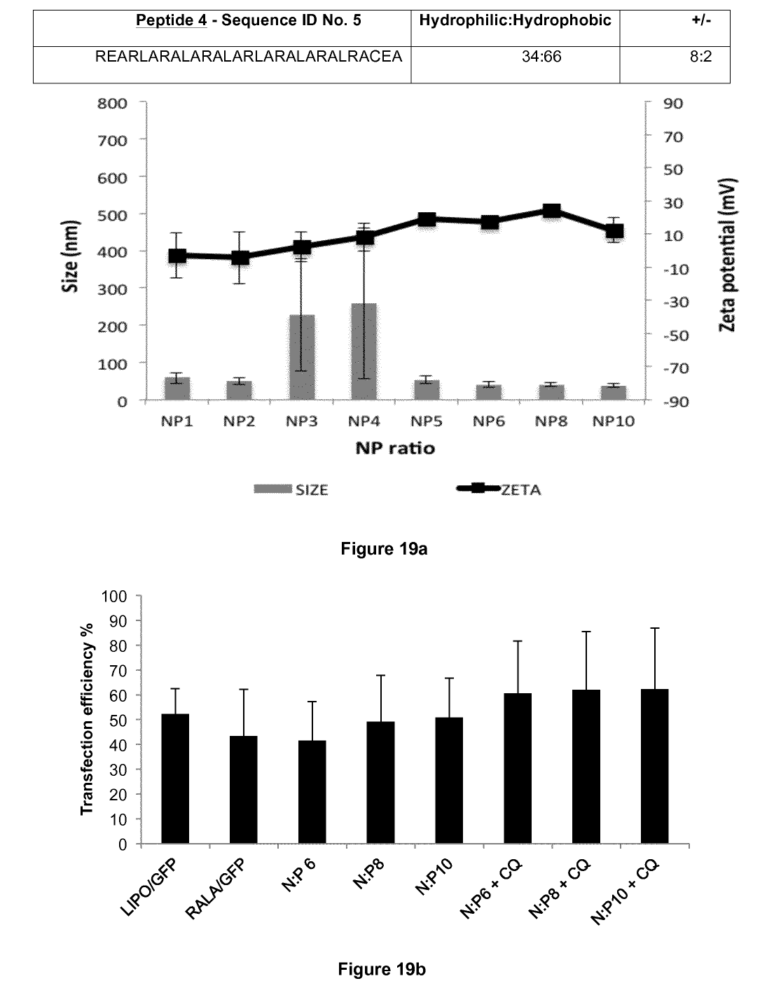

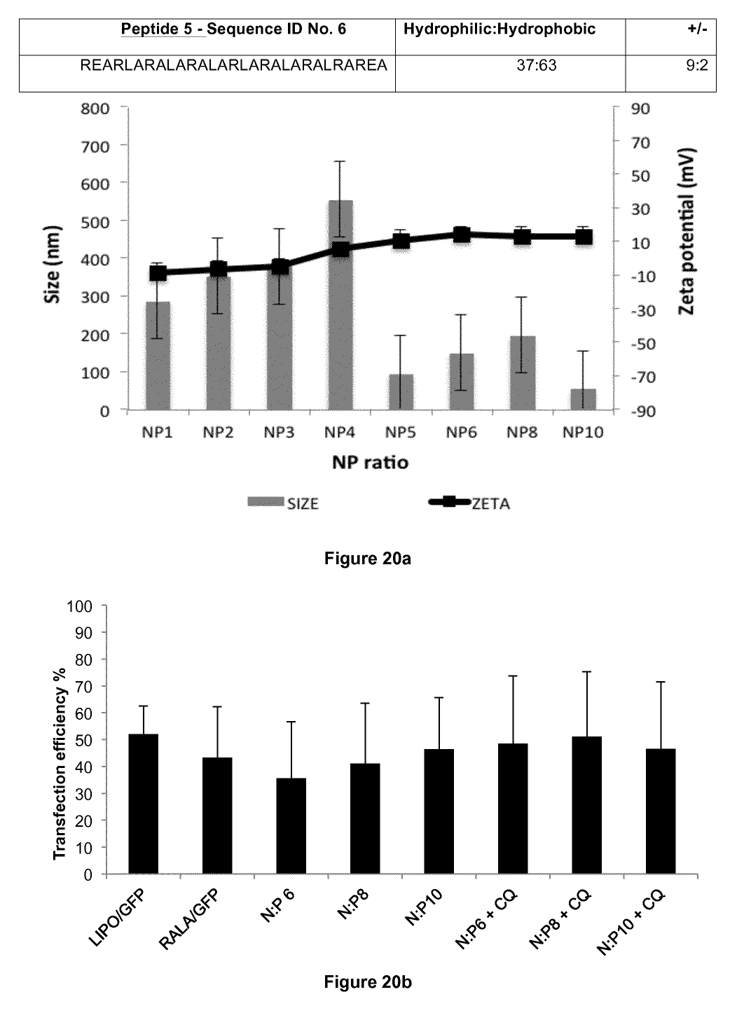

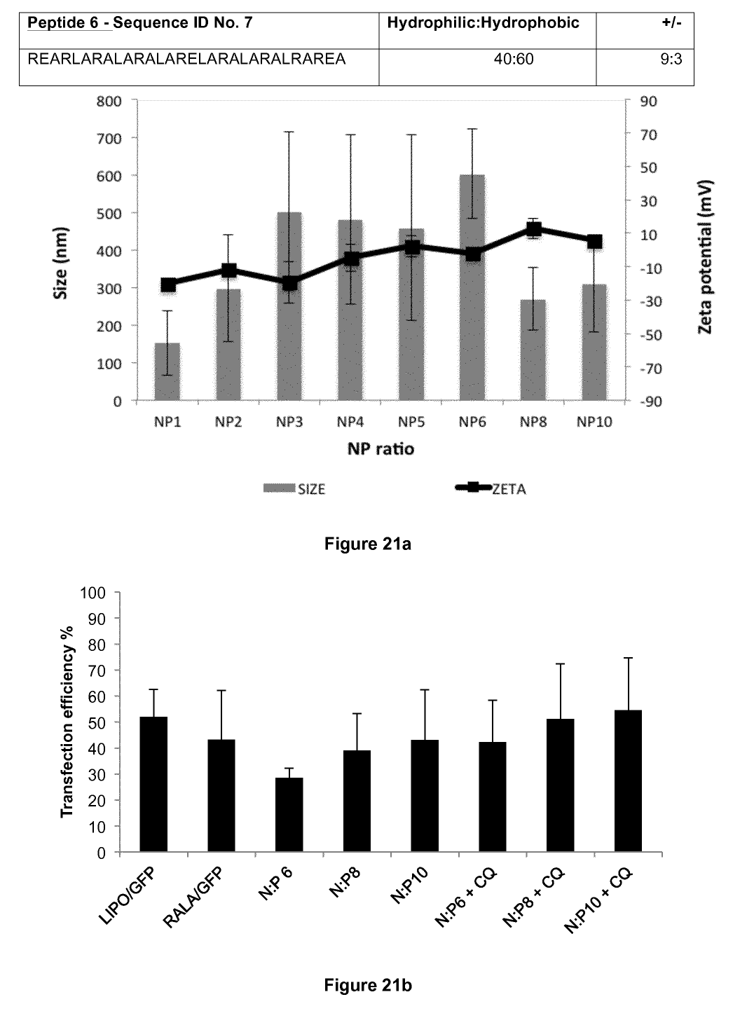

FIGS. 17 to 21 (a) and (b): Nanoparticle size and charge analysis of RALA peptide derivatives with sizes less than 150 nm enabling transport across the cellular membrane. A positive charge of ˜10 mV indicates that the nanoparticles are positively charged and will enter cells. Transfection efficiencies of Peptides 2-6 nanoparticles (N:P 8-10) in PC-3 prostate cancer cells. Lipofectamine/GFP and RALA/GFP were controls. Transfections were also performed in the presence of chloroquine to assess endosomal disruption. Transfection efficiency was measured via FACS analysis. Data is the mean of three independent experiments+/−S.E.

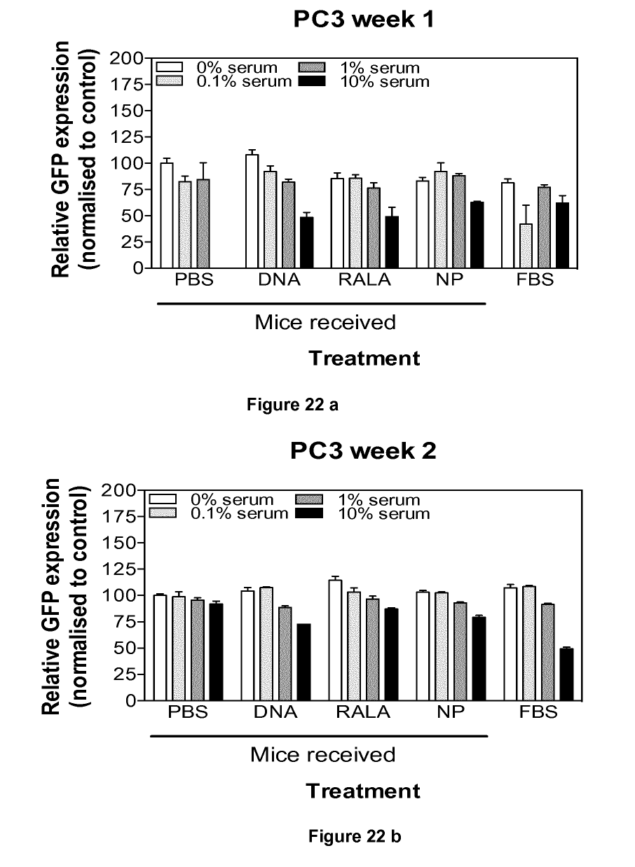

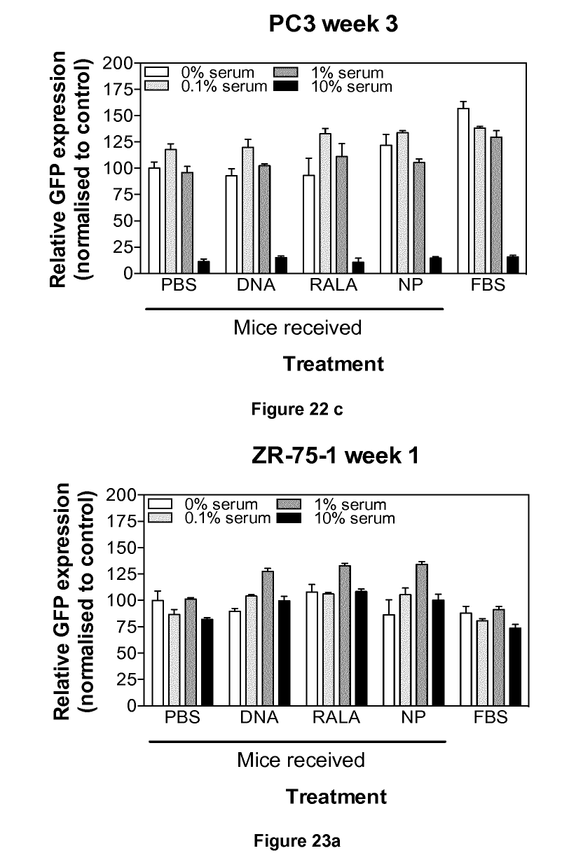

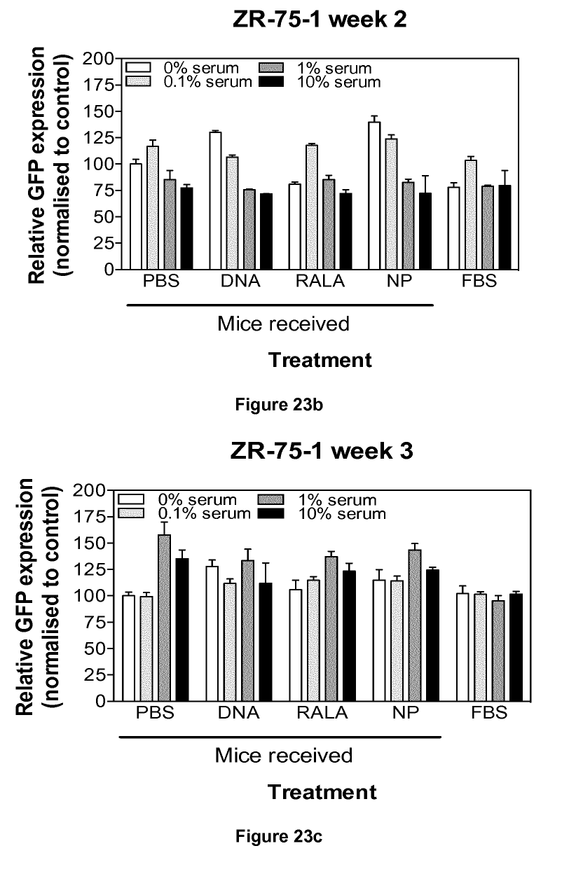

FIGS. 22 and 23: Vector Neutralisation assay was performed to ensure that RALA/pEGFP nanoparticles are not subject to neutralisation by host immune system in FIG. 22 PC-3 prostate cancer cells from weeks 1 to 2 and FIG. 23 ZR-75-1 breast cancer cells from weeks 1 to 2. C57/BL6 mice received either one/two/three intravenous injection treatments of PBS/DNA/RALA/pEGFP-N1-RALA nanoparticles, were sacrificed, blood isolated, heat-inactivated, incubated with fresh pEGFP-RALA nanoparticles and PC3 and ZR-75-1 cells were transfected. Transfection was normalised to controls and quantified using FACS analysis with 4% gating. Data are the mean of three independent experiments+/−S.E.

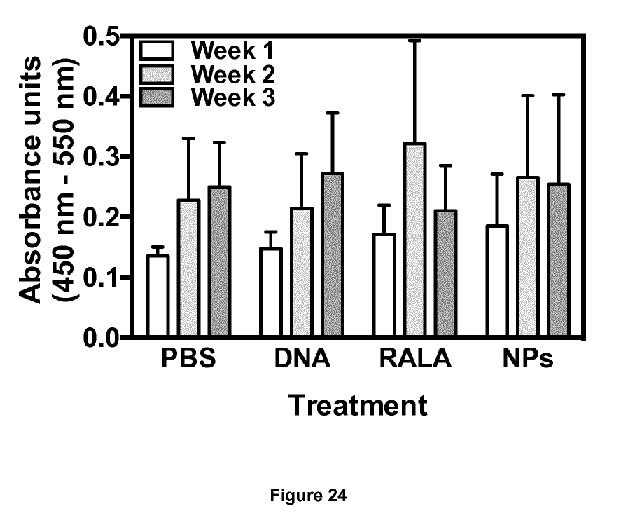

FIG. 24: C57/BL6 mice received PBS, 10 μg pEGFP-N1, 14.5 μg RALA, or nanoparticles equivalent to 10 μg pEGFP-N1 complexed at N:P 10 with 14.5 μg RALA. To assess anti-RALA antibody content of mouse serum, the sera used in vector neutralization assays were analysed in ELISA studies. 96 well ELISA plates were coated with pEGFP-N1/RALA nanoparticles as the presentation antigen in PBS overnight at 4° C. Wells were washed with PBS, and non-specific binding to antigen was minimized by blocking with PBS/bovine serum albumin for 1 h at room temperature. The wells were probed with mouse sera (1:100 dilution) for 1 h at room temperature, followed by three washes with ELISA wash buffer. Wells were probed with an anti mouse secondary antibody conjugated to streptavidin. Following three further washes, the ELISA will be completed by addition of the substrate (TMB), completion of the reaction, and quantification of the colorimetry using an ELISA plate reader.

No immunoreactivity was observed compared to the controls. Data are the mean of three independent experiments+/−S.E.

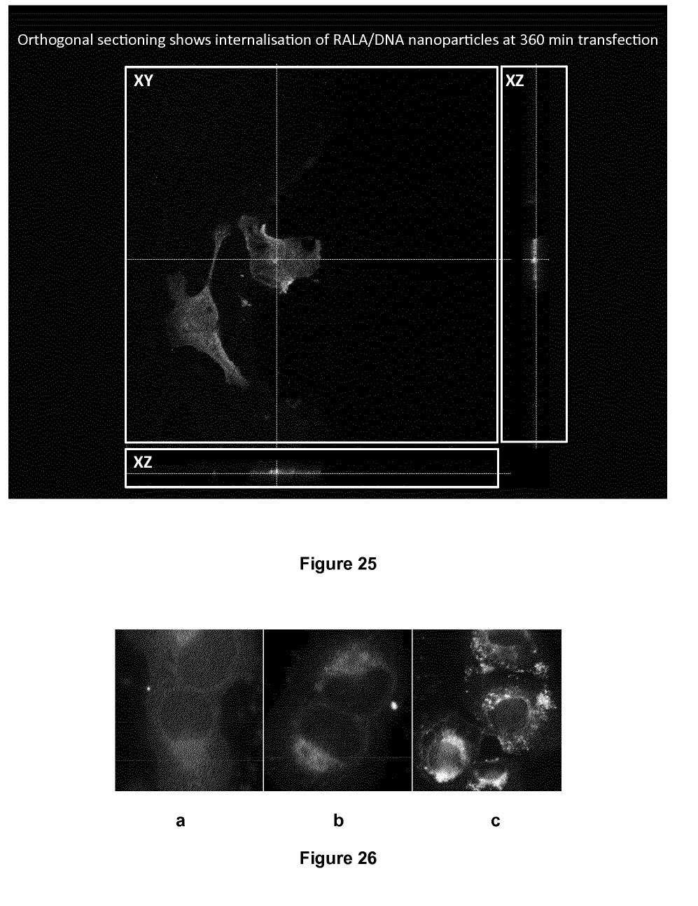

FIG. 25: Representative confocal image of Intranuclear Cy3-DNA/RALA nanoparticles following 360 min transfection in ZR-75-1. Orthogonal sectioning of Z slice at 5.2 μm. In the image, the nucleus appears blue, and Cy3-DNA/RALA nanoparticles appear red. The positioning of the crosshairs was set at a position of interest (in this case an area of intense red staining) in the XY image; the confocal software subsequently generates corresponding XZ and YZ images, allowing for accurate determination of subcellular nanoparticle location.

FIG. 26: Cytoviva—Hyperspectral scanned images of MDA-MB-231 cells. A) Untreated control cells. B) 5 nm phosphorylated Gold Nanoparticles. C) 5 nm phosphorylated RALA wrapped Gold Nanoparticles.

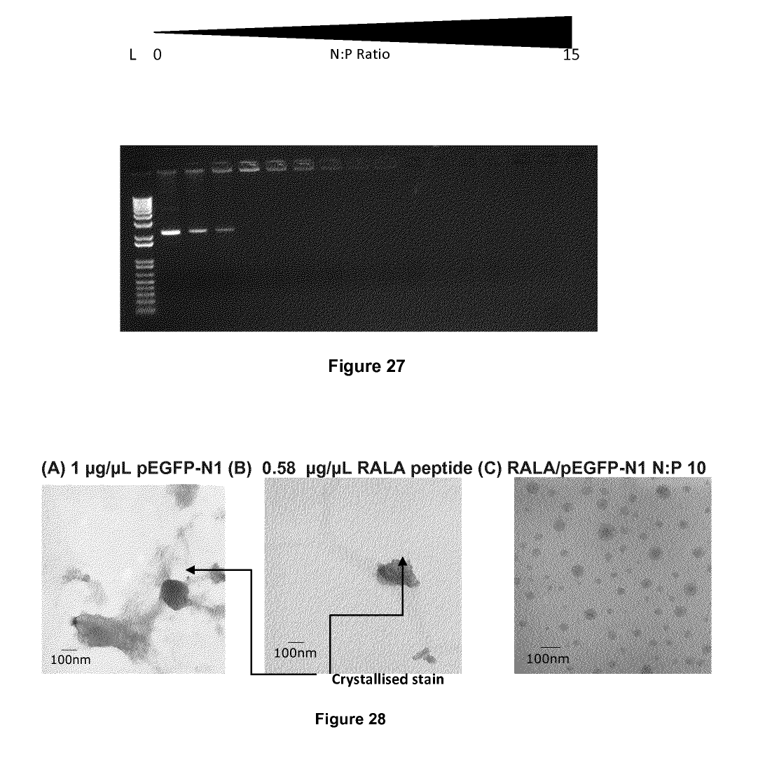

FIG. 27: Gel retardation assay of RALA/pORF-mIL4 nanoparticles over a range of N:P ratios (0-15). RALA/pORF-mIL4 complexes were prepared at N:P ratios 0-15 and incubated at room temperature for 30 minutes. Following incubation 30 μL of samples were electrophoresed through a 1% agarose gel containing 0.5 μg/mL ethidium bromide to visualize DNA. A current of 80 V was applied for 1 hour and the gel imaged. L=1 Kb Plus DNA Ladder (Invitrogen, UK). Gel images are representative of three independent studies.

FIG. 28: TEM images of air dried aqueous uranyl acetate (5%) stained Formvar/Carbon mesh grid loaded with (i) 1 μg/μL pEGFP-N1, (ii) 0.58 μg/μL RALA peptide and (iii) RALA/pEGFP-N1 N:P 10 nanoparticles. Three Formvar/Carbon mesh grids were loaded with 10 μL of each sample and left to dry overnight. The grids were then stained for 5 minutes with 5% aqueous uranyl acetate at room temperature and imaged immediately following staining. The grids were imaged using a JEOL 100CXII transmission electron microscope at an accelerating voltage of 80 kV and magnification 50,000×.

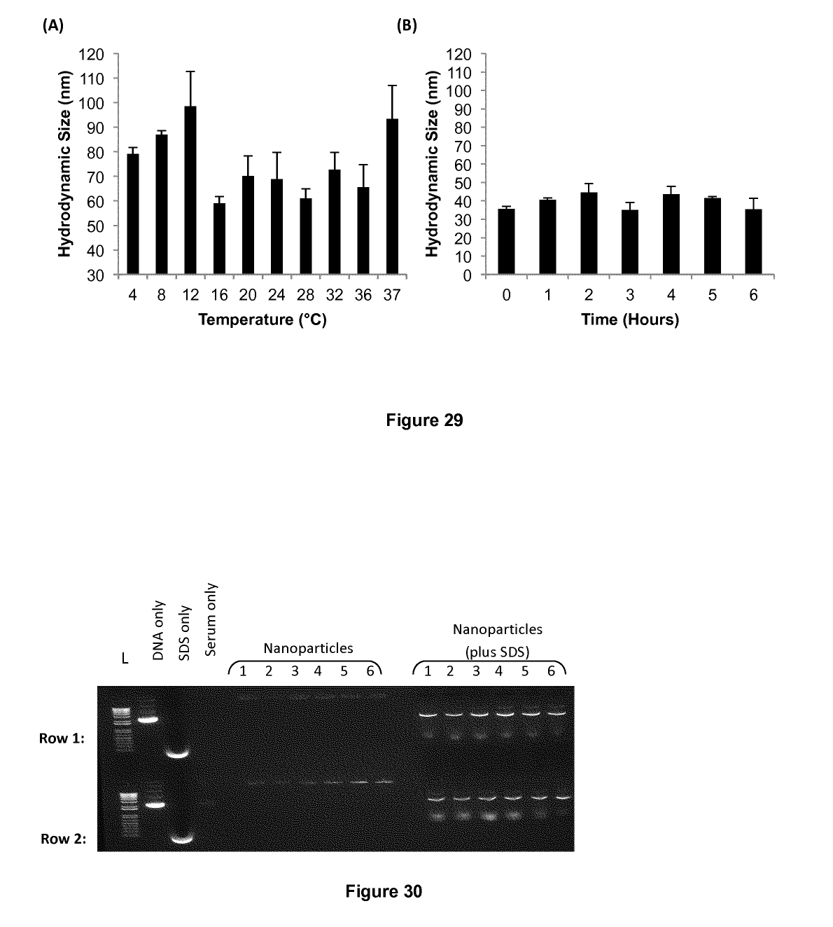

FIG. 29: Characterisation of RALA/pORF-mIL4 nanoparticles N:P 10 via hydrodynamic size analysis following (A) incubation at temperatures 4-37° C. and (B) incubation at room temperature for up to 6 h.

Following preparation of RALA/pORF-mIL4 nanoparticles N:P 10 they were characterised over a range of temperatures (4-37° C.) and following incubation at room temperature for up to 6 h using the Malvern Zetasizer NanoZS with DTS software. The measurements are reported as mean±SEM, (n=3).

FIG. 30: Agarose gel analysis of serum stability assay of peptide/pORF-mIL4 nanoparticles at N:P ratio 10 Row 1: Peptide/pORF-mIL4 nanoparticles N:P 10 incubated in water at 37° C. for 1-6 hours and decomplexed with SDS for 10 minutes; Row 2: Peptide/pORF-mIL4 nanoparticles N:P 10 incubated in 10% serum at 37° C. for 1-6 hours and decomplexed with SDS for 10 minutes. Following incubation 30 μL of samples were electrophoresed through a 1% agarose gel containing 0.5 μg/mL ethidium bromide to visualise DNA. A current of 80 V was applied for 1 h and the gel imaged. L=1 kb plus DNA ladder. Gel images are representative of three independent studies.

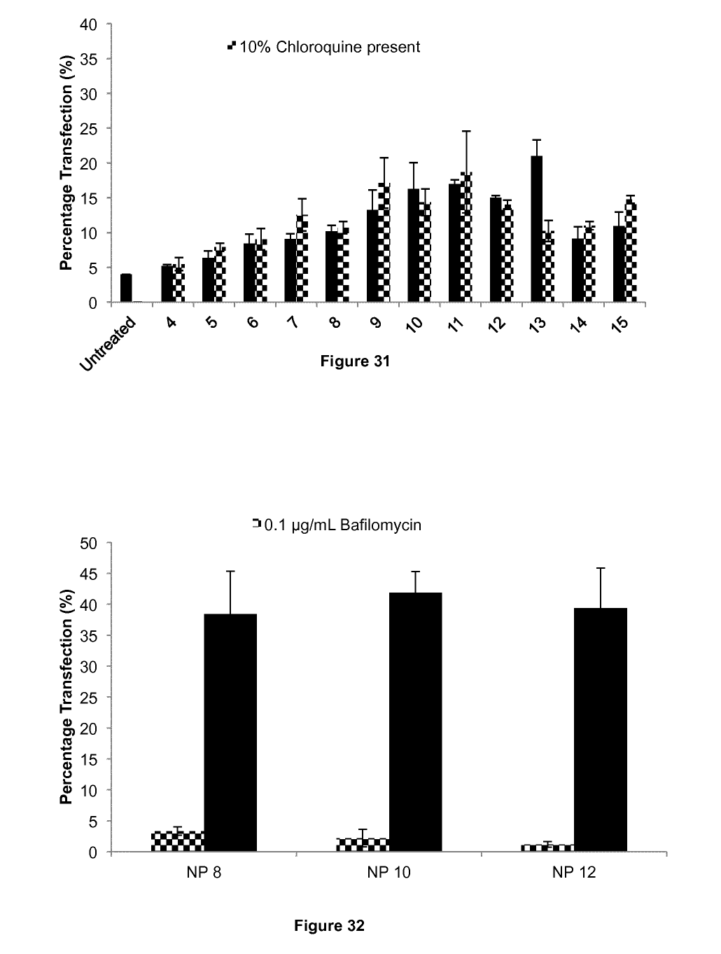

FIG. 31: Flow cytometric analysis of GFP expression 48 h post transfection in ZR-75-1 cell line with peptide/pEGFP-N1 nanoparticles N:P ratios 4-15. ZR-75-1 cells were conditioned for 2 h in 100 μL Opti-MEM serum free media which was then supplemented with 50 μL peptide/DNA complexes N:P ratios 4-15 containing 1 μg pEGFP-N1. Following transfection for 6 h the media was removed and replaced with RPMI 1640 containing 10% FBS. ZR-75-1 cells were imaged by fluorescence microscopy and fixed in formaldehyde for flow cytometry. The experiment was repeated with the addition of 10% chloroquine. Measurements are reported as mean±SEM, (n=3).

FIG. 32: Flow cytometric analysis of GFP expression 48 h post transfection in ZR-75-1 cell line with RALA/pEGFP-N1 nanoparticles N:P ratios 8, 10 and 12 in the presence and absence of Bafilomycin. ZR-75-1 cells were conditioned for 2 h in 100 μL Opti-MEM serum free media which was then supplemented with 50 μL peptide/DNA complexes N:P ratios 8, 10 and 12 containing 1 μg pEGFP-N1. Following transfection for 6 h the media was removed and replaced with RPMI 1640 containing 10% FBS. ZR-75-1 cells were fixed in formaldehyde for flow cytometry. The experiment was repeated with the addition of 0.1 μg/mL bafilomycin. Measurements are reported as mean±SEM, (n=3)

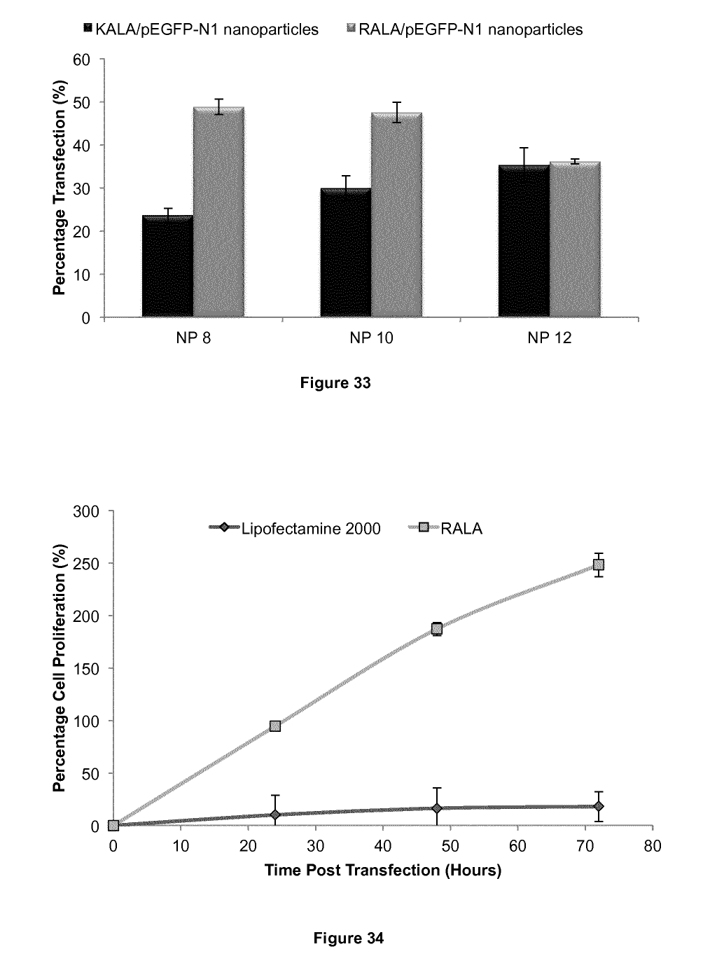

FIG. 33: Flow cytometric analysis of GFP expression 48 hours post transfection in ZR-75-1 cell line with KALA or RALA/DNA nanoparticles at N:P ratios 8, 10 and 12. ZR-75-1 cells were conditioned for 2 hours in 100 μL Opti-mem serum free media which was then supplemented with 50 μL peptide/pEGFP-N1 complexes N:P ratios 8, 10 and 12 containing 1 μg pEGFP-N1. After 6 hours the media was removed and replaced with RPMI 1640 containing 10% FBS. ZR-75-1 cells were fixed in formaldehyde for flow cytometry. The measurements are reported as mean±SEM, (n=3).

FIG. 34: Cell proliferation over time following transfection with Lipofectamine 2000/pEGFP-N1 and RALA/pEGFP-N1 N:P ratio 10. ZR-75-1 cells were conditioned for 2 hours in 100 μL Opti-mem serum free media which was then supplemented with 50 μL RALA/pEGFP-N1 complexes N:P 10 containing 1 μg pEGFP-N1. After 6 hours the media was removed and replaced with RPMI 1640 containing 10% FBS. Cells were trypsinised and analysed via cell count analysis 24, 48 and 72 hours post transfection. The measurements are reported as mean±SEM, (n=3).

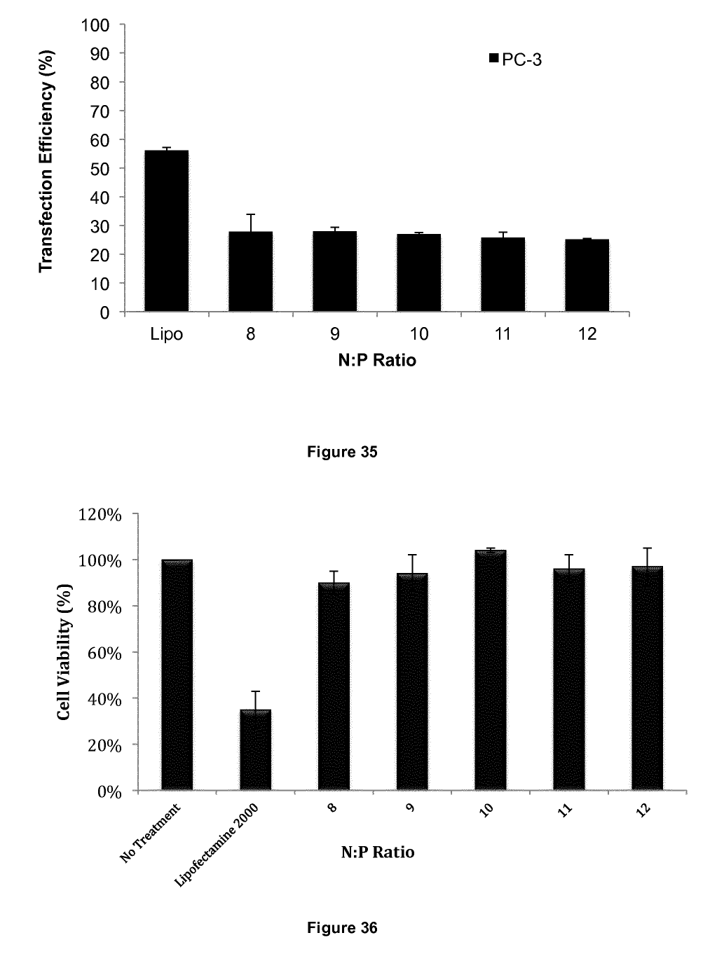

FIG. 35: Flow cytometric analysis of GFP expression 48 hours post transfection in PC-3 prostate cancer with RALA/pEGFP-N1 nanoparticles at N:P ratios 8-12. Cells were conditioned for 2 hours in 100 μL Opti-mem serum free media which was then supplemented with 50 μL RALA/pEGFP-N1 complexes N:P ratios 8-12 containing 1 μg pEGFP-N1. After 6 hours the media was removed and replaced with RPMI 1640 containing 10% FBS. Cells were fixed in formaldehyde for flow cytometry. The measurements are reported as mean±SEM, (n=3).

FIG. 36: WST-1 assay to measure cytotoxicity in PC-3 prostate cancer cells 48 hours post-transfection with a range of RALA/pEGFP-N1 N:P ratios. Cells were conditioned for 2 hours in 100 μL Opti-mem serum free media which was then supplemented with 50 μL RALA/pEGFP-N1 complexes N:P ratios 8-12 containing 1 μg pEGFP-N1. After 6 hours the media was removed and replaced with RPMI 1640 containing 10% FBS. The data was normalised against the untreated control which was considered 100% viable. The measurements are reported as mean±SEM, (n=3).

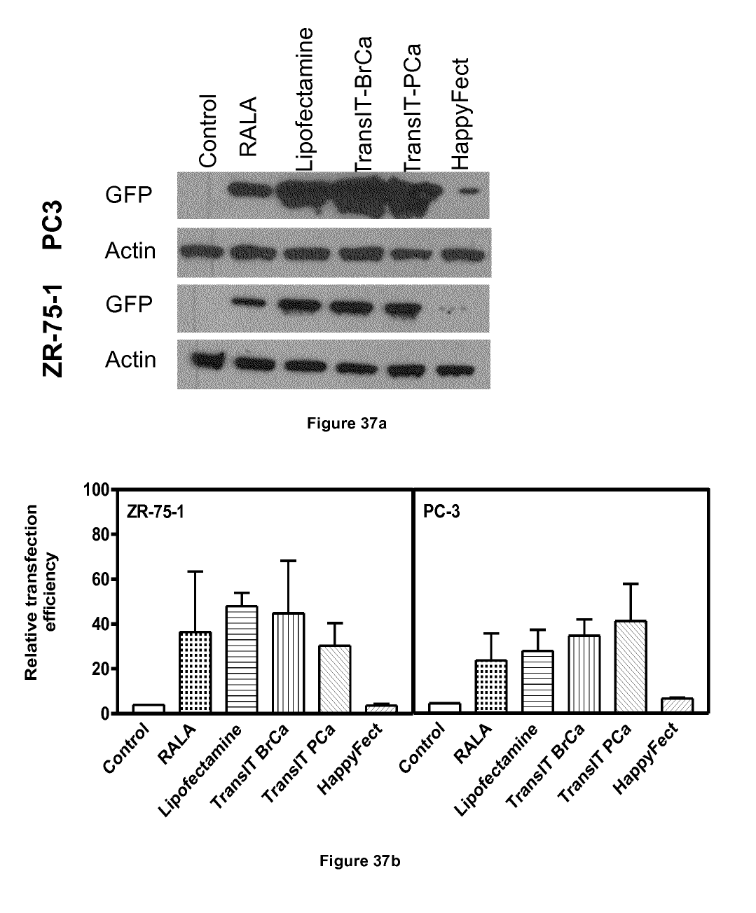

FIG. 37: Assessment of RALA's transfection ability in PC-3 and ZR-75-1 cancer cells in comparison to commercially available transfection reagents. 1.5×105 ZR-75-1 or 1×105PC-3 were seeded into wells of 24 well plates and incubated overnight. Cells were transfected with 0.5 μg pEGFP-N1 per well for 6 h, before transfection complexes were removed and medium replaced with normal growth medium. Cells were analysed for EGFP expression 60 h post-transfection using a) immunoblotting and b) flow cytometry. N=3.

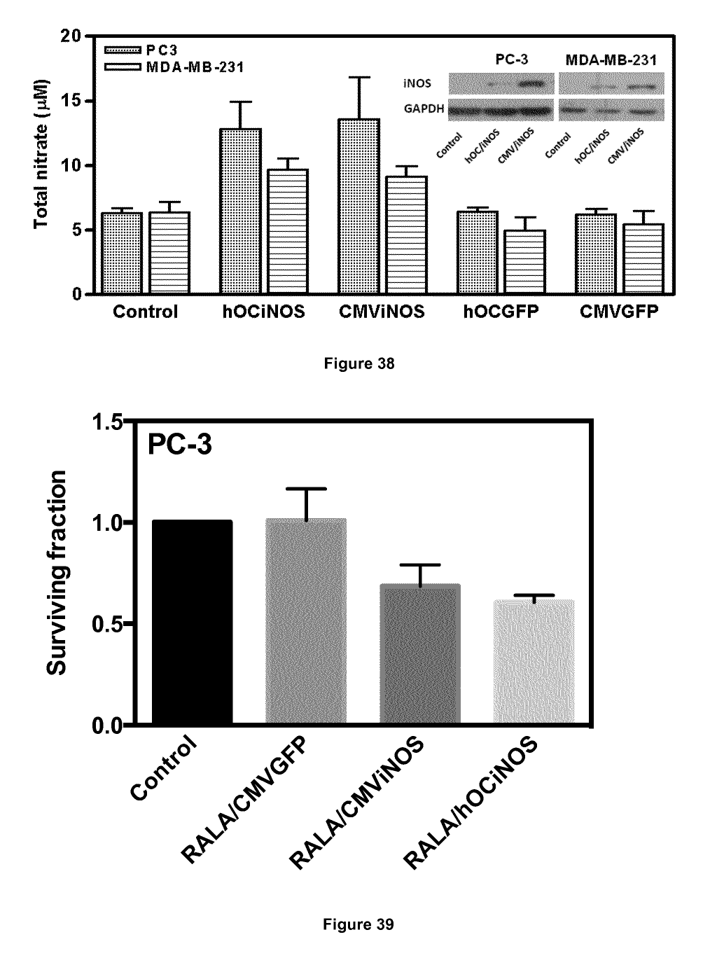

FIG. 38: Validation of CMV and hOC-driven iNOS plasmids. 1.5×10⁵ ZR-75-1 or 1×10⁵PC-3 were seeded into wells of 24 well plates and incubated overnight. Cells were transfected with 0.5 μg CMV-iNOS or hOC-iNOS complexed with RALA per well for 6 h, before transfection complexes were removed and medium replaced with MEM. Cells were analysed for iNOS expression 48 h post-transfection using immunoblotting, and the functionality of the iNOS product was confirmed using Greiss test for nitrate production (B). (N=3+/−SD).

FIG. 39: iNOS gene therapy reduces the clonogenicity of PC-3 prostate cancer cells. Transfection with RALA/CMV-iNOS or RALA/hOC-iNOS nanoparticles reduced the clonogenic survival of PC-3s. 3×105 PC-3s in 6-well plates were transfected with 5 μg DNA complexed with RALA at N:P10. 24 h later, cells were plated into 6 well plates (200/500 per well). Following 10 days incubation, colonies were stained using crystal violet and enumerated. N=2.

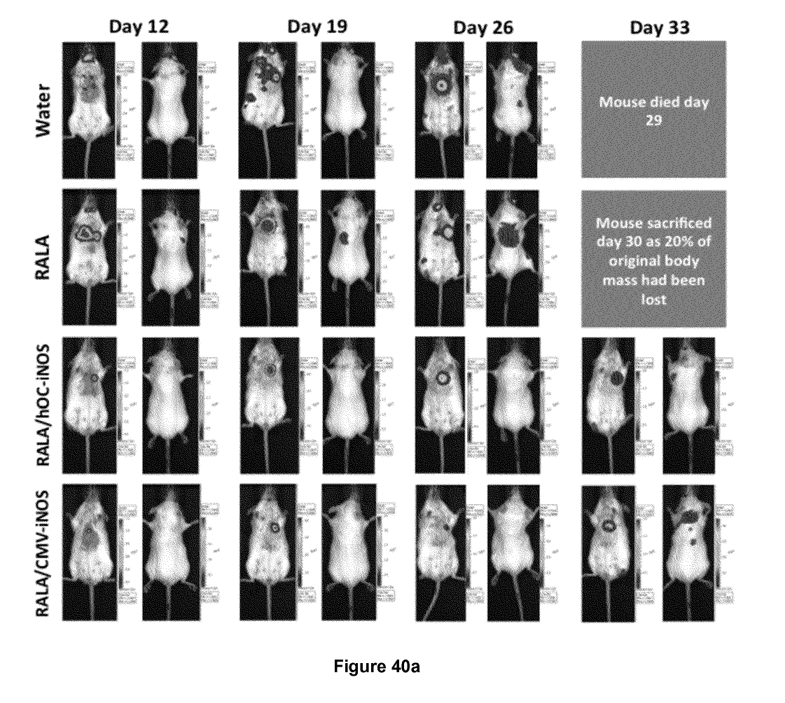

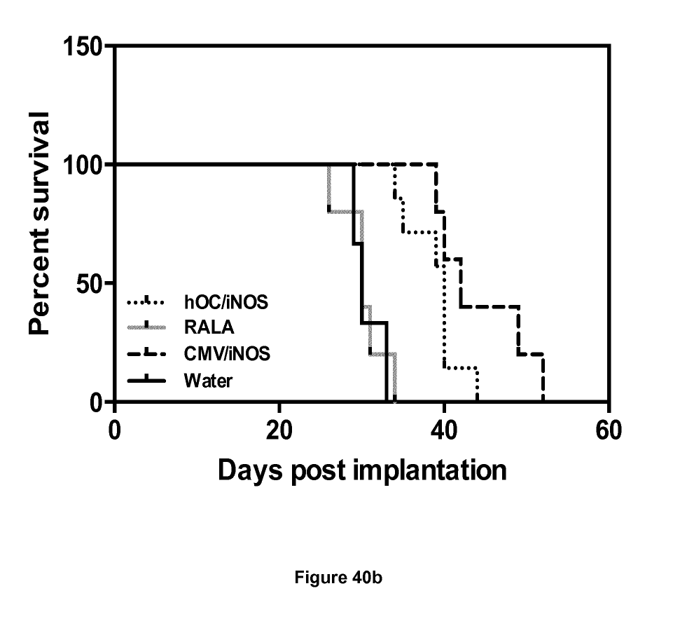

FIG. 40 (a & b): In vivo efficacy of the RALA/iNOS nanoparticles in a metastatic model of breast cancer. Female BALB/c SCID mice were inoculated via the left ventricle with 2×105 MDA-MB-231-luc2. 48 h later, mice received 10 μg plasmid CMV-iNOS or hOC-iNOS complexed with RALA (7 mice/group), and continued to receive therapy twice weekly for five treatments (Day 16); control mice received water only, or 100 μl of 1.45 mg/ml RALA (corresponding to the amount of RALA in the gene therapy treatments). Figure a) contains bioluminescence images of 4 representative mice at 12, 19, 26 and 33 days post inoculation; at each time point, the degree of bioluminescence in the RALA only, RALA/CMV-iNOS and RALA/hOC-iNOS treated mice was standardised against the degree of bioluminescence in the time-matched water-treated mouse, thereby facilitating comparison of luminescence. In the case of the day 33 mice, for whom no time-matched water-treated control was available, bioluminescence was standardised using the scale parameters of the water-treated mouse as on day 26. Figure b) contains a Kaplan-Meier curve detailing the survival of mice that received the indicated treatment. Mice that received CMV-iNOS or hOC-iNOS gene therapy survived significantly longer than mice that received water treatment (P=0.001 and 0.024 respectively); survival of mice that received RALA therapy without therapeutic DNA payload was not significantly different from that of water-treated mice (P=0.881).

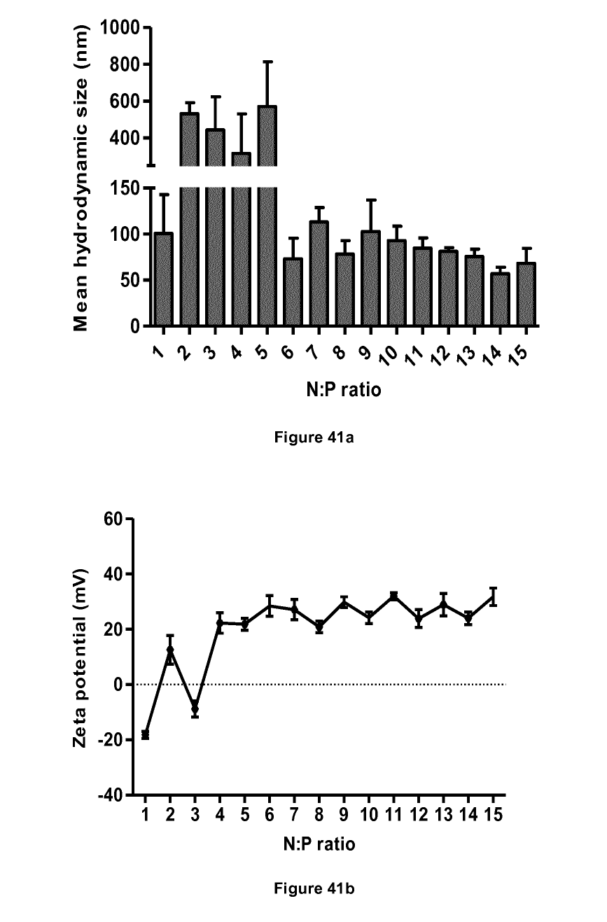

FIG. 41: Mean hydrodynamic size and zeta potential of RALA/Runx2 siRNA nanoparticles prepared at a range of N:P ratios from 1-15. The nanoparticles were incubated for 30 min on ice before their hydrodynamic size (A) and corresponding zeta potential (B) were measured using a Malvern Zetasizer Nano ZS. Results are displayed as mean±SEM, n=3, where n represents the number of independent batches prepared for measurement.

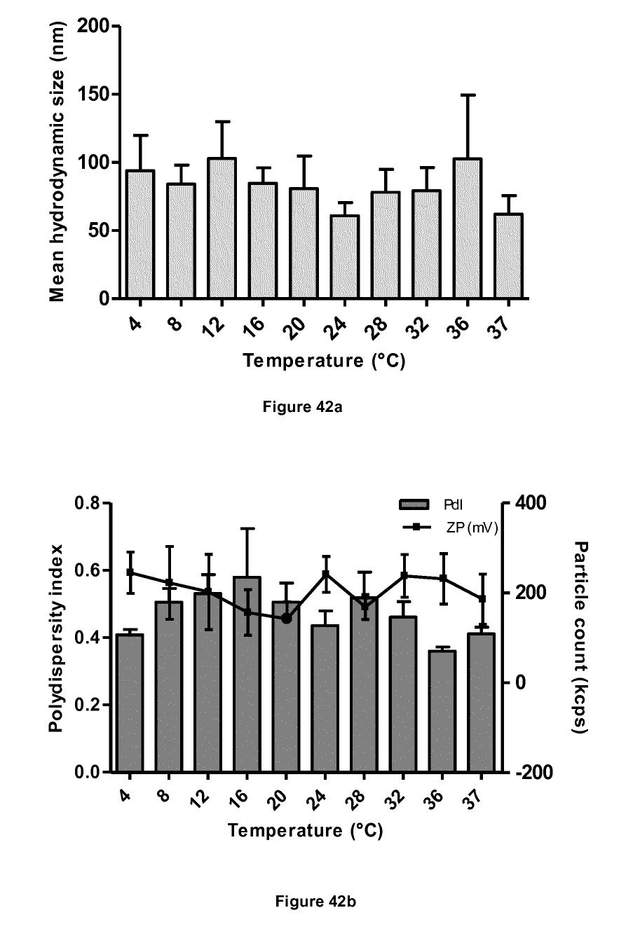

FIG. 42 (a & b): A: Mean hydrodynamic size of RALA/Runx2 siRNA nanoparticles was determined to assess stability of the particles across a temperature range. RALA/Runx2 siRNA nanoparticles were prepared at N:P 12 such that they contained 0.5 μg Runx2 siRNA and 7.25 μg RALA and incubated on ice for 30 min. The mean hydrodynamic size of the nanoparticles was then measured at 4° C. intervals, from 4° C. to 37° C., using a Malvern Zetasizer Nano ZS. B: The corresponding PDI and particle count were also recorded. Results are displayed as mean±SEM, n=3, where n represents the number of independent batches prepared for measurement.

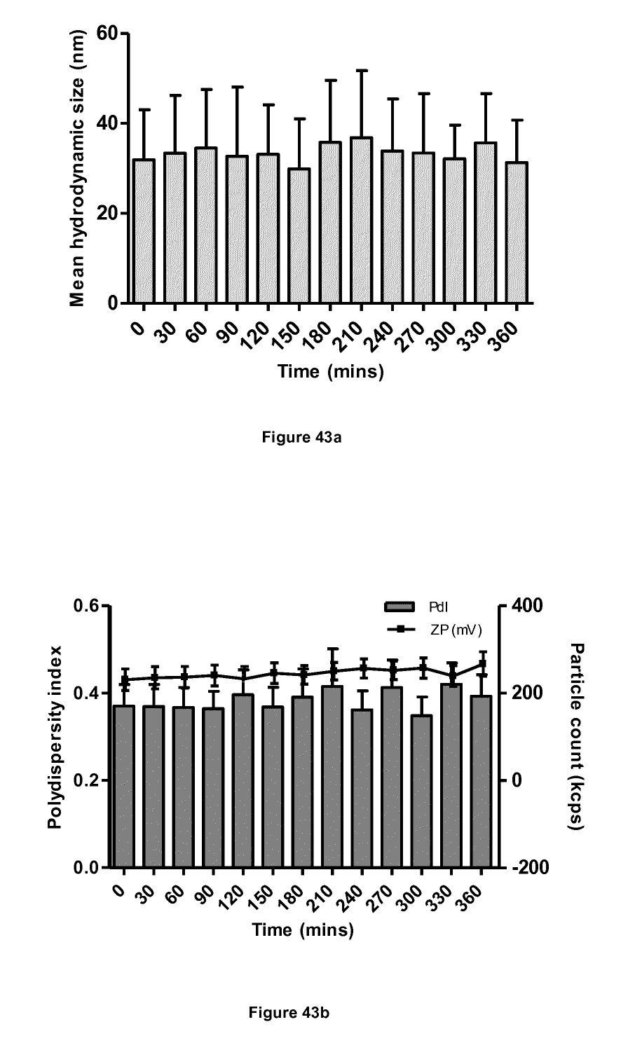

FIG. 43 (a & b): A: Mean hydrodynamic size of RALA/Runx2 siRNA nanoparticles was determined to assess stability of the particles across a 6 h time period. RALA/Runx2 siRNA nanoparticles were prepared at N:P 12 such that they contained 0.5 μg Runx2 siRNA and 7.25 μg RALA. The mean hydrodynamic size of the nanoparticles was measured at 30 min intervals, starting immediately after formulation until 6 h after, using a Malvern Zetasizer Nano ZS. B: The corresponding PDI and particle count were also recorded. Results are displayed as mean±SEM, n=3, where n represents the number of independent batches prepared for measurement.

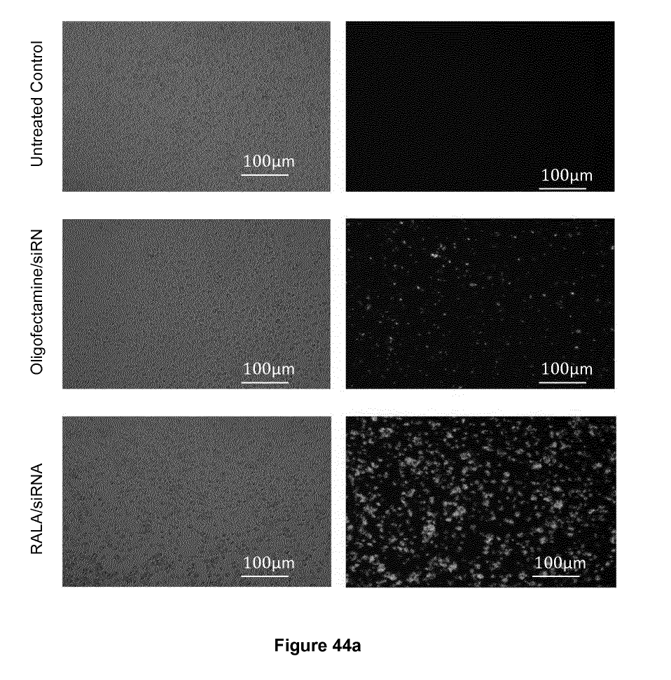

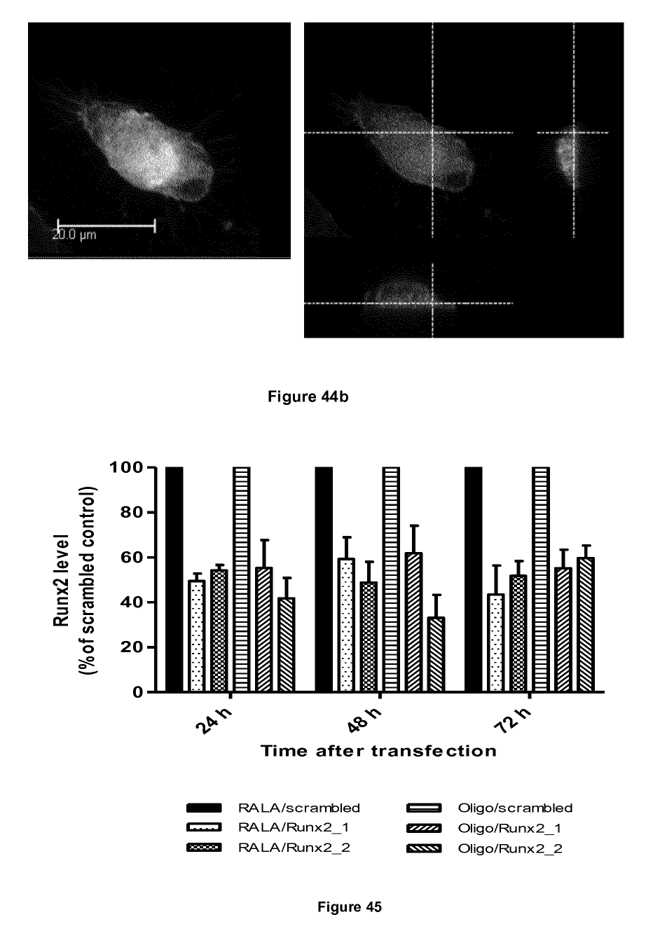

FIG. 44 (a & b): A: Transfection efficiency of RALA/fluorescent siRNA nanoparticles was assessed in a prostate cancer cell line. PC-3 prostate cancer cells were transfected for 4 h with RALA/fluorescent siRNA nanoparticles prepared at N:P 12 or Oligofectamine. Following the transfection the medium was removed and replaced with RPMI 1640 supplemented with 10% FCS and allowed to incubate for up to 72 h. B: PC3 prostate cancer cells were allowed to adhere to a coverslip overnight having been seeded at a density of 50,000 cells per coverslip prior to transfection with RALA/fluorescent siRNA nanoparticles (green) for 4 h. The cells were then fixed using 2% formaldehyde and stained with Wheat Germ Agglutinin, Alexa Fluor conjugate 488 (red) followed by Hoechst stain (blue), each for 20 min. The coverslips were subsequently mounted onto slides using ProLong Gold Antifade Reagent and sealed the following day. A Leica TCS SP8 confocal microscope was used to image the cells and produce orthogonal sectioning and a Z-stack using Leica software.

FIG. 45: Quantification of western blotting using image J software to determine the Runx2 knockdown. PC-3 prostate cancer cells were transfected with a 100 nM concentration of Runx2_1, Runx2_2 or non-targeting scrambled siRNA using either RALA peptide or Oligofectamine. Cell lysates were collected 24, 48 and 72 h following the 4 h transfection and run on 8% acrylamide gels. Results are obtained from at least 2 independent repeats.

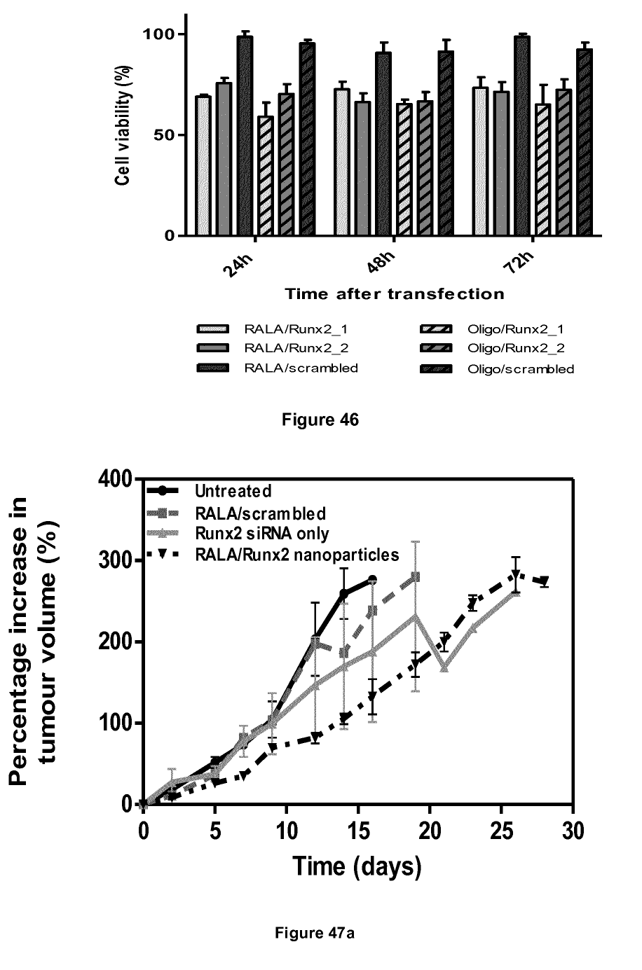

FIG. 46: The effect of Runx2 knockdown on cell proliferation was evaluated using two different delivery systems, namely RALA peptide and the commercial siRNA transfection reagent, Oligofectamine. Transfection with RALA/siRNA nanoparticles at N:P 12 and with Oligofectamine was for 4 h. Medium was then supplemented with RPMI 1640 containing 30% FCS for up to 72 h such that the final concentration of FCS was 10%. Subsequently cells were trypsinised and counted using a haemocytometer. Untreated cells were considered to be 100% viable and viability under all other conditions was calculated based on this. Results are reported as mean±SEM, n=3, where n represents the number of independent batches prepared for analysis.

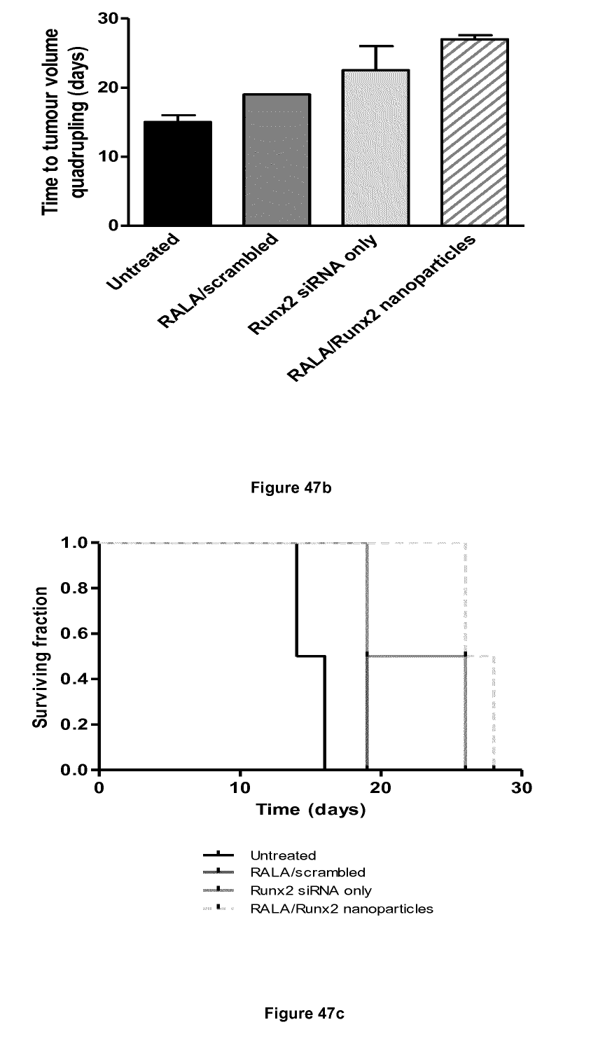

FIG. 47: A PC-3 prostate cancer cell xenograft model was used for the in vivo assessment of RALA as a delivery system for siRNA and the effects of Runx2 knockdown on tumour cell proliferation. Tumours were implanted on the rear dorsum of BALB-C SCID mice and grown until the volume reached approximately 150 mm³. Treatments were once weekly for three weeks via intratumoural injection with mice being assigned randomly to either a water only, RALA/scrambled siRNA nanoparticles, Runx2 siRNA only or RALA/Runx2 siRNA nanoparticles treatment group. Runx2_1 and Runx2_2 siRNA were pooled for the purposes of in vivo analysis. As this was a pilot study the numbers within each group ranged from 1-4. The experimental endpoint was quadrupling of tumour volume. A: Percentage increase in tumour volume over time is presented showing a lower rate of tumour growth when tumours were treated with RALA/Runx2 siRNA nanoparticles. The rate of growth in Runx2 siRNA treated mice was slower than control groups; however, there was a large amount of variability. B: Time taken for tumour growth to quadruple is displayed with high statistical significance in overall survival time between RALA/Runx2 siRNA nanoparticles and water only treated mice (unpaired one-tailed t test p<0.001). However, there is no statistical significance in the difference in survival times of Runx2 siRNA only and RALA/Runx2 siRNA nanoparticles due to the variability of the survival time in Runx2 siRNA only treated mice (unpaired one-tailed t test p>0.05). C: Kaplan-Meier plot demonstrating the survival of tumour-bearing mice for each of the treatment groups from the start of dosing until the time at which the tumour volume quadruples. Censoring was not required as all animals left the study due to the experimental endpoint being reached.

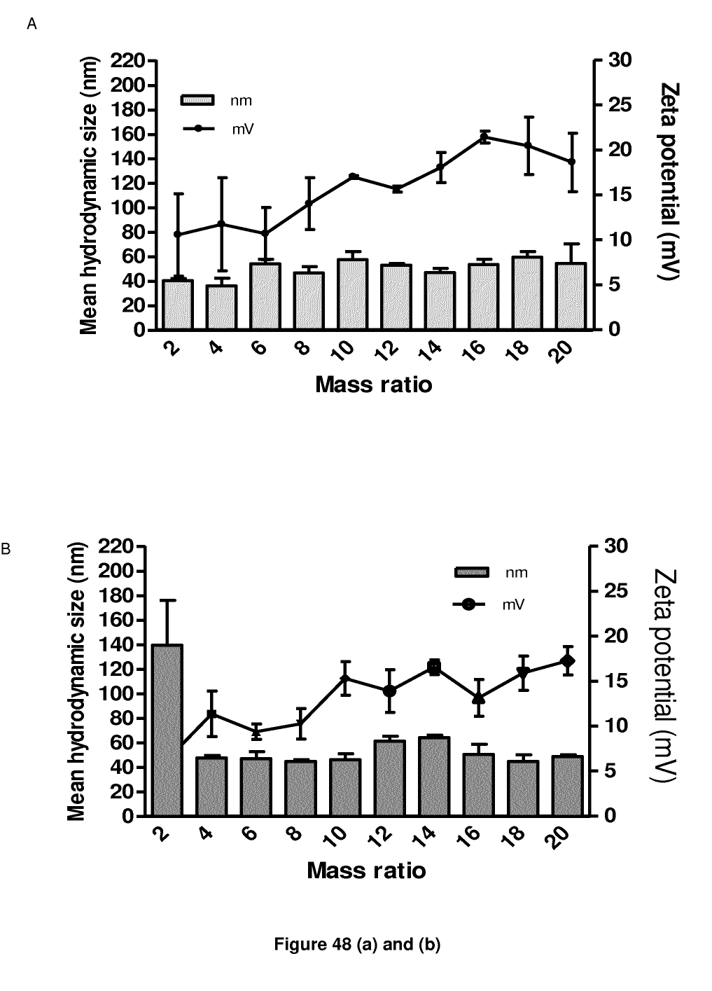

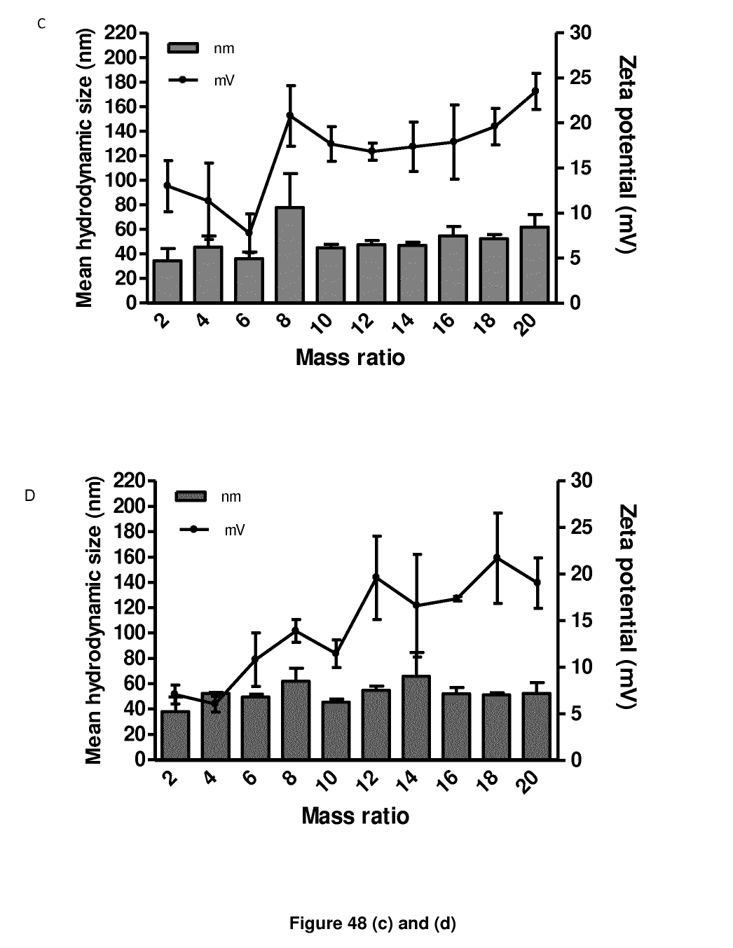

FIG. 48: Mean hydrodynamic size and zeta potential of A: RALA/alendronate nanoparticles, B: RALA/etidronate nanoparticles, C: RALA/risedronate nanoparticles and D: RALA/zoledronate nanoparticles. RALA/BP nanoparticles were prepared at a range of mass ratios, such that for a mass ratio of 10:1 the nanoparticles contained 1 μg BP and 10 μg RALA. The nanoparticles were incubated for 30 min before their hydrodynamic size and zeta potential were measured using a Malvern Zetasizer Nano ZS. Results are displayed as mean±SEM, n=3, where n represents the number of independent batches prepared for measurement.

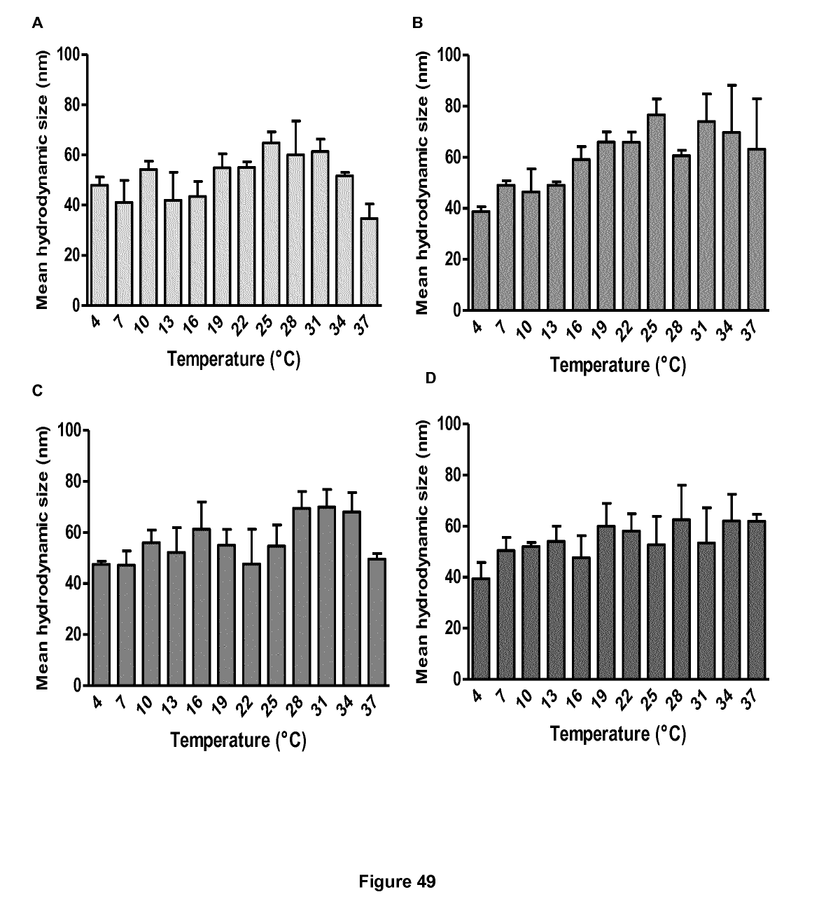

FIG. 49: Mean hydrodynamic size of RALA/BP nanoparticles was determined to assess thermal stability over a range of temperatures. RALA/BP nanoparticles were prepared at a range of mass ratios, such that for a mass ratio of 10:1 the nanoparticles contained 1 μg BP and 10 μg RALA. The nanoparticles were incubated for 30 min before the mean hydrodynamic size was measured using a temperature trend function on the Malvern Zetasizer Nano ZS. Results are displayed as mean±SEM, n=3, where n represents the number of independent batches prepared for measurement. A: RALA/alendronate nanoparticles, B: RALA/etidronate nanoparticles, C: RALA/risedronate nanoparticles and D: RALA/zoledronate nanoparticles.

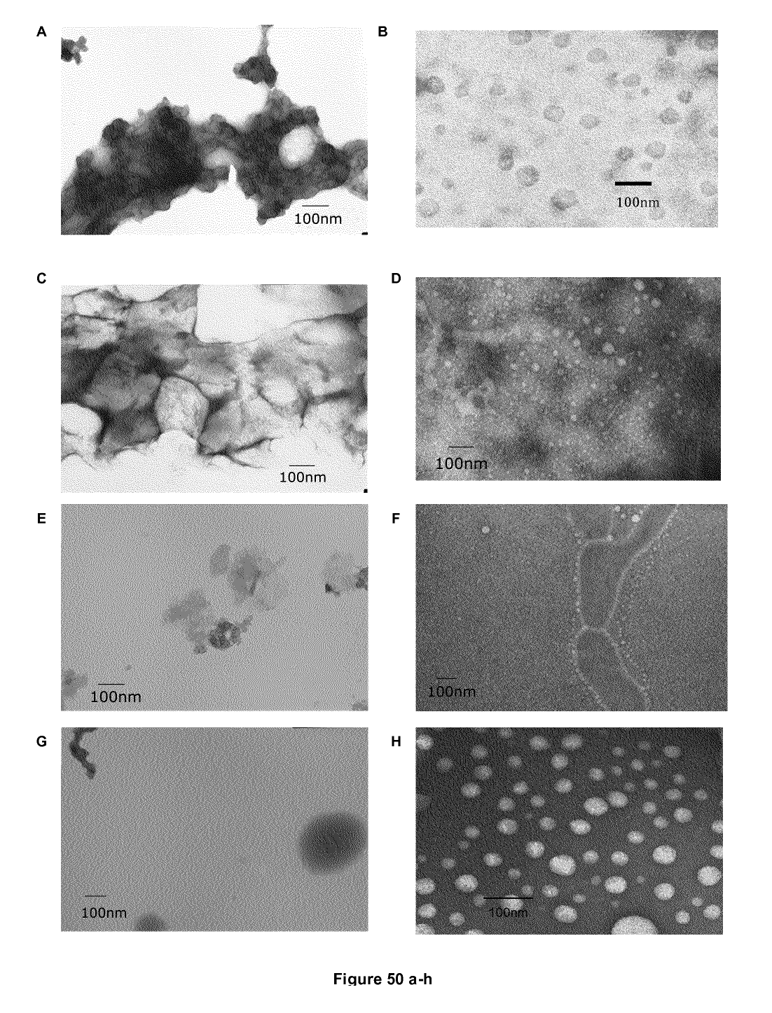

FIG. 50: TEM of the RALA/BP Nanoparticles. Nanoparticles were prepared at a mass ratio of 10:1 and allowed to incubate for 30 min before being loaded onto carbon reinforced formvar coated copper grids. Samples were allowed to dry before being stained with 5% uranyl acetate for 5 min at room temperature. The nanoparticles were imaged using a JEOL 100CXII transmission electron microscope at an accelerating voltage of 80 kV and images were captured onto Kodak 4489 Electron Microscope Film. This was developed using Kodak D19 developer, fixed with Universal fixer, washed and dried. The negatives were then scanned onto a PC as JPEG images. A: Blank grid with no stain; B: Stained blank grid; C: RALA only; D: Alendronate only; E: RALA/alendronate nanoparticles; F: Etidronate only; G: RALA/etidronate nanoparticles; H: Risedronate only; I: RALA/risedronate; nanoparticles; J: Zoledronate only; K: RALA/zoledronate nanoparticles.

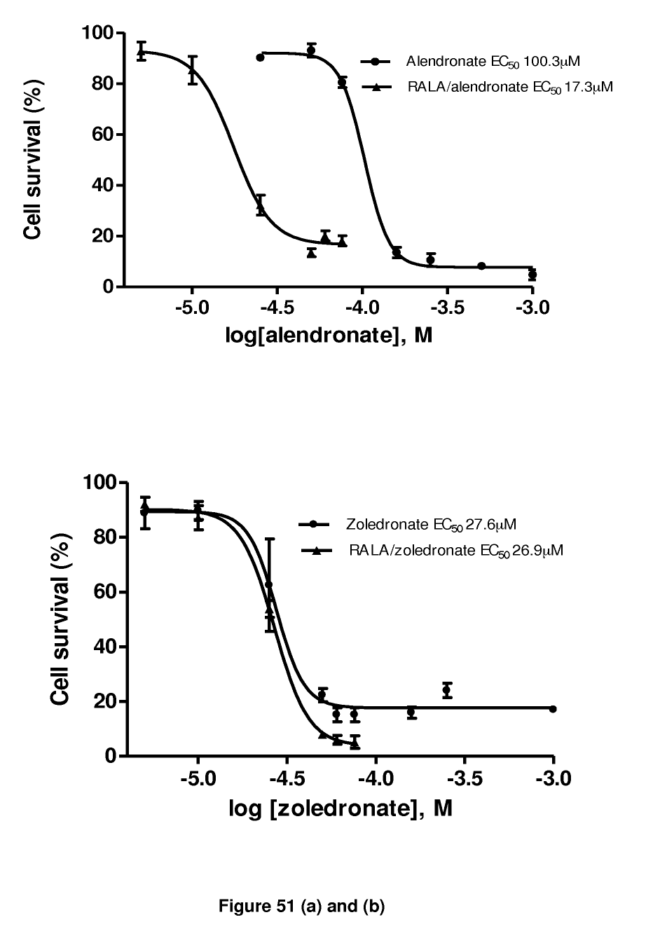

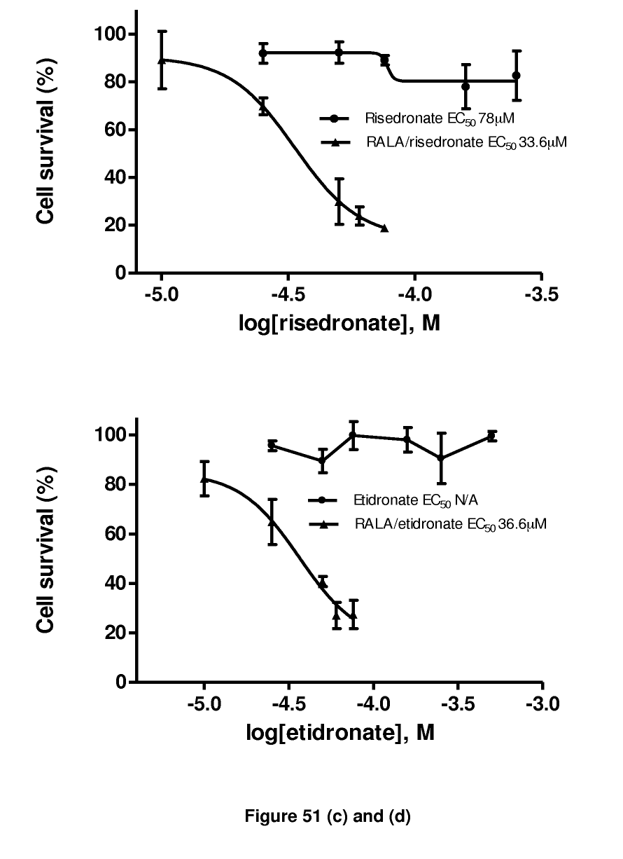

FIG. 51: Cell viability was evaluated by manual counting of the viable adherent cells using a haemocytometer. PC-3 prostate cancer cells were seeded in a 96-well flat-bottom tissue culture plate at a density of 1×104 cells per well and incubated in complete culture medium for 24 h. Two hours prior to transfection the cells were conditioned in OptiMEM serum-free medium and subsequently treated with solutions of BP to achieve a final exposure concentration of 5 μM to 1 mM. RALA/BP nanoparticles were prepared using a mass ratio of 10:1 such that the final concentration of BP per well was in the range 5 μM to 75 μM. Cells were incubated at 37° C. with 5% CO2 for 6 h before medium was replaced with completed culture medium and left to incubate for 72 h. Following incubation the cells were trypsinised and counted. Cell viability was expressed as a percentage of the untreated control where the untreated control is considered to be 100% viable. Dose-response curves were obtained for free BP and RALA/BP allowing determination of EC50 values for each. EC50 values refer to the concentration that induces a response halfway between the baseline and the maximum plateau obtained. A: RALA/alendronate and alendronate treated cells; B: RALA/zoledronate and zoledronate treated cells; C: RALA/risedronate and risedronate treated cells; D: RALA/etidronate and etidronate treated cells.

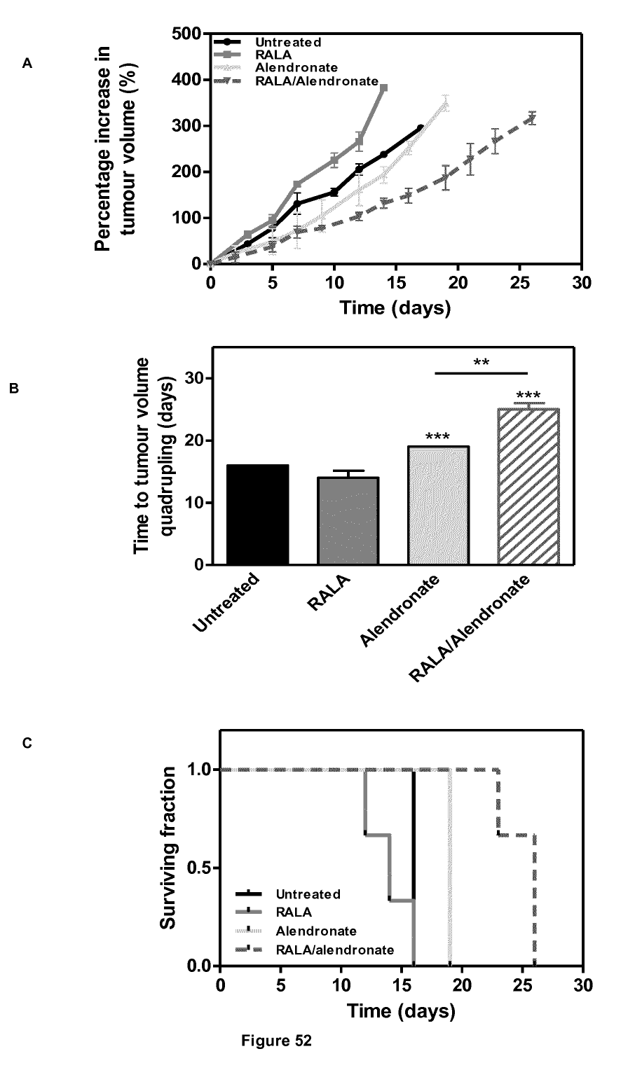

FIG. 52: PC-3 prostate cancer cell xenograft model was used for the in vivo assessment of RALA as a delivery system for BPs. Tumours were implanted on the rear dorsum of BALB-C SCID mice and grown until the volume reached approximately 100 mm3. Treatments were three times weekly for three weeks via intratumoural injection with mice being assigned randomly to either an untreated, RALA only, free alendronate or RALA/alendronate treatment group. Each treatment group consisted of three mice which allowed statistical significance in the outcomes to be observed. The experimental endpoint was quadrupling of tumour volume. A: Percentage increase in tumour volume over time is presented showing a low rate of tumour growth when tumours were treated with RALA/alendronate nanoparticles. The rate of growth in the controls was considerably higher. B: Time taken for tumour growth to quadruple is displayed with high statistical significance in overall survival time between free alendronate and untreated control, and RALA/alendronate and untreated control (both p<0.001). Furthermore, there is statistical significance in the difference in survival times of free alendronate and RALA/alendronate (p<0.01). C: Kaplan-Meier plot demonstrating the survival of tumour-bearing mice for each of the treatment groups from the start of dosing until the time at which the tumour volume quadruples. Censoring was not required as all animals left the study due to the experimental endpoint being reached

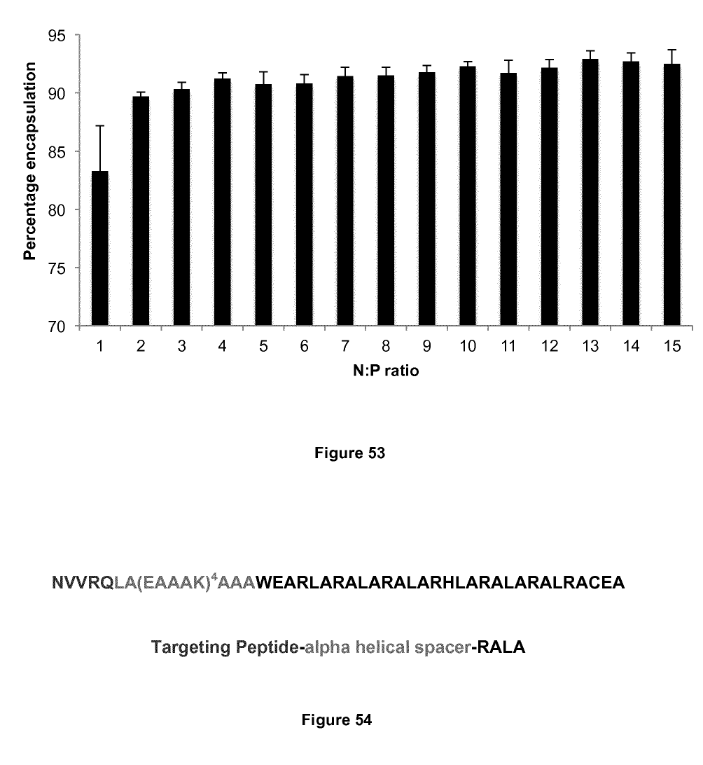

FIG. 53: Encapsulation assay of RALA/pEGFP-N1 nanoparticles over a range of N:P ratios (0-15). RALA/pEGFP-N1 nanoparticles were incubated for 30 mins with Picogreen and the fluorescence intensity of the resulting complexes measured at 520 nm using a spectrofluorometer. The measurements are reported as mean±SEM, (n=3).

FIG. 54: The amino acid sequence of the RAT peptide consisting of three moieties, each with a specialist role to fulfil in delivering therapeutic DNA to target cells (e.g. PC-3); a TMTP-1 metastatic targeting peptide (TP) for specificity, an alpha helical spacer and RALA.

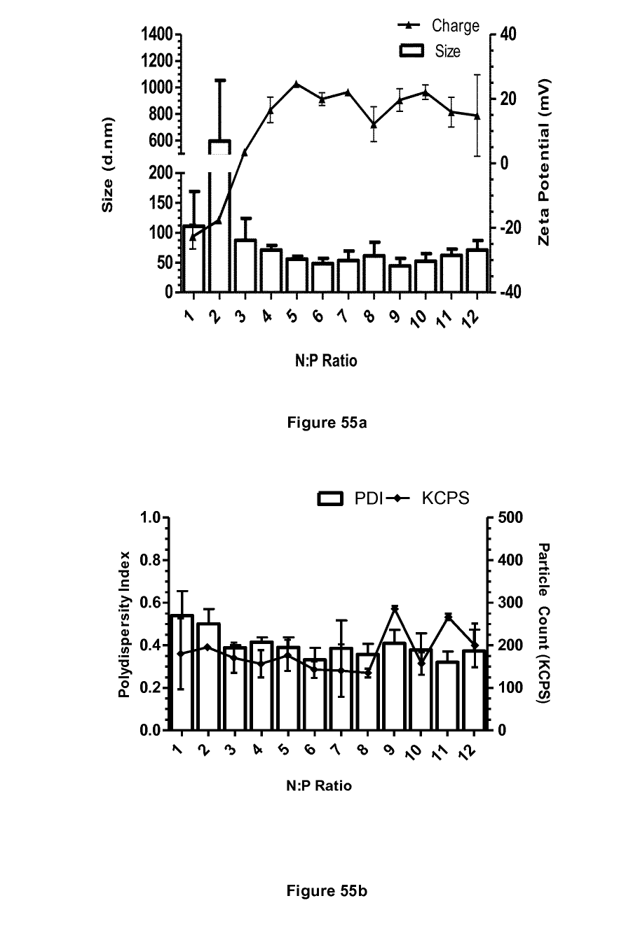

FIG. 55: Zetasizer analysis of RAT/pEGFP-N1 nanoparticles over a range of nitrogen[peptide]:phosphate[DNA] (NP) ratios. a) Hydrodynamic size (nm) and surface charge (mV) b) Count rate (kilo counts per second and Poly-dispersity Index. Nanoparticles less than 200 nm are formed at N:P ratios of 3-12 with a charge in the 20 mV range. N:P12=71.03 nm±11.36 nm; 17.49 mV±11.92. Mean±SEM (n=3). A minimum of 15 measurement runs per repeat.

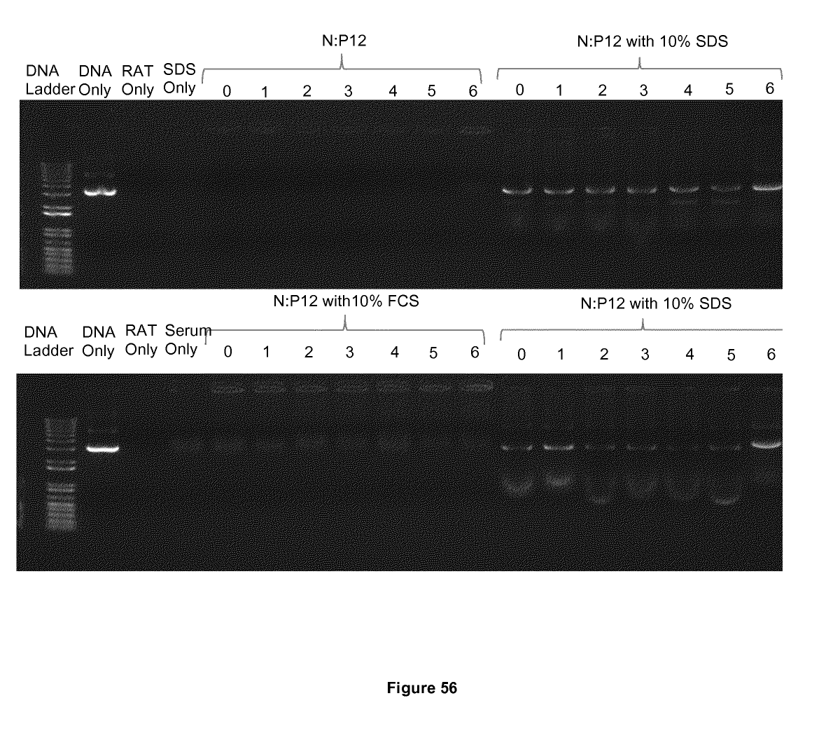

FIG. 56: N:P12 RAT/pEGFP-N1 nanoparticles incubated for 0-6 h (labelled 0-6) with and without the presence of 10% foetal calf serum. Replicates were de-complexed with 10% sodium dodecyl sulphate or 10 min to confirm the integrity of DNA. Nanoparticles were run on a 1% agarose gel for 1 h at 100 volts. Representative image of three experiments.

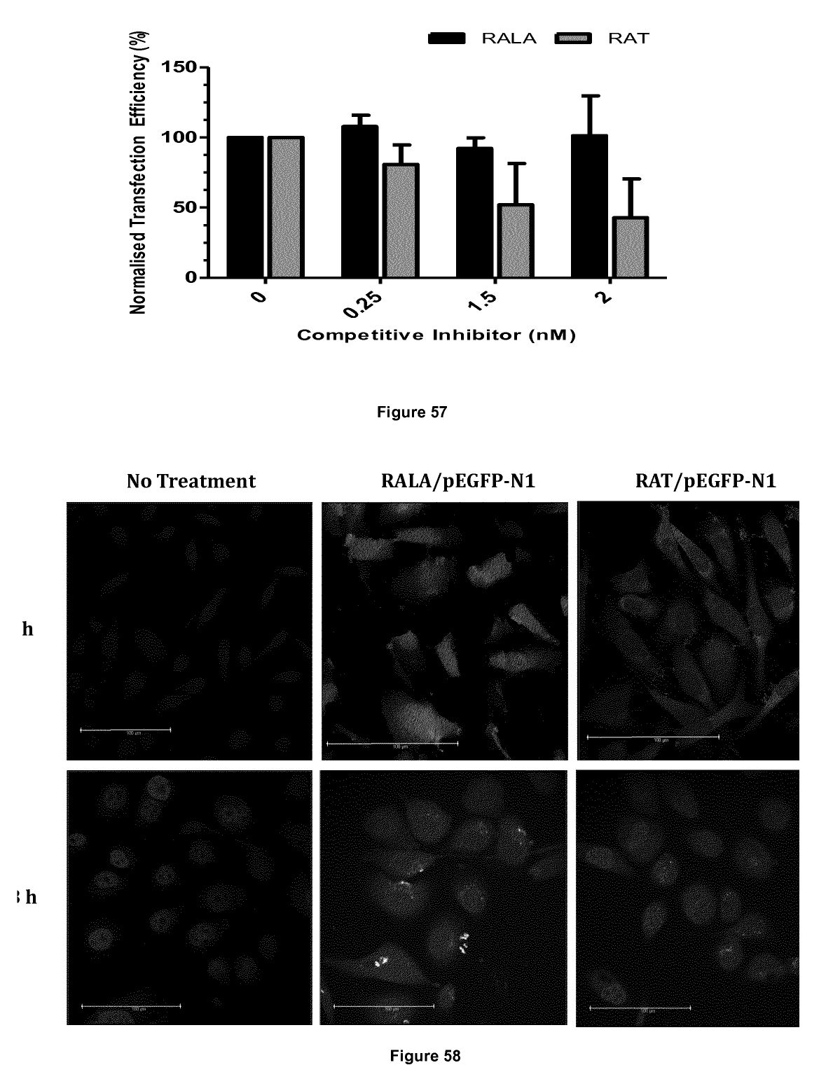

FIG. 57: Cells were transfected with RAT/pEGFP-N1 N:P12 nanoparticles with the additional of inhibiting peptide, TMTP1, and control peptide, scrambled TMTP1 (0.25 nM, 1.5 nM and 2 nM). Cells were fixed in formaldehyde for flow cytometry. The measurements are reported as mean±SEM, (n=3).

FIG. 58: Confocal microscopy of RAT and RALA/pEGFP-N1 transfected cells at 6 and 48 h. PC3 prostate cells were allowed to adhere to a coverslip overnight having been seeded at a density of 50, 000 cells, per coverslip, prior to transfection with RALA/Cy3 labeled pEGFP-N1 DNA (red) or RAT/Cy3 labeled pEGFP-N1 DNA (red) for 6 and 48 h. The cells were then fixed for 10 minutes using 2% formaldehyde and stained with Hoeschst stain (blue) for 2 minutes. The coverslips were subsequently mounted onto slides using ProLong Gold Antifade Reagent and sealed. A Leica TCS SP8 confocal microscope was used to image the cells and produce a Z-stack. Gene expression produced by pEGFP-N1 (green) is distinguishable at certain time points

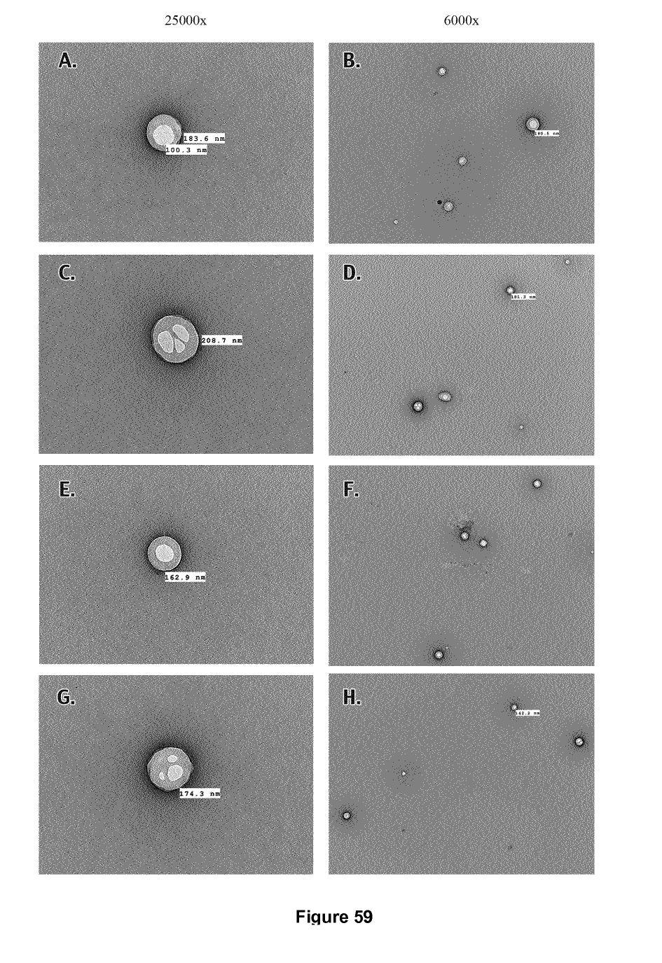

FIG. 59: Transmission electron microscopy of various composite nanoparticles. A. & B. PLGA; C & D: PLA10-PEG2; E & F: PLA25-PEG5; G & H: PLA50-PEG5. Images on the left were taken at 25,000×, images on the right were taken at 6,000× magnification.

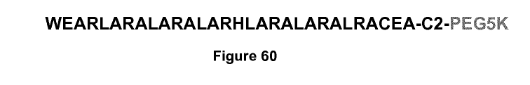

FIG. 60: The amino acid sequence of a. RALA and b. PEGylated RALA. PEG will potentially minimize opsonisation and also increase tumour targeting via the enhanced permeation and retention effect (EPR).

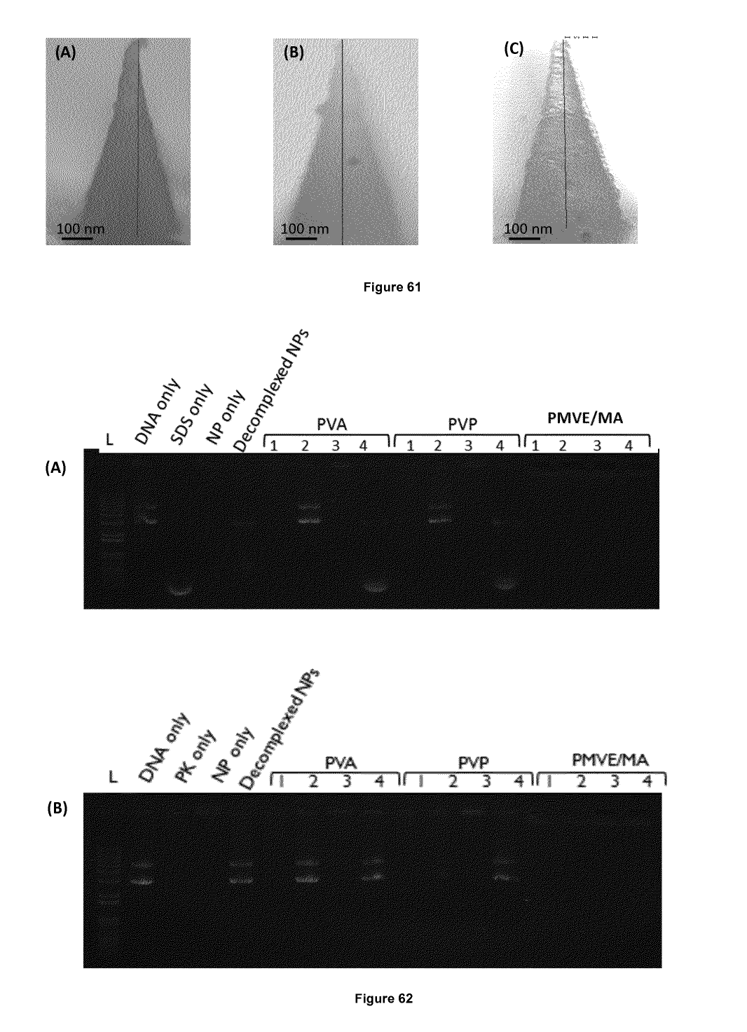

FIG. 61: Representative digital microscope images of MNs fabricated in micro-moulding process from different polymers Images of individual MNs fabricated from (A) 20% Polyvinyl alcohol (PVA); (B) 20% Polyvinylpyrrolidone (PVP); (C) 20% Gantrez® AN-139 poly(methylvinylether/maleic acid) (PMVE/MA) examined using a GE-5 digital microscope (Laboratory Analysis Ltd, UK) under magnification 180×.

FIG. 62: Agarose gel analysis of DNA and NP release from polymeric formulations using (A) 10% SDS; (B) 20% proteinase K to decomplex the NPs. Lane 1: Polymer only; Lane 2: Polymer and pEGFP-N1; Lane 3: Polymer and RALA/pEGFP-N1 NPs; Lane 4: Polymer and RALA/pEGFP-N1 NPs decomplexed. Polymeric samples were dissolved in 200 μL water 24 h following incorporation of the NPs. Lysing agent added to the necessary samples following dissolution and 30 μL loaded onto the 1% agarose gel for electrophoresis. Gel images are representative of three independent studies.

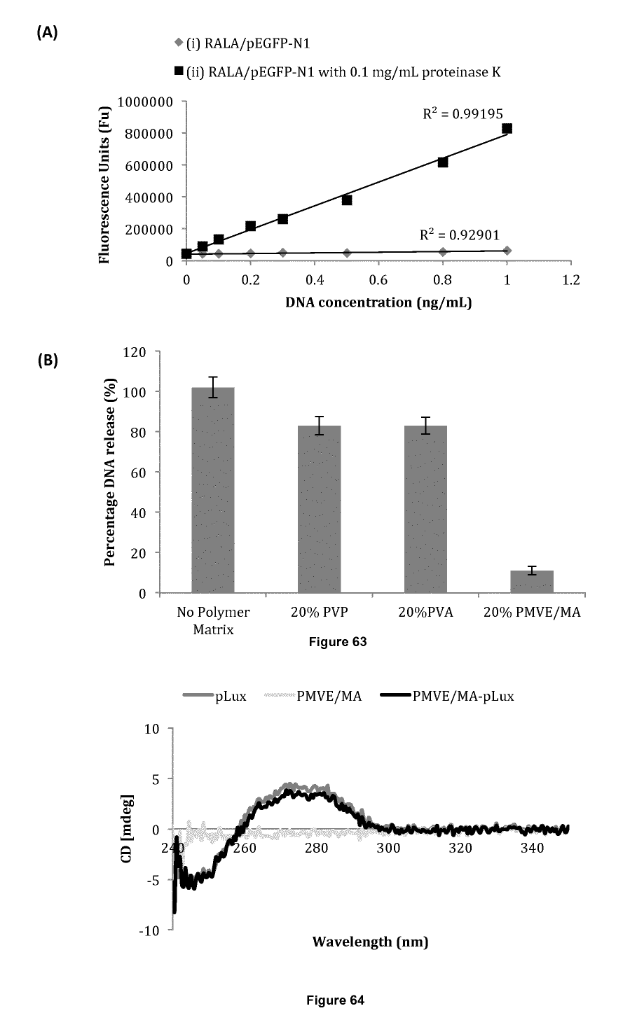

FIG. 63: (A) Standard curve to determine the release of DNA from the RALA/pEGFP-N1 nanoparticles N:P ratio 10 using the Picogreen® reagent; (B) Percentage DNA release 24 h following nanoparticle encapsulation within polymer matrices after addition of 0.1 mg/mL proteinase K. (A) (i) Fluorescence intensity of RALA/pEGFP-N1 nanoparticles N:P ratio 10; (ii) Fluorescence intensity of RALA/pEGFP-N1 nanoparticles N:P ratio 10 following 30 min incubation with 20% proteinase K. (B) 20% PVP, 20% PVA and 20% PMVE/MA polymer matrices encapsulating RALA/pEGFP-N1 nanoparticles containing 1 μg DNA were prepared and dissolved in 10 mM Tris buffer. 0.1 mg/mL proteinase K was subsequently added and samples incubated at 37° C. for 30 mins. Quant-iT™ Picogreen® reagent was then added and the samples analysed by excitation at 480 nm and the fluorescence emission intensity was measured at 520 nm using a spectrofluorometer. The measurements are reported as mean±SEM, (n=3).

FIG. 64: Determination of pDNA secondary structure by circular dichroism. CD spectra were obtained with Jasco J-185 spectopolarimeter equipped with a temperature controller. CD spectra were collected at 20° C. using a 1 cm quartz cell over the wavelength range of 240-350 nm. Samples for CD analysis were prepared by dissolution in PBS. The spectra of PMVE/MA-pLux samples with concentrations of 50 μg/ml were compared with that of pLux and Gantrez only samples. The measurements are reported as n=1.

FIG. 65: WST-1 Cell viability assay following cell exposure to polymer matrices for 6 h. NCTC-929 fibroblast cells were cultured in MEM containing 10% FHS for 24 h. Media was then supplemented with 0, 5, 10 or 20 mg/mL of either 20% PVA, 20% PVP or 20% PMVE/MA and incubated for 6 h at 37° C. and 5% CO₂. Following this incubation 10% WST-1 reagent was added to the media and the cells incubated for a further 2 h. Subsequently the plates were shaken for 1 min and absorbance measured at 450 nm on an EL808 96-well plate reader. The measured absorbance values are expressed as a percentage of the control where the control is defined as 100% viable. The measurements are reported as mean±SEM, (n=3). *p<0.05, **p<0.01, ***p<0.001.

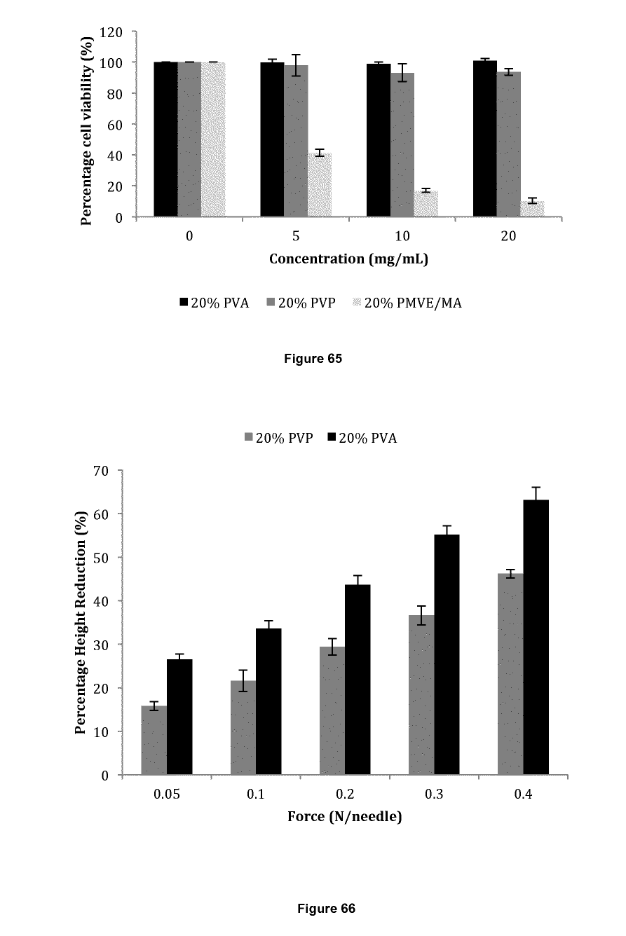

FIG. 66: Percentage reduction in height of MNs fabricated from 20% PVP and 20% PVA following application of increasing axial load. MN arrays of 3×3 MNs were fabricated from 20% PVA and 20% PVP and subjected to axial compression forces of 0.05, 0.1, 0.2, 0.3 and 0.4 N/needle using a TA-XT2 Texture Analyser. Percentage height reductions of MNs were calculated by measuring MN height before and after force application using a GE-5 digital microscope under magnification 180×. The measurements are reported as mean±SEM, (n=3). **p<0.01.

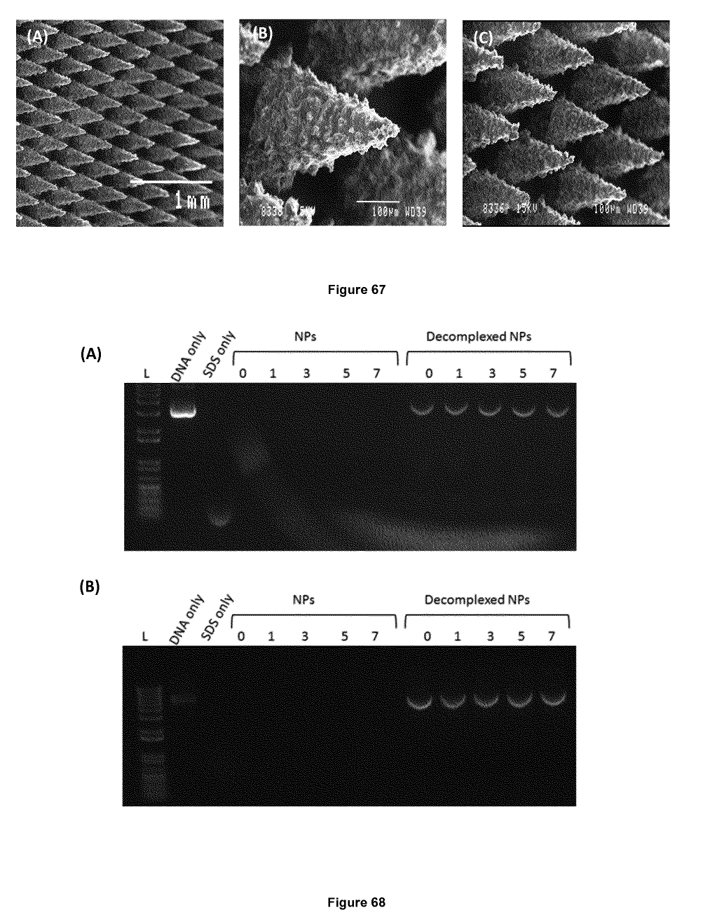

FIG. 67: Scanning electron microscope images of RALA/pEGFP-N1 nanoparticle-containing 20% PVP microneedles. MN arrays containing 10 μg RALA/pEGFP-N1 NPs were mounted onto aluminium stubs, sputter coated with gold and then visualised 24 h later using a JEOL JSM840 scanning electron microscope (JEOL, UK). (A) MN array imaged at ×20 magnification; (B) MN array imaged at ×100 magnification; (C) MN array imaged at ×250 magnification.

FIG. 68: Stability assay of RALA/pEGFP-N1 nanoparticles in 20% PVP for up to 7 days (A) 20° C., 46% RH and (B) 45° C., 75% RH. RALA/pEGFP-N1 nanoparticles N:P ratio 10 containing 1 μg pEGFP-N1 incorporated into 20% PVP were incubated at (A) 20° C., 46% RH and (B) 45° C., 75% RH for 0, 1, 3, 5 and 7 days were analysed via gel electrophoresis. Some samples were decomplexed with SDS for 10 min prior to electrophoresis. Gel images are representative of three independent studies.

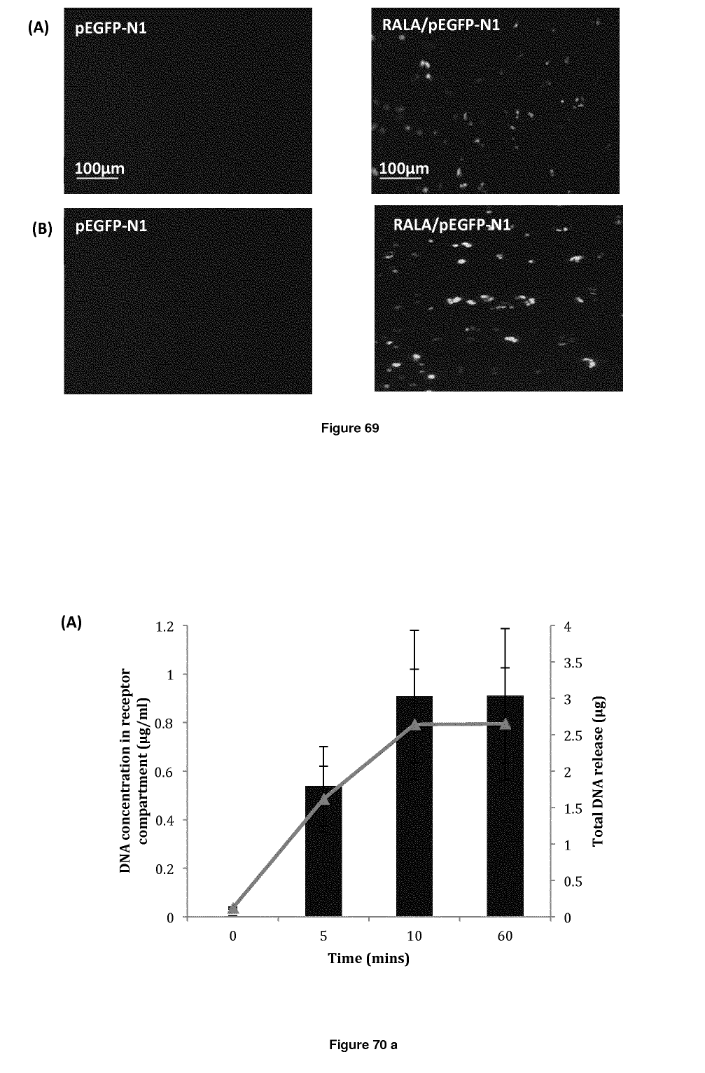

FIG. 69: Fluorescent microscope analysis of GFP expression 24 hours post transfection in NCTC-929 cell line with RALA/pEGFP-N1 nanoparticles released from polymer formulation following (A) 1 h incubation in 20% PVP matrix; (B) 7 day incubation in 20% PVP matrix at room temperature. NCTC-929 cells were conditioned for 2 h in 100 μL Opti-MEM serum free media which was then supplemented with 50 μL RALA/pEGFP-N1 N:P ratio 10 NPs which have been incorporated into the 20% PVP matrix and subsequently dissolved in PBS. Following transfection for 6 h the media was removed and replaced with MEM containing 10% FHS. Images are representative of three independent studies.

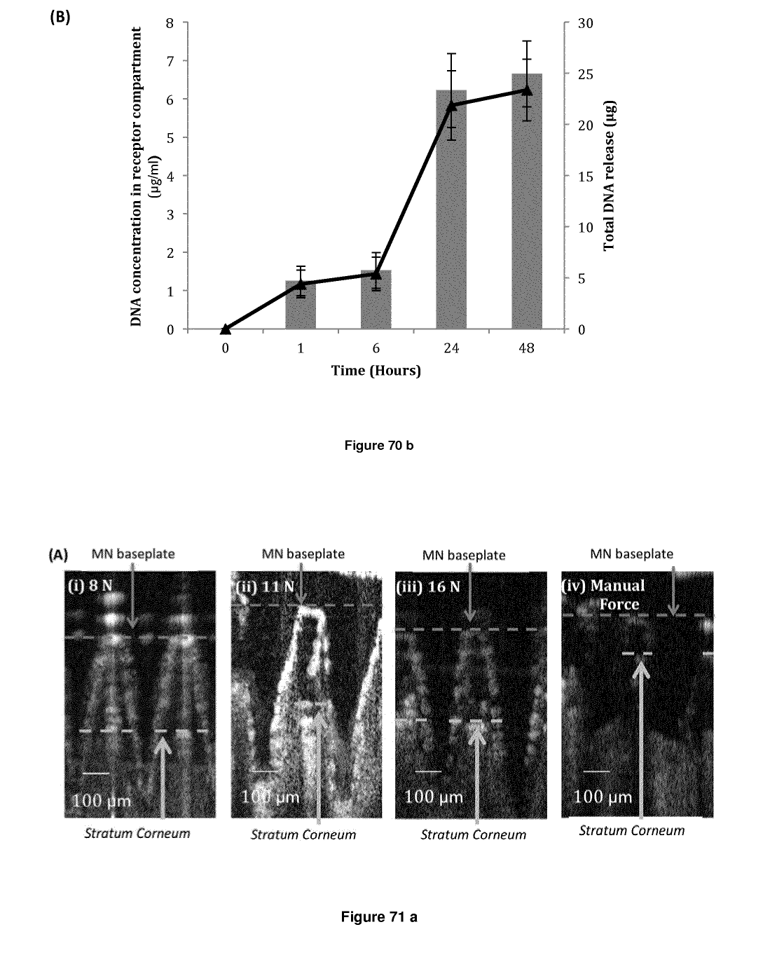

FIG. 70: In vitro release profile of DNA released from NPs encapsulated in 20% PVP MN array across neonatal porcine skin (300 μm) (A) 0-60 mins; (B) 0-48 h. The neonatal porcine skin was sandwiched between a receptor and donor compartment. The MN arrays loaded with RALA/pGFP-N1 N:P 10 nanoparticles containing approx. 27 μg pEGFP-N1 were pressed into the membrane and the membrane then placed in contact with the receptor compartment containing 10 mM Tris buffer. 200 μL samples from the receptor compartment were withdrawn at pre-determined time intervals (0, 5 min, 10 min, 30 min, 1 h, 6 h, 24 h and 48 h) and the volume taken was replaced by the same volume of fresh receptor medium to maintain constant conditions. These samples were then incubated with proteinase K for 30 min, followed by the addition of Picogreen® reagent and the fluorescence intensity of the resulting complexes measured at 520 nm using a spectrofluorometer. The measurements are reported as mean±SEM, (n=3).

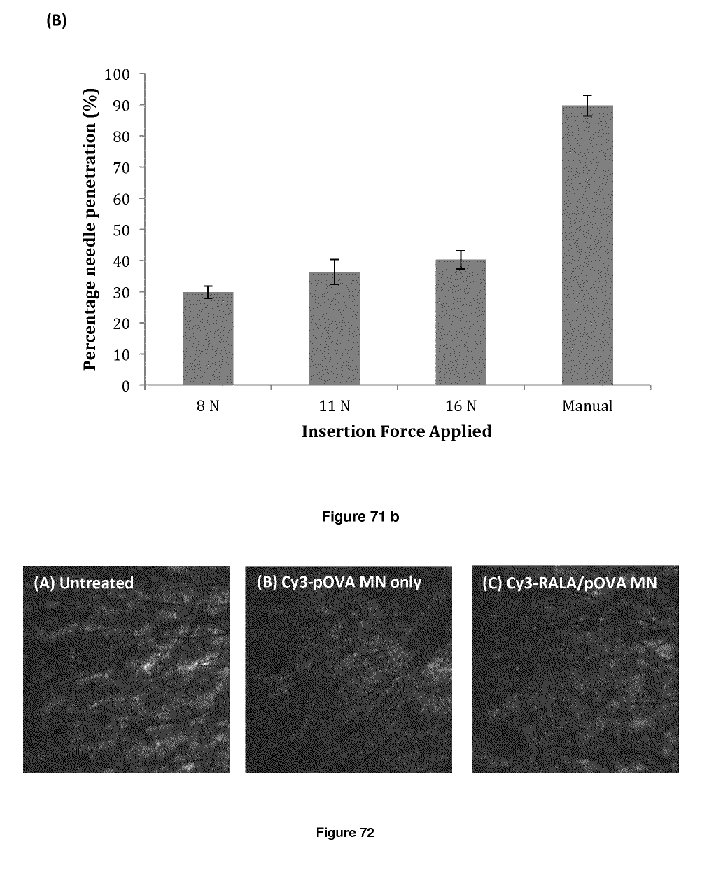

FIG. 71: (A) 2D optical coherence tomography (OCT) image showing 20% PVP polymeric microneedles containing RALA/μLux NPs (approximately 27 μg pLux) inserted into full thickness porcine skin following application with forces of (i) 8N; (ii) 11N; (iii) 16 N; (iv) Manual force; (B) Percentage penetration of 20% PVP containing RALA/μLux NPs (27 μg pLux) inserted into porcine skin following application with forces of 8N; 11N; 16 N; Manual force. Full thickness porcine skin was thawed in PBS for 30 min at 37° C. to restore conditions resembling the in vivo state. MN arrays were then inserted using varying forces and analysed using OCT immediately to assess penetration depth of the MNs. Images were analysed using Image J software. Measurements are reported as mean±SEM, (n=3).

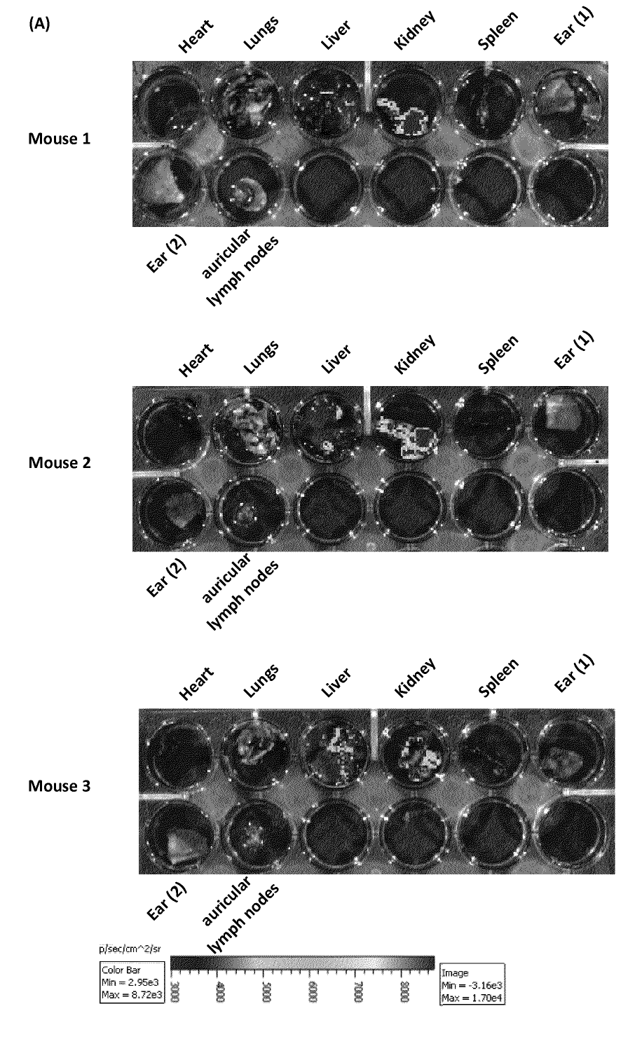

FIG. 72: Confocal microscopic images of (A) untreated mouse ear tissue; (B) mouse ear tissue 1 h following application of 20% PVP MN array loaded with 18 μg Cy-3 labelled pOVA; (C) mouse ear tissue 1 h following application of 20% PVP MN array loaded with Cy-3 labelled RALA/pOVA NPs containing 18 μg DNA. MNs were applied to the ear of C57BL/6 mice and left in situ for 1 h. Following this the mice were sacrificed and the ear tissue removed and stored in 4% formaldehyde solution. Ear tissue was then mounted onto a microscope slide using 100% glycerol and analysed using a TCS SP5-Leica Microsystems confocal microscope using the 10× objective.

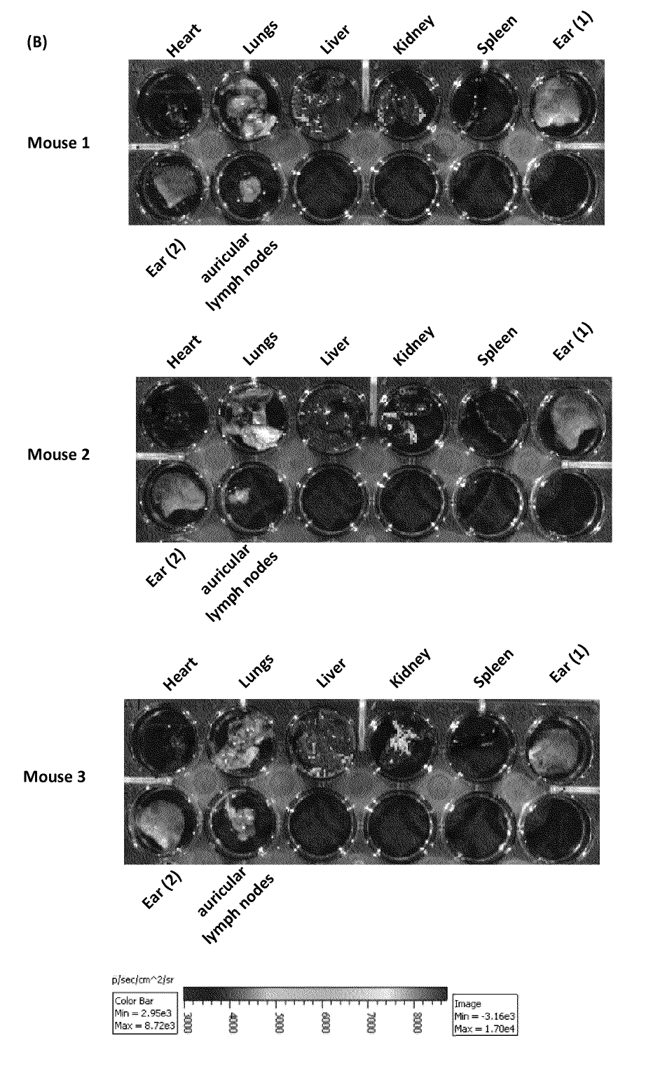

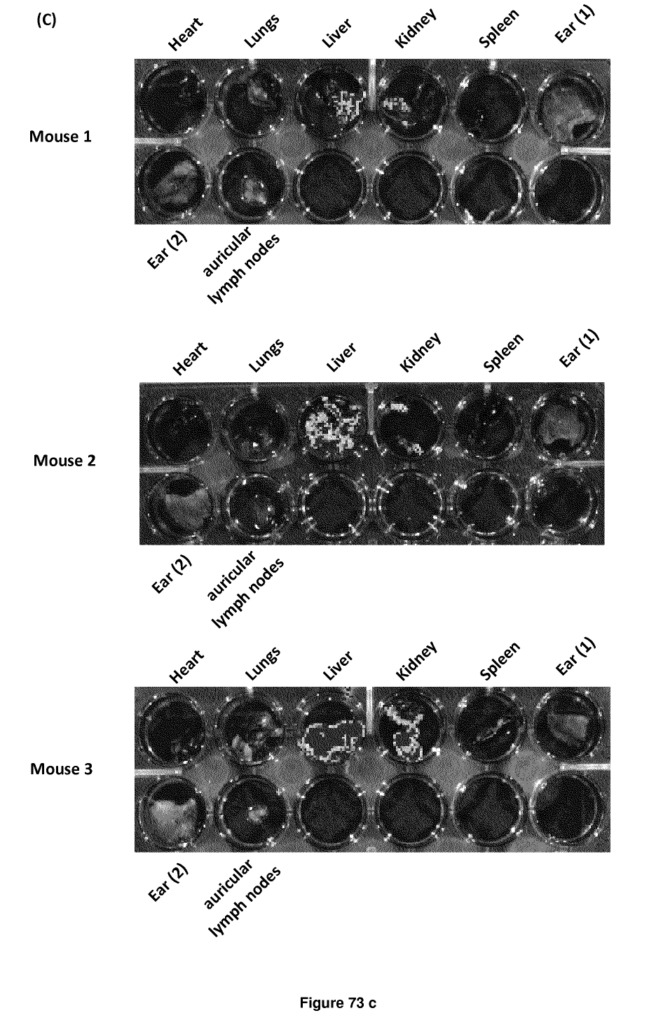

FIG. 73: IVIS detection of luciferase expression (A) 6 h, (B) 24 h and (C) 48 h following application of MN arrays encapsulating RALA/μLux NPs containing approx. 27 μg of DNA. C57BL/6 mice were treated via the application of 1 20% PVP MN per ear loaded with RALA/μLux NPs. 3 mice were sacrificed 6 h post application and their organs, ear tissue and auricular lymph nodes removed and bathed in D-Luciferin Potassium Salt in PBS (15 mg/mL) for 10 min and then imaged using the Xenogen IVIS 200 Imaging System. This was repeated at 24 h and 48 h post MN application.

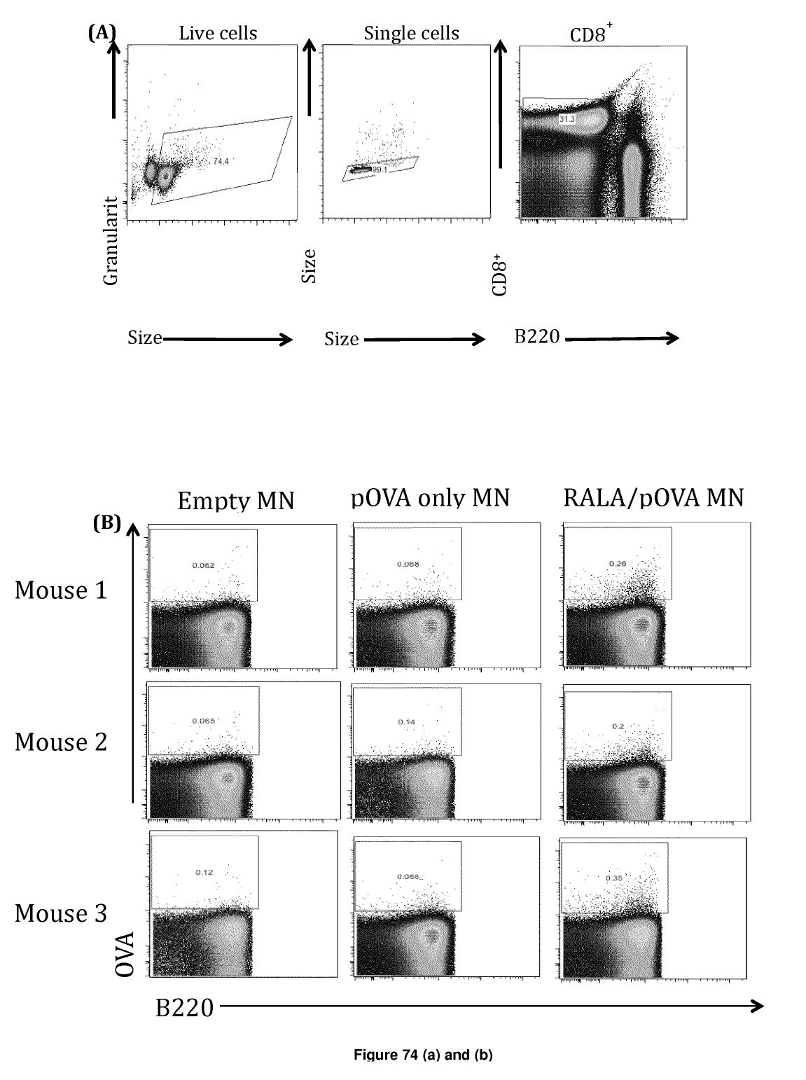

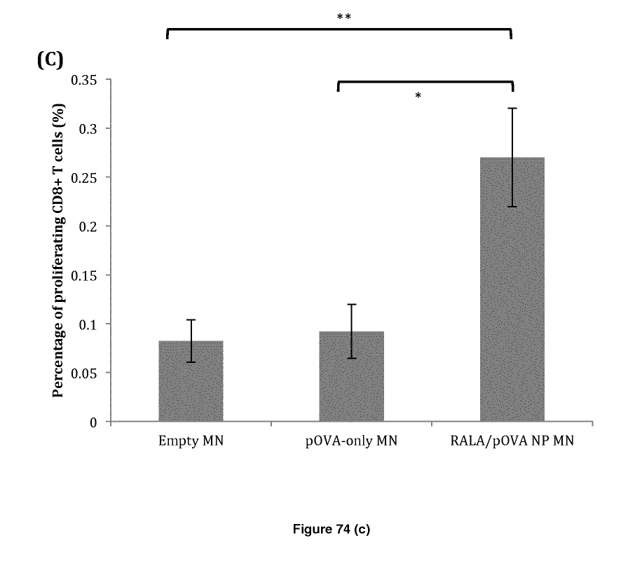

FIG. 74: Flow cytometric analysis of OVA-specific CD8⁺ T-cells detected 10 days post microneedle immunization with pOVA and RALA/pOVA nanoparticles. (A) Flow cytometry dot plot illustrating back-gating strategy for detection of OVA-specific CD8⁺ T-cells isolated from mouse auricular lymph nodes; (B) Dot plot analyses showing the percentage of OVA-specific CD8⁺ T-cells among the CD8⁺ T-cells isolated; (C) Percentage of proliferating CD8⁺ T-cells which express OVA specific T-cell receptors on their surface. Measurements are reported as mean±SEM, (n=3). *p<0.05, **p<0.01.

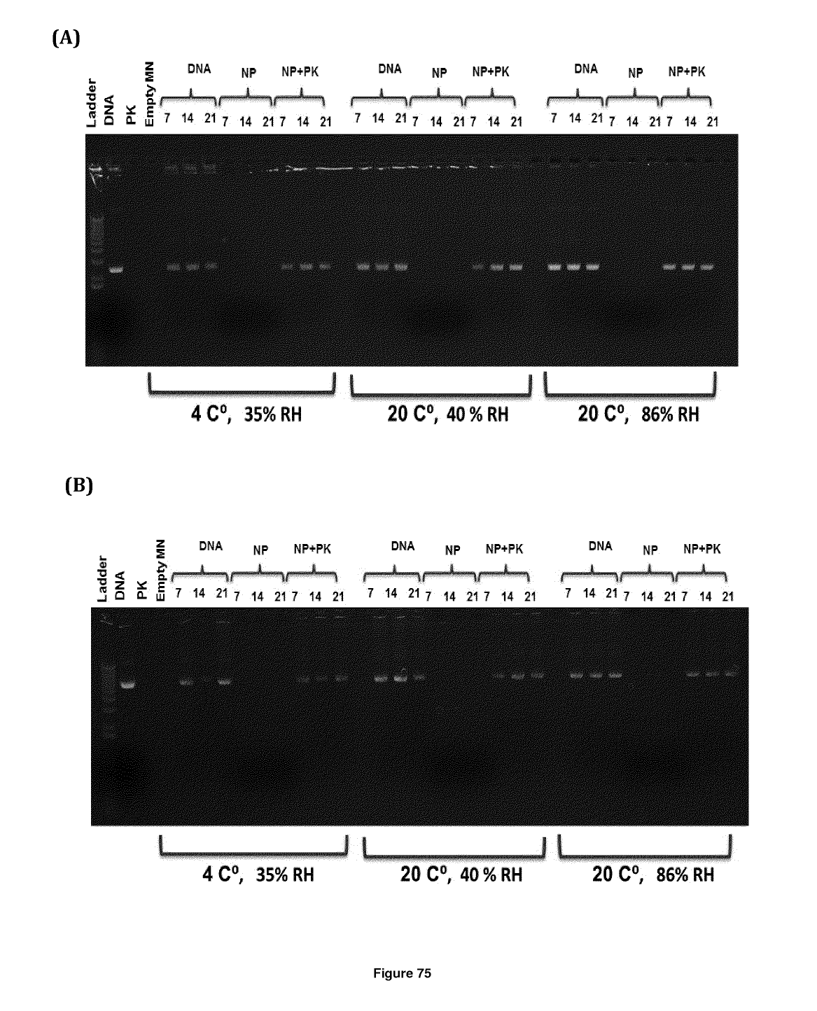

FIG. 75: Agarose gel determination of RALA/HPV-16 E6 and RALA/HPV-16 E7 NP stability in 20% PVP polymer matrix for up to 21 days. (A) RALA/HPV-16 E6; (B) RALA/HPV-16 E7 NPs loaded-20% PVP microneedles for up to 21 days. Microneedles were incubated either at 4° C., 35% relative humidity (RH) or 20° C., 40% RH, or 20° C., 86% RH for 7, 14, or 21 days. Microneedles were dissolved and RALA/HPV-16 E6 or RALA/HPV-16 E7 nanoparticles N:P ratio 10 containing 1 μg HPV-16 E6 or HPV-16 E7 DNA were analysed via gel electrophoresis. Some samples were de-complexed with proteinase K for 30 min prior to electrophoresis. Gel images are representative of three independent studies.

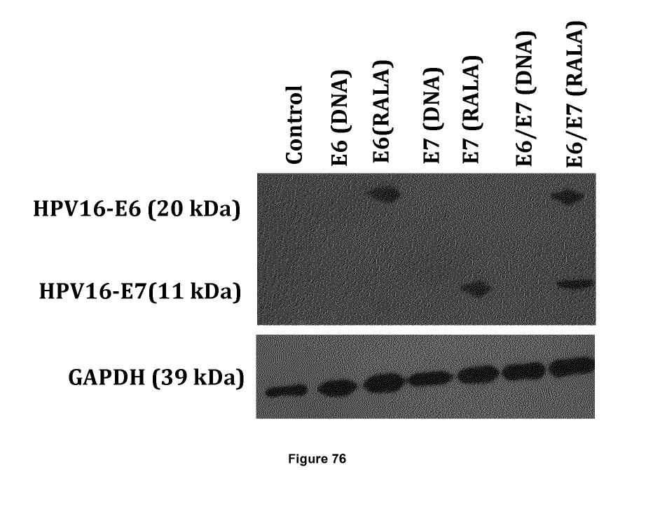

FIG. 76: Determination of functionality of RALA/HPV-16 E6, RALA/HPV-16 E7, and RALA/HPV-16 E6/E7 N:P ratio 10 NPs encapsulated within 20% PVP matrix. Western blot analysis of HPV-16 E6, HPV-16 E7, and HPV-16 E6/E7 protein levels. NCTC-929 cells were transfected with RALA/HPV-16 E6, RALA/HPV-16 E7, and RALA/HPV-16 E6/E7. 24 h post transfection, cell lysates were electrophoresed through a SDS-PAGE and transferred onto nitro-cellulose membrane according to standard procedures. The membrane was probed with both HPV-16 E6 and E7 antibodies and developed using chemiluminesce kit. The blot is representative of 3 independent experiments.

FIG. 77 is an elevational view in partial cross-section of a cross-linked array of micro protrusions as constructed using the principles of the present invention and forming part of a transdermal delivery system for the delivery of a beneficial substance (11—protective backing layer, 12—reservoir containing beneficial substance as either a solution or suspension in a pharmaceutical vehicle, 13—cross-linked array of micro protrusions, 14—stratum corneum of skin, 15—viable epidermis);

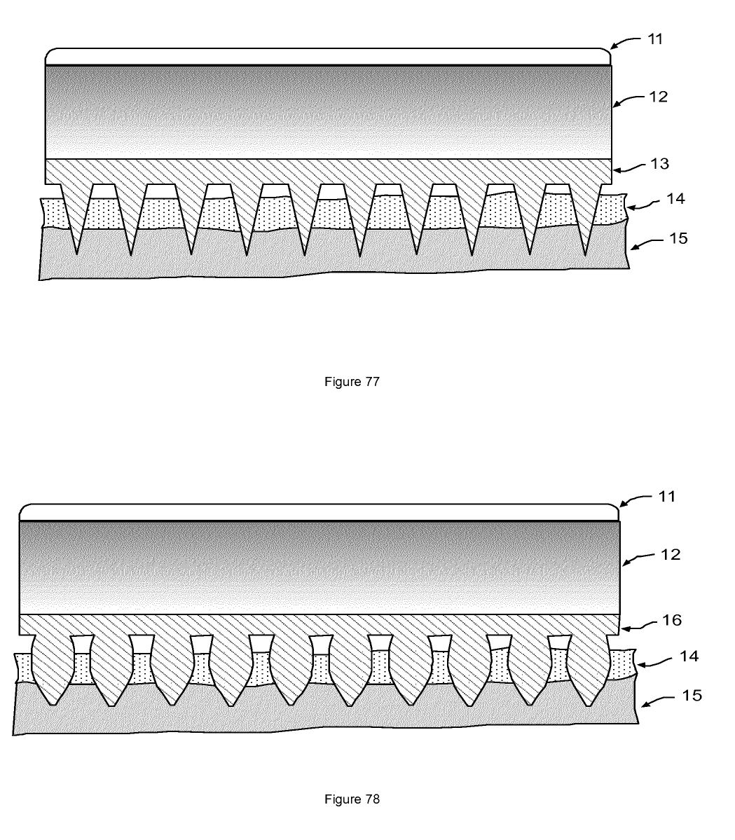

FIG. 78 is an elevational view in partial cross-section of a cross-linked array of micro protrusions as constructed using the principles of the present invention and shown in FIG. 7 that have undergone a hydration-induced alteration of physical attributes (11—protective backing layer, 12—reservoir containing beneficial substance as either a solution or suspension in a pharmaceutical vehicle, 16—cross-linked array of micro protrusions that have undergone an hydration-induced alteration in geometric shape and alteration in substance diffusivity, 14—stratum corneum of skin, 15—viable epidermis).

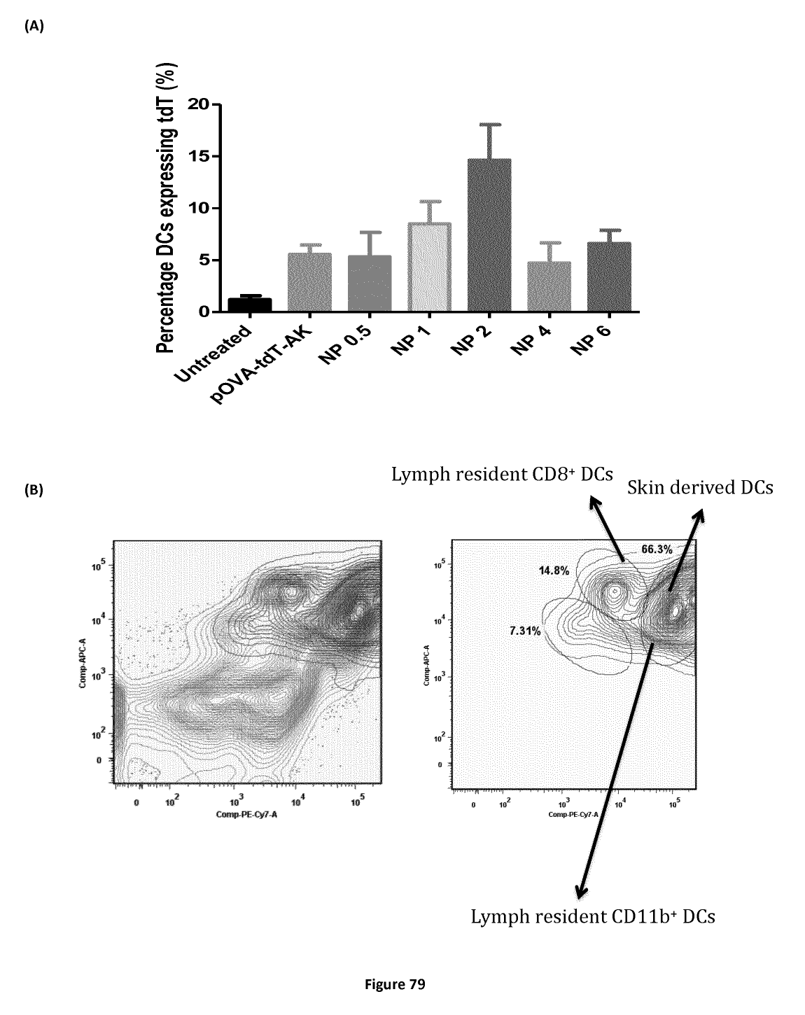

FIG. 79: Flow cytometric analysis of tdT expression of dendritic cells (DCs) detected 4 days post microneedle (MN) delivery of pOVA-tdT-AK and RALA/pOVA-tdT-AK nanoparticles. (A) Percentage DCs isolated from skin draining lymph nodes expressing tdT 4 days post MN delivery to the back of C57/BL6 mice. The measurements are reported as mean±SEM (n=4 untreated, pOVA-tdT-AK, NP 0.5, NP 1, NP 2, NP 4; n=2 NP 6); (B) Analysis of the DC populations positive for tdT expression i.e. have taken up the DNA cargo and expressed the reporter gene encoded.

FIG. 80: ELISA analysis of serum E6/E7-specific IgG levels 10 days post 2^{nd} and 3^{rd} immunizations in C57BL/6 mice. (A) Example image of ELISA plate illustrating colour change (yellow to orange) indicating presence of E6/E7-specific IgG antibodies; (B) Quantification of serum levels of E6/E7-specific IgG antibodies present 10 days following 2^{nd} and 3^{rd} immunizations. The measurements are reported as mean±SEM (n=4).

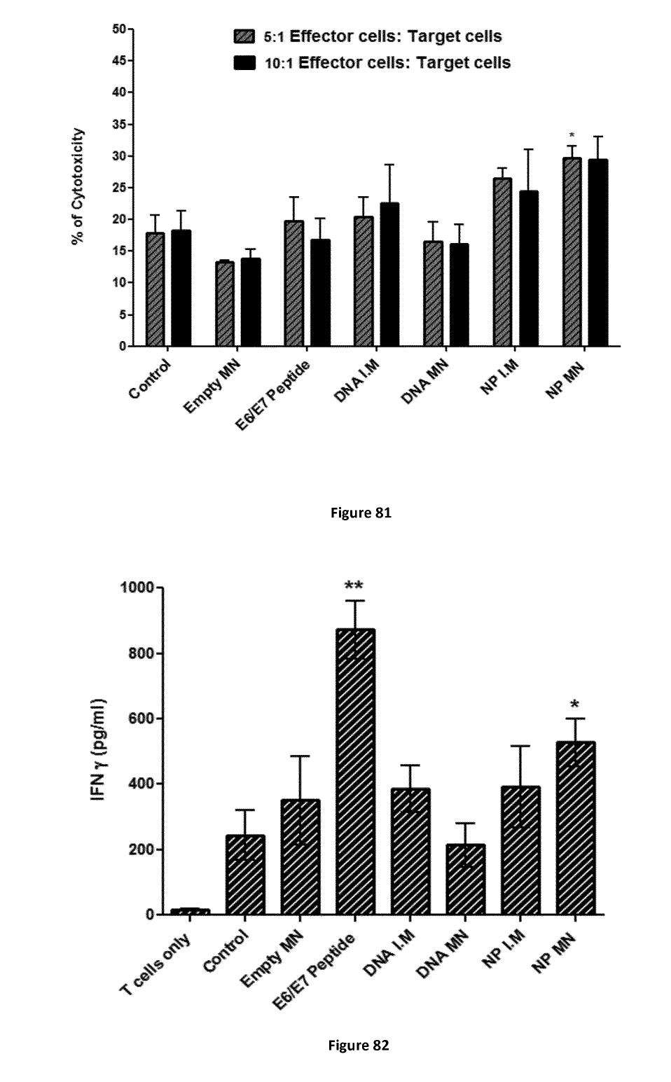

FIG. 81: Cytotoxicity of spleen-resident T cells in immunized C57BL/6 mice against E6/E7-expressing TC-1 cells ex vivo 10 days post 3^{rd} immunisation. Ratios 5:1 and 10: of the effector T cells to the target TC-1 cells were investigated. Isolated T cells were cultured with irradiated TC-1 cells for 6 days and subsequently incubated with healthy TC-1 cells for 5 h. The cytotoxicity was measured via a cytotoxicity detection kit. The measurements are reported as mean±SEM (n=4).

FIG. 82: ELISA determination of IFN-γ release from splenocytes isolated from immunised C57BL/6 mice restimulated ex vivo with E6/E7-expressing TC-1 cells. The measurements are reported as mean±SEM (n=4).

FIG. 83: (A) Kaplein Meier survival plot of immunised C57BL/6 mice post TC-1 tumour cell challenge. Mice were immunised, followed by two boosts, each two weeks apart. Mice were then challenged with 1×10⁵ TC-1 tumour cells. E6/E7-expressing tumour cells implanted intradermally on the rear dorsum. (B) Plots illustrating TC-1 cell tumour quadrupling time in immunised C57BL/6 mice following cell implantation. 50 μg of E6/E7 peptides were used as a positive control (n=9).

FIG. 84: Therapeutic response of E6/E7 vaccination. TC-1 tumours were implanted on the rear dorsum of C57/BL6 mice. When tumours reached 100 mm³, mice were vaccinated three times, one week apart. Tumour weight measurements and images illustrating differences in tumours among C57BL/6 mice treated with naked 100 μg E6/E7 DNA, or RALA/HPV-16 E6/E7 NPs delivered I.M. or via MN patches 21 days post tumuor challenge. The measurements are reported as mean±SEM (n=3).

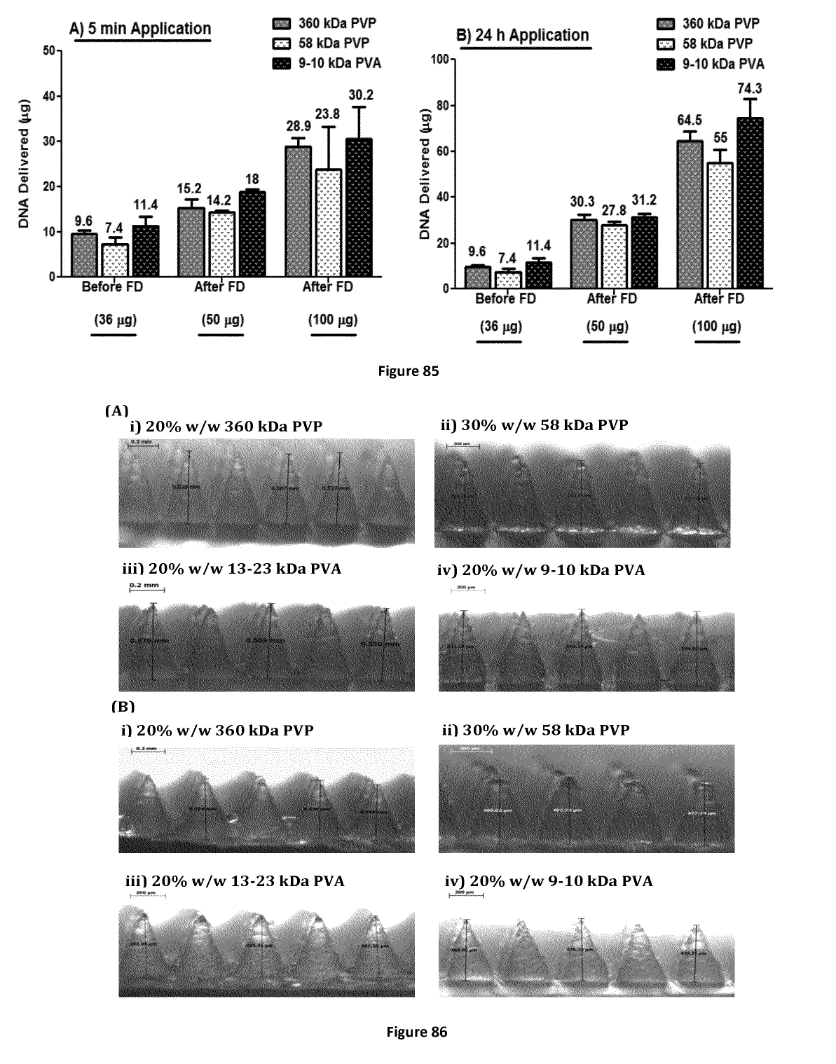

FIG. 85: Quantification of HPV-16 E6/E7 DNA delivered in vivo following application of polymeric MN arrays for (A) 5 min and (B) 24 h to C57BL/6 mice ears. MN arrays were formulated to contain either 36, 50, or 100 μg pHPV-16 E6/E7. Studies were performed both before and after freeze-drying (FD). The measurements are reported as mean±SEM (n=3).

FIG. 86: Sideview of dissolving polymer microneedle patches loaded with RALA/DNA nanoparticles (A) prior to compression and (B) following 45 N compression. Microneedles were fabricated by dilution of RALA/pDNA nanoparticles with concentrated polymer stock to give i) 20% w/w 360 kDa PVP ii) 30% w/w 58 kDa PVP iii) 20% w/w 13-23 kDa PVA and iv) 20% w/w 9-10 kDa PVA polymer/pDNA blends. Needles were imaged using a GXMGE-5 digital microscope×35 magnification. Following imaging arrays were adhered to the moveable arm of a TA-XT2 Texture Analyser and subjected to 45 N axial compression for 30 sec. Following compression needles were imaged as above. Each image is a representative image of three independent studies.

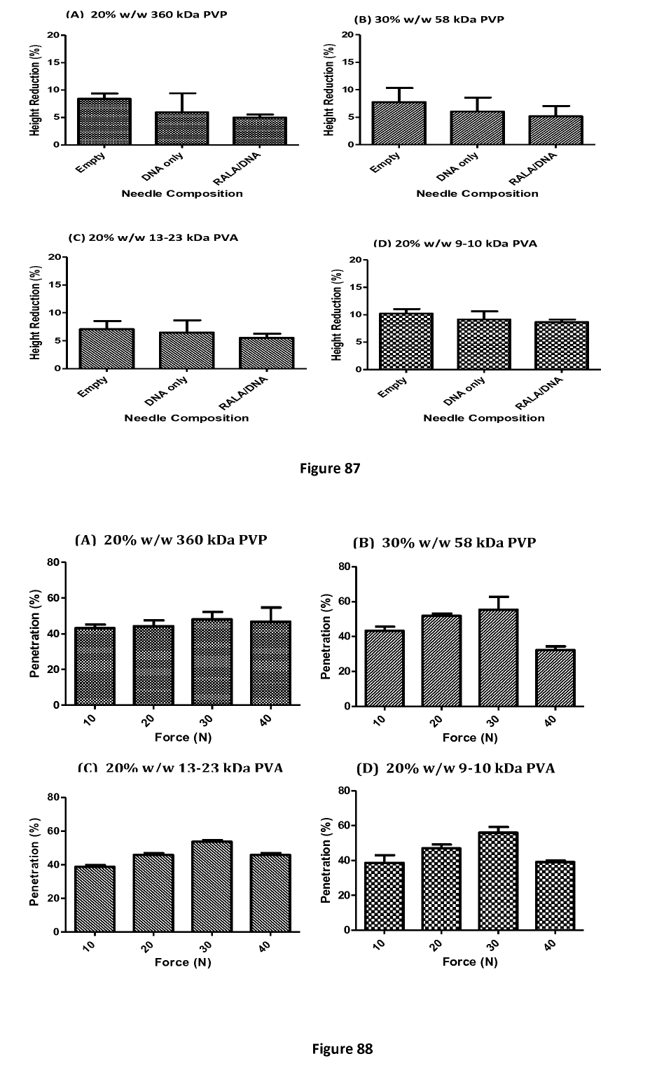

FIG. 87: Percentage Height Reduction of Polymeric Microneedles following application of a 45 N axial force. Microneedles arrays of 19×19 needles were fabricated from (A) 20% w/w 360 kDa PVP; (B) 30% w/w 58 kDa PVP; (C) 13-23 kDa PVA and (D) 9-10 kDa PVA stock solution or stock solution loaded with pDNA or RALA/pDNA solution. Needles were imaged and measured using a digital microscope and subjected to a compression force of 45 N (0.125 N/needle) for 30 sec using the TA-XT2 Texture Analyser. Following compression arrays were re-imaged and measured and percentage height reduction calculated. Measurements reported as mean±SEM (n=3).

FIG. 88: Percentage penetration of polymers inserted into mouse ears following application of forces 10 N; 20 N; 30 N and 40 N. 19×19 MN arrays were fabricated from 20% w/w (A) 360 kDa PVP; (B) 30% w/w 58 kDa PVP; (C) 13-23 kDa PVA and (D) 9-10 kDa PVA. Mouse ears were equilibrated in PBS for 30 min at 37° C. to restore conditions resembling the in vivo state. Arrays were then inserted into mouse ears with a TA-XT2 Texture Analyser using varying forces (10-40 N) for 30 sec and analysed using Optical Coherence Tomography (OCT) immediately to assess penetration depth of the MNs. Images were analysed using Image J software. Measurements are reported as mean±SEM, (n=3).

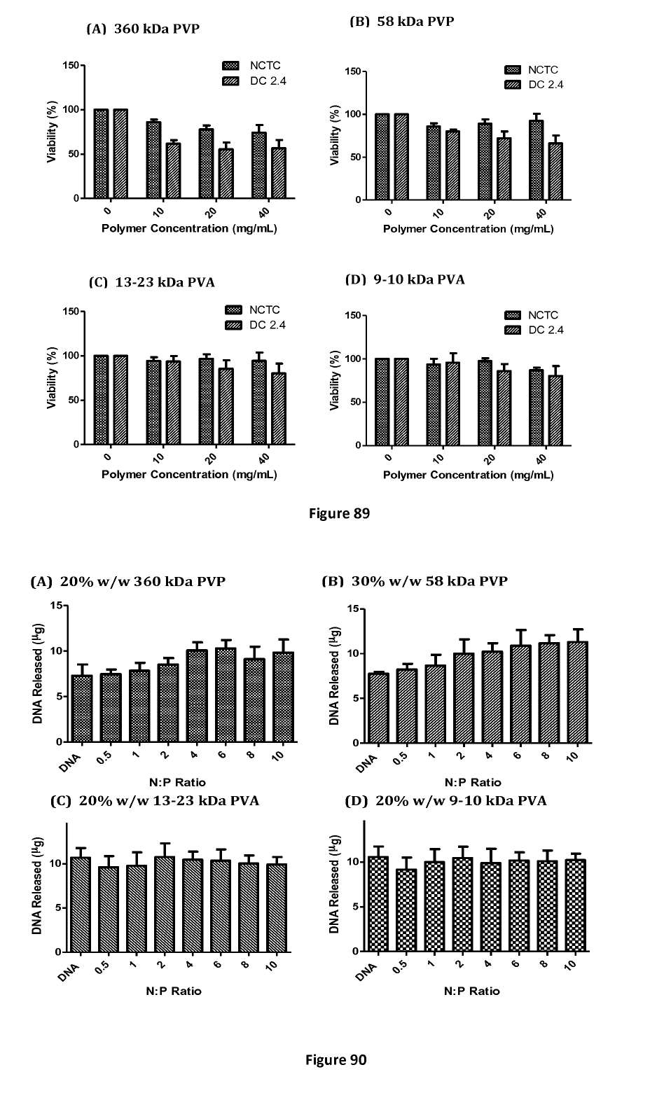

FIG. 89: MTS cell viability assay following cell exposure to polymer matrices for 24 h. Cells were cultured in serum supplemented media for 24 h. Media was then supplemented with 0, 10, 20 or 40 mg/mL of either (A) 360 kDa PVP; (B) 58 kDa PVP; (C) 13-23 kDa PVA or (D) 9-10 kDa PVA and incubated for 24 h at 37° C. and 5% CO₂. Following incubation 10% MTS reagent was added to the media and the cells incubated at 37° C. for a further 2 h. Subsequently absorbance measured at 490 nm on a EL808 96-well plate reader. The measured absorbance values are expressed as a percentage of the control where the control is defined as 100% viable. The measurements are reported as mean±SEM, (n=3).

FIG. 90: Quantity of plasmid DNA released from polymer gels dissolved in Tris Buffer. 250 mg polymer gels incorporating 10 μg of plasmid DNA were prepared by diluting concentrated polymer stock 3:2 with RALA/pDNA (N:P ratios 0-10) and drying at room temperature for 48 h. Gels were dissolved in 7 mL Tris buffer and 50 μL samples were plated in triplicate into a black 96 well plate, incubated with 50 μL 0.2 mg/mL Proteinase K for 120 min at 37° C. and then incubated with 50 μL Quanti-iT Picogreen™ reagent for 30 min. Samples were then excited at 480 nm and fluorescent emission at 520 nm was measured using a Synergy 2 Multi-Mode Microplate Reader (BioTek Instruments Inc, UK). Measurements reported as mean±SEM (n=3).

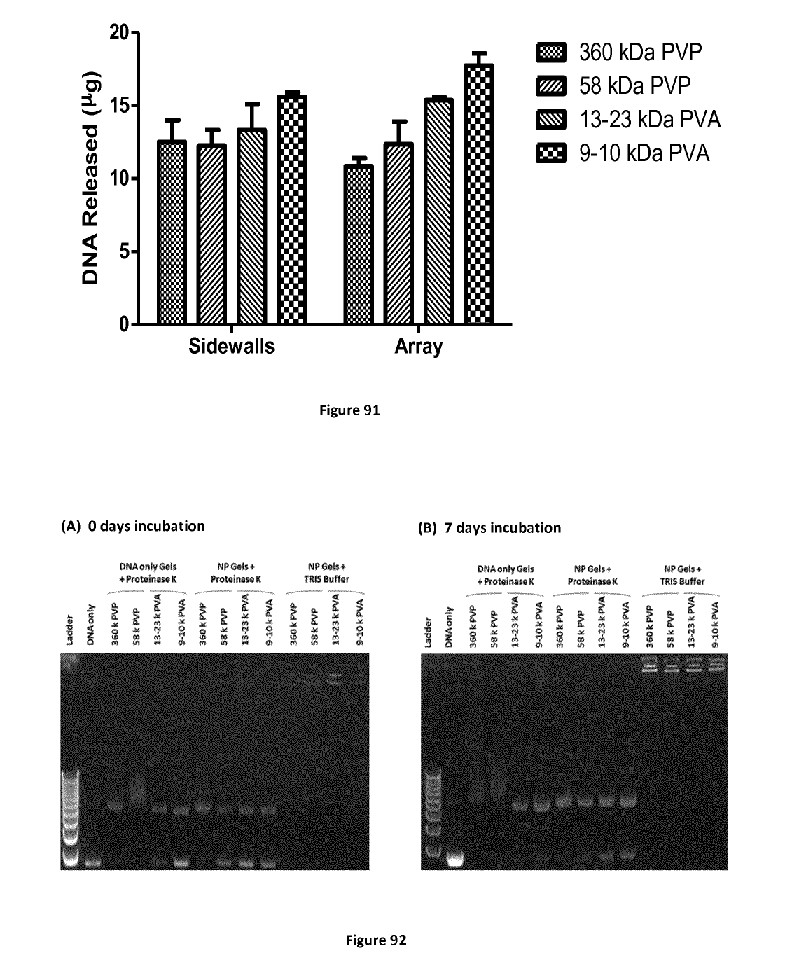

FIG. 91: Quantity of plasmid DNA released from MN arrays dissolved in Tris Buffer. 20% w/w 360 kDa PVP, 13-23 kDa and PVA, 9-10 kDa PVA stock solution or 30% w/w 58 kDa PVP. 19×19 MN arrays containing 32 μg pDNA were fabricated by dilution of concentrated 50% w/w 360 kDa PVP, 363 kDa PVA, 9-10 kDa PVA stock solution stock to 20% w/w or by dilution of concentrated 75% w/w 58 kDa PVP stock solution stock to 30% w/w RALA/pDNA solution (N:P ratio 10). Sidewalls were removed from arrays using a heated scalpel and analysed separately from the baseplate and needles (arrays). Arrays and sidewalls were dissolved in 7 mL Tris buffer and 50 μL samples were plated in triplicate into a black 96 well plate, incubated with 50 μL 0.2 mg/mL Proteinase K for 120 min at 37° C. and then incubated with 50 μL Quanti-iT Picogreen™ reagent for 30 min. Samples were then excited at 480 nm and fluorescent emission at 520 nm was measured using a Synergy 2 Multi-Mode Microplate Reader (BioTek Instruments Inc, UK). Fluorescence was used to determine the concentration of pDNA in solution using a standard curve. Measurements reported as mean±SEM (n=3).

FIG. 92: DNA integrity study following incubation of plasmid DNA in polymer gels±RALA for (A) 0 or (B) 7 days. Concentrated polymer stock was diluted with 30 μg of RALA/pDNA (N:P ratio 0 or 6) and left to dry overnight. Gels were dissolved in Tris buffer. Samples containing DNA only (Lanes 3-6) were incubated with Proteinase K for 2 h at 37° C. Samples containing RALA/pDNA (N:P ratio 6) were incubated with Proteinase K (Lanes 7-10) or Tris buffer (Lanes 11-14) for 2 h at 37° C. Following incubation, 20 μL samples were electrophoresed on a 1% agarose gel containing 0.2 μg/mL EtBr as a visualising agent. A 100 V current was applied to the gel for 1 h and the gel was then imaged under UV light. Each gel is a representative image of three independent studies.

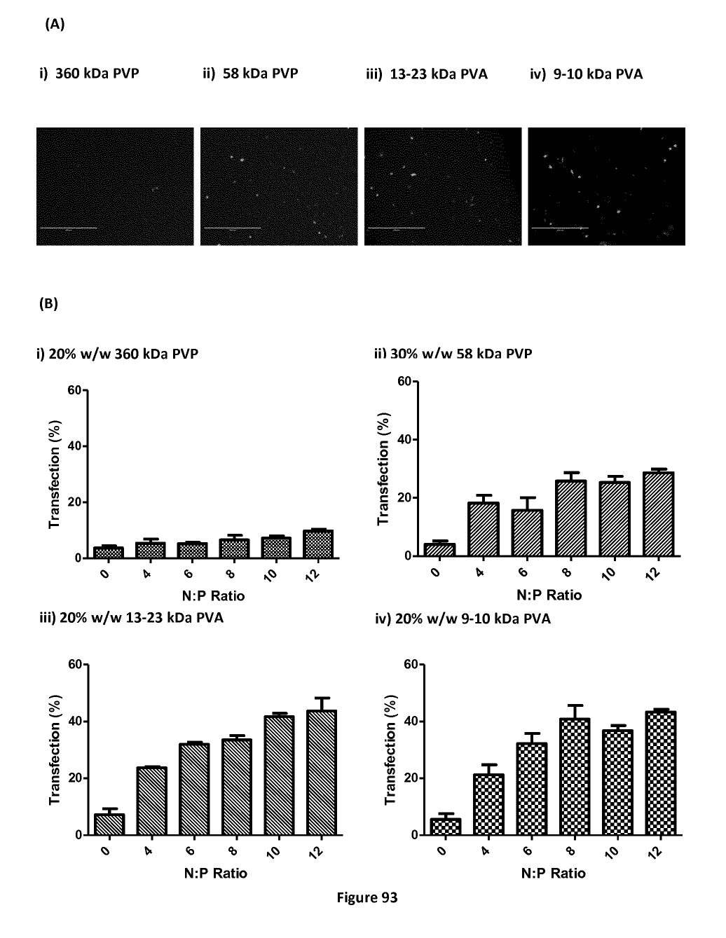

FIG. 93: (A) Fluorescent microscope images and (B) Flow cytometric analysis of GFP expression 48 h post transfection with RALA/pEGFPN-1 complexes following dissolution of polymeric gels in the NCTC 929 fibroblast cell line. Gels were fabricated by dilution of concentrated polymer with RALA/pEGFPN-1 complexes. Following drying overnight, gels were dissolved in 1 mL OptiMEM for 1 h. Following dissolution, 250 μL of OptiMEM was added to cells and incubated for 4 h under standard tissue culture conditions. Following incubation OptiMEM was removed and cells were incubated in complete media. Fluorescent microscope images were taken 48 h post transfection. Each image is a representative image of three independent studies (N:P ratio 12). Following imaging cells were harvested for flow cytometric analysis. Measurements are reported as mean±SEM, (n=3).

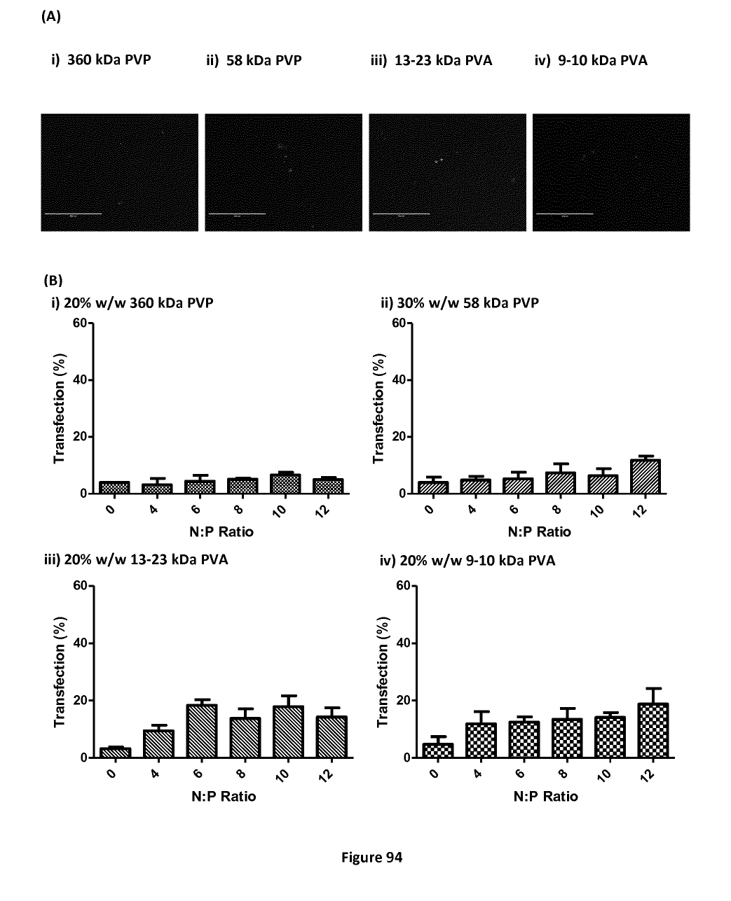

FIG. 94: (A) Fluorescent microscope images and B) Flow cytometric analysis of GFP expression 48 h post transfection with RALA/pEGFPN-1 complexes following dissolution of polymeric gels in the RAW 264.7 cell line. Gels were fabricated by dilution of concentrated polymer with RALA/pEGFPN-1 complexes. Following drying overnight gels were dissolved in 1 mL Opti-MEM for 1 h. Following dissolution, 250 μL of Opti-MEM was added to cells and incubated for 4 h under standard tissue culture consitions. Following incubation Opti-MEM was removed and cells were incubated in complete media. Fluorescent microscope images were taken 48 h post transfection. Each image is a representative image of three independent studies (N:P ratio 12). Following imaging cells were harvested for flow cytometric analysis. Measurements are reported as mean±SEM, (n=3).

### EXAMPLE 1: GENERATION OF CELL PENETRATING AMPHIPATHIC PEPTIDE—“RALA” PEPTIDE

The following peptide (called “RALA” herein) was synthesised commercially in accordance with conventional techniques with the amino acid sequence
WEARLARALARALARHLARALARALRACEA

RALA arrives in a lyophilised form and is reconstituted with molecular grade water to a desired concentration, aliquotted out and stored at −20° C. until further use. An aliquot is then taken as needed and defrosted on ice.

### EXAMPLE 2: FORMATION AND IN-VITRO/IN-VIVO TESTING OF RALA/SIRNA NANOPARTICLES

### Materials and Methods

### Calculation of N:P Ratio

DNA was complexed with either the RALA peptide at various N:P ratios (the molar ratio of positively charged nitrogen atoms to negatively charged phosphates in DNA). As the number of positive side-groups in a protein side chain depends upon the sequence, different proteins will have differing numbers of positive charges per unit mass. In order to calculate this, the following equation was used:

NP=Mₚᵣₒₜₑᵢₙ/M_{DNA}C_{NP}

Where M protein is the mass of a protein, M DNA is the mass of DNA and C NP is the N:P constant. The N:P constant is the ratio of the protein's side chain positive charge density to the DNAs backbone density, with the charge density being the charge of a substance divided by its molecular mass. For the protein, lysine, arginine and histidine side groups are counted. For the DNA the average mass of one single base pair, and the charge of the phosphate group are used. For RALA an N:P ratio of 1 is 1.45 μg of RALA: 1 μg of DNA.

### Formation of the Nanoparticles

The DNA/siRNA was diluted in molecular grade water to 200 μg/ml. 1 μg of DNA was added to a 1.5 ml eppendorf centrifuge tube. For 1 μg of DNA the final volume was 50 μl. The appropriate volume of protein to use to make the desired N:P ratio was added to a separate tube and the volume made up to 50 μl with molecular grade water. The 50 μl solution containing the protein was added to the 50 μl containing the DNA. The molecular grade water was added to the DNA before the protein. The tube was flicked five times in order to mix the content. The complexes were allowed to incubate for 30 minutes at room temperature prior to use. The results are shown in FIGS. 2 & 28.

### Gel Retardation Assay

RALA/DNA complexes were prepared at N:P ratios 1-15. Following incubation at room temperature for 30 minutes, 30 μL of the samples (corresponding to 0.6 μg of DNA) were electrophoresed through a 1% agarose gel containing 0.5 μg/mL ethidium bromide (EtBr) (Sigma, UK) to visualize DNA. A current of 80 V was applied for 1 h and the gel imaged using a Multispectrum Bioimaging System (UVP, UK). The purpose of this assay is to determine which N:P ratio/s neutralise the DNA. The assay works upon the principle that when complexes are formed with an excess positive charge DNA remains in the wells or migrates up the gel, hence, no DNA band will be visible following gel electrophoresis. However, DNA alone or complexed to give a net negative charge will migrate down the gel (FIG. 27).

### Nanoparticle Size and Charge Analysis

In order to obtain particle size and charge distributions the mean hydrodynamic particle size measurements RALA complexes were performed using Dynamic Light Scattering (DLS). Dynamic Light Scattering is based upon the principle that when particles are illuminated with a laser, due to Brownian motion there will be scattering of the light. The intensity of the scattered light fluctuates as a result of this Brownian motion caused by bombardment of the particles by solvent molecules. A correlation curve reflecting the decay rate is generated based on fluctuations of the scattered light where a slower correlation decay rate represents a slower moving particle. Based on the Stokes-Einstein equation larger particles move more slowly and, thus, the correlation function can be used to determine the size distribution of the particles. dynamic light scattering (DLS) was used.

Surface charge measurements of the RALA nanoparticles were determined by Laser Doppler Velocimetry. The zeta potential of the particles was measured using disposable foltable zeta cuvettes. Zeta cuvettes for the measurement of zeta potential were first washed with 70% ethanol, followed by two rinses with double distilled H₂O prior to loading the sample. Enough diluted sample used for size measurement was used for determination of zeta potential.

The nanoparticles were made up at an appropriate range of N:P ratios with at least using 2 μg of DNA in each sample. Nanoparticles were analysed using either and analysis was completed on either the Zetasizer-HS3000 (Malvern Instruments) or the Zetasizer-Nano instrument with DTS software (Malvern Instruments, UK). Zetasizer-Nano (Malvern Instruments) (FIGS. 3, 9, 17-21, 41, 48 & 55).

### Incubation Stability Study of RALA Nanoparticles

This assay is designed to illustrate the stability of RALA complexes to indicate the optimal time period for nanoparticle formation. Following incubation at room temperature for 30 min the mean hydrodynamic size and zeta potential were measured using the Malvern Zetasizer NanoZS with DTS software at 15 or 30 min intervals over a period of 360 min. Size and zeta potential are reported as mean±SEM, n=3, where n represents the number of independent batches prepared for measurement (FIGS. 29b, & 43).

### Temperature Stability Study of RALA Complexes

This assay determines the stability of the nanoparticles over a range of temperatures. Following preparation of the nanoparticles by incubation at room temperature for 30 min the mean hydrodynamic size and zeta potential were measured over a temperature range of 4-37° C. in 4° C. intervals using the Malvern Zetasizer NanoZS with DTS software. The sample was allowed to equilibrate at each temperature for 120 sec before measurements were taken in triplicate. Results are reported as mean±SEM, n=3, where n represents the number of independent batches prepared for measurement (FIG. 29a, 42, 49).

### Serum Stability Assay

In order to determine the stability of the RALA nanoparticles when exposed to serum the following procedure was carried out. Six replicates of the complexes at NP ratios 5, 10 and 15 were made. Each N:P ratio was split into 3 aliquots or in the case of RALA 18 aliquots. 10% foetal calf serum was added to 12 of the aliquots. The 18 aliquots were incubated at 37° C. Every 55 min SDS (sodium dodecyl sulphate (Sigma, UK)) was added to one of aliquots containing serum for each N:P ratio which were then incubated for a further 5 min. For RALA the stability was assessed over a 6 h time course. Loading dye (Ficoll (Sigma, UK), Tris-HCl, bromophenol blue (Sigma, UK) in ddH2O) was added to all the aliquots prior to loading onto an ethidium bromide prestained 0.8% agarose-TAE gel. A current of 80V was applied for 1 h and the gel was visualised using a Multispectrum Bioimaging System (UVP, UK). (FIG. 5, 7a (with trehalose) 10 (with siRNA), 30, 56 (RAT nanoparticles))

### Transmission Electron Microscopy

In an attempt to confirm the results obtained by DLS and obtain additional information about the structure of the nanoparticles Transmission Electron Microscopy was employed. The RALA complexes were prepared as perf or standard conditions and 5 μl was pipetted onto formvar coated copper grids (Agar Scientific, UK) and allowed to air dry overnight. Subsequently samples were stained with 5% aqueous 5% uranyl acetate for 5 minutes and allowed to dry overnight before visualisation. The nanoparticles were imaged using JEOL 100CXII transmission electron microscope at an accelerating voltage of 80 kV (FIG. 2 (siRNA), 15 (Alendronate), 28 (GFP DNA), 50 (Bisphosponates)).

### Freeze Drying of the Nanoparticles

700 μl of RALA-pEGFP-N1 nanoparticles were subject to freezing for 1 h at −40° C. This was followed by primary drying at −40° C. and 60 mTorr for 24 h. This was followed by the secondary drying program; 3 h at −35° C. and 120 mTorr, 3 h at −30° C. and 190 mTorr, 3 h at −25° C. and 190 mTorr and 6 h at 20° C. (FIG. 7b).

Transfection of ZR-75-1 & PC-3 Cells in 96 Well Plates with the RALA Nanoparticles

In order to test the RALA in vitro, small scale transfections were performed carried out. 5×104 cells were seeded onto each well of a 96 well plate and the cells incubated under with complete medium standard conditions for 48 hours. The medium was subsequently removed from the plates and 100 μl of transfection medium (Optimem Invitrogen, UK) was added to each well. Cells were incubated for 2 hours at 37° C. and 5% CO2 standard conditions. In the meanwhile complexes were made up using 1 μg of plasmid DNA with the RALA vector and added to the cells when the two hours had passed. 100 μl of the each N:P ratio were added to each well of the cells. Cells were then incubated for a further 4 hours under standard conditions and the medium with RPMI-1640 supplemented with +10% FCS. (FIG. 4, 31(+Chloroquine), 32 (+Bafilomycin), 33 (+KALA NPs), 34 (+Lipofectamine), 35).

### Flow Cytometry to Quantify Fluorescent Intensity

ZR-75-1 & PC-3 cells that were transfected with RALA/pEGFP-N1 complexes were trypsinised and washed twice with 2% formaldehyde in phosphate buffered saline. The expression of green fluorescent protein was measured by flow cytometry using FACS calibur system (BD Bioscience, UK). The data was analysed using the Flo-Jo software program and fluorescent intensity is reported at 4% gating. (FIG. 7b, 11, 31, 32, 33, 34, 35, 57 (+RAT/pEGFP-N1)).

### Cell Proliferation Assay

Cell viability was evaluated by manual counting of the viable adherent cells using a haemocytometer as described in. PC-3 prostate cancer cells were seeded in a 96-well flat-bottom tissue culture plate at a density of 1×104 cells per well and incubated in complete culture medium for 24 h. Two hours prior to transfection the cells were conditioned in OptiMEM serum-free medium (Invitrogen, UK) optimised for transfection. Cells were treated with solutions of BP to achieve a final exposure concentration of 5 μM to 1 mM. RALA/BP nanoparticles were prepared using a mass ratio of 10:1 such that the final concentration of BP per well was in the range 5 μM to 75 μM. Cells were incubated at 37° C. with 5% CO2 for 6 h before medium was replaced with completed culture medium and left to incubate for 72 h. Following incubation the cells were trypsinised and counted. Cell viability was expressed as a percentage of the untreated control where the untreated control is considered to be 100% viable. Dose-response curves were obtained for free BP and RALA/BP allowing determination of EC50 values for each. EC50 values refer to the concentration that induces a response halfway between the baseline and the maximum plateau obtained (FIG. 46, 51).

### WST-1 Cell Viability Assay

The WST-1 assay is a colorimetric assay that can analyse the number of viable cells present and hence, indicate the toxicity of complexes added to cells in vitro. The assay is based on the cleavage of tetrazolium salts that are added to the culture medium. The stable tetrazolium salt WST-1 is cleaved to a soluble formazan by a cellular mechanism that occurs primarily at the cell surface. This WST-1 cleavage is dependent on the glycolytic production of NAD(P)H in viable cells, therefore, the amount of formazan dye formed directly correlates to the number of metabolically active cells in the culture.

Cells were transfected and the complete medium was discarded at a range of time points and replaced with 100 μL Opti-MEM with 10% WST-1 reagent (Roche, UK). Cells were incubated for 2 h under standard cell culture conditions. Subsequently the plates were shaken for 1 min and absorbance measured at 450 nm on an EL808 96-well plate reader (Biotek, USA). The measured absorbance values are expressed as a percentage of the control where the control is defined as 100% viable (FIG. 36).

### Intradermal Tumour Model in BALB-C SCID Mice

ZR-75-1 or PC-3 cells were trypsinised until they had detached and 8 ml of medium was added per flask. The cell suspension was transferred into 20 ml universal tubes. The cells were and centrifuged for 5 minutes at 80 g. Cells were resuspended in RPMI+10% FCS and counted using a Coulter Counter (Beckman Coulter, UK). Cells were subsequently centrifuged as before, and resuspended at 108 cells per ml in PBS before being diluted 1: in 1 in matrigel (BD Biosciences, UK). The matrigel cell suspension was loaded into syringes and kept on ice until implantation. Matrigel was only required for the ZR-75-1 cells. Balb-C SCID mice were anaesthetised with isofluorane (Abbott, UK) and the rear dorsum was shaved. Subsequently the skin on the rear dorsum was pinched between forefinger and thumb and 5×106 cells (100 μl) were injected intradermally using with a 26 G needle (BD Biosciences, UK) at the prepared site. Mice were observed while recovering from the anaesthesia and then subsequently returned to their box (FIG. 6, 47, 52).

### Tumour Size Measurements

The length (L), width (W) and depth (D) of the tumour was measured using vernier with calipers. Subsequently the volume of the tumour was estimated by using the equation, V=πLWD/6, an approximation of V=4/3πr3.

### Intra-Tumoural Injections

Mice were anaesthetised with isofluorane and a 26 G needle (BD Biosciences, UK) was inserted bevel side down into the tumour. 100 μl of the nanoparticle treatment was injected slowly before rotating the needle and removing very slowly. For the multiple dose regimen used in this study a ‘round the clock’ system of injections was used. Recovery of mice from anaesthesia was monitored (47,52).

### Intra-Venous Injections

Mice were placed into a heat box at 36° C. for 5 minutes or until both of the tail veins were clearly visible. They were then moved into a heavy brass restrainer and injected with 50-100 μl of treatment into the tail vein with an insulin syringe (BD Biosciences, UK) equipped with 28 G needle. Mice were then replaced into the cage and monitored for signs of suffering associated with the injection. Mice found to be suffering or dying were euthanized by a schedule one protocol. (FIG. 6, 40).

### Harvesting Blood Via Cardiac Puncture and Collection of Serum

For the harvesting of blood and intraperitoneal macrophages, cervical dislocation was the preferred method of euthanasia. Cardiac puncture was performed using a 21 G gauge needle (BD Biosciences, UK). The needle was placed horizontally slightly to the left side of the sternum to go up through the diaphragm. The needle was then withdrawn very slowly until 500 μl of blood was collected and placed in an eppendorf. The eppendorf was then stored at room temperature with an open lid to facilitate coagulation. After 30 min the eppendorfs were centrifuged at 2000 rpm for 10 min. The supernatant containing the serum was carefully decanted and placed into a clean eppendorf and stored at −20° C. until further use. When harvesting intraperitoneal macrophages an incision was made and the peritoneal cavity was flushed out with 30% sucrose (Sigma, UK) solution. The macrophages were stored at 4° C. until they could be cultured (FIG. 8).

Western Blots with In Vitro and In Vivo Samples

Organs were homogenised and lysed in RIPA overnight. The samples were centrifuged at 5000 g for 10 minutes and the supernatant transferred to a fresh eppendorf tube. The lysate was diluted 1:2 in laemmli buffer, boiled for 10 minutes and loaded onto a Bis-Tris gel. Cells were put directly into laemmli buffer. The gel was run at 120V till the dye reached the bottom. The gel was and transferred into a western cassette. The protein was subsequently transferred for 2.5 hours at 25V onto a nitrocellulose membrane (Amersham, Biosciences, UK). Protein transfer was visualised by staining with Ponceau stain (Sigma, UK). The membrane was then subsequently incubated with primary antibody in blocking solution (PBS (Invitrogen, UK), 0.1% Tween (Sigma, UK), Skimmed milk (Merck, Germany)). Subsequently the membrane was then rinsed twice within Tween-PBS and once within PBS before being incubated in secondary antibody for 1.5 hours. The membrane was then was rinsed again, twice with Tween-PBS and once within PBS before the application of Immobilon reagent (Millipore, UK). Western blots were quantified using imageJ software (FIG. 6, 37, 38, 45).

### Vector Neutralisation Assay

Female C57/BL6 mice (5-6 weeks old) were treated with one of;
PBS (control)
RALA alone
DNA alone (CMV/GFP)
RALA/DNA nanoparticles

Mice receiving DNA received 10 μg total. Nanoparticles were formulated with an N:P ratio of 10. Mice receiving RALA alone received an amount of vector equivalent to that received in the RALA/DNA group. Treatments were administered by tail vein injection performed over a three week period. There was 15 mice per treatment group, with 5 mice per time point. All animals received the relevant treatment on Day 0. Following 7 days, five mice from each group were sacrificed and blood from each will be isolated by cardiac puncture. Serum was isolated, serum from the five mice per group was pooled, heat-inactivated at 56° C. for 30-60 min, and serially diluted in Opti-MEM to produce serum concentrations of 10% v/v, 1% v/v and 0.1% v/v, plus a 0% control.

To these serum dilutions, fresh RALA/DNA nanoparticles (as above) were added at a DNA concentration of 1 μg/200 μl (the standard concentration for RALA/DNA transfection in 96 well plate format), and incubated at 37° C. for 1 h. This pre-incubated mix was then transferred to ZR-75-1 breast cancer cells previously seeded in 96 well plates (104 cells/well) on Day 6, and transfection was performed in the usual manner. Transfection of the GFP construct was assessed by FACS analysis after 24 h.

On Day 7, the remaining 10 mice received a second administration of the appropriate treatment. On Day 14, five mice left the experiment and were treated as above, while the remaining five mice per group received a final administration of the appropriate treatment, and on Day 21, followed by the previously outlined treatment (FIG. 22, 23).

### Enzyme-Linked Immunosorbent Assay

These assays were performed on the serum collected from immunocompetent C57/BL6 mice following either 1, 2, or 3 intravenous injection with the RALA/pEGFP-N1 nanoparticles. IgG, IgM, 11-12, IL-6, and TNF-6, ELISAs were performed using the ENZO ELISA Kits in accordance with the recommended protocol (FIG. 8).

### For the Neutralising Antibody ELISA the Following Method Applied;

Nunc Maxisorp ELISA plates were coated with RALA-pEGFP nanoparticles equivalent to 1 μg DNA per well. The wells were subsequently blocked with PBS/5% BSA. Wells were probed for 1 h with sera from mice diluted (1:500) in PBS/0.5% BSA at room temperature. (NB the sera came from the mice treated in the vector neutralisaiton assay). The wells were washed with PBS/0.5% Tween 20 and then probed for 30 min with HRP-conjugated anti-mouse secondary antibody. Wells were then washed again and probed with TMB substrate for 30 min. Colour development was measured at 450 nm with a reference wavelength of 550 nm (FIG. 24).

### Confocal Microscopy

5000 ZR-75-1 breast cancer or PC-3 prostate cancer cells were grown on cover slips and transfected with Cy3 labelled RALA/pEGFP (lacks the promoter contained in the construct used in the neutralisation assay) or fluorescent siRNA. Confocal microscopy was used to determine subcellular localisation of RALA/Cy3-pEGFP nanoparticles (FIGS. 25, 44b & 58).

### Gold Nanoparticle Experiment

5 nm phosphorylated gold nanoparticles were incubated with RALA peptide at a ratio of approximately 1:10 for 30 mins before being added to MDA-MB-231 breast cancer cells for 24 hours. The MDA-MB-231 cells (5000) had been seeded onto a coverslip. After 24 h the cells were fixed with 50% methanol and 50% acetone and sent to Cytoviva (Auburn, Ala.) for imaging (FIG. 26).

### Greiss Test

Cells seeded in multiwell plates (6 or 24 well) were transfected with various amounts of pDNA (CMV/iNOS or hOC/iNOS) complexed with RALA at N:P 10 for 6 h, following which, transfection complexes were removed, and cells returned to normal growth medium (Minimum Essential Medium—MEM). After 48 h, 70 μl aliquots of conditioned MEM were assayed for their total nitrate (an indirect indicator of nitric oxide content) content using a Nitric Oxide Quantitation kit (Active Motif) following the manufacturer's instructions. A standard curve (using 0-35 μM sodium nitrate) was constructed and used to quantify nitrate content in sample wells of the assay plate. After incubation of standards and unknown samples with nitrate reductase and co-factors, Greiss reagents A and B were added to wells, and after a 20 min incubation to allow colour development, the absorbance of each well at 540 nm was determined (FIG. 38).

### Clonogenic Assay

PC-3s grown in T25 tissue culture flasks were starved of serum by Opti-MEM incubation for 2 h before transfection with 10 μg of pDNA (CMV/iNOS, hOC/iNOS or CMV/GFP) for 6 h. Following transfection, media were replaced with MEM, and the cells incubated overnight. The next day, cells were trypsinised, resuspended in growth medium, enumerated, and plated in triplicate into 6 well plates (200 or 500 cells per well). Plates were incubated for 14 days to allow clonogenic growth, following which, medium was aspirated, colonies were stained with crystal violet and counted manually. Percentage cell survival was calculated by comparison with untransfected cells (FIG. 39).

### Intracardiac Metastases Model

Female Balb/c SCID mice (5-8 weeks old) were inoculated via the left cardiac ventricle with 2×105 MDA-MB-231-luc2 breast cancer cells that express firefly luciferase. Mice then received an intraperitoneal injection of 200 μl D-luciferin (15 mg/ml) and were imaged (following 10 min) using IVIS imaging; successful left ventricular delivery was confirmed by whole body luminescence immediately following intracardiac delivery. Mice possessing luminescence limited to the thoracic cavity were sacrificed at this point. Remaining successfully inoculated mice were randomly assigned to one of four treatment groups (water, RALA only, RALA-CMV/iNOS or RALA-hOC/iNOS), and received five treatments twice weekly commencing two days post inoculation. Gene therapy mice received 10 μg pDNA complexed with RALA at N:P 10, RALA only mice received the corresponding amount of RALA dissolved with water; treatments were of 100 μl, and were delivered via the tail vein. Mice were routinely imaged twice weekly as described above, were observed daily by experienced animal husbandry experts, and body mass was monitored as an indicator of general health. A loss of 20% of original body mass was considered indicative of poor health of the mice, and this combined with a moribund appearance was determined to be a humane experimental end point (FIG. 40).

### Effect of Runx2 Knockdown on Cell Proliferation

The effects of Runx2 knockdown on cell proliferation were evaluated at different time-points following transfection with RALA/Runx2 siRNA nanoparticles. Nanoparticles were prepared such that the final concentration of Runx2 siRNA was 100 nM and based on a N:P ratio of 12. Two Silencer Select Runx2 siRNAs were used and a Silencer Select non-coding siRNA (Invitrogen, UK). Cells were serum starved for 2 h prior to transfection. Transfections were carried out with both RALA peptide and Oligofectamine for a duration of 4 h in serum-free RPMI 1640 before RPMI 1640 containing 30% FCS was added to achieve a final FCS concentration of 10%. After 24, 48 and 72 h cells were detached using 2× trypsin and subsequently neutralised with RPMI 1640 containing 10% FCS. Cells were counted manually using a haemocytometer as described in 3.2.11.2 and the cell viability determined based on the assumption of a 100% viability of the untreated cells. Results are reported as mean±SEM, n=3, where n represents the number of independent batches prepared for analysis (FIG. 46).

### Western Blotting for Runx2 Protein

To assess the ability of RALA/Runx2 siRNA nanoparticles to successfully inhibit Runx2 protein expression a range of siRNA concentrations and time-points following transfection were evaluated by Western blotting. PC-3 prostate cancer cells were seeded at a density of 150,000 cells per well in a 12-well plate. Transfections were initially carried out with various amounts of two types of Silencer Select Runx2 siRNA and Silencer Select non-targeting control siRNA such that the final siRNA concentration in the well was 50, 100 or 200 nM. Transfection was for 4 h followed by 48 h incubation. Following optimisation of the concentration the optimal time following transfection was determined using 100 nM concentrations. Cells were washed with ice-cold tris buffered saline (TBS) and lysed in a direct lysis buffer supplemented with MG-132 (Calbiochem, UK) and protease inhibitor cocktail (Roche, UK) (Appendix 1). Lysed samples were stored at −20° C. until required. Samples were run on 8% acrylamide gels at 100 V for 15 min followed by 150 V until the dye front reached the bottom of the gel in a tris-glycine running buffer. Subsequently the protein was transferred to PVDF membranes at 200 mA for 90 min in a tris-glycine transfer buffer. Membranes were blocked for up to 1 h in 2% blocking solution before leaving in primary antibody overnight at 4° C. with rocking. Runx2 primary antibody (MBL International, Woburn, Mass.) was used at a concentration of 1:200 and β-actin (Abcam, UK) at a concentration of 1:5000. Membranes were washed in TBS-tween (TBS-T) for 30 min before applying anti-mouse secondary antibody at 1:5000 for 1 h at room temperature. Membranes were washed vigorously in TBS-T for 30 min before developing. The chemiluminescent used for Runx2 protein was Thermo Scientific SuperSignal West Dura Chemiluminescent Substrate (Thermo Fisher Scientific, Waltham, Mass.) and for β-actin Thermo Scientific SuperSignal West Pico Chemiluminescent Substrate (Thermo Fisher Scientific, Waltham, Mass.) (FIG. 45).

Studies with the RAT Peptide

RAT was synthesised from a commercial company and is a fusogenic, consisting of RALA with an alphahelical concatemeric spacer, (EAAAK)4, and the TMTP1 (NVVRQ) metastatic prostate cancer targeting peptide (FIG. 54).

### RALA-PEG5K

A pegylated version of RALA has been synthesised (FIG. 60).

### Composite RALA Nanoparticles

RALA nanoparticles were prepared using desalted peptide in MOPS buffer at 50° C. to give a concentration of 50 μg/ml of DNA. PLGA and a series of PLA-PEG block copolymers were synthesized with various PEG chain length and LA/EG ratio (PLA10-PEG2; PLA25-PEG5; PLA50-PEG5) and formulated into composite nanoparticles (diameter <200 n m and PDI <0.2000) containing the RNPs. 100 μl of RALA nanoparticles was added to 0.5 ml 4% w/v copolymeric polymeric solution in dichloromethane under vortex and probe sonicated (120 Sonic Dismembrator with 3 mm probe, Fisher Scientific, USA) for 60 seconds at 50% of amplitude. This water-in-oil (w/o) emulsion was added to 2.5 ml of 5% w/v PVA solution in distilled water under vortex and probe sonicated as before in an ice bath for 2 minutes. The resultant emulsion was stirred overnight to form the composite nanoparticles. These were collected by centrifugation at 30,000 g for 30 min (3K30, Sigma Centrifuge, UK) and washed twice with distilled water, before suspending in 1 ml 5% w/v trehalose in water and were freeze-dried (Advantage, VirTis, Gardiner, N.Y., USA). TEM (JEOL JEM1400 transmission electron microscope at an accelerating voltage of 80 kV) was performed by loading samples onto a copper grid (Formvar/Carbon 200 mesh, Agar scientific). Osmium tetraoxide was incorporated by adding it to the organic phase during preparation of the composite nanoparticles.

### Results

### Particle Characterisation.

As shown in FIG. 2, RALA condensed siRNA to form discrete spherical nanoparticles formed at N:P 12. RALA also condensed DNA at N:P 10 and bisphosphonates giving spherical particles (FIG. 28, 50). This indicates that the RALA is condensing the nucleic acid in a uniform manner. Whether siRNA, DNA or bisphosphonates, the overall positive charge also ensures that the particles are discrete and repel each other which avoids aggregation and ensures a homogenous population.

Particle formation between DNA and RALA was studied by gel retardation assays and dynamic light scattering. It was found that RALA fully condensed DNA at N:P ratios above 4 (FIG. 27). Dynamic light scattering revealed that particle sizes were below 100 nm at N:P ratios above 4 and around 1 μm at N:P 2 and 3 (FIGS. 3,9, 17 to 21, 42). The zeta potential of the particles at N:P ratios 2 and 3 was −15 mV and −5 mV respectively. The zeta potential was only positive at N:P ratios greater than 4. A near-zero zeta potential also means that there is little surface repulsion between particles and as a result the large aggregates are observed which is reflected in the size of the nanoparticles at N:P ratios 2 and 3. Above N:P 4, on the other hand, particles have a diameter below 100 nm and as a result may theoretically enter cells via endocytosis. The RALA/pEGFP-N1 nanoparticles at a ratio of N:P 10 were dried and stained with 5% uranyl acetate and transmission electron microscopy at 80 kV further confirmed the presence of spherical particles in the region of 100 nm in diameter (FIG. 27). From N:P 3 upwards the encapsulation efficiency of the RALA/pEGFP-N1 nanoparticles was greater that 90% (FIG. 53).

Additionally, serum stability of particles at N:Ps of 5, 10 and 15 showed that the nanoparticles are stable in the presence of 10% serum and dissociate in 1% SDS revealing that the integrity of the DNA remains intact (FIGS. 5, 7a, 10 & 30). As the nanoparticles were found to be stable for up to 6 (FIG. 30, 33). Nanoparticles were also stable in range of temperatures 4-37° C. (FIG. 29, 42).

### In Vitro Transfection Efficacy & Cytotoxicity.

ZR-75-1 cells were transfected with RALA/pEGFP-N1 nanoparticles. Epifluorescence microscopy showed a high transfection efficacy of ZR-75-1 cells, when transfected with RALA/pEGFP at N:P of 10 with and without chloroquine. Chloroquine is a known endosomal disrupter and will increase transfection if the nanoparticles are inefficient endosome disrupters. At N:P 10 this is clearly not the case. Flow cytometry was then used to further analyse the effect of N:P on transfection efficacy and revealed an optimal transfection efficacy of around 30% between N:P ratios 8-12. More importantly though the WST-1 cell viability assay revealed minimal toxicity of the nanoparticles over a range of N:P ratios. Cell viability was 90% for N:P 4 and 80% at N:P 10. Indeed when cellular proliferation was examined there was significant difference between lipofectamine 2000 and RALA/pEGFP-N1 transfected cells (FIG. 34). Similar effects were also observed for PC-3 cells in respect of transfection and also cell viability (FIG. 35, 36). In addition transfection with RALA peptide derivative 2-6 showed transfection efficiencies of at least 40% (FIGS. 17 to 21, 31). Percentage transfection with these nanoparticles in the absence and presence of bafilomycin (FIG. 32) showed a significant reduction at all N:P ratios investigated (p=0.0037, 0.0002 and 0.0021 respectively for N:P ratios 8, 10 and 12 with RALA/pEGFP-N1 using an unpaired one-tailed t-test), indicating the acidic pH is essential for nanoparticle release from the endosome.

To determine if RALA/pEGFP-N1 nanoparticles are significantly more efficient at eliciting cellular transfection in comparison to KALA/pEGFP-N1 nanoparticles a transfection experiment with both peptide-based nanoparticles was carried out in parallel (FIG. 33). It is possible to see that percentage transfection achieved with RALA/pEGFP-N1 nanoparticles N:P ratio 8 and 10 is significantly higher than those achieved with the KALA/pEGFP-N1 nanoparticles (p=0.0002 and 0.0048 using a one-tailed unpaired t-test). The RALA/pEGFP-N1 nanoparticles were more efficient than the commercially available happyfect (FIG. 37).

Confocal microscopy also confirmed successful transfection with a time course revealing diffuse pattern of distribution of nanoparticles that focus into distinct foci with increasing duration of transfection (FIG. 25). Internalisation of RALA/DNA nanoparticles has been demonstrated. There was also increased internalisation and disruption of the endosomes when RALA was used to deliver gold nanoparticles (FIG. 26). When fluorescent siRNA was delivered it could also be seen that it was internalized into the cytosol and indeed proved more efficacious than the commercially available oligofectamine (FIG. 44).

### Lyophylisation of RALA.

As RALA/pEGFP-N1 nanoparticles transfect cells efficiently and are non-toxic, it was decided to use these nanoparticles as a model of a potentially therapeutic peptide based polyplex. It is well know that a major problem with gene therapy protocols is storage as both peptide and DNA degrade if stored in aqueous solutions at room temperature for prolonged periods of time. As such, the nanoparticles were lyophylised with a range of concentrations of trehalose as a lyoprotectant. Transfections, as well as serum stability assays were performed before and after freeze-drying. Serum stability assays were performed on all formulations up to 6 h. All formulations were found to be as stable upon incubation with 10% serum as the fresh particles without trehalose (FIG. 7a). In addition, decomplexation with 1% SDS disrupted the nanoparticles in all cases and revealed no significant DNA damage in the reconstituted samples (FIG. 7a). It was also found that the RALA based polyplexes are equally efficient at transfecting cells both before and after lyophylisation (FIG. 7b). Increasing the concentration of trehalose did not improve transfection, but rather seemed to decrease transfection efficiency at high concentrations although there was no significant difference between fresh and freeze-dried nanoparticles. It was also found that those nanoparticles without trehalose still retained activity post freeze-drying (FIG. 7b). Although the freeze dried particles in this formulation tended to stick to the glass vials and needed more time to resuspend.

Overall these results highlight the stability of RALA/pEGFP-N1 nanoparticles as well as the ease with which dried formulations can be stored, even without lyoprotection. These data indicate that the RALA could be lyophilised, stored and reconstituted prior to administration without losing activity.

### Transfection Efficacy & Immunogenicity of RALA In Vivo.

As RALA has proven highly effective in vitro, the next logical step would be to test its transfection efficacy and distribution and most importantly, bio-compatability in vivo. As such, ZR-75-1 tumour bearing BALB/C-SCID mice were injected intravenously with 50 μl of N:P 10 RALA/pEGFP-N1 or RALA/phOCMetLuc nanoparticles carrying a total of 10 μg of plasmid DNA per dose. Western blots showed transfection in all organs with the pEGFP-N1 carrying nanoparticles and in the tumour, surrounding tissue and liver with phOCMetLuc nanoparticles (FIG. 6). Immunoperoxidase staining of organs sections revealed low levels of transfection in the tumour, liver, lungs and kidney, with transfection being undetectable in heart and peritumoural tissue for the RALA/pEGFP-N1 treated animals (FIG. 6).

In order to determine whether the RALA based nanoparticles would be safe for repeated administration, immunocompetent C57/BL6 mice were treated once a week with either 50 μl of PBS, PEI, RALA, pEGFP-N1, PEI/pEGFP-N1 or RALA/pEGFP-N1 for 3 weeks. In each instance the dose of plasmid DNA delivered was 10 μg. Blood was collected via cardiac puncture and ELISA's were performed for IgGs, IgMs, TNFα, IL6 and IL1β, alongside a Greiss test for increased nitric oxide concentrations. No morbidity or visible immune response was seen upon inspection of the live animals. ELISAs for interleukins yielded no statistically significant differences between groups of treatments (FIG. 8) Concentrations of nitrates were found to be elevated in the third week compared to previous weeks (p<0.01), but no significant differences were seen between treatments (FIG. 8). The change in TNFα concentrations with repeated treatments were found to be highly statistically significant, with higher concentrations in the first week, and lower concentrations following subsequent treatments, this was especially prominent in the PBS only and PEI only treatment groups (p<0.05 and p<0.01 respectively). In addition, the RALA only treated mice had a significantly lower initial response of TNFα than the PEI only treated animals (p<0.05) (FIG. 8). Concentrations of IgM were found to be significantly lower with repeated administration (p<0.001). This effect was more pronounced in the PBS only treatment group (p<0.05). Changes in IgG concentrations depended heavily on both the treatment group and the treatment applied (p<0.01). Naked DNA induced a strong IgG response on the third week (p<0.05), which was significantly higher than that induced by RALA only, RALA/pEGFP-N1, PEI/pEGFP-N1 and PBS only (p<0.05, p<0.01, p<0.05, and p<0.01 respectively), indicating that naked DNA induces an adaptive immune response, while neither the RALA or PEI based nanoparticles cause this kind of response (FIG. 8). This in turn indicates that RALA and PEI shield the plasmid DNA from detection by the immune system This data set clearly indicates that systemic delivery of RALA/pEGFP-N1 nanoparticles does not induce a significant immune response either innate or adaptive even after multiple injections.

Furthermore multiple injections of the RALA nanoparticles did not evoke neutralising antibodies that would prevent RALA from delivering its payload. FACS analysis of PC3 and ZR-75-1 cells indicated that transfection of both cell types was hampered by the presence of 10% serum, but this occurred with the FBS controls as well eliminating the activation of an immune response (FIGS. 22 and 23). This was further confirmed by the ELISA on the sera samples which showed that there was there was no significant difference in immunoreactivity between the different treatment groups (FIG. 25).

### Systemic Delivery of RALA/iNOS Nanoparticles

Transfection of PC-3 and MDA-MB-231 with plasmid iNOS constructs complexed with RALA evoked nitric oxide production (as determined by total nitrate content of growth media—an indirect method of nitric oxide quantification). PC-3s and MDA MB-231s transfected with the inducible hOC/iNOS plasmid produced significantly more nitrates than were present in control (P=0.038 and 0.048 respectively), and those transfected with the constitutively active CMV/iNOS also produced levels of nitrates considerably higher than seen in control. Nitrate content of media of cells transfected with green fluorescent protein constructs under the control of the same promoters were consistent with control (FIG. 38).

Transfection of PC-3s with hOC/iNOS complexed with RALA prior to clonogenic assay resulted in significantly lower clonogenic survival compared to control (P=0.004). Transfection of the same cells with CMV/iNOS resulted in a roughly similar loss of clonogenic survival (0.69±0.08 vs 0.61±0.03), while transfection with CMV/GFP did not affect clonogenic survival of PC-3s (surviving fraction of 1.01±0.11) (FIG. 39). This experiment has been performed twice; it is likely that a third replicate will resolve the significance of CMV/iNOS treatment, and further support that of hOC/iNOS.

Metastatic deposits were established in female BALB/c SCID mice by inoculation with 2×105 MDA-MB-231-D3H1 that express luciferase via the left ventricle of the heart. Metastatic development was monitored routinely by IVIS imaging of bioluminescence (FIG. 40). Control treatment for these mice was water (the vehicle for gene therapy treatments); mice receiving water treatment had a median survival of 30 days post inoculation. The median survival for mice receiving RALA only treatment was also 30 days (P=0.76 compared with water control). Treatment with hOC/iNOS or CMV/iNOS complexed with RALA resulted in a significant improvement of post inoculation survival, with mice receiving hOC/iNOS having a median survival of 40 days (P=0.001 compared with water), and those that received CMV/iNOS having a median survival of 42 days (P=0.004).

### Delivery of RALA/siRUNX2 as a Therapeutic

To confirm that Runx2 protein expression could successfully be knocked down using the RALA, PC-3 prostate cancer cells were transfected and the cell lysate collected for Western blotting. Two types of Runx2 siRNA were used as well as a non-targeting scrambled siRNA. Furthermore, Oligofectamine was used as a positive control for comparison. Initially the concentration of siRNA required to achieve knockdown was assessed followed by the optimal incubation time post-transfection. Densitometry of the Western blots using Image J software enabled the degree of knockdown of protein expression to be quantified by assuming the scrambled control siRNA results in 0% knockdown.

FIG. 45 shows the optimisation of the time required following transfection to achieve optimal knockdown of Runx2 protein expression. This was assessed using a siRNA concentration of 100 nM as determined previously. It can be seen clearly that there is substantial knockdown of protein expression at each timepoint. There was no increase in knockdown with increasing time as confirmed by one-tailed unpaired t tests which found no significance between the knockdown at each timepoint as well as no difference in knockdown between each of the delivery systems (p>0.05). As such it can be confirmed that 24 h is sufficient time to detect optimal knockdown. Furthermore, there is no significant difference in the effectiveness of each of the two Runx2 siRNAs across any of the concentrations or timepoints with both of the transfection reagents used (p>0.05) as determined by one-tailed unpaired t test.

RALA peptide was able to achieve comparable levels of knockdown to the commercial RNA transfection reagent, Oligofectamine. Analysis of the transfection profile of RALA and Oligofectamine using fluorescent siRNA showed a peak in transfection immediately after transfection with RALA but it took 24 h to reach a peak with Oligofectamine.

To determine the effects of Runx2 knockdown on prostate cancer cell proliferation, PC-3 prostate cancer cells were transfected with 100 nM Runx2_1, Runx2_2 or non-targeting scrambled siRNA using RALA or Oligofectamine as a positive control. Where RALA was used nanoparticles were prepared at N:P 12 and Oligofectamine was used as per the manufacturer's guidelines. Cells were trypsinised and counted using a haemocytometer at 24, 48 and 72 h following the 4 h transfection. Untreated cells were assumed to have 100% viability and the percentage viability for all other treatments was based on this.

Cell viability was significantly lower with Runx2_1 compared to Runx2_2 24 h following transfection with RALA peptide (p=0.0376). However, no significant difference between the two siRNAs is seen at any other timepoint or following delivery using Oligofectamine (p>0.05) as determined by two-way ANOVA. Furthermore, there is no significant difference in cell viability following transfection of Runx2_1 and Runx2_2 across the timepoints studied up to 72 h (p>0.05) when determined by two-way ANOVA. RALA/Runx2_1 siRNA nanoparticles resulted in a significant reduction in cell viability when compared to RALA/scrambled siRNA nanoparticles at each of the 24, 48 and 72 h timepoints evaluated (p<0.001, 0.05 and 0.01 respectively). Similar results were found with RALA/Runx2 siRNA nanoparticles (p<0.01, 0.01 and 0.001 respectively). These results were consistent with the positive control, Oligofectamine, which also resulted in a significant decrease in cell viability compared to the scrambled control with Runx2_1 (p<0.001, 0.01 and 0.001 at 24, 48 and 72 h respectively) and Runx2_2 (p<0.01, 0.05 and 0.01 at 24, 48 and 72 h respectively). Overall, knockdown of Runx2 protein expression results in a reduction in cell viability of approximately 30% over 72 h (FIG. 46).

Tumours were grown on the rear dorsum of BALB-C SCID mice until the volume reached approximately 150 mm3 before intratumoural treatment with either RALA/Runx2 siRNA nanoparticles, Runx2 siRNA only or RALA/scrambled siRNA nanoparticles commenced. Runx2_1 and Runx2_2 siRNA were pooled for the purposes of in vivo analysis as neither was found to be significantly better in achieving Runx2 knockdown. Dosing was once weekly until tumour quadrupling defined the endpoint of the experiment. Control tumours grew rapidly with all tumours quadrupling in volume within 16 days of the start of treatment (average 15 days). RALA/scrambled siRNA nanoparticle treatment mice follow a similar rate of growth as the untreated. The rate of growth is also similar for Runx2 siRNA treated mice until after the second treatment; following this the tumours grow at a slower rate than the untreated and RALA/scrambled siRNA groups. In mice treated with RALA/Runx2 siRNA nanoparticles, tumours grow at a slower rate than all other groups until the point of tumour volume quadrupling (FIG. 47a). It appears that the difference in survival time between the untreated mice compared to those receiving RALA/scrambled siRNA nanoparticles is small; however, it is not possible to determine the significance of this difference due to the small group numbers. Mice treated with Runx2 siRNA had a higher survival time of 22.5 days compared to 15 days for untreated mice (FIG. 47b). The Kaplan-Meier plot required no censoring of the data as no animals were euthanised or died apart from those in which the tumour volume quadrupled (experimental end-point). A significant increase in survival time of RALA/Runx2 siRNA nanoparticle treated mice of 80% was seen when compared to the untreated control group (p=0.0002) (FIG. 47c)

### Delivery of RALA/BP as a Therapeutic

In order to assess the effectiveness of RALA as a delivery agent for optimisation of the antitumour effects of BPs, PC-3 prostate cancer cells were either treated with free BP or transfected with RALA/BP nanoparticles at a range of concentrations for 6 h and then incubated for 72 h before evaluating cell viability. Cell viability was analysed by cell counting using a haemocytometer. EC50 values were determined using the dose-response curves generated from this cell viability data. The EC50 of alendronate was reduced from 100.3 μM to 17.6 μM when delivered in a RALA nanoparticle, a potentiation factor of 5.7 (FIG. 51a). The EC50 of zoledronate was reduced from 27.6 μM to 26.9 μM when delivered in RALA nanoparticles as determined by the dose-response curve. However, the maximum cell kill that could be achieved with zoledronate only was 82% compared to the 96% seen with RALA/zoledronate nanoparticles (FIG. 51b). The EC50 of risedronate was determined from the dose-response curve to be 78 μM. However, this concentration equated to the concentration required to see a 10% cell kill as the maximum cell kill observed when cells were treated with risedronate was 20%. Transfection of the cells with RALA/risedronate nanoparticles, however, saw a maximal cell kill of 86% with an EC50 of 33.6 μM (FIG. 51c). It was not possible to determine an EC50 value for etidronate using the dose-response curve as there was no reduction in percentage cell survival with increasing BP. However, when etidronate is delivered in RALA/etidronate nanoparticles a reduction in cell survival can be seen and the EC50 of RALA/etidronate nanoparticles was determined from the dose-response curve to be 36.6 μM (FIG. 51d).

Tumours were grown on the rear dorsum of BALB-C SCID mice until the volume reached approximately 100 mm3 before intratumoural treatment with RALA/alendronate, alendronate or RALA commenced. Dosing was thrice weekly until tumour quadrupling defined the endpoint of the experiment. It can be seen clearly that RALA only had no significant effect on tumour growth (p=0.0792) while alendronate and RALA/alendronate show high statistical significance when compared to the untreated control (p<0.0001 and p=0.0004 respectively) (FIG. 52a). Furthermore, the difference in time taken for tumour volume to quadruple is also significantly different between alendronate and RALA/alendronate (p=0.0019) (FIG. 52b). Control tumours grew rapidly with all tumours quadrupling in volume within 16 days of the start of treatment. This is consistent with previous results from the group on the PC-3 tumour model. Treatment with alendronate and RALA/alendronate slows tumour growth at an almost identical rate up to treatment 4. However, beyond treatment 4 the rate of growth in alendronate treated tumours changes with tumour volume beginning to increase more rapidly. RALA/alendronate tumours continue to grow at a similar rate to the beginning of treatment up to the end of the treatment course (3 times weekly dosing for 3 weeks) but then began to grow more rapidly after discontinuation of therapy. However, the rate of growth upon discontinuation of treatment is still lower than the controls and the higher rate of growth in alendronate treated mice (FIG. 52). The Kaplan-Meier plot required no censoring of the data as no animals were euthanised or died apart from those in which the tumour volume quadrupled (experimental end-point). A significant increase in survival time of RALA/alendronate nanoparticle treated mice of 56.3% was seen when compared to the untreated control group (p<0.001). The survival time of this group was also significantly higher compared to the alendronate only treated group at 32% (p<0.01) (FIG. 52c).

### RAT Results

RAT was synthesized (FIG. 54) and was able to complex pEGFP-N1 into nano-sized particles. Zetasizer analysis coupled with dynamic light scattering software analysis was performed to analyse the size, charge, particle count and polydispersity index of the RAT/pEGFP-N1 nanoparticles (FIG. 55). Through N:P ratios 1 to 4 the zeta potential increases from −17.9 mV±3.60 to 19.13 mV±3.75 and at N:P12 the nanoparticles were 71.03 nm±11.36 with a zeta potential of 17.49 mV±11.92. Taken together it is likely that N:P ratios of 4 to 12 have characteristics suitable for transfection.

A serum incubation study was used to determine if RAT/pEGFP-N1 nanoparticles were stable over a 6 h time period with and without the presence of foetal calf serum (FIG. 56). DNA migration was not observed with N:P12 nanoparticles on a 1% agarose gel when incubated for up to 6 h at 37° C.; supporting the gel retardation assay which demonstrated that DNA is neutralised by RAT from N:P3 upward. Decomplexation of the nanoparticles occurred in the presence of 10% sodium dodecyl sulphate enabling the integrity of the DNA to be assessed. In the presence of 10% serum, over a 6 h period, nanoparticles have not been disrupted as they remained within the wells of the agarose gel. The serum remains visible in all lanes indicating no aggregation with the positively charged nanoparticles. Analysis of DNA cargo, using 10% SDS to disrupt the nanoparticles, reveals that DNA integrity was not affected by serum endonucleases and protection was afforded by RAT.

The specificity of the RAT peptide was assessed using a targeting inhibition study (FIG. 57). Free targeting peptide, TMTP-1, was added at a range of concentrations prior to transfection as a competitive inhibitor of RAT/pEGFP-N1 nanoparticles and results were compared with the untargeted RALA peptide. The results show that as the concentration of competitive inhibitor increased transfection efficacy with RAT decreased. Conversely the inhibitor had no significant effect upon transfections with RALA. For example when 0.25 nM, 1.5 nM and 2 nM of inhibitor was placed upon PC-3 cells, gene expression with RAT/pEGFP-N1 was significantly reduced by 19.16%±8.00, 48%±17.00 and 57.26%±16.01 respectively (P<0.1). This indicates that the RAT nanoparticles are internalising via the TMTP-1 receptor thus conferring a degree of specificity.

TEM also confirmed the presence of the RALA nanoparticles inside the composite nanoparticles (FIG. 59). An in vitro DNA release study also demonstrated that the composite nanoparticles were able to release DNA, with 10% DNA content released in 24 hours and continuous release over 6 weeks.

In summary, the results presented show that RALA is efficient, stable, safe and a viable delivery vehicle for iNOS DNA, RUNX2 siRNA and bisphosphonate anti-cancer therapeutics.

### Conclusion

The physical properties of the RALA/pEGFP-N1 nanoparticles have been analysed and their efficacy as a transfection agent demonstrated both in vitro and in vivo. RALA was found to form stable complexes with pEGFP-N1 and facilitate the transfection of ZR-75-1 cells. Gel retardations show that complexes are formed at N:P ratios as low N:P 1, but full complexation is not seen until N:P 4, which is comparable with KALA and ppTG peptides [Rittner et al. 2002]. The RALA/pEGFP-N1 complexes cannot be defined as nanoparticles until N:P 4, as their size at N:P ratios 2 and 3 was in the micrometer range. At ratios of N:P 4 and above, RALA forms nanoparticles with pEGFP-N1 with a positive charge of 30 mV. This is in agreement with the counter-ion condensation theory, which states that particle sizes of charged complexes should be lower than those of uncharged particles, as electrostatic repulsion should prevent aggregation [de Smedt et al. 2000, Bagwe et al. 2006].

Given that at the N:P ratios which yield the highest transfection efficacy, the particles have a positive surface charge and a mean diameter below 100 nm, it is possible that they bind to the negatively charged cell surface proteoglycans non-specifically and are subsequently taken up into the endosomes.

With respect to transfection efficiency, the use of arginine in the RALA peptide has two distinct advantages; firstly arginine has consistently been shown to be the optimal amino acid for condensing DNA with arginine rich sequences binding in milliseconds (Murray et al 2001). Secondly arginine rich sequences based on the Rev sequence have the capacity to actively transport DNA into the nucleus of cells via the importin pathway (Malim et al 1989). This gives RALA a distinct advantage over conventional peptide delivery systems.

We have also shown that the RALA/pEGFP-N1 nanoparticles are not strongly cytotoxic, causing only a 20% reduction in cell viability in transfected cell monolayers. Perhaps the most important result is the confirmation of in vivo activity of the nanoparticles following systemic administration. High levels of delivery to the lungs were seen when a plasmid expressing luciferase was delivered to mice using the ppTG-1 peptide, but the liver was not examined [Rittner et al. 2002]. When fluorescently labelled siRNA was delivered with the MPG-8 peptide, it was observed in the majority of organs with high levels in the lungs and liver [Crombez et al. 2009]. No morbidity or mortality of animals was observed following treatment in the experiments described in this work, although this has not always been the case with peptide based gene delivery agents (Rittner et al. (2002) reported the death of several mice when delivering the plasmid systemically with the ppTG1 peptide.

In addition, RALA does not appear to cause a significant immune response upon repeated administration beyond the inflammation associated with tissue damage caused by the needle at the site of injection. There is also no neutralization of RALA following repeated administration. Furthermore, RALA appears to shield naked DNA from generating an adaptive immune response and does not cause an antibody response on its own. This is an encouraging result given that peptides are often used as vaccines because they share homology with viral and tumour proteins and produce a high antigenic response [Yang et al. 2009, Rodriguez and Grubman 2009]. As such, it might be expected that RALA, a peptide that is analogous to viral fusion proteins, might likewise be highly immunogenic. It appears, that as RALA uses a simple highly repetitive, artificially designed sequence that is not common in nature, its immunogenicity is low.

Part of the effectiveness of RALA as a transfection agent is probably related to its ability to protect DNA or siRNA from a hostile environments. The complexation of RALA to plasmid DNA forms nanoparticles that protect DNA from, freeze-drying and degradation in serum. While the ability to protect the cargo from degradation by serum has a bearing on transfection efficacy, the ability to act as a lyoprotectant has implications for further formulation related issues that surround transfection agents. The logistics behind supplying gene medicine to clinics are complicated by the lack of stability of most prospective vectors. Since viral vectors are notoriously difficult to store and non-viral vectors usually require lyoprotectants, which alter the final formulation, before they can be successfully freeze-dried, it is promising to see that RALA/pEGFP-N1 nanoparticles retain activity following reconstitution after lyophylisation.

RALA has also been shown to successfully condense and form nanoparticles with a range of bisphosphonates, siRNA and is an excellent tool for local delivery. It has also been used for the systemic delivery of the iNOS therapeutic to metastatic deposits of cancer with an excellent response. This indicates a wide range of applications for this peptide delivery system.

### EXAMPLE 3: ALTERNATIVE CELL PENETRATING AMPHIPATHIC PEPTIDE SEQUENCES

The following peptide sequences based on RALA (WEARLARALARALARHLARALARALRACEA) were also prepared using conventional commercial techniques as expanded on in Example 1.

The table below shows the key characteristics of RALA (WEARLARALARALARHLARALARALRACEA) derivative Peptides in ZR-75-1 breast cancer cells.

| | | | Characteristics | | | Transfection |
|---|---|---|---|---|---|---|
| | | SEQ | Length Hydro- | Best | | Efficiency |
| Peptide | | ID | philic:Hydro- | Size | Charge | in ZR-75-1 |
| N:P10 | | No. | phobic +/− | (nm) | (mV) | Cells |
| 1. | Original | 1 | 30 mer | 70 | 25 | 30% |
| | RALA | | 30:67:1 | | | |
| | | | 8:2 | | | |
| 2. | Peptide 2 | 2 | 29 mer | 76 | 22 | 55 |
| | (H Removed) | | 31:70 | | | |
| | | | 7:2 | | | |
| 3. | Peptide 3 | 4 | 30 mer | 51 | 24 | 41 |
| | (H Replaced | | 33:67 | | | |
| | with E) | | 7:3 | | | |
| 4. | Peptide 4 | 5 | 29 mer | 37 | 12 | 50 |
| | (H Removed | | 33:67 | | | |
| | and Replaced | | 8:2 | | | |
| | W replaced | | | | | |
| | with R) | | | | | |
| 5. | Peptide 5 | 6 | 29 mer | 53 | 13 | 46 |
| | (H Removed | | 37:63 | | | |
| | and W replaced | | 9:2 | | | |
| | with R and | | | | | |
| | C replaced | | | | | |
| | with R) | | | | | |
| 6. | Peptide 6 | 7 | 30 mer | 308 | 6 | 43 |
| | (H Replaced | | 40:60 | | | |
| | with E and | | 9:3 | | | |
| | W replaced | | | | | |
| | with R and | | | | | |
| | C replaced | | | | | |
| | with R) | | | | | |

### Results

The results in terms of transfection efficiency in ZR-75-1 cells are shown above. Peptides 1-5 successfully condensed the DNA into nanoparticles less than 100 nm. The exception being peptide 6, where the smallest nanoparticle measured was 308 nm. It can also be deduced that the highest transfection efficiency was with peptide 2 at 55% and as the hydrophilic ratios increase up to 40% the surface charge of the nanoparticle decreases. Furthermore the addition of glutamic residues reduces transfection efficiency as evidenced by peptide 3 and peptide 6. Nevertheless all sequences have potential as delivery vehicles for nucleic acids and hydrophilic compounds.

A 22mer WEARLARALARALARHLRACEA was also tested but was unable to condense DNA into nanoparticles and transfect cells and was therefor deemed unsuccessful.

### EXAMPLE 4: MICROPROTRUSION ARRAYS LOADED WITH NANOPARTICLES

### Materials and Methods

### Preparation of 30% Stock Solution of Polymers

Aqueous 30% stock solution of Gantrez® AN-139 poly(methylvinylether/maleic acid), (PMVE/MA) was prepared using 30 g of poly(methylvinylether/maleic anhydride), (PMVE/MAH) (ISP Corp. Ltd., Guildford, UK) which was added to 70 mL ice-cooled water and stirred vigorously to ensure complete wetting and prevention of aggregation. The mixture was then heated and maintained between 95° C. and 100° C. until a clear solution was formed. Upon cooling, the blend was then readjusted to the final concentration of 30% w/w by addition of an appropriate amount of deionised water.

Aqueous 30% stock solution of PVA (Polyvinyl alcohol), (Sigma, UK), was prepared using 30 g of PVA which was added to 70 mL ice-cooled water and stirred vigorously to ensure complete wetting and prevention of aggregation. The mixture was then heated and maintained between 95° C. and 100° C. until a clear solution was formed. Upon cooling, the blend was then readjusted to the final concentration of 30% w/w by addition of an appropriate amount of deionised water.

Aqueous 40% stock solution of PVP (Polyvinylpyrrolidone), (Sigma, UK), was prepared using 40 g of PVP which was added to ice-cooled water and stirred vigorously to ensure complete wetting and prevention of aggregation. The mixture was then heated and maintained between 95° C. and 100° C. until a clear solution was formed. Upon cooling, the blend was then readjusted to the final concentration of 40% w/w by addition of an appropriate amount of deionised water.

### Fabrication of Polymeric Microneedle Arrays

0.5 g of polymer gel was poured into a silicon mould. To ensure the polymer matrix reached the tips of the MN mould, the moulds were centrifuged at 3000 rpm for 15 min. Following centrifugation, the arrays were dried at room temperature for 48 h. Upon hardening, the arrays were released from the mould by carefully peeling it away. Arrays are shown in FIG. 61.

Agarose Gel Analysis of Nanoparticle Release from Polymeric Solutions

RALA/pEGFP-N1 complexes at N:P ratio 10 were prepared at room temperature and incubated at room temperature for 30 min. Following this incubation 50 mg of polymeric stock solution was added to the complexes and incubated at room temperature for 30 min. Subsequently, SDS (Sigma, UK) was added (10%) to the eppendorfs to decomplex DNA from the peptide. Following incubation, 30 μL of the samples (corresponding to 0.6 μg of DNA) were electrophoresed through a 1% agarose gel containing 0.5 μg/mL EtBr to visualize DNA mobility. A current of 80 V was applied for 1 h and the gel imaged using a Multispectrum Bioimaging System (UVP, UK). This experiment was repeated with proteinase K as the NP lysing agent. Results illustrated in FIG. 62.

Standard Curve for the Determination of DNA Concentration Following Release from RALA/DNA NPs

RALA/pEGFP-N1 NPs were analysed through fluorescence detection with Quant-iT™ Picogreen® Reagent (Invitrogen, UK). Quant-iT™ Picogreen® Reagent is a fluorescent nucleic acid stain for quantitating double-stranded DNA in solution. Upon addition to the solution the reagent binds to the double stranded DNA and it's fluorescence intensity increases several hundred fold, the fluorescence intensity of the resulting Picogreen/DNA complex is directly proportional to the amount of DNA in the sample.

For determination of DNA detection using this method following release from RALA/pEGFP-N1 NPs a representative standard curve was used. A solution of RALA/pEGFP-N1 NPs were made and subsequently diluted with Tris 10 mM to produce a range of NP solutions of known concentrations. 50 μL of these solutions were then pipetted into a 96-well plate and 50 μL of 0.1 mg/mL Proteinase K (Sigma, UK) subsequently added and samples incubated at 37° C. for 30 min. Quant-iT™ Picogreen® Reagent was then added to the samples and the samples analysed by excitation at 480 nm and the fluorescence emission intensity measured at 520 nm using a Synergy 2 Multi-Mode Microplate Reader (BioTek Instruments Inc, UK). Results are shown in FIG. 63 (A).

Quantification of NP Release from Polymer Matrices

RALA/pEGFP-N1 N:P 10 NPs containing 1 μg DNA were incorporated into the stock solutions of the polymeric matrices to form 20% polymeric solutions. These NP/polymer mixtures were incubated at room temperature for 1 h and subsequently dissolved in 1 mL Tris buffer (10 mM) for 1 h. 50 μL samples of these solutions were then pipetted into a 96-well plate and 50 μL of 0.1 mg/mL Proteinase K (Sigma, UK) subsequently added and samples incubated at 37° C. for 30 min. Quant-iT™ Picogreen® Reagent was then added to the samples and the samples analysed using a Synergy 2 Multi-Mode Microplate Reader (BioTek Instruments Inc, UK). Results are shown in FIG. 63 (B).

Determination of pDNA Secondary Structure in the Presence of PMVE/MA by Circular Dichroism

To examine the secondary structure of pDNA after incorporation into PMVE/MA samples of PMVE/MA only, pLux only and PMVE/MA-pLux were made. The samples were then dissolved in 2 mL PBS so the final concentration of pLux in solution was 50 μg/mL. CD spectra were obtained with Jasco J-185 spectopolarimeter equipped with a temperature controller. CD spectra were collected at 20° C. using a 1 cm quartz cell over the wavelength range of 240-350 nm. Results are shown in FIG. 64.

### WST-1 Cell Viability Assay Following Exposure to Polymer Matrices

NCTC-929 fibroblast cells were seeded at a density of 30,000 cells per well onto 96-well tissue culture plates (VWR, UK) for 24 h prior to the assay. Media was then supplemented with 0, 5, 10 or 20 mg/mL of either 20% PVA, 20% PVP or 20% PMVE/MA and incubated for 6 h under standard cell culture. Following this incubation 10% WST-1 reagent (Roche, UK) was added to the cell media and the cells were incubated for a further 2 h. Subsequently the plates were shaken for 1 min and absorbance measured at 450 nm on an EL808 96-well plate reader (BioTek Instruments Inc, UK). The measured absorbance values are expressed as a percentage of the control (untreated cells) where the control is defined as 100% viable. Results are shown in FIG. 65.

### Measurement of MN Height Reduction Following Application of an Axial Load

To determine the axial forces (i.e. the force applied parallel to the MN vertical axis) necessary for mechanical fracture of the MNs, the TA-XT2 Texture Analyser (Stable Microsystems, U.K) was employed. MN arrays of 3×3 MNs were used. The arrays were attached to the moveable cylindrical probe of the Texture Analyser using double-sided adhesive tape. An axial compression load was applied to the MN arrays to deduce the changes that occur to the structure of the MNs upon force application. The test station pressed the MN arrays against a flat aluminium block of dimensions 9.2×5.2 mm at a rate 0.5 mm per sec with defined forces of 0.05, 0.1, 0.2, 0.3 and 0.4 N/needle for 30 s. Before and after fracture testing, 3 MNs of each array were examined by a digital microscope (GE-5 USB Digital Microscope) under magnification 180× to determine the height of the MNs after testing. The MN height was measured using the ruler function of the microscope software so the percentage reduction in the MN height could be calculated. Results shown in FIG. 66.

### Fabrication of Dissolvable RALA/pEGFP-N1 NP Loaded 20% PVP MN Arrays

RALA/pEGFP-N1 loaded MNs were prepared using the micromoulding process, MNs manufactured from aqueous blends of 20% PVP encapsulating RALA/pEGFP-N1 NPs were prepared by diluting the 40% stock solution 50:50 with NP solution. 0.2 g of the polymeric gel containing the RALA/pEGFP-N1 NPs was weighed into the moulds and centrifuged at 3000 rpm for 10 min to ensure the MN cavities were filled. A further 0.3 g of 20% PVP polymer was added to the moulds to form the baseplate to which the microneedles are attached and centrifuged again at 3000 rpm for 10 min. The arrays were left to dry at room temperature and after 48 h, were manually released from the moulds and the polymeric side walls removed using a heated scalpel. Each MN array was either composed of 9 (3×3) or 361 (19×19) needles perpendicular to the baseplate depending on the mould used for fabrication. The MNs were of conical shape, 600 μm high with base width of 300 μm and 300 μm interspacing.

### Scanning Electron Microscopy of Polymeric MN Arrays

RALA/pEGFP-N1 NP loaded 20% PVP microneedle arrays were fabricated and mounted onto metal stubs with double sided carbon tape and sputter coated with gold and allowed to dry overnight. Arrays were visualised using a Jeol JSM-840A scanning microscope (Jeol, UK). Images shown in FIG. 67.

### Agarose Gel Determination of RALA/pEGFP-N1 NP Stability in 20% PVP Polymer Matrix for Up to 7 Days

20% PVP polymer loaded with RALA/pEGFP-N1 N:P 10 NPs containing 1 μg DNA were prepared and incubated at (A) 20° C., 46% relative humidity (RH) for 0, 1, 5, 3 or 7 days. At each time point the polymeric formulations were dissolved in 50 μL distilled water (Gibco, UK) and separated into 25 μL samples, to which 10% SDS was added to one sample to decomplex the NPs present in solution. 30 μL samples of each solution were then electrophoresed through a 1% agarose gel containing 0.5 μg/mL ethidium bromide (EtBr) (Sigma, UK) to visualize DNA. A current of 80 V was applied for 1 h and the gel imaged using a Multispectrum Bioimaging System (UVP, UK). Results are shown in FIG. 68.

Determination of Functionality of RALA/pEGFP-N1 N:P Ratio 10 NPs Encapsulated within 20% PVP Matrix Up to 7 Days

20% PVP polymers loaded with either pEGFP-N1 or RALA/pEGFP-N1 N:P 10 NPs containing 1 μg DNA were prepared and incubated at room temperature for 1 h and 7 days. Following these incubations the polymeric formulations were dissolved in 200 μL PBS for 1 h.

NCTC-929 cells were prepared for transfection by seeding at a density of 30,000 cells per well onto 96-well tissue culture plates (VWR, UK) for 24 h prior to transfection. Cells were conditioned for 2 h in Opti-MEM serum free media (Gibco, UK) which was then supplemented with 100 μL of polymer/NP solution. Following incubation for 6 h the media was removed and replaced with serum supplemented culture media. Cells were imaged using the Nikon Eclipse TE300 inverted microscope with epifluorescence attachment (Nikon, USA) and images captured using a Nikon DXM1200 digital camera (Nikon, USA) using a ×200 magnification 24 h post transfection. Images are displayed in FIG. 69.

### Fabrication of MN Arrays Containing Concentrated NPs

MNs were manufactured from aqueous blends of 20% w/w PVP encapsulating pDNA and RALA/pDNA NPs were prepared by diluting the stock solution of 40% PVP 50:50 with the appropriate amount of pDNA/NP solution. When fabricating MNs loaded with concentrated RALA/pDNA NPs the RALA and pDNA were combined initially and incubated at room temperature for 30 min before incorporation into the PVP matrix.

25 mg of the polymeric solution containing the DNA or NPs was weighed into the moulds and centrifuged at 3000 rpm for 5 mins, this was repeated twice to ensure the microneedle cavities were filled. A further 0.5 g of 20% PVP solution was added to the moulds to form the baseplate to which the microneedles are attached and centrifuged again at 3000 rpm for 10 min. The arrays were left to dry at room temperature and after 48 h, were manually released from the moulds and the polymeric side walls removed using a heated scalpel. Each MN array was composed of 361 (19×19) needles perpendicular to the baseplate.

Quantification of RALA/pEGFP-N1 N:P Ratio 10 NPs Encapsulated within the MN Array

In order to determine the quantity of DNA present in the MNs themselves the MNs were fabricated containing RALA/pEGFP-N1 NPs. The needles were sheared from the baseplate and both components of the array dissolved in 4 mL 10 mM Tris buffer for 1 h. 50 μL samples of these solutions were then pipetted into a 96-well plate and 50 μL of 0.1 mg/mL Proteinase K (Sigma, UK) subsequently added and samples incubated at 37° C. for 30 min. Quant-iT™ Picogreen® Reagent was then added to the samples and the samples analysed. Results are detailed in Table 1.

Evaluation of RALA/pEGFP-N1 Nanoparticle Release from 20% PVP MNs Across Neonatal Porcine Skin

Neonatal porcine skin was obtained from stillborn piglets and immediately (<24 hours after birth) excised, trimmed to a thickness of 300±50 μm using dermatome and frozen in liquid nitrogen vapour. Skin was then stored in aluminium foil at −20° C. until further use. Shaved skin samples were mounted on the receptor compartment with stratum corneum (SC) side of the skin exposed to ambient conditions and dermal side in contact with the release medium. 20% PVP MN arrays containing concentrated RALA/pDNA NPs were pressed into the porcine skin using a syringe plunger to ensure insertion of the MNs into the SC. Samples were withdrawn from the receptor compartment at pre-determined time intervals and the volume taken was replaced by the same volume of fresh receptor medium to maintain constant conditions. 50 μL samples of these solutions were then pipetted into a 96-well plate and 50 μL of 0.1 mg/mL Proteinase K (Sigma, UK) subsequently added and samples incubated at 37° C. for 30 min. Quant-iT™ Picogreen® Reagent was then added to the samples and the samples analysed. Results are shown in FIG. 70.

Optical Coherence Tomographic Assessment of MN Penetration into Full Thickness Neonatal Porcine Skin

Optical coherence tomography (OCT) was used to determine the penetration characteristics of 19×19 20% PVP MN arrays loaded with RALA/μLux NPs following insertion into excised full thickness neonatal porcine skin using either spring-activated applicator or manually using gentle thumb pressure. Neonatal full thickness porcine skin was prepared and equilibrated in PBS for 30 min at 37° C. to restore conditions resembling the in vivo state. The skin was then placed onto a sheet of dental wax for support with the SC side facing towards the environment. MN arrays were inserted into the skin using an applicator, at forces of 8 N, 11 N and 16 N. To use the applicator, firstly the required spring was loaded into the piston shaft. The flat base of the piston was then pushed up towards the piston shaft until it locked into place. This applicator could then be activated by simply pressing a release button, which drives the piston towards the target surface. This process was repeated three times to ensure proper MNs insertion into the skin. The skin was immediately viewed using the OCT scanner and images were analysed using Image J software.

To investigate the insertion of the MNs using gentle thump pressure, the skin was prepared as described previously and the MN array inserted into the full thickness porcine skin by applying gentle thumb pressure against the array for 30 sec. The skin was immediately viewed using OCT Scanner and images were analysed using Image J software. Results are shown in FIG. 71.

Confocal Microscopy of Murine Ears Following Application of MN Arrays Containing Concentrated Cy-3 Labelled pOVA and Cy-3 Labelled RALA/pOVA NPs

pOVA was fluorescently labelled with the Cy-3 fluorophore (Mirus, USA) according to the manufacturer's instructions. Briefly, 100 μg pOVA was labelled with 20 μL Cy-3 Label IT Reagent at 37° C. for 1 h. The labelled DNA was then concentrated using ethanol precipitation to produce Cy-3 labelled pOVA at a concentration of 5 μg/μL. This DNA was then used to form to form 20% PVP MN arrays containing either Cy-3 pOVA or Cy-3 RALA/pOVA NPs.

25 mg of the polymeric solution containing the Cy-3 labelled pOVA or RALA/pOVA NPs was weighed into the moulds and centrifuged at 3000 rpm for 5 mins, this was repeated twice to ensure the microneedle cavities were filled. A further 0.5 g of 20% PVP solution was added to the moulds to form the baseplate to which the microneedles are attached and centrifuged again at 3000 rpm for 10 min. The arrays were left to dry at room temperature and after 48 h, were manually released from the moulds and the polymeric side walls removed using a heated scalpel. Each MN array was composed of 361 (19×19) needles perpendicular to the baseplate.

The MNs were applied to the mouse ear for 1 h, then the animals were sacrificed. Following harvesting of ear tissue from sacrificed animals the tissue was stored in 4% formaldehyde solution overnight. Ear tissue was then mounted into a microscope slide (VWR, UK) using 100% glycerol (Sigma, UK) and imaged using a TSC SP5-Leica Microsystems confocal microscope (Leica, UK). Images were analysed using LAS AF Lite Software (Leica, UK). Images shown in FIG. 72.

### Application of Microneedles In Vivo

Prior to application of the MN arrays the mice were anaesthetized via intraperitoneal (i.p.) injection of Rompun and Ketaset. The dorsal ear skin of the mice was wetted with 10 μL of water and the MN arrays manually inserted by holding in place for 5 min into both ears of each animal. In order to keep the MN arrays in place micropore tape was used to secure the arrays to the ear tissue. MN arrays were removed 24 h following application.

### Imaging of Harvested Tissue Using the Xenogen IVIS 200 Imaging System

Following harvesting of the organs from sacrificed animals they were subsequently placed in a 6-well plate (VWR, UK) bathed in D-Luciferin Potassium Salt (PerkinElmer, UK) in PBS (15 mg/mL) for 10 min. The organs were then transferred to a 24-well plate (VWR, UK) and imaged using the Xenogen IVIS 200 Imaging System (PerkinElmer, UK). Images were analysed using Living Image® 3.2 software (Leica, UK). Results shown in FIG. 73.

Flow Cytometric Analysis of Harvested Auricular Lymph Nodes Following Immunisation with RALA/pOVA NPs Via MN Application

Animals were sacrificed 10 days post immunization and the auricular lymph nodes were harvested and collected into small petri dishes (VWR, UK) with 1 ml of RPMI media (Gibco, UK) and manually dissociated by compression through a nylon membrane. The resulting cell suspensions were centrifuged at 500 g for 5 mins and the cell pellet resuspended in 1 mL PBS. The cells were then stained with SIINFEKL/H-2 Kb pentamers conjugated to APC for 20 mins in accordance with the manufacturers instructions (Pro-Immune Limited, UK). The cells were then stained using fluorochrome-conjugated antibodies for CD8 and B220 (BD Biosciences and eBioscience, UK) to determine the T and B-cell populations respectively. Data was collected on FACS Canto II (BD Biosciences) and analyzed using FlowJo software (Tree Star). Results are shown in FIG. 74.

### Agarose Gel Determination of RALA/HPV-16 E6 and RALA/HPV-16 E7 NP Stability in 20% PVP Polymer Matrix for Up to 21 Days

Microneedles loaded with RALA/HPV-16 E6 or RALA/HPV-16 E7 N:P 10 NPs containing 1 μg DNA were prepared and left 48 h to dry. Microneedles were incubated either at 4° C., 35% relative humidity (RH) or 20° C., 40% RH, or 20° C., 86% RH for 7, 14, or 21 days. At each time point microneedles were dissolved in 500 μL distilled water (Gibco, UK) and separated into 250 μL samples, to which proteinase K (0.5 mg/mL) was added (10%) to one sample to decomplex the NPs present in solution. 30 μL samples of each solution were then electrophoresed through a 1% agarose gel containing 0.5 μg/mL ethidium bromide (EtBr) (Sigma, UK) to visualize DNA. A current of 80 V was applied for 1 h and the gel imaged using a Multispectrum Bioimaging System (UVP, UK). Results are shown in FIG. 75.

Determination of Functionality of RALA/HPV-16 E6, RALA/HPV-16 E7, and RALA/HPV-16 E6/E7 N:P Ratio 10 NPs Encapsulated within 20% PVP Matrix

20% PVP polymers loaded with either HPV-16 E6, HPV-16 E7, HPV-16 E6/E7, RALA/HPV-16 E6, RALA/HPV-16 E7 or RALA/HPV-16 E6/E7 NPs containing 1 μg DNA were prepared and incubated at room temperature for 48 h to dry. Following incubation, microneedles were dissolved in 200 μL PBS for 1 h. NCTC-929 cells were prepared for transfection by seeding at a density of 30,000 cells per well onto 96-well tissue culture plates (VWR, UK) for 24 h prior to transfection. Cells were conditioned for 2 h in Opti-MEM serum free media (Gibco, UK) which was then supplemented with 100 μL of polymer/NP solution. Following incubation for 6 h the media was removed and replaced with serum supplemented culture media. After 24 h, cells were harvested with Laemelli buffer (Sigma, UK). Cell lysates were electrophoreised through a SDS-PAGE gel and transferred onto nitro-cellulose membrane according to standard procedures. The membrane was probed with both HPV-16 E6 and E7 antibodies and developed using chemiluminesce kit (Millipore, UK) according to the manufacturer's instructions. Results shown in FIG. 76.

### Determination of Gene Expression in DCs (Dendritic Cells) in Draining Lymph Nodes Post MN (Microneedle) Application

MNs loaded with pDNA encoding the tdTomato fluorophore were applied to a hairless area of skin on the dorsum of C57BL/6 mice for 24 h as described previously. 4 days post MN application the animals were sacrificed and the draining lymph nodes harvested and enzymatic degradation performed with Collagenase, Type IV (Gibco, Cat no: 17104-019). Using sharp scissors the lymph nodes were cut for 10 min until completely liquefied. Using RPMI media (5 ml) the cells were washed to the bottom of a 15 ml falcon tube and warmed to 37° C. 170 μl of collagenase (30 mg/ml) was added to the 5 ml and the cells pipetted vigorously for 20 mins, another 170 μl of collagenase was added and pipetting continued for another 10 mins. The cell suspension was then filtered through a 100 μm mesh filter into a clean 15 ml falcon tube. The tube and mesh were then rinsed with another 2 ml RPMI and centrifuged at 600 rpm, 4° C. for 10 min. The cells were then resuspended in 1 ml PBS and transferred to flow tubes. The falcon was rinsed with a further 1 ml PBS which was also transferred to the corresponding flow tubes followed centrifuged again. The cells were then stained in a two-step process. Step 1: MHC class-II stain i.e. 1 ml PBS, 1 μl MHC class-II biotin antibody (eBioscience, Cat no: 13-5321-82) and 20 μl MHC class-II antibody (BD Pharmingen, Cat no: 556999) for 20 mins on ice followed by step 2: CD11c and Streptavidin mix i.e. 1 ml PBS, 2 μl CD11c antibody (eBioscience, cat no: 51-0114-82) and 1 μl Streptavidin-PEcy7 antibody (eBioscience, cat no: 25-4317-82) and incubated on ice for a further 20 mins. The cells were resuspended in 200 μl PBS and analysed by flow analysis on the FACS Canto II and using FlowJo software (FIG. 79).

Determination of Circulating HPV-16 E6/E7 IgG Antibody Levels Generated Following Immunisation with Plasmid DNA Expressing HPV-16 E6/E7 Antigens

C57BL/6 mice (n=4) were immunized 3 times, at fortnightly intervals. Each immunization involved delivering 50 μg plasmid DNA encoding HPV-16 E6/E7 antigens±g pla via i.m. and MN delivery. Circulating levels of HPV-16 E6/E7 IgG antibodies were determined by ELISA analysis of serum collected 10 days post 2^{nd} and 3^{rd} immunisations. 96-microwell plate was coated with 100 μl (0.5 mg/ml) HPV-16 E7/E6 peptides incubated at 4° C. overnight. The wells were then blocked with PBS containing 20% fetal bovine serum and incubated at 4° C. for 16 h. Serum samples diluted in PBS (1:100) were added and incubated at 37° C. for 2 h. The plate is incubated with a 1:2000 dilution of a goat antimouse IgG HRP-conjugated antibody at room temperature for 1 h. Subsequently an enzyme substrate (OPD, Sigma) was added for colour development. Immunoreactivity is detected with an ELISA plate reader at a wavelength of 450 nm. Quantification IgG was performed using Easy titer IgG assay kit (Thermo scientific, UK) (FIG. 80).

Determination of Generation of HPV-16 E6/E7-Specific Cytotoxic T Cells Following Immunisation with Plasmid DNA Expressing HPV-16 E6/E7 Antigens

Spleens were harvested from immunised C57BL/6 mice 10 days post 3^{rd} immunisation. Each immunization involved delivering 50 μg plasmid DNA encoding HPV-16 E6/E7 antigens±RALA via i.m. and MN delivery. Spleens are removed aseptically, homogenised and resuspended in red blood cell (RBC) lysis buffer to remove RBCs. Following RBC lysis, isolated splenocytes from the same group are pooled and re-suspended in RPMI 1640 medium (TC-1 medium) and counted. T cells (used as the effecter cells) were co-cultured in RPMI-1640 medium containing irradiated TC-1 cells (10⁴ per well) (used as the target cells) in 24-well plate. Media was supplemented with 20 units of interleukin-2 (Peprotech) and incubated under standard tissue culture conditions (37° C., 5% CO₂) for 6 days. Dead T cells were removed by centrifugation with Percoll solution (Amersham Biosciences). Viable T cells are seeded with non-irradiated TC-1 cells in the ratios of 5:1 and 10:1 in an assay medium (1% BSA medium) in triplicates and incubated under standard tissue culture conditions (37° C., 5% CO₂) for 5 h. Supernatant was harvested and cytotoxicity determined using cytotoxicity detection kit (LDH) (Roche) according to manufacturers protocol. The colour change was detected by plate reader analysis at a wavelength of 450 nm and the cytotoxicity calculated by the following equation:

$$Cytotoxicity ( \% ) = \frac{( Experimental value - effecter cell control ) - low control}{High control - low control} \times 100 \%$$

“High control”=the total LDH released from the target cells, after lyzing TC-1 cells with 1% Triton X-100 in assay medium.
“Low control”=the natural release of LDH from the target cells, which is obtained by adding TC-1 cells only in the assay medium.
“T-cell control”=use to measure the natural release of LDH from T cells was obtained by adding the different ratios of T cells only in the assay medium (FIG. 81).
Determination of Interferon-Gamma Secretion from Splenocytes Restimulated with E6/E7-Expressing TC-1 Cells Ex Vivo

Spleens are removed aseptically, homogenised and resuspended in red blood cell (RBC) lysis buffer to remove RBCs. Following RBC lysis, isolated splenocytes from the same group are pooled and re-suspended in RPMI 1640 medium (TC-1 medium) and counted. T cells (used as the effecter cells) were co-cultured in RPMI-1640 medium containing irradiated TC-1 cells (104 per well) (used as the target cells) in a ratio of 10:1, and media supplemented with 20 units of interleukin-2 (Peprotech) in 24-well plates. The cells were cultured in standard tissue culture conditions (37° C., 5% CO₂) for 4 days, then media was harvested for ELISA analysis of interferon-gamma (IFN-γ) (PeproTech, Cat no: 900-K98) (FIG. 82).

For ELISA assay, the capture antibody was diluted with PBS to a concentration of 1.0 μg/ml. and immediately added (100 μl) to each ELISA plate well. The plate was sealed and incubated overnight at room temperature. Following washing of the excess capture antibody from the wells 300 μl of blocking buffer was added to each well and Incubated for 1 h at room temperature. The harvested cell media was added to the prepared ELISA plate in triplicate and incubated at room temperature for 2 h. The detection antibody was diluted to a concentration of 0.25 μg/ml, and added 100 μl per well. Plate was incubated at room temperature for 2 h. This was followed by further washing, then Avidin Peroxidase (diluted 1:2000) was added and plate was incubated for 30 min at room temperature. ABTS substrate was added to each well, and incubated at room temperature for colour development. The plate was read using ELISA plate reader at 405 nm.

Determination of Efficacy of Prophylactic Immunisation with Plasmid DNA Expressing HPV-16 E6/E7±RALA Against Establishment of Tumour Following Implantation of TC-1 Cells In Vivo

C57BL/6 mice (n=9) were immunised 3 times, at fortnightly intervals. Each immunization involved delivering 100 μg plasmid DNA encoding HPV-16 E6/E7 antigens±RALA via i.m. and MN delivery. One week post 3^{rd} immunisation mice were challenged with 1×10⁵ E6/E7-expressing TC-1 cells per mouse via intradermal implantation on the dorsum The mice were monitored for evidence of tumour growth by palpation and tumour growth measured three times per week (FIG. 83).

Determination of Efficacy of Therapeutic Immunisation with Plasmid DNA Expressing HPV-16 E6/E7±RALA Against Growth of Established TC-1 Tumour

C57BL/6 mice (n=3) were implanted subcutaneously with 1×10⁵ E6/E7-expressing TC-1 cells per mouse, When tumour volume reached 50 mm³ the mice were immunised 3 times, at weekly intervals. Each immunization involved delivering 100 μg plasmid DNA encoding HPV-16 E6/E7 antigens±RALA via i.m. and MN delivery. The mice were monitored for evidence of tumour growth by palpation and measurement three times per week (FIG. 84).

### Quantification of Freeze-Dried RALA/pHPV-16 E6/E7 NP Ratio 6 Nanoparticles Delivered Following Application to C57BL/6 Mouse Ears

RALA/pHPV-16 E6/E7 (N:P ratio of 6) nanoparticles were freeze-dried using Advantage, VirTis freeze dryer and 5% w/v trehalose was used as cryoprotectant. MN arrays were formulated using 3 polymers, 360 kDa PVP, 58 kDa PVP and 9-10 kDa PVA, to contain RALA/pHPV-16 E6/E7 (N:P ratio of 6) nanoparticles encapsulating either 50 or 100 μg DNA. MN arrays were applied to the dorsal side of C57BL/6 mice ears for 5 min or 24 h followed by removal of the array and quantification of the HPV-16 E6/E7 DNA remaining in the array by Quant-iT™ PicoGreen® dsDNA quantification (Life Technologies, UK). Delivery of DNA from MNs formulated to contain 36 μg DNA (as used in previous in vivo studies) was also performed as a comparison. Following application of the MN arrays for (A) 5 min or (B) 24 h, the remaining array was removed and subsequently dissolved in 5 mL Tris buffer (10 mM) for 1 h. 50 μL samples of these solutions were then pipetted into a 96-well plate and 50 μL of 1 mg/mL Proteinase K subsequently added and samples incubated at 37° C. for 2 h. Quant-iT™ Picogreen® Reagent was then added to the samples and the samples analysed by excitation at 480 nm and the fluorescence emission intensity measured at 520 nm using a Synergy 2 Multi-Mode Microplate Reader (BioTek Instruments Inc, UK) (FIG. 85).

Fabrication of MN Arrays from PVP and PVA Polymers of a Range of Molecular Weights Loaded with RALA/pDNA Nanoparticles

50% w/w 360 kDa PVP, 13-23 kDa PVA and 9-10 kDa PVA stock solutions were manufactured by thoroughly mixing 5 g of lyophilised polymer with 5 g of refrigerated double distilled molecular grade water (Invitrogen, UK). Stock solution was then heated to 80° C. and mixed hourly until a homogenous, clear polymeric solution was formed. 75% w/w 58 kDa PVP stock solution was produced by thoroughly mixing 7 g of PVP powder with 3 g of refrigerated double distilled molecular grade water.

20% w/w MNs (360 kDa PVP, 13-23 kDa and 9-10 kDa PVA) and 30% w/w MNs (58 kDa PVP) containing RALA/pDNA were fabricated by mixing 50% w/w (360 kDa PVP, 13-23 kDa and 9-10 kDa PVA) or 75% w/w (58 kDa PVP) polymer solutions with RALA/pDNA solution at a ratio of 2:3. 25 mg of polymer-RALA/pDNA solution was then weighed into silicon moulds and centrifuged at 4000 rpm for 10 min to ensure complete filling of MN cavities. For fabrication of 360 kDa PVP, 13-23 kDa and 9-10 kDa PVA MN arrays, following centrifugation 0.5 g of 20% w/w polymer stock was weighed onto moulds to form a baseplate attached to the RALA/pDNA-loaded MNs and moulds were centrifuged again at 4000 rpm for 10 min. MN arrays were incubated at room temperature for 48 h for solidification and then peeled carefully from the mould. For fabrication of 58 kDa PVP MN arrays, 0.2 g of 30% w/w 58 kDa PVP solution was weighed into moulds which were centrifuged again as above and left to solidify by incubation at room temperature for 24 h. Subsequent to this incubation, 0.5 g of 20% 360 kDa PVP was weighed into moulds which were centrifuged again as detailed above. MN arrays were incubated at room temperature for 48 h to solidify and then peeled carefully from the mould (FIG. 86).

Measurement of MNs, Fabricated from PVP and PVA Polymers of a Range of Molecular Weights, Percentage Height Reduction Following Application of an Axial Force

MN arrays with 361 (19×19) needles were fabricated as detailed above, imaged and MN height measured prior to compression using a light microscope at ×35 magnification. MN arrays were then adhered to the movable probe of the TA-XT2 Texture Analyser (Stable Microsystems, UK) with double-sided sticky tape and a compression force of 45 N (0.125 N/needle) was then applied uniformly to the needles against a flat aluminium block. Following compression, MNs were re-imaged and measured using a light microscope at ×35 magnification. Percentage height reduction was calculated as the difference in MN height following compression divided by the original height×100 (FIG. 87).

Optical Coherence Tomographic Analysis of MN Penetration Mouse Ears Following Fabrication from PVP and PVA Polymers of a Range of Molecular Weights

20% w/w 360 kDa PVP, 13-23 kDa PVA, 9-10 kDa PVA and 30% w/w 58 kDa PVP MN arrays with 19×19 needles were fabricated as previously described. Murine ears were equilibrated in PBS for 30 min at 37° C. prior to insertion of MNs. Following equilibration the skin was placed on a sheet of dental wax with the epidermis facing externally. MNs were pressed into skin using the movable probe of the TA-XT2 Texture Analyser applying a range of forces (Stable Microsystems, UK) (10 N, 20 N, 30 N and 40 N). Following application of MNs, the skin was analysed using the optical coherence tomography (OCT) scanner. Images were then analysed and MN penetration depth measured using Image J software (FIG. 88).

### Cytotoxicity Analysis of PVP and PVA Polymers of a Range of Molecular Weights, to Fibroblast and Dendritic Cell Lines In Vitro

Fibroblast NCTC-929 and dendritic DC 2.4 cell lines were seeded in a 96-well plate at densities of 10,000 and 17,500 cells/well respectively. Cells were left to adhere overnight and the following day media was supplemented with polymer at concentrations of 0-40 mg/mL. Following 24 h incubation under standard tissue culture conditions, 10% MTS reagent (CellTiter 96 AQeous One Solution Reagent, Promega, UK) was added per well and cells were incubated for a further 2 h. Subsequently absorbance at 490 nm was measured using a EL808 96-well plate reader (Biotek Instruments Inc, UK). Measured absorbance values are expressed as a percentage of the absorbance of untreated control cells, where the control represents 100% viability (FIG. 89).

Quantification of RALA/pDNA Release from PVP and PVA Polymers of a Range of Molecular Weights

250 mg 20% w/w polymeric gels, 20% 360 kDa PVP, 20% 13-23 kDa PVA, 20% 9-10 kDa PVA and 30% w/w 58 kDa PVP loaded with 10 μg pDNA were fabricated by mixing 50% w/w (360 kDa PVP, 13-23 kDa and 9-10 kDa PVA) or 75% w/w (58 kDa PVP) polymer solutions with RALA/pDNA solution [N:P ratios (0-10)] at a ratio of 2:3. Following solidification of gels by incubation at room temperature for 48 h, gels were dissolved in 10 mM Tris buffer pH 8.0 for 1 h with stirring. Following dissolution, 50 μL samples of solution were transferred in triplicate to wells in a black 96 well plate. Samples were then incubated with 50 μL 0.2 mg/ml Proteinase K for 120 min at 37° C. Samples were then incubated at room temperature for 30 min with Quanti-iT Picogreen™ reagent and fluorescent emission at 520 nm was quantified using a Synergy 2 Multi-Mode Microplate Reader (BioTek Instruments Inc, UK) following excitation at 480 nm. The fluorescence of samples was used to determine concentration of pDNA in the solution using a standard curve as detailed previously and total pDNA release was subsequently calculated (FIG. 90).

Quantification of RALA/pDNA Release from MNs Fabricated from PVP and PVA Polymers of a Range of Molecular Weights

20% w/w 19×19 MNs incorporating RALA/pDNA were fabricated as previously described. Arrays were produced with solutions of RALA/pDNA N:P ratio 10 encapsulating 32 μg pDNA. The sidewalls were removed from arrays using a heated scalpel. Sidewalls and the baseplate and needles were then dissolved in 10 mM Tris buffer pH 8.0 for 1 h with stirring. Following dissolution, 50 μL samples were transferred in triplicate to wells in a black 96 well plate. Samples were then incubated with 50 μL 0.2 mg/ml Proteinase K for 120 min at 37° C. Samples were further incubated at room temperature for 30 min with Quanti-iT Picogreen™ reagent and fluorescent emission at 520 nm was quantified using a Synergy 2 Multi-Mode Microplate Reader (BioTek Instruments Inc, UK) following excitation at 480 nm. The fluorescence of samples was used to determine concentration of DNA in the solution using a standard curve as detailed previously and total DNA release was subsequently calculated (FIG. 91).

Agarose Gel Analysis of pDNA Integrity Following Incorporation into PVP and PVA Polymers of a Range of Molecular Weights

250 mg 20% w/w polymer gels (360 kDa PVP, 13-23 kDa PVA and 9-10 kDa PVA) and 30% w/w 58 kDa PVP gels loaded with 30 μg pLux were fabricated by mixing 50% w/w (360 kDa PVP, 13-23 kDa and 9-10 kDa PVA) or 75% w/w (58 kDa PVP) polymer solutions with RALA/pDNA solution (N:P ratios 0 and 6) at a ratio of 2:3. Gels were incubated at room temperature for either 0 or 7 days to assess pDNA stability. Gels were then dissolved in 10 mM Tris buffer pH 8.0 for 1 h with stirring. Following dissolution, two 35 μL samples of solution were transferred into 0.5 mL eppendorfs. Samples were incubated with 35 μL 10 mM Tris buffer pH 8.0, or, 35 μL 2 mg/ml Proteinase K for 120 min at 37° C. Following incubation, 30 μL samples were transferred to 0.5 mL eppendorfs and mixed with 5 μl 5× Nucleic Acid Loading Buffer (Biorad, UK). Subsequently, 20 μl samples were loaded onto 1% agarose gels containing 0.2 μg/mL Ethidium Bromide (EtBr) as a DNA intercalating agent. Samples were electrophoresed at 100 V for 1 h in 1×TAE buffer and then visualised under UV light using a Multispectrum Bioimaging System (UVP, UK) (FIG. 92).

In Vitro Cell Transfection with RALA/pEGFP-N1 Complexes Following Release from PVP and PVA Polymers of a Range of Molecular Weights

250 mg 20% w/w polymer gels (360 kDa PVP, 13-23 kDa PVA and 9-10 kDa PVA) and 30% w/w 58 kDa PVP gels incorporating 20 μg pEGFP-N1 were fabricated by mixing 50% w/w (360 kDa PVP, 13-23 kDa and 9-10 kDa PVA) or 75% w/w (58 kDa PVP) polymer solutions with RALA/pEGFP-N1 solution (N:P ratios 0-12) at a ratio of 2:3. Fibroblast NCTC-929 and macrophage RAW 264.7 cell lines were seeded in a 24-well plate at densities of 100,000 and 140,000 cells/well respectively. The following morning cell media was removed and cells were incubated for 2 h with serum-free Opti-MEM media (Life Technologies, UK). Polymeric gels were placed 1 mL of Opti-MEM media and incubated at 37° C. for 1 h to allow dissolution. Following incubation, cells were treated for 4 h with 250 μL of Opti-MEM media containing RALA/pEGFP-N1 complexes (N:P ratio 0-12) released from dissolved polymers. Following transfection, RALA/pEGFP-N1 complexes were removed, cells were washed with PBS and placed in normal media and incubated at 37° C. and with 5% CO₂.

Fluorescent Microscopy of Cells Transfected Following Nanoparticle Release from PVP and PVA Polymers of a Range of Molecular Weights

48 h post transfection, GFP reporter-gene expression was visualised by imaging cells at ×10 magnification under epifluorescence using the EVOS FL Cell Imaging System (Life Technologies).

Flow Cytometric Analysis of GFP Expression Following RALA/pGFP-N1 Nanoparticle Release from PVP and PVA Polymers of a Range of Molecular Weights

48 h post transfection, cells were washed with PBS and trypsinised with 0.5% Trypsin (Life Technologies, UK) for 5 min. Harvested cells were centrifuged at 1500 rpm for 10 min. Supernatant was removed and the cell pellet resuspended in 500 μL 2% Paraformaldehyde (Sigma, UK). Cells were stored at 4° C. until analysis of GFP expression using the FACS caliber system (BD Biosciences, UK). Data was analysed using Cyflogic software. Fluorescent intensity is reported at 4% gating (FIGS. 93 & 94).

### Results

Fabrication of MNs from PMVE/MA, PVA and PVP

Three polymeric matrices were investigated as the potential structural polymer for the manufacture of the dissolving MN arrays, Gantrez® AN-139 poly(methylvinylether/maleic acid), (PMVE/MA), Polyvinyl alcohol (PVA) and Polyvinylpyrrolidone (PVP). FIG. 61 presents representative images of MNs fabricated from the various polymeric materials. MNs formulated from aqueous blends of 20% PMVE/MA and 20% PVP constituted exact replicates of the silicon master structures. PMVE/MA and PVP MNs were found to produce sharp MNs and a flat, solid base plate. However, the tips of 20% PVA MNs were slightly bent towards one side with very thin and flexible base plates.

Agarose Gel Analysis of Nanoparticle Release from Polymeric Solutions

The stability of pEGFP-N1 and RALA/pEGFP-N1 NPs within the polymeric matrices under investigation was evaluated 24 h post fabrication by dissolving the polymer/nanoparticle formulations is 300 μL water and loading samples onto an agarose gel for electrophoresis. As illustrated in FIG. 62 (A), pEGFP-N1 released from both PVA and PVP travelled through the agarose gel via electrophoresis, visible in lanes 2 of the PVA and PVP sections of the gel image. Comparison of the bands produced by this released DNA to those of unencapsulated stock DNA indicate no degradation of the DNA plasmid has occurred and as such the released DNA is still predominantly in its supercoiled conformation and thus should be fully functional. Furthermore, RALA/pEGFP-N1 complexes incorporated into 20% PVA and 20% PVP remained intact as shown by the inability of the complexes to migrate down the agarose gel as illustrated in lanes 3. However, upon addition of a decomplexing agent, 10% SDS, to these formulations, DNA is released from the NPs and travels down the gel (lanes 4) indicating once again that DNA is not degraded in the formulation and can be released from the polymeric matrices.

In contrast, pEGFP-N1 and RALA/pEGFP-N1 incorporated into the PMVE/MA polymer were not visible on the agarose gel. It is possible to see that the wells of the agarose gel have been degraded following electrophoresis, suggesting the polymer adversely affects this method of analysis and thus, it is not possible to determine the stability of these DNA complexes within this polymer through agarose analysis.

This experiment was repeated as illustrated in FIG. 62 (B) with the enzyme proteinase K to determine if it could be used as a decomplexing agent for the NPs within the polymeric matrices as SDS is an unsuitable chemical component for future experiments involving the detection of DNA via fluorescence as the iodide ions present in the solution caused fluorescence quenching. The iodide ions collide with the fluorophore causing it to transfer its energy via a non-radiative transition rather than through the emission of a photon for fluorescence detection.

Samples were incubated for 30 min with 20% proteinase K (0.1 mg/mL) and loaded onto a 1% agarose gel. Analysis on the gel indicates that the RALA peptide is cleaved by proteinase K thus releasing the DNA into solution and enabling it to travel down the gel without causing degradation of the encapsulated DNA.

Fluorescent Detection of DNA Released from RALA NPs

The fluorescence intensity of the Picogreen® reagent is directly proportional to the quantity of free′ or ‘naked’ DNA present in solution as chelation of the reagent with DNA causes a 1000-fold increase in fluorescence. FIG. 63 (A i) illustrates that as the fluorescent intensity of samples containing no DNA is 38481±643 Fluorescent units (Fu) and those of samples containing RALA/pEGFP-N1 NPs encapsulating 1 μg DNA is 63491±80 Fu.

Conversely, when the NPs were incubated with proteinase K for 30 min prior to the addition of Picogreen® reagent, fluorescence intensity increases proportionally to the free′ DNA content of the solution as indicated by FIG. 63 (A ii). As the quantity of DNA-containing NPs present in solution increases and are subsequently lysed with proteinase K, there is a linear increase the quantity of free′ DNA present in solution and thus a correlating increase in fluorescence intensity, increasing to 827,826±2317 Fu when 1 μg of released DNA is present in solution. This standard curve can be used to quantify the DNA release from RALA/DNA NPs and RALA/DNA NPs encapsulated within polymeric matrices to determine NP and DNA stability following MN manufacture.

Quantification of NP Release from Polymer Matrices

RALA/pEGFP-N1 nanoparticles, N:P ratio 10 containing 1 μg pEGFP-N1 were incorporated into polymer matrices to produce 20% PVA, 20% PVP and 20% PMVE/MA as described previously. Following dissolution of these polymers in 10 mM Tris buffer, proteinase K was added to lyse the NPs for 1 h and the resulting released DNA was then quantified through addition of Picogreen® reagent and subsequent fluorescence detection using a EL808 96-well plate reader (Biotek, UK). The same protocol was carried out for NPs in solution without the presence of polymer and so quantification of released DNA from these complexes, in terms of fluorescence is regarded as 100% release.

The release of pEGFP-N1 from RALA/pEGFP-N1 NPs in the 20% PVP and 20% PVA formulations was determined to be 83.3% and 83.7% respectively as illustrated in FIG. 63 (B). In contrast, the release of DNA from RALA/pEGFP-N1 NPs in the 20% PMVE/MA formulation was calculated to be only 11%. These results indicate that PVA and PVP polymers do not interfere with RALA/DNA NPs or DNA encapsulated within their matrices. The low percentage of DNA release from the NPs encapsulated within the PMVE/MA matrix suggests that the polymer backbone is binding to the pDNA or causing its degradation, thus inhibiting its release into solution and subsequent detection.

Determination of pDNA Secondary Structure in the Presence of PMVE/MA by Circular Dichroism

To further elucidate the interaction between PMVE/MA and pDNA CD was carried out. For nucleic acids, the position, polarity and intensity of the CD peaks are functions of the base-stacking interactions and helicity of the DNA. Therefore, analysing intermolecular complexes formed with DNA via CD presents an excellent indicator of changes to the secondary structure of the DNA.

It is possible to see in FIG. 64 that the spectra for PMVE/MA alone generates no peaks from 245 nm to 350 nm and as such it's presence in solution does not interfere with the peaks observed in the samples containing pDNA. The sample containing pDNA only generates a peak intensity of 4 mdeg at wavelength 275 nm however, the intensity of this peak decreases to 3 mdeg when the pDNA has been incorporated into the PMVE/MA matrix. This decrease in peak intensity is indicative of a change in the B-form of the DNA which occurs when the DNA is forming intermolecular complexes, thus suggesting that the DNA is bound to the PMVE/MA.

### WST-1 Cell Viability Assay Following Exposure to Polymer Matrices

WST-1 cell viability assay was carried out using the NCTC-929 fibroblast cell line. FIG. 65 indicates that both 20% PVA and 20% PVP polymers exhibit minimal toxicity to cells at concentrations up to 20 mg/mL compared to untreated cells. Following exposure to 20 mg/mL of these polymers for 6 h, the percentage cell viability was 101.07% and 93.66% respectively for 20% PVA and 20% PVP.

In contrast, following incubation in the presence of PMVE/MA the cells exhibited significant toxicity and with a subsequent decease in cell viability. Cells were incubated in media containing concentrations of 0, 5, 10 and 20 mg/mL of 20% PMVE/MA and the resulting percentage cell viability after 6 h were 100%, 41.29%, 16.95% and 10.4% respectively indicating that even the lowest concentration of 5 mg/mL resulted in significant toxicity.

### Measurement of Axial Fracture Force of 20% PVA and 20% PVP MN Arrays

Axial fracture force tests were performed in order to determine the mechanical strength of the polymeric MNs fabricated from 20% PVA and 20% PVP. All MNs were visually inspected before and after testing and all MNs were originally 600 μm in height. In the first stage of this experiment, in order to select the most mechanically robust material, an axial compression force of 0.05 N/needle was exerted on MNs fabricated from the two polymer matrices. The percentage decrease in the height of MNs for 20% PVA MNs was 26.4% and 15.8% for 20% PVP MNs suggesting that the 20% PVP MNs are more mechanically robust than MNs fabricated from 20% PVA.

In order to obtain further insight into the behaviour of these MNs under axial loads, additional fracture force studies were performed. Forces ranging from 0.05 to 0.4 N/needle were applied to the MNs and percentage changes in their height were recorded as illustrated in FIG. 66.

The percentage reduction in the height of 20% PVA and 20% PVP MNs under increasing axial forces revealed that MNs exhibited progressive deformation without dramatic breakage at any point i.e. the MN protrusions did not break from the backing plate of the array. Within the range of forces applied, MN height decreased with increasing force exerted. For example, the mean percentage height reduction for arrays fabricated from 20% PVP was 15.8, 21.6, 29.4, 36.6 and 46.2% when the applied forces were 0.05, 0.10, 0.20, 0.30 and 0.40 N/needle respectively. Reductions in height over the same range of forces from MNs fabricated from 20% PVA were 26.4, 33.4, 45.2, 53.2 and 63.1% respectively which are significantly different to those detected with the PVP matrix at all forces investigated.

### SEM Analysis of 20% PVP MN Array Containing RALA/pEGFP-N1 N:P Ratio 10 NPs

MN images displayed in FIG. 67 are SEM images of a 20% PVP MN array encapsulating RALA/pEGFP-N1 N:P ratio 10 NPs loaded with 10 μg of pEGFP-N1. These images show that MNs fabricated in this manner produce uniform MN arrays of consistent length and width that are exact replicates of the mould used to fabricate them.

Short-Term Stability Study of RALA/pEGFP-N1 N:P Ratio 10 NPs Encapsulated within 20% PVP Matrix Up to 7 Days

To evaluate the storage stability of RALA/pEGFP-N1 nanoparticles N:P ratio 10 encapsulated within 20% PVP matrix these complexes were stored in temperature controlled environments of 20.0±1.0° C. and exposed to relative humidities (RH) of 46% to represent bench-top conditions in the laboratory and 45.0±1.0° C. and exposed to RH of 75% for up to 7 days representing a warmer climate and humidity encountered in the outdoor environment.

The stability studies revealed that under both conditions investigated the RALA/pEGFP-N1 nanoparticles were still intact following incubation for 0, 1, 3, 5 and 7 days investigated as indicated by an absence of DNA running through the gel in the lanes labelled ‘NPs’ in FIGS. 68 (A) and (B). Furthermore, when SDS is added to the NP solutions (Decomplexed NP wells) it is possible to see the pEGFP-N1 migrate through the gel producing a band consistent with the bands seen in the DNA only′ control lane of both gels. This indicates that there has been no change in the DNA plasmid conformation following these incubations and as such the RALA/pEGFP-N1 NPs should be functional following incubation of up to 7 days under these conditions.

Short-Term Stability Study of the Functionality of RALA/pEGFP-N1 N:P Ratio 10 NPs Encapsulated within 20% PVP Matrix Up to 7 Days

To evaluate the storage stability of RALA/pEGFP-N1 nanoparticles (N:P 10) encapsulated within the 20% PVP matrix these complexes were stored at room temperature for 7 days. As illustrated by FIG. 69 (B ii) transfection achieved by the RALA/pEGFP-N1 NPs following 7 days incubation in 20% PVP at room temperature is comparable to that achieved with NPs which have been incubated in 20% PVP at room temperature for 1 h (FIG. 69 A ii). This indicates that there has been no change in functionality of the NPs during this period of incubation and as such, the NPs are stable in 20% PVP at room temperature for up to 7 days.

A further transfection study was carried out to investigate the GFP expression following incorporation of naked pEGFP-N1 within the 20% PVP matrix as illustrated in FIGS. 69 (A i) and (B i). It is possible to see that no transfection was achieved following incorporation of pEGFP-N1 into the polymer following either the 1 h or 7 day incubation suggesting that the naked DNA and polymer did not form polyplexes suitable for nucleic acid delivery in vitro and that RALA is essential for functional nanoparticles.

Quantification of DNA Encapsulated in Tips and Baseplate of MN Arrays Loaded with RALA/μLux N:P 10 NPs Containing 36 μg DNA

Concentrated NPs were formulated and incorporated into the PVP matrix for MN manufacture. As such, not all of this DNA will be present in the MN tips of the array due to their small capacity (approx. 5 mg). In order to determine the quantity of DNA present in the MNs themselves and the baseplate of the array a quantification assay was used. The MNs were sheared from the baseplate and both components of the array dissolved in 4 mL 10 mM Tris buffer. The amount of DNA present was then assessed using the Picogreen® assay.

As detailed in Table 1 the MNs contained 9.4 μg DNA in the MNs and 17.5 μg in the baseplate of the array suggesting that the rest of the DNA has been removed from the array when cutting off the sidewalls of the array. This suggests that 74.7% of the NPs loaded into the array is still present. Therefore manufacture of the MN array with concentrated NPs is a more efficient method of MN fabrication compared to that described in Chapter 4 where less that 20% of the DNA content originally loaded into the array was present following manufacture.

A large proportion, 64.8%, of the DNA still present in the array resides in the baseplate of the array rather than the MN tips, however, this is expected due to the small volume of polymer capable of being loaded into the MN tips.

Quantification of DNA Encapsulation within MN Arrays Loaded with 36 μg RALA/pEGFP-N1 NPs.

MN arrays were fabricated from 20% PVP and loaded with RALA/pEGFP-N1 N:P 10 NPs containing 36 μg DNA. Following manufacture of the array as described in section 2.2.8 the needles were sheared off the array using a scalpel and dissolved in 0.5 mL 20 mM Tris buffer, pH8 and the remaining baseplate also dissolved in 0.5 mL 20 mM Tris buffer, pH8. 50 μL volumes of the solutions were then pipetted into a black 96-well plate and 50 μL 0.1 mg/mL proteinase K added and the plate incubated for 1 h at 37° C. Subsequently, 50 μL picogreen reagent is added and the plate incubated for a further 30 min. The plate was then shaken for 1 min and absorbance measured at 450 nm on an EL808 96-well plate reader (Biotek, USA).

| | Mean DNA | Standard |
|---|---|---|
| | concentration | error mean |
| | (μg) | (n = 3) |
| Microneedles | 9.4 | 0.43 |
| Baseplate | 17.5 | 0.51 |

Ex Vivo Release Profile of RALA/μLux N:P 10 NPs Released from 20% PVP MN Arrays Across Neonatal Porcine Skin

The release of RALA/μLux (N:P 10) NPs from 20% PVP MN arrays which contain approximately 27 μg was investigated through a release profile across neonatal porcine skin, 300 μm in thickness.

Detectable amounts of NPs were present in the receptor compartment of the apparatus following 5 min (Illustrated in FIG. 70 (A)) and the cumulative DNA release was calculated using a picogreen calibration plot and the appropriate dilution factor to convert the concentration of DNA (μg/mL) to total amount of DNA released into the 4 mL franz cell receptor compartment. The total DNA release after 24 h was equivalent to NPs containing 23.3 μg pLux being released across the membrane, as illustrated in FIG. 70 (B), which is equal to 86% of the total loading of NPs present in the MN the array as calculated in Table 1 suggesting that although the loading of the NPs into the array is not optimal because there is a loss in the side walls, there is efficient delivery of the NPs encapsulated within the MN array across the ex vivo porcine skin and not just those NPs localised into the MN tips.

Determination of Force Required to Insert RALA/μLux NP-Loaded 20% PVP MN Array into Full Thickness Neonatal Porcine Skin Via OCT

Having ascertained that RALA/DNA NPs can be incorporated into and subsequently released from MNs fabricated from 20% PVP without dissociation, or loss of functionality it is necessary to determine if the MNs are of sufficient strength to penetrate full thickness neonatal porcine skin and what force is required for efficient breach of the SC and penetration into the dermis.

The OCT images in FIG. 71 (A) enable the depth of insertion of the MNs into the skin to be determined through image analysis using Image J software and identification of the SC barrier and the baseplate of the array as labelled on the images. The Image J software allows measurement of the depth of needle penetration into the skin and the pore width formed by the needles and thus it is possible to calculate the percentage needle penetration as illustrated in FIG. 71 (B). It is possible to see that following application of forces of 8 N, 11 N and 16 N (FIG. 71 (A) (i), (ii) and (iii) respectively) that even though the needle tip breaches the SC they are not fully inserted into the skin and a significant distance remains between the baseplate of the array and the SC. However, following application of manual force 89.7% of the needle length is embedded in the skin suggesting this is a more appropriate force to employ for MN insertion into skin and is suitable for the insertion of these MNs in vivo to optimise conditions for MN dissolution and cargo release.

Confocal Microscopic Study of NP Release into Mouse Ear Tissue Following Insertion MN Loaded with Cy-3 Labelled RALA/pOVA NPs

To further analyse the dissolution of the 20% PVP MN array in vivo and the release of RALA/DNA NPs a study was carried out to visualise the NP distribution within the mouse ear tissue following MN application. pOVA was fluorescently labelled with the Cy-3 fluorophore and then used to form RALA/pOVA NPs which were subsequently encapsulated to form 20% PVP MN arrays. The array was applied using manual force to a mouse ear in vivo and 1 h following application the animal was sacrificed and the ear removed for confocal analysis which is illustrated in FIG. 72.

FIG. 72 (A) represents confocal analysis of an untreated mouse ear which was excised for use as a negative control. It is possible to see that there is no auto-fluorescence visible from the tissue. FIG. 72 (B) is a confocal microscopic image of ear tissue following application of a MN array containing naked Cy-3 labelled pOVA. It is not possible to visualise the labelled DNA in the tissue suggesting it has dispersed rapidly through the ear tissue. FIG. 72 (C) represents ear tissue 1 h following application of a MN array loaded with Cy-3 labelled RALA/pOVA NPs. It is possible to see the NP release in the ear, indicated by localised red areas of NP deposition in the tissue. The ability to visualise the NP release and not that of the naked DNA suggests that the NPs take longer to disperse through the tissue as would be expected due to the macromolecular dimensions of the NPs and also the NPs may be transported intracellularly in the ear and thus are visible locally, whereas naked DNA is not taken up intracellularly due to poor transfection efficacy without a delivery vehicle.

### Determination of Luciferase Expression Detectable Using the IVIS In Vivo Imaging System 6 h, 24 h and 48 h Post Application of RALA/μLux NP-Loaded 20% PVP MN Array In Vivo

FIG. 73 illustrates luciferase expression detected via the IVIS in vivo imaging system 6 h, 24 h and 48 h following application of 20% PVP MN arrays containing approximately 27 μg of RALA/μLux N:P 10 NPs to both ears of C57BL/6 mice. It is possible to see in (A) that there are detectable levels of luciferase expression in all 3 mice 6 h post MN application in the liver and kidneys, (B) illustrates that 24 h post MN application expression is still detectable in the liver and kidneys and at increased levels as indicated by the increase in photon intensity visible. It is possible to see in (C) that 48 h post MN application luciferase expression persists in the liver and kidneys with a significant increase in expression in the liver compared to the previous time points.

This study confirms that the delivery of RALA/DNA NPs via a 20% PVP MN array results in protein expression in vivo. Although expression has only been detected in the liver and kidneys of each animal it is possible that there are lower levels of protein expression in other organs and tissues which are lower than the limit of detection of the IVIS system employed in this study.

Analysis of OVA-Specific CD8⁺ T-Cells Detected 10 Days Post Microneedle Immunization with pOVA and RALA/pOVA Nanoparticles

A number of research groups developing MN delivery systems have utilised it as a means to deliver nucleic acids for vaccination purposes. In order to determine if the RALA/pOVA NP-loaded MN arrays developed in this study elicited antigen expression and a subsequent CD8⁺ T-cell response to the protein expressed, MNs containing either pOVA or RALA/pOVA NPs were fabricated. C57BL/6 mice were immunized with these arrays and 10 days post immunization sacrificed, the auricular lymph nodes harvested and stained for the OVA-specific CD8⁺ surface receptor followed by antibody staining for CD8⁺ and B220 and then analysed using flow cytometry.

FIG. 74 (A) describes the back-gating process used to analyse the cells isolated from the auricular lymph nodes. Initially, the live cell population was selected, then from this population the ‘single cell’ population was further isolated. Of this single cell population it is possible to identify the T-cell and B-cell populations via the fluorophores attached to the CD8⁺ and B220 antibodies, the CD8⁺ T-cells were then selected for analysis.

FIG. 74 (B) illustrated the identification of the OVA-specific CD8⁺ surface receptor. The cell population visible in these dot-plots are CD8⁺ T-cells and those gated in the top left-hand portion of the dot-plot are those which exhibit the APC-label which is conjugated to the antibody stain used to detect the OVA-specific CD8⁺ surface receptor. It is possible to see that there is an increase in this gated population in the lymph nodes isolated following immunization with MNs containing RALA/pOVA NPs compared to those containing naked pOVA or empty 20% PVP arrays. FIG. 74 (C) illustrated the quantification of this increase in the OVA-specific CD8⁺ T-cell population. It is possible to see that compared to mice treated with empty 20% PVP MN arrays there is a 3-fold increase in APC labelled cells indicating a significant increase in the proliferation of OVA-specific CD8⁺ T-cell (p=0.0084 using a one-tailed, unpaired t-test). Immunization using naked pOVA loaded into the MN arrays yielded no significant increase in proliferation of OVA-specific CD8⁺ T-cells compared to the empty 20% PVP MN treated mice (p=0.3836 using a one-tailed, unpaired t-test). As such, when comparing the increase in OVA-specific CD8⁺ T-cell proliferation observed between the mice treated with pOVA MNs and RALA/pOVA MNs there is a significant difference (p=0.0116 using a one-tailed, unpaired t-test) suggesting once again that the RALA delivery peptide is an essential component for the enhancement of gene expression in vivo.

Agarose Gel Determination of the Stability of RALA/HPV-16 E6 and RALA/HPV-16 E7 NPs Encapsulated within 20% PVP Matrix Up to 21 Days

To evaluate the storage stability of RALA/HPV-16 E6 and RALA/HPV-16 E7 nanoparticles N:P ratio 10 encapsulated within 20% PVP matrix these complexes were stored in temperature controlled environments of 4° C., 35% relative humidity (RH), 20° C., 40% RH, or 20° C., 86% RH for 7, 14 and 21 days.

The stability studies revealed that under all conditions investigated the RALA/HPV-16 E6 and RALA/HPV-16 E7 nanoparticles were still intact following incubation for 7, 14 and 21 days investigated as indicated by an absence of DNA running through the gel in the lanes labelled ‘NP’ in FIGS. 75 (A) and (B). Furthermore, when Proteinase K is added to the NP solutions (NP+PK wells) it is possible to see the DNA migrate through the gel producing a band consistent with the bands seen in the DNA′ control lane of both gels. This indicates that there has been no change in the DNA plasmid conformation following these incubations and as such the NPs should be functional following incubation of up to 21 days under these conditions.

Determination of Functionality of RALA/HPV-16 E6, RALA/HPV-16 E7, and RALA/HPV-16 E6/E7 N:P Ratio 10 NPs Encapsulated within 20% PVP Matrix

To evaluate the functionality of RALA/HPV-16 E6, RALA/HPV-16 E7 and RALA/HPV-16 E6/E7 N:P ratio 10 NPs encapsulated within 20% PVP matrix the polymers were dissolved in PBS and the solution used to transfect NCTC-929 fibroblast cells. Transfection efficacy was determined via western blot analysis as illustrated in FIG. 76. It is possible to see that following transfection with plasmids E6, E7 and E6/E7 without encapsulation in RALA there was no protein expression as illustrated by the absence of bands in FIG. 76. These results confirm the necessity of the RALA peptide for cellular transfection and indicate that the plasmids do not complex with the PVP matric upon incorporation. However, there are bands illustrating protein expression following cellular transfection with RALA/HPV-16 E6, RALA/HPV-16 E7 and RALA/HPV-16 E6/E7 NPs. These indicate once again that RALA/DNA NPs retain functionality upon incorporation into the PVP matrix and successfully transfect cells eliciting protein expression.

### Determination of Gene Expression in DCs in Draining Lymph Nodes Post MN Application

As illustrated in FIG. 79 (A), there is a significant increase in the percentage of tdT expression in DCs in the draining lymph nodes in the pOVA-tdT-AK, N:P 1, N:P 2 and N:P 6 treatment groups (p=0.0022, 0.0075, 0.0039 and 0.0024 respectively using an unpaired, one-tailed t-test). Additionally, there was an increase in tdT expression in the treatment group N:P 2 compared to the delivery of pOVA-tdT-AK alone suggesting that for this N:P ratio the formulation of complexes with the RALA peptide resulted in a statistically significant increase in gene expression in vivo (p=0.0208 using an unpaired, one-tailed t-test). It is possible in FIG. 6.2 (B) that there are 3 identifiable populations of DCs expressing the tdT reporter gene to varying degree with almost 70% of the tdT positive-DCs derived from the skin tissue indicating the nanoparticles were taken up by the DCs in the skin tissue followed by migration to the draining lymph nodes.

Determination of Circulating HPV-16 E6/E7 IgG Antibody Levels Generated Following Immunisation with Plasmid DNA Expressing HPV-16 E6/E7 Antigens

As illustrated in FIG. 80, the levels of HPV-16 E6/E7 IgG antibodies detected in serum were significantly elevated following delivery of RALA/pHPV-16 E6/E7. nanoparticles at both time points by both i.m. injection and MN application. However, following delivery of ‘naked’ pHPV-16 E6/E7 by either route, the levels of antigen-specific antibodies was not significantly elevated compared to negative control untreated mice. This confirms the utility of the RALA peptide to condense and delivery the DNA intracellularly in vivo is essential for the generation of antigen expression and resultant humoural immune responses.

Determination of Generation of HPV-16 E6/E7-Specific Cytotoxic T Cells Following Immunisation with Plasmid DNA Expressing HPV-16 E6/E7 Antigens

As illustrated in FIG. 81, the percentage cytotoxicity achieved was higher in the treatment groups than the negative control groups, i.e. untreated mice and those treated with ‘empty’ MNs. The most significant percentage cytotoxicity was observed from splenocytes isolated from C57BL/6 mice immunized with RALA/pHPV-16 E6/E7 nanoparticles by both i.m. and MN routes. This indicates that DNA delivered encapsulated within RALA nanoparticles generated elevated levels of E6/E7-specific cytotoxic CD8+ T cells compared to those receiving ‘naked’ DNA.

Determination of Interferon-Gamma Secretion from Splenocytes Restimulated with E6/E7-Expressing TC-1 Cells Ex Vivo

FIG. 82 illustrates the IFN-γ secretion from splenocytes restimulated ex vivo with E6/E7 expressing TC-1 cells following isolation from immunised C57/BL6 mice. Elevated IFN-γ secretion is indicative of CD4+ and CD8+ T cell proliferation. Thus, the elevated levels of IFN-γ detected in the groups immunised with the positive control-E6/E7 peptide and RALA/DNA nanoparticles via MN administration indicate that antigen-specific CD4+ and/or CD8+ T cell responses were generated following the immunisation regimen. As such, these results, in combination with those illustrated in FIGS. 80 and 81 clearly demonstrate both humoural and cellular immune responses were elicited consistently with the group receiving RALA/pHPV-16 E6/E7 nanoparticles delivered by dissolving polymeric MN application.

Determination of Efficacy of Prophylactic Immunisation with Plasmid DNA Expressing HPV-16 E6/E7±RALA Against Establishment of Tumour Following Implantation of TC-1 Cells In Vivo

The data displayed in FIGS. 83 (A) and (B) indicates that progression of tumour growth and animal death observed in mice immunised in a prime-boost-boost regimen receiving 100 μg pHPV-16 E6/E7±RALA by intramuscular injection and MN administration. It's possible to determine that following prophylactic immunisation with RALA/pHPV-16 E6E7 nanoparticles, by MN administration, tumour formation was completely inhibited in 4 out of 9 mice, this was the group which demonstrated the most protection against TC-1 tumour formation. By comparison intramuscular delivery of the RALA/pHPV-16 E6E7 nanoparticles resulted in protection in only 2 of 9 mice. The delivery of ‘naked’ DNA by both routes resulted in the death of 8 of the 9 mice. These results indicate that MN delivery of the RALA/pHPV-16 E6E7 nanoparticles resulted in the best survival rate and slowest tumour progression indicating it was the most effective means of vaccination against the E6/E7-expressing TC-1 tumour.

Determination of Efficacy of Therapeutic Immunisation with Plasmid DNA Expressing HPV-16 E6/E7±RALA Against Growth of Established TC-1 Tumour

A therapeutic study was also performed to determine whether immunization can induce therapeutic antitumor immunity, i.e. if a pre-established tumour could be treated by immunisation. As illustrated in FIG. 84, while tumours grew to the maximal permissible size by day 21 in mice immunized with ‘naked’ DNA, delivered by both i.m. injection and MN administration, there was a marked retardation in tumour growth in mice immunized with RA:A/pHPV-16 E6/E7 nanoparticles. These results suggest that immunisation with RALA/pHPV-16 E6/E7 nanoparticles are an effective therapeutic approach to treat pre-established TC-1 hours. Additionally, those nanoparticles delivered by MN application showed an increased delay in growth compared to those treated by i.m. administration of the nanoparticles, suggesting intradermal MN delivery is a superior means of administration.

### Quantification of Freeze-Dried RALA/pHPV-16 E6/E7 NP Ratio 6 Nanoparticles Delivered Following Application to C57BL/6 Mouse Ears

It's possible to see in FIG. 85 that DNA delivered from whole MN arrays is estimated to be 10 μg from those loaded with 36 μg following 5 min application. DNA delivery from MNs fabricated from 9-10 kDa PVA polymer superior compared to the 2 PVP polymers analysed Some loss in DNA can be accounted for by residue remaining in the MN mould following MN removal and in the side walls of the array, removed prior to MN application. Additionally, it is thought that ‘naked’ DNA encapsulated in PVP may interact with the polymeric backbone, impeding its release from the MN array and resultant detection via picogreen assay. Following freeze-drying of RALA/E6-E7 DNA nanoparticles, DNA delivery is estimated to be 30 μg from 50 μg initially loaded MNs (60%) and 60 μg from 100 μg (60%) of that loaded MNs, after 5 min application. Furthermore FIG. 85 (B) illustrates that approximately 90% of DNA loaded into the polymeric MNs is delivered following 24 h of MN application to the mouse ear. Thus, freeze-drying of RALA/pHPV-16 E6/E7 nanoparticles increased the dosing per MN array and additionally results in increased percentage DNA delivery following MN application

Measurement of MNs, Fabricated from PVP and PVA Polymers of a Range of Molecular Weights, Percentage Height Reduction Following Application of an Axial Force

All polymers formed strong, sharp needles that were a replica of the silicon mould (FIG. 86 (A)). MNs formulated with 360 kDa PVP, 13-23 kDa and 9-10 kDa PVA formed strong, flexible baseplates. Baseplates formulated with 58 kDa PVP were brittle and prone to fracture when peeling from the silicon mould. Therefore, MNs were formulated with 30% w/w 58 kDa PVP blends to form needles and 20% w/w 360 kDa PVP to form a more flexible baseplate. To determine the mechanical strength of the manufactured MNs, an axial force of 45 N was applied to arrays for 30 sec. Following compression, arrays showed a slight deformation of the needle tips (FIG. 86 (B)), but did not suffer from significant fracture/breakage. The percentage height reduction was determined by dividing the height of MNs following compression by the height of MNs prior to compression and multiplying by 100. Arrays were found to be mechanically robust with a height reduction of <10% across all formulations as illustrated in FIG. 87. There was no significant difference in the strengths of formulations as determined by unpaired one-tailed t test (p<0.05). To determine the effect of the incorporation of pDNA or RALA/pDNA complexes into MNs, arrays were fabricated incorporating 32 μg pDNA by diluting concentrated polymer stock with DNA or RALA/pDNA solutions. This aqueous blend was added to moulds and a baseplate of polymer was added after the needle cavities were filled. MN strength was not compromised by the incorporation of pDNA or RALA/pDNA solutions as determined by unpaired one-tailed t test (p<0.05) (FIG. 87).

Optical Coherence Tomographic Analysis of MN Penetration into Mouse Ears Following Fabrication from PVP and PVA Polymers of a Range of Molecular Weights

19×19 arrays were fabricated using 20% w/w 360 kDa PVP, 20% 13-23 kDa PVA, 20% 9-10 kDa PVA and 30% w/w 58 kDa polymer stock solutions. To determine whether MNs had sufficient strength to penetrate the SC arrays were applied into mouse ears using the TA-XT2 Texture Analyser (10-40 N) for 30 sec. Following application, skin was immediately analysed using the optical coherence tomography (OCT) scanner to determine needle penetration depth, images were analysed and measured using Image J software (FIG. 3). All MN arrays were able to penetrate the SC, even with the lowest application pressure. Maximal penetration was achieved with all MN arrays with an application force of 30 N. MN applications with higher insertion forces reduced penetration depths due to the elasticity of the skin repelling the MN projections. There was no statistically significant difference between penetration depths observed, although, maximal penetration was achieved using 20% w/w 9-10 kDa PVA needles with an insertion force of 30 N (56.00%).

### Cytotoxicity Analysis of PVP and PVA Polymers of a Range of Molecular Weights, to Fibroblast and Dendritic Cell Lines In Vitro

The affect of polymers on cell viability of the fibroblast NCTC-929 and DC 2.4 dendritic cell lines was assessed by MTS assay. It was determined that following 24 h incubation 360 kDa and 58 kDa PVP caused significant cellular toxicity from the lowest polymer concentration analysed (10 mg/mL) to NCTC-929 and DC 2.4 cell lines. However, 13-23 kDa and 9-10 kDa PVA were not found to cause significant toxicity to either cell line at any concentration investigated indicating they would be suitable to develop further as an in vivo delivery system.

Quantification of RALA/pDNA Release from PVP and PVA Polymers of a Range of Molecular Weights

To determine the effect of polymer and N:P ratio on pDNA release, gels were formulated with 10 μg loading of pDNA by diluting concentrated polymer with RALA/pDNA (N:P ratios 0-10) aqueous solutions. The quantity of pDNA released was then assessed using a standard curve following incubation with Quanti-iT Picogreen™ reagent and Proteinase K (FIG. 90). pDNA release from gels composed of 360 kDa and 58 kDa PVP increased with increasing N:P ratio from N:P 0, (7.293±1.24 μg and 7.741±0.212 μg respectively) to N:P 4 where release was maximal, (10.068±0.888 μg and 10.217±0.943 μg respectively) (p<0.05). In contrast, pDNA release at N:P 0 was maximal (10.691±1.085 μg and 10.561±1.180 μg respectively) for gels composed of 13-23 kDa and 9-10 kDa PVA and no significant difference in pDNA release across the N:P ratios investigated (p<0.05).

Arrays were formulated with a 32 μg loading of pDNA per MN array by diluting concentrated polymer with RALA/pDNA (N:P ratio 10) aqueous solution. Not all of the pDNA loaded into the MN array shall be present in the baseplate and needles of the array and therefore available for delivery across the SC. Therefore, to determine the quantity of pDNA loaded in the baseplate and MN projections of arrays, potentially available for delivery, the sidewalls of arrays were removed with a heated scalpel to allow separate quantification. The baseplate and MN projections of the array and the sidewalls were dissolved in 10 mM Tris buffer pH 8. The quantity of pDNA released was then assessed as by Picogreen assay, described previously. pDNA release from the baseplate and microneedles of 9-10 kDa PVA arrays (17.74 μg) was found to be significantly greater than from release from 13-23 kDa PVA (p=0.0497), 58 kDa PVP (p=0.0376) and 360 kDa PVP arrays (p=0.0023) as illustrated in FIG. 91.

Agarose Gel Analysis of pDNA Integrity Following Incorporation into PVP and PVA Polymers of a Range of Molecular Weights

As illustrated in FIG. 92, following incubation of RALA/pDNA nanoparticles (N:P 6) in a range of polymeric matrices nanoparticles remained intact for up to 7 days, as demonstrated by the well retention of nanoparticles incubated with Tris buffer. Decomplexation of the RALA/pDNA nanoparticles with Proteinase K resulted in migration of pDNA through the agarose gel, producing distinct bands which were similar to the pDNA only control wells. This indicates that pDNA is intact following release from all polymer matrices when encapsulated within RALA nanoparticles and thus should retain functionality. DNA incorporated into polymeric matrices in the absence of RALA (N:P 0) was released undamaged from PVA matrices following 0 and 7 days incubation as shown by the migration of DNA through the agarose gel to produce distinct bands similar to the DNA only control. In contrast, pDNA released from 360 kDa PVP matrices shows a slight smearing on the agarose gel following 0 days incubation, indicating DNA degradation. This smearing (damage) is more pronounced following 7 days incubation. Similarly, pDNA released from 58 kDa PVP matrices shows smearing on the agarose gel lane indicating DNA damage at 0 and 7 days. These results indicate that RALA is necessary for the protection of DNA encapsulated within PVP gels as PVP is interacting with, and damaging, pDNA.

In Vitro Cell Transfection Efficacy of RALA/pEGFP-N1 Complexes Following Release from PVP and PVA Polymers of a Range of Molecular Weights

Microscopic analysis of cells 48 h post transfection to detect fluorescent reporter-gene expression (FIGS. 93 (A) and 94 (A)) demonstrate that RALA/pEGFP-N1 complexes released from polymeric matrices retain functionality and are capable of transfecting NCTC-929 and RAW 246.7 cells following dissolution. In contrast, no cells expressing GFP are detectable following incubation with “naked” DNA (N:P 0) released from dissolved polymer gels indicating the pDNA does not form polyplexes with the polymeric matrices used to fabricate MN arrays.

Flow cytometric analysis of GFP reporter-gene expression demonstrates that RALA is necessary to achieve transfection in vitro. The most efficient transfection in fibroblast NCTC-929 cells was achieved with RALA/pEGFP-N1 nanoparticles (N:P ratio 12) released from 13-23 kDa PVA gels (43.687%). The percentage transfection was similar following release of nanoparticles at the same ratio from 9-10 kDa PVA gels (43.347%) (p<0.05). However, transfection of cells with nanoparticles at the same N:P ratio was significantly lower following release from 360 kDa (9.817%) and 58 kDa (28.623%) PVP gels (p=0.0009 and 0.017 respectively). The maximum transfection achieved in RAW 246.7 cells was with RALA/pEGFP-N1 nanoparticles (N:P ratio 12) released from 9-10 kDa PVA gels (18.783%). The percentage transfection of achieved was lower with RALA/pEGFP-N1 nanoparticles at the same N:P ratio released from 13-23 kDa PVA (14.280%, p=0.2582) and 58 kDa PVP (11.753%, p=0.1414) gels and significantly lower following release from 360 kDa PVP gels (4.973%, p=0.0333). These results indicate that the transfection efficacy achieved is cell-line dependent but in all cases, the inclusion of the RALA peptide significantly increased reporter-gene expression in all cases.

### CONCLUSIONS

The aim of this research was the development of a polymeric MN array using a mechanically robust polymeric matrix suitable for low-cost manufacture of the arrays that will not compromise the transfection efficacy of the bioactive RALA/DNA cargo. The fabricated 20% PVP arrays have proven to be mechanically strong at room temperature for insertion into full thickness neonatal porcine skin and mouse ear tissue indicating they are viable devices for insertion into human skin clinically. It was also shown that the NP-loaded arrays remain stable following short-term storage and manufacture under ambient conditions suggesting these devices circumvent the need for ‘cold chain’ storage.

Additionally, the combination of the novel amphipathic delivery RALA/DNA NPs and the dissolving polymeric MN array resulted in the production of a delivery device capable of eliciting gene delivery and resultant protein production in vivo. Furthermore, it has been proven that when DNA encoding a model antigen is delivered in this manner an antigen-specific CD8⁺ T-cell response is elicited, suggesting this delivery system had potential to not only transform the field of gene therapy but more specifically, DNA vaccination. Further investigation has determined that RALA/pDNA nanoparticles delivered intradermally using this delivery system are primarily taken up by skin-resident DCs which subsequently travel to the skin-draining lymph nodes for antigen-presentation to the lymph-resident T cell populations.

Following these encouraging results it was hypothesised the delivery system could be utilised for the delivery of plasmids to elicit protection against herpes simplex virus (HPV) causing cervical cancer, namely, HPV-16 E6, HPV-16 E7 and HPV-16 E6.E7. As such, it has been established that MN arrays encapsulating RALA/HPV-16 E6, RALA/HPV-16 E7 and RALA/HPV-16 E6/E7 NPs are stable following storage under adverse conditions for prolonged periods of time and the functionality of the NPs following encapsulating within the PVP matrix. Thus, further studies investigating the efficacy of this delivery system at eliciting protection against HPV-16 E6/E7-expressing tumours was demonstrated using both a prophylactic and therapeutic vaccination regimen. The positive results from both of these studies indicate that i) this delivery platform may be used to protect non-infected patients against establishment of an E6/E7-expressing tumour and ii) this delivery platform is capable of inhibiting progression of pre-established E6/E7-expressing tumours and can cause a reduction in tumour burden.

Moreover, investigation into further optimisation of the delivery system has demonstrated that a range of non-cytotoxic polymeric matrices may be used for fabrication of the dissolving nanoparticle-loaded MN arrays. PVP and PVA polymers, formulated at a range of molecular weights have demonstrated capabilities to fabricate robust MNs and release functional nanoparticles in vitro. Additionally, loading efficacy and subsequent delivery of the RALA/DNA cargo can be improved by freeze-drying the nanoparticles prior to incorporation with the polymeric matrices. These advances in formulation indicate that this nanoparticle-loaded dissolving MN delivery system may be capable of producing even more promising results in vivo.

The focus of this research was on developing a suitable delivery vehicle for the intracellular delivery of DNA and then incorporating these complexes into dissolvable MNs to facilitate non-invasive delivery of the DNA cargo in vivo. The RALA peptide has been demonstrated as an efficient delivery vehicle for pDNA both in vitro and in vivo, overcoming both the extracellular and intracellular barriers against gene expression as demonstrated by its superior transfection profile when compared to ‘naked’ DNA delivery. Furthermore, the formulation methods for PVP MN fabrication employed in this study are straightforward and avoid complex and time-consuming coating processes such as those described in the literature for the manufacture of similar delivery systems. Moreover, the polymer excipients used are cheap, non-toxic and can be processed at room temperature. Importantly, the MNs dissolve rapidly upon insertion into the skin and consequently the MN arrays cannot be reused following removal from a patient and there is no requirement for specific disposal arrangements. All these advantages suggest that the NP-loaded dissolvable MN arrays fabricated from PVP using laser-engineered moulds have vast potential for clinical use.

All documents referred to in this specification are herein incorporated by reference. Various modifications and variations to the described embodiments of the inventions will be apparent to those skilled in the art without departing from the spirit of the invention.

1. A cell delivery system comprising
a microprotrusion array for use in the transport of a material across a biological barrier in which the array comprises a plurality of microprotrusions composed of a swellable and/or dissolvable polymer composition;
the material comprises nanoparticles formed from a nucleic acid or a negatively charged or hydrophilic compound complexed with an amphipathic cell penetrating peptide;
wherein the microprotrusion array is loaded with the nanoparticles.

2. The cell delivery system according to claim 1 wherein the nucleic acid comprises a DNA vaccine.

3. The cell delivery system according to claim 1 wherein the nucleic acid is adapted for gene therapy, and is optionally a DNA, mRNA, miRNA or siRNA molecule.

4. The cell delivery system according to claim 1, wherein the amphipathic cell penetrating peptide comprises less than approximately 50 amino acid residues with at least 6 arginine residues (R), at least 12 Alanine Residues (A), at least 6 leucine residues (L), optionally at least one cysteine residue (C), and at least two but no greater than three glutamic acids (E).

5. The cell delivery system according to claim 1 comprising an amphipathic cell penetrating peptide wherein
the arginine (R) residues are evenly distributed along the length of the peptide;
the ratio of arginine (R) to negatively charged glutamic acid (E) residues is from at least 6:2 to 9:2; and
the ratio of hydrophilic amino acid residues to hydrophobic amino acid residues at pH 7 is at least 30:67 to 40:60.

6. The cell delivery system according to claim 1 wherein amphipathic cell penetrating peptide comprises the consensus sequence EARLARALARALAR (SEQ ID No. 15).

7. The cell delivery system according to claim 1 wherein amphipathic cell penetrating peptide comprises less than approximately 40 amino acid residues.

8. The cell delivery system according to claim 1 wherein the amphipathic cell penetrating comprises at least 24 amino acids.

9. The cell delivery system according to claim 1 wherein the peptide according to any of the preceding claims comprising the consensus sequences EARLARALARALAR and LARALARALRA (SEQ ID No. 16).

10. The cell delivery system according to claim 1 wherein the amphipathic cell penetrating peptide comprises the amino acid sequence X-EARLARALARALAR-Y-LARALARALRA-Z-EA (SEQ ID No. 17), wherein X is W or R; Y is optional or selected from H or E; and Z is C or R; or a sequence with at least 99%, 98%, 97%, 96%, 95%, 94%, 93%, 92%, 91%, 90%, 85%, 80% sequence identity or homology.

11. The cell delivery system according to claim 1 wherein said microprotrusions can puncture the stratum corneum of mammalian skin and, in use, upon insertion into the skin, said microprotrusions swell.

12. The cell delivery system according to claim 1 adapted for transdermal or intradermal administration to a subject which upon contact with the interstitial fluid of a subject releases the material comprising nanoparticles from the microprotrusion array into the extracellular space for subsequent transport intracellularly to the antigen presenting cells (APC) of the subject.

13. The cell delivery system according to claim 1 wherein the microprotrusions are approximately 1-3000 μm in height and/or have a diameter of approximately 50-300 μm.

14. The cell delivery system according to claim 1 wherein the polymer is selected from one or more of poly(vinylalcohol), poly(vinylpyrrolidone), poly(hydroxyethylmethacrylate) and derivatives thereof, poly(methylvinylether/maleic acid) and derivatives thereof, poly(methylvinylether/maleic anhydride) and derivatives thereof, poly(acrylic acid), poly(caprolactone), hydroxyethylcellulose and derivatives thereof, poly(ethyleneglycol) and derivatives thereof, hyaluronic acid, chitosan and/or carbohydrates and derivatives thereof.

15-18. (canceled)

19. The cell delivery system according to claim 1 wherein the amphipathic cell penetrating peptide comprises one of the following amino acid sequences:
(SEQ ID No. 1)
WEARLARALARALARHLARALARALRACEA
(SEQ ID No. 2)
WEARLARALARALARLARALARALRACEA
(SEQ ID No. 3)
WEARLARALARALARLARALARALRACEA
(SEQ ID No. 4)
WEARLARALARALARELARALARALRACEA
(SEQ ID No. 5)
REARLARALARALARLARALARALRACEA
(SEQ ID No. 6)
REARLARALARALARLARALARALRAREA
(SEQ ID No. 7)
REARLARALARALARELARALARALRAREA
or a fragment thereof.

20. The cell delivery system according to claim 1 wherein the amphipathic cell penetrating peptide consists of one of the following amino acid sequences:
(SEQ ID No. 1)
WEARLARALARALARHEARALARALRACEA
(SEQ ID No. 2)
WEARLARALARALARLARALARALRACEA
(SEQ ID No. 3)
WEARLARALARALARLARALARALRACEA
(SEQ ID No. 4)
WEARLARALARALARELARALARALRACEA
(SEQ ID No. 5)
REARLARALARALARLARALARALRACEA
(SEQ ID No. 6)
REARLARALARALARLARALARALRAREA
(SEQ ID No. 7)
REARLARALARALARELARALARALRAREA.

21. The cell delivery system according to claim 1 wherein the amphipathic cell penetrating peptide comprises or consists of the following amino acid sequence WEARLARALARALARHLARALARALRACEA (SEQ ID No. 1).

22. The cell delivery system according to claim 1 wherein the amphipathic cell penetrating peptide is coupled to a polyethylene glycol (PEG) molecule.

23. The cell delivery system according to claim 1 wherein the amphipathic cell penetrating peptide further comprises a cell targeting motif sequence conjugated to the amphipathic cell penetrating peptide via a spacer sequence.

24. The cell delivery system according to claim 23 wherein cell targeting motif is a metastasis targeting peptide, optionally a prostate cancer targeting peptide TMTP-1 (NVVRQ), and the spacer is an alpha helical spacer, preferably comprising from 1 to 4 repeats of the sequence EAAAK.

25. The cell delivery system according to claim 1 wherein the nucleic acid is DNA and comprises an inducible nitric oxide synthase (iNOS) plasmid DNA under control of a tumour specific promoter.

26. The cell delivery system according to claim 1 wherein the negatively charged or hydrophilic compound is a phosphate or lipophilic based drug.

27. (canceled)

28. The cell delivery system according to claim 1 wherein the nanoparticle is a discrete spherical nanoparticle with a diameter less than approximately 150 nm.

29-31. (canceled)

32. A method of inducing an immune response in a subject comprising the administration of the cell delivery system according to claim 1 to a subject in need thereof and comprising the steps of
applying the microprotrusion array to the skin such that the microprotrusions protrude through or into the stratum corneum;
allowing the microprotrusions to swell; and
allowing the microprotrusions to dissolve and release the material into the skin.

33. A method for the treatment and/or prophylaxis of an infection or cancer comprising the administration of the cell delivery system according to claim 1 to a subject in need thereof comprising the steps of
applying the microprotrusion array to the skin such that the microprotrusions protrude through or into the stratum corneum,
allowing the microprotrusions to swell,
allowing the microprotrusions to dissolve and release the material into the skin.

34. (canceled)

35. The cell delivery system according to claim 1 wherein the nucleic acid comprises a DNA vaccine in the form of plasmid DNA encoding an antigen for a disease.
